SEQUENCE LISTING (93 sequences)

SEQ ID NO: 1 - DNA, 22 bp, synthetic construct
  source 1..22: mol_type = other DNA
gatgggcaactgtacctgactg

SEQ ID NO: 2 - DNA, 19 bp, synthetic construct
  source 1..19: mol_type = other DNA
ctgggctcctcttggaatg

SEQ ID NO: 3 - DNA, 20 bp, synthetic construct
  source 1..20: mol_type = other DNA
ggcctcaagaagacaactgc

SEQ ID NO: 4 - DNA, 20 bp, synthetic construct
  source 1..20: mol_type = other DNA
gcaaaaggcattgaagaagc

SEQ ID NO: 5 - DNA, 19 bp, synthetic construct
  source 1..19: mol_type = other DNA
gccgatgcagtagtggagg

SEQ ID NO: 6 - DNA, 22 bp, synthetic construct
  source 1..22: mol_type = other DNA
ctgccttgaatctccatggtac

SEQ ID NO: 7 - DNA, 20 bp, synthetic construct
  source 1..20: mol_type = other DNA
agcaagagattccgaaggat

SEQ ID NO: 8 - DNA, 20 bp, synthetic construct
  source 1..20: mol_type = other DNA
agtggaggactgagtgcaag

SEQ ID NO: 9 - DNA, 20 bp, synthetic construct
  source 1..20: mol_type = other DNA
aggcttgaagacccttccat

SEQ ID NO: 10 - DNA, 20 bp, synthetic construct
  source 1..20: mol_type = other DNA
acaggcagactggtttcagg

SEQ ID NO: 11 - DNA, 20 bp, synthetic construct
  source 1..20: mol_type = other DNA
catctttgtccccagagagc

SEQ ID NO: 12 - DNA, 20 bp, synthetic construct
  source 1..20: mol_type = other DNA
gcaccagctccacacatcta

SEQ ID NO: 13 - DNA, 20 bp, synthetic construct
  source 1..20: mol_type = other DNA
tggtcatcgcttactgtctc

SEQ ID NO: 14 - DNA, 18 bp, synthetic construct
  source 1..18: mol_type = other DNA
tgctgcatgttgccttcg

SEQ ID NO: 15 - DNA, 20 bp, synthetic construct
  source 1..20: mol_type = other DNA
ggcgacctggaagtccaact

SEQ ID NO: 16 - DNA, 20 bp, synthetic construct
  source 1..20: mol_type = other DNA
ccatcagcaccacagccttc

SEQ ID NO: 17 - DNA, 20 bp, synthetic construct
  source 1..20: mol_type = other DNA
gaagaggatctgttgcagga

SEQ ID NO: 18 - DNA, 20 bp, synthetic construct
  source 1..20: mol_type = other DNA
caggactcatgcagccttac

SEQ ID NO: 19 - DNA, 20 bp, synthetic construct
  source 1..20: mol_type = other DNA
gacgagaacatcggcggcgt

SEQ ID NO: 20 - DNA, 20 bp, synthetic construct
  source 1..20: mol_type = other DNA
gtggcagcggttgggcttct

SEQ ID NO: 21 - DNA, 23 bp, synthetic construct
  source 1..23: mol_type = other DNA
ttcgcagaagcagcagtgaagag

SEQ ID NO: 22 - DNA, 23 bp, synthetic construct
  source 1..23: mol_type = other DNA
agccagagacagcgggattcctt

SEQ ID NO: 23 - DNA, 20 bp, synthetic construct
  source 1..20: mol_type = other DNA
tcaccaggcagagatatgga

SEQ ID NO: 24 - DNA, 20 bp, synthetic construct
  source 1..20: mol_type = other DNA
gcaagtctcatttcccatca

SEQ ID NO: 25 - DNA, 24 bp, synthetic construct
  source 1..24: mol_type = other DNA
gtgcccttgacaaagttcctggaa

SEQ ID NO: 26 - DNA, 24 bp, synthetic construct
  source 1..24: mol_type = other DNA
gcagaacctagggatgggtcatgc

SEQ ID NO: 27 - DNA, 24 bp, synthetic construct
  source 1..24: mol_type = other DNA
gaattcttggaagcacaggaaaca

SEQ ID NO: 28 - DNA, 24 bp, synthetic construct
  source 1..24: mol_type = other DNA
cagggacctaggagctgagcaggg

SEQ ID NO: 29 - DNA, 21 bp, synthetic construct
  source 1..21: mol_type = other DNA
cttcctatggctgggagtgag

SEQ ID NO: 30 - DNA, 20 bp, synthetic construct
  source 1..20: mol_type = other DNA
cccaactggtacatcagcac

SEQ ID NO: 31 - DNA, 20 bp, synthetic construct
  source 1..20: mol_type = other DNA
tctgctcattcacgaaaagg

SEQ ID NO: 32 - DNA, 20 bp, synthetic construct
  source 1..20: mol_type = other DNA
ctaccccaatttccaatgct

SEQ ID NO: 33 - DNA, 20 bp, synthetic construct
  source 1..20: mol_type = other DNA
accacagtgaggaatgtcca

SEQ ID NO: 34 - DNA, 22 bp, synthetic construct
  source 1..22: mol_type = other DNA
ccgaagtcatagccacactcaa

SEQ ID NO: 35 - DNA, 20 bp, synthetic construct
  source 1..20: mol_type = other DNA
caagggagcttcagggtcaa

SEQ ID NO: 36 - DNA, 24 bp, synthetic construct
  source 1..24: mol_type = other DNA
tgacttcaagaacatccagagctt

SEQ ID NO: 37 - DNA, 26 bp, synthetic construct
  source 1..26: mol_type = other DNA
cttgagagtggctatgacttctgtct

SEQ ID NO: 38 - DNA, 22 bp, synthetic construct
  source 1..22: mol_type = other DNA
tttgagaccatccagagcttga

SEQ ID NO: 39 - DNA, 25 bp, synthetic construct
  source 1..25: mol_type = other DNA
ccttgagagtggctatgacttctgt

SEQ ID NO: 40 - DNA, 19 bp, synthetic construct
  source 1..19: mol_type = other DNA
gcgccatatggagctgaca

SEQ ID NO: 41 - DNA, 20 bp, synthetic construct
  source 1..20: mol_type = other DNA
cgtggaatcttccggctgta

SEQ ID NO: 42 - DNA, 20 bp, synthetic construct
  source 1..20: mol_type = other DNA
cagcagcaagtgctccaatc

SEQ ID NO: 43 - DNA, 20 bp, synthetic construct
  source 1..20: mol_type = other DNA
cacacacttggcggttcctt

SEQ ID NO: 44 - DNA, 18 bp, synthetic construct
  source 1..18: mol_type = other DNA
tggtgcctggtctgatga

SEQ ID NO: 45 - DNA, 21 bp, synthetic construct
  source 1..21: mol_type = other DNA
gtggtaaccgctcaggtgttg

SEQ ID NO: 46 - DNA, 19 bp, synthetic construct
  source 1..19: mol_type = other DNA
gctccaagcagatgcagca

SEQ ID NO: 47 - DNA, 18 bp, synthetic construct
  source 1..18: mol_type = other DNA
ccggatgtgaggcagcag

SEQ ID NO: 48 - DNA, 21 bp, synthetic construct
  source 1..21: mol_type = other DNA
gcaccttacacctaccagagt

SEQ ID NO: 49 - DNA, 21 bp, synthetic construct
  source 1..21: mol_type = other DNA
aaacttctgcctgacgagctt

SEQ ID NO: 50 - DNA, 20 bp, synthetic construct
  source 1..20: mol_type = other DNA
cagccagatgcagttaacgc

SEQ ID NO: 51 - DNA, 23 bp, synthetic construct
  source 1..23: mol_type = other DNA
gcctactcattgggatcatcttg

SEQ ID NO: 52 - DNA, 19 bp, synthetic construct
  source 1..19: mol_type = other DNA
attacccgcccgagaaagg

SEQ ID NO: 53 - DNA, 21 bp, synthetic construct
  source 1..21: mol_type = other DNA
tcgcagcaaagatccacacag

SEQ ID NO: 54 - DNA, 20 bp, synthetic construct
  source 1..20: mol_type = other DNA
gagttaggggctggctctac

SEQ ID NO: 55 - DNA, 20 bp, synthetic construct
  source 1..20: mol_type = other DNA
gaccaagaaagagcacctca

SEQ ID NO: 56 - DNA, 19 bp, synthetic construct
  source 1..19: mol_type = other DNA
cttgctacggaatgaaaga

SEQ ID NO: 57 - DNA, 20 bp, synthetic construct
  source 1..20: mol_type = other DNA
ttacccagggaaagaggatg

SEQ ID NO: 58 - DNA, 20 bp, synthetic construct
  source 1..20: mol_type = other DNA
agtcgtggagctagggaagt

SEQ ID NO: 59 - DNA, 20 bp, synthetic construct
  source 1..20: mol_type = other DNA
acattgctctgcagttgtca

SEQ ID NO: 60 - DNA, 20 bp, synthetic construct
  source 1..20: mol_type = other DNA
caccttggagttcacccagt

SEQ ID NO: 61 - DNA, 20 bp, synthetic construct
  source 1..20: mol_type = other DNA
accactcgtacttgggatgc

SEQ ID NO: 62 - DNA, 24 bp, synthetic construct
  source 1..24: mol_type = other DNA
aggcggtggagttcacctttaaga

SEQ ID NO: 63 - DNA, 24 bp, synthetic construct
  source 1..24: mol_type = other DNA
agcttgcattccttggcatgatgg

SEQ ID NO: 64 - DNA, 22 bp, synthetic construct
  source 1..22: mol_type = other DNA
taagttctgagtgtgaccgaga

SEQ ID NO: 65 - DNA, 22 bp, synthetic construct
  source 1..22: mol_type = other DNA
gctctgtctgtagggaggtagg

SEQ ID NO: 66 - DNA, 20 bp, synthetic construct
  source 1..20: mol_type = other DNA
accaagccggatttgcgatt

SEQ ID NO: 67 - DNA, 21 bp, synthetic construct
  source 1..21: mol_type = other DNA
acttgcacttgttcctcgtgg

SEQ ID NO: 68 - DNA, 20 bp, synthetic construct
  source 1..20: mol_type = other DNA
atggaccgtagcatccctcc

SEQ ID NO: 69 - DNA, 20 bp, synthetic construct
  source 1..20: mol_type = other DNA
gtaggtgcgtaggttctggt

SEQ ID NO: 70 - DNA, 23 bp, synthetic construct
  source 1..23: mol_type = other DNA
atgatggcttattacagtggcaa

SEQ ID NO: 71 - DNA, 20 bp, synthetic construct
  source 1..20: mol_type = other DNA
gtcggagattcgtagctgga

SEQ ID NO: 72 - DNA, 21 bp, synthetic construct
  source 1..21: mol_type = other DNA
cctgaaccttccaaacatggc

SEQ ID NO: 73 - DNA, 21 bp, synthetic construct
  source 1..21: mol_type = other DNA
ttcaccaggcaagtctcctca

SEQ ID NO: 74 - DNA, 20 bp, synthetic construct
  source 1..20: mol_type = other DNA
acagcacagggcgttttatc

SEQ ID NO: 75 - DNA, 20 bp, synthetic construct
  source 1..20: mol_type = other DNA
gagcccagcattttctcttg

SEQ ID NO: 76 - DNA, 20 bp, synthetic construct
  source 1..20: mol_type = other DNA
aaccctccctaaccaaccac

SEQ ID NO: 77 - DNA, 20 bp, synthetic construct
  source 1..20: mol_type = other DNA
gttgcagttgctgttgctgt

SEQ ID NO: 78 - DNA, 20 bp, synthetic construct
  source 1..20: mol_type = other DNA
ggaatacagggctttcgatt

SEQ ID NO: 79 - DNA, 20 bp, synthetic construct
  source 1..20: mol_type = other DNA
ctctgtggagctgaagcaat

SEQ ID NO: 80 - DNA, 20 bp, synthetic construct
  source 1..20: mol_type = other DNA
taccatctacctgggcttcg

SEQ ID NO: 81 - DNA, 20 bp, synthetic construct
  source 1..20: mol_type = other DNA
gctccataaggaagcactcg

SEQ ID NO: 82 - DNA, 20 bp, synthetic construct
  source 1..20: mol_type = other DNA
atgttgctgagccctacaag

SEQ ID NO: 83 - DNA, 20 bp, synthetic construct
  source 1..20: mol_type = other DNA
gctgtcgctttgatggtact

SEQ ID NO: 84 - DNA, 21 bp, synthetic construct
  source 1..21: mol_type = other DNA
caagcagagtacacacagcat

SEQ ID NO: 85 - DNA, 22 bp, synthetic construct
  source 1..22: mol_type = other DNA
tgctccacttttaacttgagcc

SEQ ID NO: 86 - DNA, 20 bp, synthetic construct
  source 1..20: mol_type = other DNA
caccatcctggtggaactct

SEQ ID NO: 87 - DNA, 20 bp, synthetic construct
  source 1..20: mol_type = other DNA
tccaggataccttggaccag

SEQ ID NO: 88 - DNA, 29 bp, synthetic construct
  source 1..29: mol_type = other DNA
gttcctggagcatgtacttcaacggctgc

SEQ ID NO: 89 - DNA, 29 bp, synthetic construct
  source 1..29: mol_type = other DNA
gcagccgttgaagtacatgctccaggaac

SEQ ID NO: 90 - DNA, 24 bp, synthetic construct
  source 1..24: mol_type = other DNA
gcatgacccatccctaggttctgc

SEQ ID NO: 91 - DNA, 152 bp, synthetic construct
  source 1..152: mol_type = other DNA
gtgcccttgacaaagttcctggaagcaaaggaaacaacatgtcctagggagactaagtgaacatctgtggggacagaaattaaattaagatcagagggccgcctgccttattattgctaatggttctagcatgacccatccctaggttctgc

SEQ ID NO: 92 - DNA, 24 bp, synthetic construct
  source 1..24: mol_type = other DNA
ccctgctcagctcctaggtccctg

SEQ ID NO: 93 - DNA, 141 bp, synthetic construct
  source 1..141: mol_type = other DNA
gaattcttggaagcacaggaaacaacatgccacataggggttgagtaagcatctctggggccacaaattaaattaagctttcagggccgcctgccttgttattgctaatggttctagccctgctcagctcctaggtccctg

(19) United States
(12) Patent Application Publication
(10) Pub. No.: US 2026/0355199 A1
(43) Pub. Date: Nov. 12, 2026

(54) COMPOSITIONS AND METHODS FOR TREATING OR PREVENTING NONALCOHOLIC STEATOHEPATITIS (NASH), ANOREXIA, DEPRESSION, ENDOMETRIOSIS, AND OTHER DISEASES OR DISORDERS

(71) Applicant: Yale University, New Haven, CT (US)

(72) Inventor: Yingqun Huang

(21) Appl. No.: 19/118,194

(22) PCT Filed: Oct. 3, 2023
(86) PCT No.: PCT/US2023/075802

§ 371 Date: Apr. 3, 2025

Related U.S. Application Data
(60) Provisional application No. 63/378,121, filed on Oct. 3, 2022.
(60) Provisional application No. 63/492,868, filed on Mar. 29, 2023.

(51) Int. Cl. A61K 31/513; A61P 1/16; A61P 3/00; A61P 15/00; A61P 25/22; A61P 25/24; A61P 35/00

(52) U.S. Cl. CPC A61K 31/513; A61P 1/16; A61P 3/00; A61P 15/00; A61P 25/22; A61P 25/24; A61P 35/00

(57) ABSTRACT

The present invention provides compositions and methods for reducing the level and/or activity of a TET protein, for treating or preventing a disease and disorder associated with an increased level and/or activity of a TET protein, for reducing, stopping, or reversing weight loss, for inducing apoptosis in disease-associated macrophage (e.g., endometriosis-associated macrophage and tumor-associated macrophage) and/or cancer-associated fibroblast, and/or for preventing or treating various diseases or disorder (e.g., endometriosis, non-alcoholic steatohepatitis, inflammatory disease or disorder, chronic inflammatory disease or disorder, inflammatory bowel disease, Alzheimer's disease, Parkinson's disease, cancer, and/or cancer-associated diseases or disorders). In various embodiments, the method comprises administering a degrader of TET protein, inhibitor of TET protein, or a combination thereof as a therapeutic.

### CROSS-REFERENCE TO RELATED APPLICATIONS

This application claims priority to U.S. Provisional Application Ser. No. 63/492,868, filed Mar. 29, 2023, and U.S. Provisional Application Ser. No. 63/378,121, filed Oct. 3, 2022, the disclosures of which are hereby incorporated by reference herein in their entireties.

### STATEMENT REGARDING FEDERALLY SPONSORED RESEARCH OR DEVELOPMENT

This invention was made with government support under DK119386 and DK124321 awarded by National Institutes of Health. The government has certain rights in the invention.

### REFERENCE TO SEQUENCE LISTING SUBMITTED ELECTRONICALLY

This application contains a sequence listing, which is submitted electronically as an XML formatted sequence listing with a file name “047162-5332-00WO_Sequence_Listing.xml” creation date of Oct. 3, 2023 and having a size of 83,385 bytes. The sequence listing submitted is part of the Specification and is herein incorporated by reference in its entirety.

### BACKGROUND OF THE INVENTION

Macrophages are tissue-resident or infiltrated immune cells that play critical roles in the development and progression of chronic inflammatory diseases. However, targeting these disease-associated macrophages (DAMs) for therapy has remained extremely challenging, largely owing to their high heterogeneity both molecularly and phenotypically (Skytthe et al., 2020, Int J Mol Sci., 21; Ardura et al., 2019, Front Pharmacol., 10:1255). Nonalcoholic steatohepatitis (NASH) and endometriosis are two major chronic inflammatory diseases of distinct types. NASH is characterized by inflammation of the liver that causes fibrosis and predisposes to cirrhosis and hepatocellular carcinoma (HCC) (Younossi et al., 2018, Nat Rev Gastroenterol Hepatol., 15:11-20; Barreby et al., 2022, Nat Rev Endocrinol., 18:461-472). Endometriosis is defined as the growth of endometrial-like tissue outside of the uterus. It causes pain and infertility and is associated with an increased risk of ovarian cancer (Pearce et al., 2012, Lancet Oncol., 13:385-394). DAMs are known to be critical drivers of both NASH and endometriosis (Barreby et al., 2022, Nat Rev Endocrinol., 18:461-472; Cai et al., 2020, Cell Metab., 31:406-421; Hogg et al., 2020, Front Endocrinol (Lausanne), 11:7). Despite high unmet medical needs, there are no FDA-approved drugs for the treatment of NASH, and treatment options for endometriosis are hormonal and of limited effectiveness (Younossi et al., 2018, Nat Rev Gastroenterol Hepatol., 15:11-20; Zondervan et al., 2020, N Engl J Med., 382:1244-1256).

Anorexia nervosa (AN) is a psychiatric illness with the highest mortality. Individuals with AN frequently exhibit stress symptoms including anxiety, depression, and obsessive-compulsive disorders. Current treatment options have been limited to psychotherapy and nutritional support, with low efficacy and high relapse rates (Scharner et al., 2020, Front Hum Neurosci., 14:596381; van Eeden et al., 2021, Curr Opin Psychiatry, 34:515-524).

Thus, there is a need in the art for compositions and methods for effective treatment and prevention of various diseases and disorders associated with food-intake, anxiety, depression, or chronic inflammation and other pathologies. The present invention addresses and meets these and other needs.

### SUMMARY OF THE INVENTION

In one aspect, the present invention relates, in part, to a method of treating or preventing a disease or disorder associated with the level of at least one TET protein in a subject in need thereof. In some embodiments, the method comprises administering to the subject a therapeutically effective amount of a degrader of TET protein, inhibitor of TET protein, or a combination thereof, or a composition thereof.

In some embodiments, the disease or disorder associated with the level of at least one TET protein is a disease or disorder associated with an increased level of at least one TET, disease or disorder associated with an increased activity of at least one TET, disease or disorder associated with an increased expression of at least one TET, disease or disorder associated with an increased function of at least one TET, or any combination thereof. In some embodiments, the disease or disorder associated with the level of at least one TET protein is an eating disorder, disease or disorder associated with reduced food intake, anorexia nervosa, cancer-induced anorexia, gynecological disease, endometriosis, anxiety, stress-related disorder, depression, cancer-induced depression, postpartum depression, major depression, depression-related illness, non-alcoholic fatty liver disease (NAFLD), non-alcoholic steatohepatitis (NASH), liver fibrosis, cancer, liver cancer, ovarian cancer, acute myeloid leukemia (AML), pancreatic cancer, glioma, bladder cancer, lung cancer, breast cancer, inflammatory disease or disorder, chronic inflammatory disease or disorder, or any combination thereof. In one embodiment, the disease or disorder associated with reduced food intake is induced by a treatment of cancer. In one embodiment, the gynecological disease is an endometriosis.

In some embodiments, the at least one TET protein is TET1 protein, TET2 protein, TET3 protein, or any combination thereof. In one embodiment, the at least one TET protein is TET3.

In some embodiments, the degrader of TET protein is a degrader of TET1 protein, degrader of TET2 protein, degrader of TET3 protein, or any combination thereof. In one embodiment, the degrader of TET protein is a degrader of TET3 protein. In some embodiments, the degrader of TET3 protein decreases the level or activity of TET3 protein. In some embodiments, the degrader of TET3 protein decreases the level or activity of TET3 protein and simultaneously does not affect the level or activity of TET2 protein. In some embodiments, the degrader of TET3 protein is 4-amino-1-[1,1′-biphenyl]-3-yl-5-chloro-2(1H)-pyrimidinone or a derivative, analog, pharmaceutically acceptable salt, hydrate, or prodrug thereof.

In some embodiments, the inhibitor of TET protein is an inhibitor of TET1 protein, inhibitor of TET2 protein, inhibitor of TET3 protein, or any combination thereof. In one embodiment, the inhibitor of TET protein is an inhibitor of TET3 protein. In some embodiments, the inhibitor of TET3 protein decreases the level or activity of TET3 protein. In some embodiments, the inhibitor of TET3 protein decreases the level or activity of TET3 protein and simultaneously does not affect the level or activity of TET2 protein.

In some embodiments, the degrader of TET protein, inhibitor of TET protein, or a combination thereof decreases the activity of at least one TET protein, decreases the level of at least one TET protein, decreases the expression of at least one TET protein, decreases the function of at least one TET protein, decrease the stability of at least one TET protein, increases the degradation of at least one TET protein, or any combination thereof.

In some embodiments, the degrader of TET protein, inhibitor of TET protein, or a combination thereof decreases the activity of at least one TET protein in at least one agouti-related peptide (AgRP) neuron, disease-associated macrophage (DAM), such as tumor-associated macrophage (e.g., lung cancer-associated macrophage, ovarian cancer-associated macrophage, leukemia-associated macrophage, AML cancer-associated macrophage, breast cancer-associated macrophage, pancreatic cancer-associated macrophage, etc.), cancer-associated fibroblast (CAF) (e.g., lung cancer-associated fibroblast, ovarian cancer-associated fibroblast, leukemia-associated fibroblast, AML cancer-associated fibroblast, breast cancer-associated fibroblast, pancreatic cancer-associated fibroblast, etc.), cancer cell (e.g., ovarian cancer cell, leukemia cancer cell, AML cancer cell, breast cancer cell, lung cancer cell, pancreatic cancer cell, etc.), or any combination thereof, decreases the level of at least one TET protein in at least one AgRP neuron, DAM, such as DAM associated with ovarian cancer, leukemia, AML, breast cancer, lung cancer, and/or pancreatic cancer, CAF, such as CAF associated with ovarian cancer, leukemia, AML, breast cancer, lung cancer, and/or pancreatic cancer, ovarian cancer cell, leukemia cancer cell, AML cancer cell, breast cancer cell, lung cancer cell, pancreatic cancer cell, or any combination thereof, decreases the expression of at least one TET protein in at least one AgRP neuron, decreases the function of at least one TET protein in at least one AgRP neuron, DAM, such as DAM associated with ovarian cancer, leukemia, AML, breast cancer, lung cancer, and/or pancreatic cancer, CAF, such as CAF associated with ovarian cancer, leukemia, AML, breast cancer, lung cancer, and/or pancreatic cancer, ovarian cancer cell, leukemia cancer cell, AML cancer cell, breast cancer cell, lung cancer cell, pancreatic cancer cell, or any combination thereof, decrease the stability of at least one TET protein in at least one AgRP neuron, DAM, such as DAM associated with ovarian cancer, leukemia, AML, breast cancer, lung cancer, and/or pancreatic cancer, CAF, such as CAF associated with ovarian cancer, leukemia, AML, breast cancer, lung cancer, and/or pancreatic cancer, ovarian cancer cell, leukemia cancer cell, AML cancer cell, breast cancer cell, lung cancer cell, pancreatic cancer cell, or any combination thereof, increases the degradation of at least one TET protein in at least one AgRP neuron, DAM, such as DAM associated with ovarian cancer, leukemia, AML, breast cancer, lung cancer, and/or pancreatic cancer, CAF, such as CAF associated with ovarian cancer, leukemia, AML, breast cancer, lung cancer, and/or pancreatic cancer, ovarian cancer cell, leukemia cancer cell, AML cancer cell, breast cancer cell, lung cancer cell, pancreatic cancer cell, or any combination thereof, or any combination thereof.

In some embodiments, the degrader of TET protein, inhibitor of TET protein, or a combination thereof further increases the level of at least one AGRP peptide, NPY peptide, vesicular GABA transporter (VGAT), or a combination thereof, increases the activity of at least one AGRP peptide, NPY peptide, VGAT, or a combination thereof, increases the function of at least one AGRP peptide, NPY peptide, VGAT, or a combination thereof, or increases the expression of at least one AGRP peptide, NPY peptide, VGAT, or a combination thereof.

In one aspect, the present invention relates, in part, to a method of reducing, stopping, or reversing a weight loss in a subject in need thereof. In some embodiments, the method comprises administering to the subject a therapeutically effective amount of a degrader of TET protein, inhibitor of TET protein, or a combination thereof, or a composition thereof.

In one aspect, the present invention also provides a method of treating or preventing a disease or disorder associated with the level of at least one DAM, CAF, or a combination thereof in a subject in need thereof. In some embodiments, the method comprises administering to the subject a therapeutically effective amount of a modulator of a DAM, CAF, or a combination thereof.

In some embodiments, the modulator of a DAM induces apoptosis of at least one DAM. In some embodiments, the modulator of a DAM is 4-amino-1-[1,1′-biphenyl]-3-yl-5-chloro-2(1H)-pyrimidinone or a derivative, analog, pharmaceutically acceptable salt, hydrate, or prodrug thereof.

In some embodiments, the modulator of a DAM further decreases the activity of at least one TET protein, decreases the level of at least one TET protein, decreases the expression of at least one TET protein, decreases the function of at least one TET protein, decrease the stability of at least one TET protein, increases the degradation of at least one TET protein, or any combination thereof.

In some embodiments, the modulator of a CAF induces apoptosis of at least one CAF. In some embodiments, the modulator of a CAF is 4-amino-1-[1,1′-biphenyl]-3-yl-5-chloro-2(1H)-pyrimidinone or a derivative, analog, pharmaceutically acceptable salt, hydrate, or prodrug thereof.

In some embodiments, the modulator of a CAF further decreases the activity of at least one TET protein, decreases the level of at least one TET protein, decreases the expression of at least one TET protein, decreases the function of at least one TET protein, decrease the stability of at least one TET protein, increases the degradation of at least one TET protein, or any combination thereof.

In some embodiments, the disease or disorder associated with the level of at least one DAM, CAF, or a combination thereof is a disease or disorder associated with increased level of at least one DAM, CAF, or a combination thereof, disease or disorder associated with increased activity of at least one DAM, CAF, or a combination thereof, disease or disorder associated with increased expression of at least one DAM, CAF, or a combination thereof, disease or disorder associated with increased function of at least one DAM, CAF, or a combination thereof, or any combination thereof. In one embodiment, the disease or disorder associated with the level of at least one DAM, CAF, or a combination thereof is endometriosis, non-alcoholic steatohepatitis (NASH), inflammatory disease or disorder, chronic inflammatory disease or disorder, inflammatory bowel disease (IBD), Alzheimer's disease, Parkinson's disease, cancer, cancer-associated disease or disorder, or any combination thereof.

In some embodiments, the DAM is an endometriosis-associated macrophage (EAM). In some embodiments, the method comprises administering to the subject a therapeutically effective amount of a modulator of an EAM.

In one aspect, the present invention also provides a method of treating or preventing a disease or disorder associated with the level of at least one agouti-related peptide (AgRP) neuron in a subject in need thereof. In some embodiments, the method comprises administering to the subject a therapeutically effective amount of a modulator of at least one AgRP neuron.

In some embodiments, the modulator of at least one AgRP neuron increases the level of at least one AGRP peptide, neuropeptide Y (NPY) peptide, vesicular GABA transporter (VGAT), or a combination thereof, increases the activity of at least one AGRP peptide, NPY peptide, VGAT, or a combination thereof, increases the function of at least one AGRP peptide, NPY peptide, VGAT, or a combination thereof, or increases the expression of at least one AGRP peptide, NPY peptide, VGAT, or a combination thereof. In some embodiments, the modulator of at least one AgRP neuron is 4-amino-1-[1,1′-biphenyl]-3-yl-5-chloro-2(1H)-pyrimidinone or a derivative, analog, pharmaceutically acceptable salt, hydrate, or prodrug thereof. In some embodiments, the modulator of at least one AgRP neuron further decreases the activity of at least one TET protein, decreases the level of at least one TET protein, decreases the expression of at least one TET protein, decreases the function of at least one TET protein, decrease the stability of at least one TET protein, increases the degradation of at least one TET protein, or any combination thereof.

In some embodiments, the disease or disorder associated with the level of at least one AgRP neuron is a disease or disorder associated with decreased level of at least one AgRP neuron, disease or disorder associated with decreased activity of at least one AgRP neuron, disease or disorder associated with decreased expression of at least one AgRP neuron, disease or disorder associated with decreased function of at least one AgRP neuron, or any combination thereof. In one embodiment, the disease or disorder associated with the level of at least one AgRP neuron is eating disorder, mood disorder, cancer-associated disease or disorder, cachexia, cancer-associated cachexia, depression, anxiety, hypophagia, or any combination thereof.

In one aspect, the present invention also provides a method of modulating at least one pathway involved in transforming growth factor beta (TGF-β) signaling, metabolic reprogramming, pyroptosis, apoptosis, or any combination thereof in a subject in need thereof. In some embodiments, the method comprises inhibiting at least one pathway involved in TGF-βsignaling, metabolic reprogramming, or a combination thereof. In some embodiments, the method comprises activating at least one pathway involved in apoptosis. In some embodiments, the method comprises administering to the subject a therapeutically effective amount of a degrader of TET protein, inhibitor of TET protein, or a combination thereof, or a composition thereof.

In one aspect, the present invention also provides a method of reducing or inhibiting at least one TET protein, transforming growth factor beta (TGF-β), interleukin-1 beta (IL-1β), interleukin 6 (IL-6), or any combination thereof in a subject in need thereof by administering to the subject a therapeutically effective amount of 4-amino-1-[1,1′-biphenyl]-3-yl-5-chloro-2(1H)-pyrimidinone or a derivative, analog, pharmaceutically acceptable salt, hydrate, or prodrug thereof.

In some embodiments, the method comprises reducing or inhibiting the level or activity of TET3 protein, TGF-β, IL-1β, IL-6, or any combination thereof. In some embodiments, the method comprises reducing or inhibiting the level or activity of TET3 protein, TGF-β, IL-1β, IL-6, or any combination thereof and simultaneously not effecting the level or activity of TET2 protein.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1, comprising FIG. 1A through FIG. 1C, depicts representative imaging and quantification of Bobcat339 treatment and siRNA-mediated TET3 knockdown. FIG. 1A depicts representative photomicrographs showing increased AGRP peptide (red) in the arcuate nucleus of hypothalamus (ARC) of mice treated with Bobcat339 vs. vehicle. TET3 (green) is a predominantly nuclear protein. As AgRP neurons were not labeled, the TET3-positive cells represent both AgRP and non-AgRP cells. 3V. third ventricle. FIG. 1B depicts representative quantification of Tet3 and Tet2 mRNAs in GT1-7, a mouse hypothalamic cell line, transfected with non-targeting control siRNA (NT siRNA) or siRNA specifically targeting mouse Tet3 (Tet3 siRNA) for 48 h. n=3 per group. Data: mean±SEM. **, p<0.01, by 2-tailed Student's t tests. FIG. 1C depicts representative photomicrographs showing reduced TET3 protein (red) in GT1-7 cells treated with Tet3 siRNA vs. NT siRNA for 48 h. Cell nuclei (blue) were labeled by DAPI.

FIG. 2, comprising FIG. 2A through FIG. 2C, depicts representative results demonstrating decreases in TET3 expression in AgRP neurons during fasting. FIG. 2A depicts representative levels of Agrp, Tet3 and Tet2 mRNAs in the ARCs of fed and fasted mice. N=4 mice per group. *P<0.05, by 2-tailed Student's t tests. FIG. 2B depicts representative photomicrographs of AgRP neurons (green) and AGRP peptide (red) showing that AGRP expression markedly increased in the ARC of fasted mice. FIG. 2C depicts representative photomicrographs and corresponding statistical analysis of TET3 (red)-positive AgRP neurons (green) showing decreased TET3 expression in AgRP neurons by fasting. N=5 mice per group. ***P<0.001, by 2-tailed Student's t tests. 3V, third ventricle. Scale bars: 50 μm. Data: mean±SEM.

FIG. 3, comprising FIG. 3A through FIG. 3I, depicts representative results demonstrating increase in AGRP expression and AgRP neuronal activity when TET3 was knocked down. FIG. 3A depicts a schematic representation of AAV-sgTet3 (top) with sgRNA design for targeting the mouse Tet3 genomic locus (bottom). FIG. 3B depicts a schematic representation of bilateral virus injection into the ARC of Cas9+ mice. FIG. 3C depicts representative photomicrographs of AgRP neurons (green) expressing injected AAV-sgTet3 (red). FIG. 3D depicts representative photomicrographs and corresponding statistical analysis of TET3 (green)-positive AgRP neurons (red) showing decreased TET3 expression in AgRP neurons in AAV-sgTet3 injected mice. N=5 mice per group. Data: mean±SEM. ***P<0.001, by 2-tailed Student's t tests. FIG. 3E depicts representative photomicrographs of AgRP neurons (red) and AGRP peptide (green) showing a marked increase in AGRP peptide in the ARC of ad libitum-fed mice injected with AAV-sgTet3. FIG. 3F depicts representative results demonstrating increased expression of Agrp mRNA in the ARC of mice injected with AAV-sgTet3. N=6-7 mice per group. Data: mean±SEM. **P<0.01, by 2-tailed Student's t tests.

FIG. 3G depicts representative quantification of AgRP neurons in the ARCs of Cas9+ mice injected with AAV or AAV-sgTet3 showing no significant difference between the groups. N=5 mice per group. Data: mean±SEM; 2-tailed Student's t tests. FIG. 3H depicts representative traces of membrane and action potentials recorded under current-clamp in AgRP neurons of Cas9+ mice injected with AAV or AAV-sgTet3. FIG. 3I depicts representative bar graphs showing the frequency of spontaneous Aps (left panel) and AP threshold (right panel) in AgRP cells in control and TET3 knockdown mice. N=10-11 neurons from 3-4 mice per group. Data: mean±SEM. *P<0.05, by 2-tailed Student's t tests. Scale bars, 50 μm.

FIG. 4, comprising FIG. 4A and FIG. 4B, depicts representative mRNA quantification data demonstrating TET3 negatively regulates Agrp expression in both mouse and human cell lines. Data: mean±SEM. FIG. 4A depicts representative levels of Tet3, Agrp, and Tet2 mRNAs in a mouse mHypoE-N11 embryonic hypothalamus cell line transfected with NT siRNA or Tet3 siRNA. RNAs were extracted at 24 h (for Tet3 and Tet2) or at 48 h (for Agrp). N=3 per group. **, p<0.01; ***, p<0.001; by 2-tailed Student's t tests. FIG. 4B depicts representative levels of Tet3, Agrp, and Tet2 mRNAs in a human SH-Sy5Y neroblastoma cell line transfected with NT siRNA or Tet3 siRNA. RNAs were extracted at 24 h (for Tet3 and Tet2) or at 48 h (for Agrp). N=3 per group. **, p<0.01; ***, p<0.001; by 2-tailed Student's t tests.

FIG. 5, comprising FIG. 5A through FIG. 5I, depicts representative results demonstrating that TET3 is required for leptin-induced repression of AGRP expression in cell lines. FIG. 5A depicts a schematic representation of a post-fast refeeding study of Cas9+ mice injected with AAV or AAV-sgTet3. FIG. 5B depicts representative food intake of mice injected with AAV at the indicated time points following administration of leptin or saline. N=6 mice per group. Data: mean±SEM. *P<0.05, **P<0.01, by 2-tailed Student's t tests. FIG. 5C depicts representative food intake of mice injected with AAV-sgTet3 following administration of leptin or saline. N=6 mice per group. Data: mean±SEM. FIG. 5D depicts representative RNA quantification of mouse GT1-7 cells maintained in a high leptin concentration (Lept H, 1×10⁻⁸ M) that were switched to a low leptin concentration (Lept L, 1×10⁻¹⁰ M), followed by RNA extraction and qPCR of Tet3 and Agrp mRNAs at 24 h after the switch. N=3 per group. Data: mean±SEM. *P<0.05, ***P<0.001, by 2-tailed Student's t tests. FIG. 5E depicts representative qPCR of Tet3 and Agrp mRNAs from GT1-7 cells maintained in Lept L and then switched to Lept H for 24 h. n=3 per group. Data: mean±SEM. **P<0.01, ***P<0.001, by 2-tailed Student's t tests. FIG. 5F depicts representative quantification of RNA in GT1-7 cells transfected with NT siRNA and maintained in Lept L (NT siRNA/Lept L) or Lept H (NT siRNA/Lept H); or transfected with Tet3 siRNA and maintained in Lept H (Tet3 siRNA/Lept H). RNAs were extracted at 12 h (for Tet3) or 36 h (for Agrp) following the switch and analyzed by qPCR. Data: mean±SEM. *P<0.05, **P<0.01, ***P<0.001, by 1-way ANOVA with Tukey post-test. FIG. 5G depicts representative immunoblots for TET3 and AGRP from GT1-7 cells treated as in FIG. 5F. GAPDH as a loading control. Proteins were isolated at the 36 h time point. FIG. 5H depicts representative quantification of RNA in human SH-SY5Y neuroblastoma cells transfected with NT siRNA and maintained in Lept L (NT siRNA/Lept L) or Lept H (NT siRNA/Lept H); or transfected with TET3 siRNA and maintained in Lept H (TET3 siRNA/Lept H). RNAs were extracted at 24 h following the switch and analyzed by qPCR. N=3 per group. Data: mean±SEM. *P<0.05, **P<0.01, by 1-way ANOVA with Tukey post-test. FIG. 5I depicts representative immunoblots for TET3 and AGRP from SH-SY5Y cells treated as in FIG. 5H. Proteins were isolated at the 36 h time point.

FIG. 6, comprising FIG. 6A through FIG. 6M, depicts representative results demonstrating that TET3 promoted the association of a chromatin-modifying complex with the Agrp/AGRP promoters. FIG. 6A depicts a schematic representation of mouse Agrp proximal promoter with STAT3 and FOXO1 binding sites labeled in red and green, respectively. Numbers depict the starting and ending positions of nucleotides in the chromosome. The zoom-in sequences of the ChIP/hMeDIP region show STAT3 site in red and FOXO1 site in green, with PCR primers for the ChIP/hMeDIP region underlined. FIG. 6B depicts a schematic representation of human AGRP proximal promoter. FIG. 6C depicts a schematic representation of the experiments performed. Cas9+ mice were injected with AAV-sgTet3 or AAV into the ARC on day 1 (D1). Three weeks later (D21), the mice were fasted for 22 h and treated with leptin or saline for 2 h, followed by ARC isolation and ChIP-qPCR analyses. FIG. 6D depicts representative results demonstrating that ARCs from 4 mice in each group were pooled for ChIP-qPCR analyses using antibodies specific for STAT3, TET3, NCOR1, or HDAC4. Mice were treated as described in FIG. 6C. Preimmune IgG was used as a negative control. Data are presented as % input. N=3 per group in technical replicates. *P<0.05, **P<0.01, ***P<0.001, by 1-way ANOVA with Tukey post-test. FIG. 6E depicts representative results of human SH-SY5Y cells transfected with NT siRNA were maintained in Lept L (NT siRNA/Lept L) or Lept H (NT siRNA/Lept H); or transfected with TET3 siRNA and maintained in Lept H (TET3 siRNA/Lept H). Cells were collected at 36 h post-transfection and ChIP-qPCR experiments were performed. Data are presented as % input. Data are presented as % input. N=3 per group in technical replicates. *P<0.05, **P<0.01, ***P<0.001, by 1-way ANOVA with Tukey post-test. FIG. 6F depicts representative immunoblot results of Cas9+ mice that were fasted for 22 h, followed by leptin injection and isolation of ARCs at 2 h post leptin injection. ARCs from 3 mice were pooled for co-IP using anti-TET3 or preimmune IgG. Representative immunoblots using antibodies specific for TET3, STAT3, p-STAT3, NCOR1, or HDAC4 are shown, with protein sizes in kDa on the right. The band labeled with an asterisk (top blot, lane 1) is an isoform of TET3. FIG. 6G depicts representative immunoblots of co-IP using anti-TET3 or IgG from GT1-7 cells maintained in Lept H. Samples in lanes 2 and 3 were run on the same gel but were noncontiguous. FIG. 6H depicts representative immunoblots of co-IP using anti-TET3 or IgG from SH-SY5Y cells maintained in Lept H. FIG. 6I depicts representative results of Cas9+ mice that were treated as in FIG. 6C. ARCs from 2 mice in each group were pooled for ChIP-qPCR using anti-H3K9ac. Data are presented as % input. N=3 in technical replicates. FIG. 6J depicts representative results of SH-SY5Y cells that were treated as in FIG. 6E. ChIP-qPCR analysis was performed using anti-H3K9ac. Data are presented as % input. N=3 per group in technical replicates. **P<0.01, ***P<0.001, by 1-way ANOVA with Tukey post-test. FIG. 6K depicts representative results of Cas9+ mice that were treated as in FIG. 6C. ARCs from 2 mice in each group were pooled for hMeDIP-qPCR analysis. Data are presented as % input. N=3 per group in technical replicates. **P<0.01, by 1-way ANOVA with Tukey post-test. FIG. 6L depicts representative results demonstrating hMeDIP-qPCR of SH-SY5Y cells transfected with NT siRNA or TET3 siRNA in Lept H for 36 h. Data are presented as % input. N=3 per group in technical replicates. **P<0.01, by 2-tailed Student's t Tests. FIG. 6M depicts representative micrographs and statistical analysis of p-STAT3 (green)-positive AgRP neurons (red) showing no significant difference in p-STAT3 expression in AgRP neurons between mice injected with AAV and AAV-sgTet3 under ad libitum-fed conditions. N=5 animals per group. Data: mean±SEM. Two-tailed Student's t tests.

FIG. 7, comprising FIG. 7A through FIG. 7F, depict representative results demonstrating TET3 negatively regulates expression of NPY and VGAT. FIG. 7A depicts representative RNA quantification in GT1-7 cells transfected with NT siRNA and maintained in Lept L (NT siRNA/Lept L) or Lept H (NT siRNA/Lept H); or transfected with Tet3 siRNA and maintained in Lept H (Tet3 siRNA/Lept H). RNAs were extracted at 12 h (for Tet3) or 36 h (for Npy and Slc32a1) following the switch and analyzed by qPCR. Data: mean±SEM. N=3 per group in technical replicates. **P<0.01, ***P<0.001, by 1-way ANOVA with Tukey post-test. FIG. 7B depicts representative immunoblots for TET3, NPY and VGAT from GT1-7 cells treated as in FIG. 7A. Proteins were isolated at the 36 h time point. FIG. 7C depicts RNA quantification in SH-SY5Y cells transfected with NT siRNA and maintained in Lept L (NT siRNA/Lept L) or Lept H (NT siRNA/Lept H); or transfected with TET3 siRNA and maintained in Lept H (TET3 siRNA/Lept H). RNAs were extracted at 12 h (for TET3) or 36 h (for NPY and SLC32A1) following the switch and analyzed by qPCR. Data: mean±SEM. N=3 per group in technical replicates. **P<0.01, ***P<0.001, by 1-way ANOVA with Tukey post-test. FIG. 7D depicts representative immunoblots for TET3, NPY and VGAT from SH-SY5Y cells treated as in FIG. 7C. Proteins were isolated at the 36 h time point. FIG. 7E depicts representative micrographs of NPY (green) and VGAT (green) in the ARCs of Cas9+ mice injected with AAV or AAV-sgTet3. AgRP neurons were labeled red from the injected viruses. Scale bars: 50 μm. FIG. 7F depicts representative micrographs of NPY (green) and VGAT (green) in the ARCs of Cas9+ mice injected with AAV or AAV-sgTet3. AgRP neurons were labeled red from the injected viruses. Scale bars: 50 μm.

FIG. 8 depicts representative levels of Agrp and Pomc mRNAs in the ARCs of ad libitum-fed Cas9+ mice injected with AAV or AAV-sgTet3. N=5 mice per group. Data: mean±SEM. ***, p<0.001; by 2-tailed Student's t tests.

FIG. 9, comprising FIG. 9A through FIG. 9P, depicts representative experimental results demonstrating that TET3 knockdown in AgRP neurons in female mice induces hyperphagia, obesity, and diabetes, and reduces stress-like behaviors. Data: mean±SEM. FIG. 9A depicts representative food intake of Cas9+ mice injected with AAV-sgTet3 or AAV bilaterally into the ARC at the age of 6 weeks, which became hyperphagic at 2 weeks post-injection. N=8 animals per group. *P<0.05, **P<0.01, by 2-tailed Student's t tests. FIG. 9B depicts representative images of mice at 8 weeks post-injection. FIG. 9C depicts representative body weight changes of mice post-injection. N=8 animals per group. **P<0.01, by 2-tailed Student's t tests. FIG. 9D depicts representative fat mass of mice at 8 weeks post-injection. N=8 animals per group. ***P<0.001, by 2-tailed Student's t tests. FIG. 9E depicts representative energy expenditure at 2 weeks post-injection. N=8 animals per group. *P<0.05, **P<0.01, by 2-tailed Student's t tests. FIG. 9F depicts representative ad libitum-fed blood insulin at 5 weeks post-injection. N=8 animals per group. ***P<0.01, by 2-tailed Student's t tests. FIG. 9G depicts representative ad libitum-fed blood glucose at 6 weeks post-injection. N=8 animals per group. ***P<0.01, by 2-tailed Student's t tests. FIG. 9H depicts representative 9 ad libitum-fed blood leptin at 7 weeks post-injection. N=8 animals per group. **P<0.01, by 2-tailed Student's t tests. FIG. 9I depicts representative results of glucose tolerance tests (GTT) at 8 weeks post-injection. N=8 animals per group. *P<0.05, **P<0.01, ***P<0.01, by 2-way ANOVA with Sidak post-test. FIG. 9J depicts representative results of insulin tolerance tests (ITT) at 9 weeks post-injection. N=8 animals per group. *P<0.05, **P<0.01, by 2-way ANOVA with Sidak post-test. FIG. 9K depicts a schematic representation of experiments. Cas9+ mice were co-injected with AAV-sgTet3 and AAV-hM4Di bilaterally into the ARC on day 1 (D1), followed by implantation of osmotic pump containing saline or C21 on D4. Food intake measurements and ITT were performed on D5 and D9, respectively. FIG. 9L depicts representative food intake data. None: age-matched Cas9+ mice without AAV injection and osmotic pump. N=6 animals per group. **P<0.01, ***P<0.001, by 1-way ANOVA with Tukey post-test. FIG. 9M depicts representative ITT data. N=6 animals per group. *P<0.05, **P<0.01, by 1-way ANOVA with Tukey post-test. FIG. 9N depicts representative immobility scores of Cas9+ mice injected with AAV or AAV-sgTet3 in TST. N=7 animals per group. ***P<0.001, by 2-tailed Student's t tests. FIG. 9O depicts representative immobility scores of Cas9+ mice injected with AAV or AAV-sgTet3 in FST. N=7 animals per group. ***P<0.001, by 2-tailed Student's t tests. FIG. 9P depicts representative plasma corticosterone concentrations of Cas9+ mice injected with AAV or AAV-sgTet3. N=7 animals per group. ***P<0.001, by 2-tailed Student's t tests.

FIG. 10, comprising FIG. 10A through FIG. 10J, depicts representative experimental results demonstrating AgRP neuron-specific TET3 knockdown in male mice induces hyperphagia, obesity, and diabetes. Data±SEM. FIG. 10A depicts representative food intake of Cas9+ mice injected with AAV-sgTet3 or AAV bilaterally into the ARC at the age of 6 weeks, demonstrating hyperphagia at 3 weeks post injection. N=8 animals per group. *, p<0.05; by 2-tailed Student's t tests. FIG. 10B depicts representative images of mice at 8 weeks post injection. FIG. 10C depicts representative body weight changes of mice post injection. N=8 animals per group. *, p<0.05; by 2-tailed Student's t tests. FIG. 10D depicts representative fat mass of mice at 8 weeks post injection. N=8 animals per group. *, p<0.05; by 2-tailed Student's t tests. FIG. 10E depicts representative energy expenditure at 3 weeks post injection. FIG. 10F depicts representative a libitum-fed blood insulin at 6 weeks post-injection. N=8 animals per group. *, p<0.05; by 2-tailed Student's t tests. FIG. 10G depicts representative ad libitum-fed blood glucose at 7 weeks post injection. N=8 animals per group. *, p<0.05; by 2-tailed Student's t tests. FIG. 10H depicts representative ad libitum-fed blood leptin at 7 weeks post-injection. N=8 animals per group. *, p<0.05; by 2-tailed Student's t tests. FIG. 10I depicts representative results from glucose tolerance tests (GTT) at 8 weeks post injection. N=8 animals per group. *, p<0.05; by 2-way ANOVA with Sidak post-test. FIG. 10J depicts representative results from insulin tolerance tests (ITT) at 9 weeks post-injection. N=8 animals per group. *, p<0.05; **, p<0.01; by 2-way ANOVA with Sidak post-test.

FIG. 11 depicts representative imaging of AgRP neuron-specific expression of hM4Di. AAV-hM4Di was injected bilaterally into the ARC of Cas9+ mice, followed by detection of AgRP neurons (green) expressing AAV-hM4Di (red) without immunostaining.

FIG. 12, comprising FIG. 12A and FIG. 12B, depicts representative results of immunoprecipitation and Western blot analysis validation of antibodies specific for TET3 and HDAC4. FIG. 12A depicts representative results of immunoprecipitation experiments performed with mouse GT1-7 cells using anti-TET3 antibody (Active Motif, 61395) or preimmune IgG (as a negative control), showing that the anti-TET3 was able to pull down endogenous TET3 which could be detected by a different TET3 antibody (GenTex, GTX121453) in Western blot analysis. FIG. 12B depicts representative results of immunoprecipitation experiments performed with mouse GT1-7 cells using anti-HDAC4 antibody (Active Motif, 40969) showing that the antibody was able to pull down endogenous HDAC4 which could be detected by the same HDAC antibody in Western blot analysis.

FIG. 13, comprising FIG. 13A through FIG. 13D, depicts a schematic representation of a herein described model. FIG. 13A depicts a schematic representation of the herein described model in a fasted state, where leptin signaling and TET3 levels are low and there is no association of the chromatin-modifying complex with the Agrp promoter. Histones are acetylated and the chromatin is in an open state, Agrp transcription is on, and the neurons are active. The lack of inhibition of expression of Agrp, Npy, and Slc32a1 by TET3 enables sustained production and synaptic release of AGRP, NPY, and GABA. The physiological outcomes are increased food intake and decreased energy expenditure. FIG. 13B depicts a schematic representation of the herein described model in a fed state, where a rise in leptin level promotes binding of phosphorylated STAT3 to the Agrp promoter which in turn recruits TET3 and the chromatin-modifying complex. Binding of TET3 induces 5hmC modification which is required for a stable association of STAT3 and the chromatin-modifying complex with the promoter. The chromatin-modifying complex promotes histone deacetylation thereby inducing a closed chromatin state and inhibition of transcription of Agrp. Neuronal activity is also suppressed in part by a TET3-mediated mechanism presently being investigated. The expression of all three genes is reduced. The physiological outcomes are decreased food intake and increased energy expenditure. FIG. 13C depicts a schematic representation of the herein described model in a fed state without TET3 expression, where there is no 5hmC modification, activated STAT3 is unable to stably associate with the Agrp promoter to allow recruitment of the chromatin-modifying complex, histones remain acetylated, the chromatin is open, and Agrp transcription is not inhibited. In addition, the neuron remains active due to the lack of inhibition from the TET3-dependent mechanism. The lack of inhibition of expression of all three genes enables sustained production and synaptic release of AGRP, NPY, and GABA. The physiological outcomes are increased food intake and decreased energy expenditure. The sustained neuronal activity and synaptic release of AGRP, NPY, and GABA also promote anti-stress effects. FIG. 13D depicts a schematic representation of AgRP neuron-specific TET3 knockdown.

FIG. 14 depicts representative list of qPCR primer sequences and ChIP-qPCR/hMeDIP-qPCR primer sequences.

FIG. 15, comprising FIG. 15A through FIG. 15D, depicts representative experimental results demonstrating that Bobcat339 induces TET3 protein degradation and increases the expression of Agrp/AGRP, Npy/NPY, and Slc32a1/SLC32A1 in a TET3-dependent manner. FIG. 15A depicts representative quantification of TET3 levels in mouse GT1-7 cells incubated with vehicle or Bobcat339 at a final concentration of 10 μM in growth medium for 6 hours. The top panel depicts representative immunoblots for TET3 with GAPDH as a loading control, showing decreased TET3 expression at the protein level in Bobcat339-treated cells. The bottom panel depicts representative qPCR results of Tet3, showing no significant difference between the two groups. N=3 in technical replicates, statistical analysis was performed with a 2-tailed Student's t test. FIG. 15B depicts representative quantification of TET3 protein over time in GT1-7 cells. GT1-7 cells were incubated with vehicle or Bobcat339 at a final concentration of 10 μM for 3 hours followed by time course analysis of TET3 protein in the presence of cycloheximide (CHX) at a final concentration of 50 g/ml. Cells were harvested at 0, 1, 2, and 3 hours after addition of CHX. Bobcat339 was present in the growth media for a total of 6 hours. FIG. 15C depicts representative quantification of Agrp and Npy and Slc32a1 mRNA levels in GT1-7 cells incubated with vehicle plus GFP-expressing adenovirus (Veh+Ad), 10 μM Bobcat339 plus GFP-expressing adenovirus (Bc+Ad), or 10 μM Bobcat339 plus TET3-expressing adenovirus (Bc+Ad−TET3) for 48 hours, followed by RNA extraction and qPCR. There were significant increases in the Agrp/Nyp mRNAs in Bobcat339-treated cells, which was not seen when TET3 was overexpressed. N=3 per group in technical replicates; statistical analysis was performed with 1-way ANOVA with Tukey's post-test; **, p<0.01; ***, p<0.001. All data represent mean±SEM. FIG. 15D depicts representative quantification of Agrp/AGRP, Npy/NPY, and Slc32a1/SLC32A1 mRNA levels in SH-SY5Y cells incubated with vehicle plus GFP-expressing adenovirus (Veh+Ad), 10 μM Bobcat339 plus GFP-expressing adenovirus (Bc+Ad), or 10 μM Bobcat339 plus TET3-expressing adenovirus (Bc+Ad−TET3) for 48 hours, followed by RNA extraction and qPCR. There were significant increases in Agrp/NPY mRNAs in Bobcat339-treated cells, which was not seen when TET3 was overexpressed. N=3 per group in technical replicates; statistical analysis was performed with 1-way ANOVA with Tukey's post-test; **, p<0.01; ***, p<0.001. All data represent mean±SEM.

FIG. 16, comprising FIG. 16A through FIG. 16F, depicts representative results demonstrating Bobcat339 downregulated TET3 expression in AgRP neurons. FIG. 16A depicts representative microphotographs and corresponding statistical analysis of TET3⁺ (red) AgRP neurons (green) showing decreased TET3 protein in AgRP neurons in Bobcat339-treated mice. N=6 animals per group. Each dot represents an animal. FIG. 16B depicts representative qPCR of Tet3 showing no significant change in the ARCs of Bobcat339 vs. vehicle treated animals. N=8 animals per group. Each dot represents an animal. FIG. 16C depicts representative microphotographs of AGRP (red), NPY (red), and VGAT (red) showing a marked increase in their expressions in Bobcat339-treated mice. FIG. 16D depicts representative qPCR of indicated genes showing increased expressions of Agrp, Npy, and Slc32a1 in the ARCs of Bobcat339 treated animals. N=8 animals per group. Each dot represents an animal. FIG. 16E depicts representative quantification of AgRP neurons in the ARCs of mice treated with vehicle or Bobcat339 showing no significant difference between the groups. N=6 animals per group. Each dot represents an animal. FIG. 16F depicts representative microphotographs and corresponding statistical analysis of FOS' (red) AgRP neurons (green) showing increased expression of FOS in AgRP neurons in Bobcat339-treated mice. N=6 animals per group. Each dot represents an animal. All data represent the mean±SEM. *P<0.05, **P<0.01, and ***P<0.001, by 2-tailed Student's t test. 3V, third ventricle. Scale bars: 50 mm.

FIG. 17, comprising FIG. 17A through FIG. 17F, depicts representative design and results demonstrating that Bobcat339 mitigated anorexia. FIG. 17A depicts a schematic representation of the activity-based anorexia (ABA) experimental design. FIG. 17B depicts representative body weight change of mice with or without Bobcat339 treatment from the acclimation through recovery period. FIG. 17C depicts representative body mass breakdown of mice treated with or without Bobcat339 treatment. FIG. 17D depicts representative food intake during the acclimation period for mice with or without Bobcat339 treatment. FIG. 17E depicts representative food intake during the food restriction period for mice with or without Bobcat339 treatment. FIG. 17F depicts representative energy expenditure through wheel usage of mice with or without Bobcat339 treatment.

FIG. 18, comprising FIG. 18A through FIG. 18E, depicts representative behavioral test results in mice with ABA with or without Bobcat339 treatment. FIG. 18A depicts representative results of an open field test (OFT) for mice treated with or without Bobcat. FIG. 18B depicts representative results of a tail suspension test (TST) for mice treated with or without Bobcat. FIG. 18C depicts representative results of a forced swim test (FST) for mice treated with or without Bobcat. FIG. 18D depicts representative quantification of corticosterone levels in the blood of mice with or without Bobcat339 treatment. FIG. 18E depicts representative liver enzyme levels of mice with or without Bobcat339 treatment.

FIG. 19, comprising FIG. 19A and FIG. 19B, depicts representative imaging of co-expression of CD163⁺ cells with TET3 and TET2 in endometriosis lesions. FIG. 19A depicts a representative photomicrograph of human endometriosis lesions showing extensive co-expression of TET3 (red) with CD163 (green) and less so with TET2. FIG. 19B depicts representative photomicrographs of mouse endometriosis lesions. Nuclei are stained blue with DAPI. The panels on the right are zoomed-in images from the white rectangles marked on the left. Scale bars: 50 μm.

FIG. 20, comprising FIG. 20A through FIG. 20F, depicts representative experimental results demonstrating that siRNA-mediated TET3 knockdown induced apoptotic cell death. FIG. 20A depicts representative levels of mRNA in RAW 264.7 cells that were transfected with non-targeting siRNA (NT siRNA) or siRNA specifically targeting mouse TET3. RNAs were isolated at 24 hours after transfection and analyzed by qPCR. N=3 per group in technical replicates. FIG. 20B depicts representative immunoblots for TET3 from RAW 264.7 cells treated as in FIG. 20A. GAPDH was used as a loading control. Protein sizes in kDa are marked on the right. Proteins were isolated at the 48-hour timepoint. FIG. 20C depicts representative photomicrographs and TUNEL assay results of RAW 264.7 cells treated as in FIG. 20A with a TUNEL assay performed at the 48-hour timepoint. TUNEL⁺ (red) cells show a significant increase in apoptosis in TET3 knockdown cells. N=6 randomly selected areas per group. Scale bars: 50 μm. p-values were determined by 2-tailed Student's t test; *, p<0.05; **, p<0.01; ***, p<0.001. All data represented mean±SEM. FIG. 20D depicts representative results of THP-1 cells transfected with NT siRNA or siRNA specifically targeting human TET3 as in FIG. 20A. FIG. 20E depicts representative immunoblots for TET3 from THP-1 cells treated as in FIG. 20D. Proteins were isolated at the 48-hour timepoint. FIG. 20F depicts representative photomicrographs and TUNEL assay results of THP-1 cells as treated in FIG. 17D with a TUNEL assay performed at the 48-hour timepoint. TUNEL⁺ (red) cells show a significant increase in apoptosis in TET3 knockdown cells. N=6 randomly selected areas per group. Scale bars: 50 μm. p-values were determined by 2-tailed Student's t test; *, p<0.05; **, p<0.01; ***, p<0.001. All data represented mean±SEM.

FIG. 21, comprising FIG. 21A through FIG. 21G, depicts representative experimental results demonstrating that Bobcat339 promoted TET3 protein degradation and induced apoptotic cell death in a TET3-dependentg manner. FIG. 21A depicts representative quantification of TET3 mRNA and protein in RAW 264.7 cells that were incubated with vehicle or Bobcat339 at a final concentration of 10 μM in growth medium for 24 hours. The top panel depicts representative immunoblots for TET3 with GAPDH as a loading control, showing a decrease in TET3 at the protein level in Bobcat339-treated cells. The bottom panel depicts representative qPCR results of Tet3 showing no significant difference in expression between the groups by 2-tailed Student's t test; n=3 per group in technical replicate. FIG. 21B depicts representative quantification of TET3 mRNA and protein in THP-1 cells that were incubated with vehicle or Bobcat339 at a final concentration of 10 μM in growth medium for 24 hours. The top panel depicts representative immunoblots for TET3 with GAPDH as a loading control, showing a decrease in TET3 at the protein level in Bobcat339-treated cells. The bottom panel depicts representative qPCR results of Tet3 showing no significant difference in expression between the groups by 2-tailed Student's t test; n=3 per group in technical replicate. FIG. 21C depicts representative quantification of TET3 protein levels in RAW 264.7 cells incubated with vehicle or 10 μM Bobcat339 for 3 hours, followed by time course analysis of in the presence of cycloheximide (CHX) at a final concentration of 50 μg/ml. Cells were harvested at 0, 1, 2, and 3 hours after addition of CHX treatment. Bobcat339 was present in the growth media for a total of 6 hours. FIG. 21D depicts representative immunoblots for TET3 with GAPDH as a loading control, showing a decrease in TET3 at the protein level in Bobcat339-treated cells. FIG. 21E depicts representative photomicrographs and TUNEL assay results of RAW 264.7 cells incubated with vehicle plus GFP-expressing adenovirus (Veh+Ad), 10 μM Bobcat339 plus GFP-expressing adenovirus (Bc+Ad), or 10 μM Bobcat339 plus TET3-expressing adenovirus (Bc+Ad−TET3) for 48 hours followed by TUNEL assays. TUNEL⁺ (red) cells show a significant increase in apoptotic cell death in Bobcat339-treated cells, which was not seen when TET3 was overexpressed. N=6 randomly selected areas per group; p-value calculated by 1-way ANOVA with Tukey's post-test; ***, p<0.001; scale bars: 50 μm. All data represent mean±SEM. FIG. 21F depicts representative immunoblots for TET3 with GAPDH as a loading control, showing a decrease in TET3 at the protein level in Bobcat339-treated cells. FIG. 21G depicts representative photomicrographs and TUNEL assay results of THP-1 cells incubated with vehicle plus GFP-expressing adenovirus (Veh+Ad), 10 μM Bobcat339 plus GFP-expressing adenovirus (Bc+Ad), or 10 μM Bobcat339 plus TET3-expressing adenovirus (Bc+Ad−TET3) for 48 hours followed by TUNEL assays. TUNEL⁺ (red) cells show a significant increase in apoptotic cell death in Bobcat339-treated cells, which was not seen when TET3 was overexpressed. N=6 randomly selected areas per group; p-value calculated by 1-way ANOVA with Tukey's post-test; ***, p<0.001; scale bars: 50 μm. All data represent mean±SEM.

FIG. 22, comprising FIG. 22A through FIG. 22G, depicts representative experimental results demonstrating the decreased endometriosis burden resulting from treatment with Bobcat. N=5 animals per group. For FIG. 22B through FIG. 22D, p-values were calculated by 2-tailed Student's t test. For FIG. 22E through FIG. 22F, p-values were calculated by 1-way ANOVA with Tukey's post-test. *, p<0.05; **, p<0.01; ***, p<0.001. All data represent mean±SEM. FIG. 22A depicts a schematic representation of the experimental design described herein. FIG. 22B depicts representative photographs and corresponding statistical analysis of endometriosis lesions (marked by red circles) showing a significant decrease in lesion volume in Bobcat339-treated animals. FIG. 22C depicts representative photomicrographs and corresponding statistical analysis of endometriosis lesions stained with hematoxylin and eosin. Scale bars: 500 μm. FIG. 22D depicts representative photomicrographs of endometriosis lesions stained for TET3 (red) and CD163 (green). Cell nuclei were labeled blue by DAPI. Scale bar: 50 μm. FIG. 22E depicts representative body weight of mice. FIG. 22F depicts representative food intake of mice. FIG. 22G depicts representative plasma ALT, AST, and bilirubin levels in mice, showing no differences between the groups.

FIG. 23 depicts representative TET3 expression in CD163-positive cells in human patient tissue samples.

FIG. 24, comprising FIG. 24A through FIG. 24Q, depicts representative TET3 and CD163 expression in macrophages. Representative photomicrographs of immunohistochemistry of TET3 (red) and CD163 (green), with nuclei stained blue by DAPI, from human NASH liver tissues (n=4) (FIG. 24A and FIG. 24F), normal liver tissues (n=4) (FIG. 24B), peritoneal endometriosis tissues (n=3 patients) (FIG. 24C and FIG. 24F), normal eutopic endometrial tissues (n=3 patients) (FIG. 24D), lung adenocarcinoma tissues (n=5) (FIG. 24E and FIG. 24F), and matched adjacent normal tissues (n=5) (FIG. 24F). (FIG. 24G) TET2 expression in NASH, endometriosis and NSCLC. Representative photomicrographs of IHC of TET2 (red) and CD163 (green), with nuclei stained blue by DAPI, from human NASH liver tissues (n=4) (left, top), intraperitoneal endometriosis tissues (n=3) (right, top), and lung adenocarcinoma tissues (n=5) (bottom). The panels on the right are zoomed-in images from the left marked with white rectangles. Scale bar: 40 mm. (FIG. 24H) qPCR of TET3 mRNA from primary peripheral blood monocyte-derived macrophages (MDMs) treated with control media, CM-HSC, or CM-HSC plus TGF-β1 antibody for 72 h. (FIG. 24I) Representative photomicrographs and corresponding statistical analysis of immunohistochemistry of TET3 (red) and CD163 (green) in MDMs. N=3 randomly selected areas per group. (FIG. 24J) qPCR of TET3 mRNA from MDMs treated with control media, CM-Endo, or CM-Endo plus TGF-β1 antibody for 72 h. (FIG. 24K) Representative photomicrographs and corresponding statistical analysis of immunofluorescence staining of TET3 and CD163 in MDMs. N=3 randomly selected areas per group. (FIG. 24L) qPCR of TET3 mRNA from MDMs treated with control media or TGF-β1 at a final concentration of 10 ng/ml for 48 h. (FIG. 24M) Representative photomicrographs and corresponding statistical analysis of immunofluorescence staining of TET3 and CD163 in MDMs treated with control media or TGF-β1 at a final concentration of 10 ng/ml for 48 h. (FIG. 24N) qPCR of TET3 mRNA from MDMs treated with CTL or MCP1 at a final concentration of 200 ng/ml for 24 h. (FIG. 24O) Representative photomicrographs and corresponding statistical analysis of immunofluorescence staining of TET3 and CD163 in MDMs treated with CTL or MCP1 at a final concentration of 200 ng/ml for 24 h. n=3 randomly selected areas per group. (FIG. 24P) qPCR of TET3 mRNA from MDMs treated with CTL, CM-A549, or CM-A549 plus MCP1 antibody at a final concentration of 150 ng/ml for 48 h. (FIG. 24Q) Representative photomicrographs and corresponding statistical analysis of immunofluorescence staining of TET3 and CD163 in MDMs treated CTL, CM-A549, or CM-A549 plus MCP1 antibody at a final concentration of 150 ng/ml for 48 h. n=3 randomly selected areas per group. *P<0.05, **P<0.01, and ***P<0.001, by 2-tailed Student's t test. All data represent the mean±SEM. Scale bar: 40 mm.

FIG. 25, comprising FIG. 25A through FIG. 25H, depicts representative results demonstrating siRNA-mediated TET3 knockdown induced apoptosis. FIG. 25A depicts representative qPCR results of TET3 and TET2 mRNAs isolated from MDMs treated with CM-Endo and transfected with non-targeting siRNA (NT siRNA) or TET3 siRNA for 24 h. n=3 per group in technical replicates. FIG. 25B depicts representative immunoblots for TET3 from MDMs treated as in FIG. 25A. GAPDH was used as a loading control. Protein sizes in kDa are marked on the right. Proteins were isolated at the 48-hour time point post-transfection. FIG. 25C depicts representative results of M-PMBCs that were treated as in FIG. 25A, and TUNEL assay was performed at the 48-hour time point. Representative photomicrographs and corresponding statistical analysis of TUNEL+ (red) cells showing a significant increase in programmed cell death in TET3 knockdown cells. N=3 randomly selected areas per group. FIG. 25D depicts representative results of MDMs that were treated as in FIG. 25A, and caspase-1 assay was performed at the 48-hour time point. Representative photomicrographs and corresponding statistical analysis of caspase-1 positive (green) cells are shown. N=3 randomly selected areas per group. FIG. 25E depicts representative qPCR results of Tet3 and Tet2 mRNAs isolated from Raw 264.7 cells transfected with NT siRNA or Tet3 siRNA for 24 h. n=3 per group in technical replicates. FIG. 25F depicts representative immunoblots for TET3 from Raw 264.7 cells treated as in FIG. 25E. Proteins were isolated at the 48-hour time point. FIG. 25G depicts representative raw 264.7 cells that were treated as in FIG. 25E, and TUNEL assay was performed at the 48-hour time point. Representative photomicrographs and corresponding statistical analysis of TNEL+ cells are shown. N=3 randomly selected areas per group. FIG. 25H depicts representative raw 264.7 cells were treated as in FIG. 25E, and caspase-1 assay was performed at the 48-hour time point. Representative photomicrographs and corresponding statistical analysis of caspase-1 positive cells are shown. N=3 randomly selected areas per group. *P<0.05, **P<0.01, and ***P<0.001, by 2-tailed Student's t test. All data represent the mean±SEM. Scale bar: 50 μm.

FIG. 26, comprising FIG. 26A through FIG. 26O, depicts representative results demonstrating Bobcat339 promoted TET3 protein degradation and induced apoptosis in a TET3-dependent manner. FIG. 26A depicts representative results of human MDMs that were incubated with vehicle or Bobcat339 at a final concentration of 10 μM in CM-Endo for 24 hours. RNA and protein were extracted and analyzed. Top panels, representative immunoblots for TET3 and TET2 with GAPDH as a loading control, showing a decrease in TET3 (but not TET2) protein in Bobcat339 treated cells. Bottom panel, qPCR of TET3 and TET2 showing no significant differences in their expression between the two groups. N=3 per group in technical replicates. FIG. 26B depicts representative results of RAW 264.7 cells that were incubated with vehicle or Bobcat339 at a final concentration of 10 μM in growth media for 24 hours. RNA and protein were extracted and analyzed. N=3 per group in technical replicates. FIG. 26C depicts representative results of RAW 264.7 cells that were incubated with vehicle or Bobcat339 at a final concentration of 10 μM for 3 hours, followed by time course analysis of TET3 protein in the presence of cycloheximide (CHX) at a final concentration of 50 μg/ml. Cells were harvested at 0, 1, 2, and 3 hours after addition of CHX treatment. Bobcat339 was present in growth media for a total of 6 hours. FIG. 26D through FIG. 26I depicts representative results of MDMs and RAW 264.7 cells that were incubated with vehicle plus GFP-expressing adenovirus (Veh+Ad), Bobcat339 at 10 μM plus Ad (Bc+Ad), or Bobcat339 at 10 μM plus TET3-expressing adenovirus (Bc+Ad−TET3) for 48 hours, followed by TUNEL and Caspase-1 assays. Representative photomicrographs and corresponding statistical analysis of TUNEL+ (red) or Caspase-positive (green) cells showing a significant increase in pyroptotic cell death in Bobcat339-treated cells, which was not seen when TET3 expression level was restored. N=3 randomly selected areas per group. ***P<0.001, by 1-way ANOVA with Tukey's post-test. All data represent the mean±SEM. Scale bar: 50 μm. FIG. 26J depicts representative results of MDMs (treated with 10 ng/ml of TGF-β1) that were transfected with NT siRNA or TET3 siRNA, followed by RNA isolation and protein analysis at 48 h and 72 h, respectively. FIG. 26K depicts representative results of MDMs that were treated with Bobcat339 at a final concentration of 10 mM in the presence of 10 ng/ml of TGF-β1. RNA isolation and protein analysis were performed at 48 h and 72 h, respectively. *P<0.05, **P<0.01, and ***P<0.001, by 2-tailed Student's t test. FIG. 26L depicts representative qRT-PCR data of Il1b and Il6 mRNAs of cultured peritoneal macrophages isolated from Mye-Tet3 ko mice or WT controls and treated with 10 ng/ml LPS plus 20 ng/ml IFN-g. RNAs were isolated after 6 h of LPS/IFN-g stimulation. Uns, unstimulated. n=3 mice per genotype. FIG. 26M depicts representative results of ELISA analysis (after 6 h of LPS/IFN-g stimulation) of IL-1b and IL-6 of cultured peritoneal macrophages treated as in FIG. 26L. n=3 mice per genotype. FIG. 26N depicts representative results of human MDMs primed with 10 ng/ml of TGF-b1 that were transfected with NT siRNA or TET3 siRNA. After 48 h of transfection, cells were stimulated with 10 ng/mL LPS plus 20 ng/mL IFN-g for 8 h, followed by RNA extraction and qRT-PCR of IL1B and IL6 mRNAs. n=3 in biological replicates. FIG. 26O depicts representative results of ELISA analysis (after 8 h of LPS/IFN-g stimulation) of IL-1b and IL-6 of MDMs following treatment as in FIG. 26N.

FIG. 27, comprising FIG. 27A through FIG. 27E, depicts representative RNA-seq data validation. FIG. 27A depicts representative heat map showing relative levels of genes in RAW 264.7 cells transfected with NT siRNA or Tet3 siRNA for 48 h. Scale based on changes in log 2 expression. N=3 biological replicates in each group. FIG. 27B depicts representative results demonstrating top biological processes affected by TET3. FIG. 27C depicts representative log 2 expression levels of indicated genes in RAW 264.7 cells transfected with NT siRNA or Tet3 siRNA for 48 h. FIG. 27D depicts representative qPCR results of indicated genes in human MDMs in CM-Endo transfected with NT siRNA or TET3 siRNA (left panel) or in RAW 264.7 cells transfected with NT siRNA or Tet3 siRNA. RNAs were isolated at 48 hours post transfection. N=3 per group in technical replicates. FIG. 27E depicts representative nitric oxide concentration of human MDMs in Endo-CM treated with NT siRNA or Tet3 siRNA for 48 h. n=3 per group in technical replicates. *P<0.05, **P<0.01, and ***P<0.001, by 2-tailed Student's t test. All data represent the mean±SEM. (FIG. 27D and FIG. 27E) Results are representatives of at least two independent experiments.

FIG. 28, FIG. 28A through FIG. 28I, depicts representative results demonstrating depletion of TET3-expressing macrophages reduced endometriosis burden. FIG. 28A depicts representative photographs and corresponding statistical analysis of endometriosis lesions (marked by red circles) showing a significantly decrease in the lesion volume in ko animals. N=5 mice per group. FIG. 28B depicts representative photomicrographs and corresponding statistical analysis of endometriosis lesions stained with H&E. n=5 mice per group. Scale bar: 500 μm. FIG. 28C depicts representative microphotographs of endometriosis lesions co-stained for TET3 (red) with CD163 (green), TET3 (red) with CD11b (green), or TET3 (red) with F4/80 (green). Cell nuclei were labeled blue by DAPI. Scale bars: 50 μm. N=5 animals per group. FIG. 28D depicts representative schematic diagram of experimental design.

FIG. 28E depicts representative photographs and corresponding statistical analysis of endometriosis lesions showing a significantly decrease in lesion volume in Bobcat339-treated animals. N=5 mice per group. FIG. 28F depicts representative microphotographs and corresponding statistical analysis of endometriosis lesions stained with H&E. n=5 mice per group. Scale bar: 500 μm. FIG. 28G depicts representative microphotographs of endometriosis lesions co-stained for TET3 (red) with CD163 (green), TET3 (red) with CD11b (green), or TET3 (red) with F4/80 (green). Cell nuclei were labeled blue by DAPI. Scale bars: 50 μm. FIG. 28H depicts representative plasma ALT, AST, and bilirubin from mice showing no differences between the groups. N=5 animals per group. Each dot represents an animal. FIG. 28I depicts representative results demonstrating the body weight of mice. N=5 animals per group. Each dot represents an animal. All data represent the mean±SEM. (A, B, E, and F), *P<0.05 and ***P<0.001, by 2-tailed Student's t test. (FIG. 28H and FIG. 28I), **P<0.01, by 1-way ANOVA with Tukey's post-test. Ns, not statistically significant.

FIG. 29, comprising FIG. 29A through FIG. 29J, depicts representative results demonstrating Bobcat339 mitigated fibrotic NASH. FIG. 29A depicts a representative schematic diagram of experimental design. FIG. 29B depicts representative results of glucose tolerance tests performed at week 8 and week 12, respectively, on mice treated as indicated. N=5 mice per group. AUC, area under the curve. Plasma AST (FIG. 29C), liver triglycerides and liver-to-body weight ratio (FIG. 29D), NAS score (FIG. 29E), liver fibrosis stage and liver tissue hydroxyproline content (FIG. 29F) from mice treated as indicated. N=5 mice per group, each dot represents an animal. All data represent the mean±SEM. **P<0.01 and ***P<0.001, by 1-way ANOVA with Tukey's post-test. Ns, not statistically significant. FIG. 29G depicts representative Sirius Red/Fast Green- and H&E-stained liver histology images from mice (n=5 animals per group) treated as indicated (top two panels) and representative microphotographs of liver sections co-stained for CD163 (green) and TET3 (red), with cell nuclei labeled blue by DAPI (bottom panels). Scale bar: 50 μm. FIG. 29H depicts representative immunostaining of TET3 (red) in CD163+ macrophages (green) and quantification of macrophage TET3 MFI in liver tissue sections. n=5 mice per genotype. FIG. 29I depicts representative immunostaining of IL-1b (red) and CD163' macrophages (green) and quantification of macrophage IL-1b MFI in liver tissue sections. n=5 mice per genotype. FIG. 29J depicts representative immunostaining of IL-6 (red) and CD163+ macrophages (green) and quantification of macrophage IL-6 MFI in liver tissue sections. n=5 mice per genotype.

FIG. 30, comprising FIG. 30A through FIG. 30T, depicts representative results demonstrating that Bobcat339 attenuated lung cancer. FIG. 30A depicts a schematic representation of the experimental design. FIG. 30B depicts representative percent body weight changes on day 16 vs. day 0. FIG. 30C depicts representative results demonstrating body composition on day 16. FIG. 30D depicts representative results demonstrating five-day average food intake. FIG. 30E depicts representative results demonstrating lung weight on day 20.

FIG. 30F depicts representative results demonstrating the survival rate. FIG. 30G depicts representative macroscopic pictures and H&E stains (×10 magnification) of lungs of mice injected with LLC and treated with Veh (top) or Bobcat339 (bottom). FIG. 30H depicts representative photomicrographs of IHC of TET3 (red) and CD163 (green) with nuclei stained blue by DAPI. Scale bar: 40 mm. FIG. 30I depicts a schematic representation of a model that demonstrates how pathogenic disease-associated macrophages (DAMs), which overexpress TET3, rise from a heterogeneous population. Factors (e.g., TGF-β1 and MCP1) from the disease microenvironment upregulated TET3 expression in a subset of them. TET3 overexpression transformed these macrophages into a pathogenic subset of DAMs capable of producing inflammatory cytokines including TGF-β1, IL-1b and IL-6. TGF-β1 acted both as autocrine (upregulating TET3 expression in DAMs) and paracrine signaled to fuel disease progression. Bobcat339 induced TET3 degradation, thereby eradicating pathogenic DAMs and inhibiting disease progression. FIG. 30J depicts representative qRT-PCR results of let-7a in RAW 264.7 cells transfected with NT siRNA or Tet3 siRNA. RNA was isolated at 24 h post transfection. n=3 technical replicates. FIG. 30K depicts representative qRT-PCR results of let-7a in cultured peritoneal macrophages isolated from WT and Mye-Tet3 ko mice. n=3 mice per genotype. FIG. 30L depicts representative results demonstrating peritoneal macrophages (PM) that were isolated from WT mice and treated with TGF-b1 at a final concentration of 30 ng/ml. After 48 h, vehicle or Bobcat339 was added at a final concentration of 10 μM and incubation carried out for 48 h. RNAs were extracted and analyzed by qRT-PCR. n=3 mice per group. FIG. 30M depicts representative qRT-PCR results of Lin28 mRNA in PM isolated from WT and Mye-Tet3 ko mice. n=3 mice per genotype. FIG. 30N representative results of PM that were isolated from WT mice and treated with TGF-b1 at a final concentration of 30 ng/ml. After 48 h, vehicle or Bobcat339 was added at 10 μM and incubation carried out for 48 h. RNAs were extracted and analyzed by qRT-PCR. n=3 mice per group. FIG. 30O depicts representative qRT-PCR results of Il1b and 116 mRNAs of PM isolated from WT mice and transfected with control miRNA (miCon) or let-7a mimic and stimulated with 10 ng/ml LPS plus 20 ng/ml IFN-g. RNAs were isolated after 6 h of LPS/IFN-g stimulation. Uns, unstimulated. n=3 mice per genotype. FIG. 30P depicts representative ELISA results of IL-1b (after 6 h of LPS/IFN-g stimulation) and IL-6 (after 10 h of LPS/IFN-g stimulation) of PM treated as in f. n=3 mice per genotype. FIG. 30Q depicts representative relative let-7a miRNA levels in endometriosis lesions from WT and Mye-tet3 ko mice. n=4 animals per genotype. FIG. 30R depicts representative relative let-7a miRNA levels in endometriosis lesions from WT mice treated with vehicle or Bobcat339. n=4 animals per group. FIG. 30S depicts representative relative let-7a miRNA levels in liver tissues isolated from NASH mice treated with vehicle or Bobcat339. n=4 animals per group. FIG. 30T depicts representative relative let-7a miRNA levels in lung tissues isolated from vehicle or Bobcat339 treated mice. n=4 animals per group. All data represent the mean SEM. *P<0.05, **P<0.01, and ***P<0.001, by 2-tailed Student's t test.

FIG. 31 depicts representative expressions of Tet3 and Tet2 in the mouse peritoneal macrophages. qPCR of Tet3 and Tet2 showing ~90% decreased expression of Tet3 (but not Tet2) in the peritoneal macrophages of Mye-Tet3 ko mice compared to WT mice. n=3 animals per group. Each dot represents an animal. Data represent the mean±SEM. *** P<0.001, by 2-tailed Student's t test.

FIG. 32, comprising FIG. 32A and FIG. 32B, depicts representative results of steady-state myeloid linage characterization in WT and Mye-Tet3 ko mice. FIG. 32A depicts representative results of myeloid lineage analysis of spleen. Splenocytes were harvested from WT or Mye-Tet3 ko mice. Cells were stained with the indicated markers. Left: representative flow cytometry plots and the gating strategy are shown. Right: quantification of the indicated populations, with values representing the population percentages within the parent gate and total numbers in spleen. n=5 animals per group. FIG. 32B depicts representative results of similar analysis as in FIG. 32A that was performed on bone marrow cells. n=5 animals per group. Each dot represents an animal. All data represent the mean±SEM. 2-tailed Student's t test were used to compare means between groups.

FIG. 33, comprising FIG. 33A through FIG. 33C, depicts representative results demonstrating body weight, body composition, and fasting blood glucose levels of mice. Body weight, body composition, and fasting blood glucose levels of WT (n=11) and Mye-Tet3 ko (n=9) female mice at the age of 8 weeks (FIG. 33A) and 10 weeks (FIG. 33B), respectively. Each dot represents a mouse. Data represents the mean±SEM. 2-tailed Student's t test was used to compare means between groups. ns, not statistically significant. FIG. 33C depicts representative body weight of mice. n=5 animals per group. Each dot represents a mouse. Data represents the mean±SEM. 2-tailed Student's t test was used to compare means between groups. ns, not statistically significant.

FIG. 34 depicts representative results demonstrating fasting blood glucose. Mice were treated as in FIG. 29A. Fasting blood glucose concentrations were measured following a 16-h overnight fasting. n=5 mice per group. Each dot represents an animal. Data represent the mean±SEM. *P<0.05, **P<0.01, and ***P<0.001, by 1-way ANOVA with Tukey's post-test. ns, not statistically significant.

FIG. 35 depicts representative immunofluorescence staining using anti-TET3 (GeneTex, GTX121453, diluted at 1:400) that showed a significant reduction in TET3 (red) in TET3 siRNA-transfected as compared to NT siRNA-transfected cells. Human primary peripheral blood monocyte-derived macrophages (MDM) and human hepatic stellate cells (LX-2) were transfected with TET3 siRNA (siRNA specifically targeting human TET3) or NT siRNA (control nontargeting siRNA) for 48 h. Cell nuclei were stained blue by DAPI. MDMs were treated with TGF-b1 at a final concentration of 10 ng/ml to increase TET3 expression. Scale bar: 40 μm.

FIG. 36 depicts representative immunofluorescence staining of TET3 (red), CD163 (green), and nuclei (blue) on lung adenocarcinoma tissue sections from 2 patients. Images from three different tumor areas per patient and images from three non-tumor areas (Adjacent) are shown. While TET3 negative CD163 macrophages were seen both in tumor and non-tumor areas, TET3/CD163 double-positive macrophages were present only in tumor areas albeit with varying abundance between areas and patients. Scale bar: 40 μm.

FIG. 37, comprising FIG. 37A through FIG. 37E, depicts representative results of RNA-seq analysis demonstrating that TET3 negatively affected macrophage pro-apoptosis gene expression. FIG. 37A depicts representative qRT-PCR results of Bcl2l11, Bid, and Pmaip1 in unstimulated Raw 264.7 macrophages transfected with Tet3 siRNA or NT siRNA for 48 h. FIG. 37B depicts representative qRT-PCR results of BCL2L11, BID, and PMAIP1 in human MDMs primed with TGF-b1 at 10 ng/mL and transfected with TET3 siRNA or NT siRNA for 36 h. FIG. 37C depicts representative results of peritoneal macrophages (PM) that were isolated from WT mice and treated with TGF-b1 at a final concentration of 30 ng/mL. After 48 h, vehicle or Bobcat339 was added at a final concentration of 10 μM and incubation carried out for 48 h. RNAs were extracted and analyzed by qRT-PCR. FIG. 37D depicts representative results of mouse peritoneal macrophages that were treated as in FIG. 37C. Proteins were isolated for western blot analysis. Representative immunoblots of TET3 and TET2 are shown. FIG. 37E depicts representative results of mouse peritoneal macrophages that were treated as in FIG. 37C. TUNEL assays were performed after 48 h of treatment with Bobcat339 or vehicle. Representative photomicrographs and corresponding statistical analysis of TUNEL+ (red) cells are shown. n=3 randomly selected areas per group. All data represent the mean SEM. **P<0.01 and ***P<0.001, by 2-tailed Student's t test. Scale bar: 40 μm.

FIG. 38, FIG. 38A through FIG. 38D, depicts representative results demonstrating the effects of Tet3 deficiency on the expression of proinflammatory genes by cultured peritoneal macrophages. FIG. 38A depicts representative results of peritoneal macrophages that were isolated from Mye-Tet3 ko mice or WT controls (n=3 mice per genotype) and treated with 10 ng/mL LPS and 20 ng/mL IFNγ. RNAs were isolated after 10 h (Cxcl1), 6 h (Il1a, Ccl2, Ccl3, Ccl5, Cxcl2, Cxcl3, Cox2), or 4 h (Nos2 and Nlrp3) of LPS/IFNγ stimulation. mRNA levels of proinflammatory cytokines (FIG. 38A), chemokines (FIG. 38B), enzymes (FIG. 38C) and the main NLRP3 inflammasome component (FIG. 38D) were quantified by qRT-PCR. All data represent the mean±SEM. *P<0.05, **P<0.01, and ***P<0.001, by 2-tailed Student's t test.

FIG. 39, comprising FIG. 39A and FIG. 39B, depicts representative results demonstrating that PD-L1 was highly expressed in TAMs in LLC lung cancer. FIG. 39A depicts a representative immunostaining image of PD-L1 (red) in CD163⁺ macrophages (green) in tumor areas, left panel. FIG. 39A, right panel, depicts a representative immunostaining image of PD-L1 (red) in Mac2⁺ macrophages (green) in non-tumor areas. FIG. 39B depicts representative results of quantification of macrophage PD-L1 MFI in tumor and non-tumor areas. Data represent the mean±SEM. n=5 mice per group. **P<0.01, by 2-tailed Student's t test. Scale bar: 40 mm.

FIG. 40 depicts representative images of breast cancer, invasive ductal carcinoma and breast cancer, invasive lobular carcinoma. Immunofluorescence staining of TET3 (red) and FAP (fibroblast activation protein, green) and merged on human breast cancer tissue sections from two patients. Images from three different tumor areas per patient are shown. TET3/FAP double-positive CAFs were seen in both patient tissue samples. FAP is a marker for cancer-associated fibroblasts (CAFs).

FIG. 41 depicts representative qPCR primer sequences.

### DETAILED DESCRIPTION

The present invention is based, in part, on the unexpected discovery that inhibition of TET3 protein in the hypothalamic AgRP neurons induces hyperphagia. Thus, in one aspect, the present invention comprises methods of treating or preventing diseases or disorders associated with food intake, such as cancer-induced anorexia and anorexia nervosa, in a subject in need thereof by inhibiting one or more TET protein.

In another aspect, the present invention provides methods of treating or preventing a disease or disorder associated with the level of at least one TET protein, DAM, CAF, and/or AgRP neurons in a subject in need thereof. In one embodiment, the disease or disorder is a disease or disorder associated with food intake, disease or disorder associated with reduced appetite level, including cancer-induced anorexia, anorexia nervosa, gynecological disease, including endometriosis and uterine fibroids, anxiety, stress-related disorder, depressive-like behavior, depression, cancer-induced depression, postpartum depression, major depression, non-alcoholic fatty liver disease (NAFLD) including non-alcoholic steatohepatitis (NASH) and liver fibrosis, and cancer, including liver cancer, ovarian cancer, leukemia, AML, breast cancer, pancreatic cancer, lung cancer, glioma, and/or bladder cancer, disease or disorder associated with chronic inflammation, including NAFLD, cardiovascular disease, inflammatory bowel disease (IBD), Alzheimer's disease, Parkinson's disease, endometriosis, cancer, cancer-associated disease or disorder, or any combination thereof.

In some embodiments, the method comprises administering a degrader of TET protein, inhibitor of TET protein, or a combination thereof, or a composition thereof to the subject. In one embodiment, the degrader of TET protein is 4-amino-1-[1,1′-biphenyl]-3-yl-5-chloro-2(1H)-pyrimidinone or a derivative, analog, pharmaceutically acceptable salt, hydrate, or prodrug thereof.

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

The terms “patient,” “subject,” “individual,” and the like are used interchangeably herein, and refer to any animal, in some embodiments a mammal, and in some embodiments a human, having a complement system, including a human in need of therapy for, or susceptible to, a condition or its sequelae. The individual may include, for example, dogs, cats, pigs, cows, sheep, goats, horses, rats, monkeys, and mice and humans.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, AML, cervical cancer, skin cancer, pancreatic cancer, colorectal cancer, renal cancer, liver cancer, brain cancer, lymphoma, leukemia, lung cancer, and the like.

As used herein, “associated” refers to coincidence with the development or manifestation of a disease, condition, or phenotype. Association may be due to, but is not limited to, genes or gene products responsible for housekeeping functions, those that are part of a pathway that is involved in a specific disease, condition, or phenotype and those that indirectly contribute to the manifestation of a disease, condition or phenotype.

A disease or disorder is “alleviated” if the severity of at least one sign or symptom of the disease or disorder, the frequency with which such at least one sign or symptom is experienced by a patient, or both, is reduced.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, such as a human.

The terms “inhibit” and “inhibition,” as used herein, means to reduce, suppress, diminish or block an activity or function by at least about 10% relative to a control value. In some embodiments, the activity is suppressed or blocked by at least about 50% compared to a control value. In some embodiments, the activity is suppressed or blocked by at least about 75%. In some embodiments, the activity is suppressed or blocked by at least about 95%.

As used herein, the term “treatment” or “treating” is defined as the application or administration of a therapeutic agent, i.e., a compound of the invention (alone or in combination with another pharmaceutical agent), to a patient, or application or administration of a therapeutic agent to an isolated tissue or cell from a patient (e.g., for diagnosis or ex vivo applications), who has a disease or disorder contemplated herein, a sign or symptom of a disease or disorder contemplated herein or the potential to develop a disease or disorder contemplated herein, with the purpose to cure, heal, alleviate, relieve, alter, remedy, ameliorate, improve or affect a disease or disorder contemplated herein, the signs or symptoms of a disease or disorder contemplated herein or the potential to develop a disease or disorder contemplated herein. Such treatments may be specifically tailored or modified, based on knowledge obtained from the field of pharmacogenomics.

As used herein, “treating a disease or disorder” means reducing the frequency and/or severity of a sign and/or symptom of the disease or disorder is experienced by a patient.

A “therapeutic” treatment is a treatment administered to a subject who exhibits signs or symptoms of pathology disease or disorder, for the purpose of diminishing or eliminating those signs or symptoms.

The term “compound,” as used herein, unless otherwise indicated, refers to any specific chemical compound disclosed herein. In one embodiment, the term also refers to stereoisomers and/or optical isomers (including racemic mixtures) or enantiomerically enriched mixtures of disclosed compounds.

As used herein, the term “analog,” “analogue,” or “derivative” is meant to refer to a chemical compound or molecule made from a parent compound or molecule by one or more chemical reactions. As such, an analog can be a structure having a structure similar to that of the small molecule therapeutic agents described herein or can be based on a scaffold of a small molecule therapeutic agents described herein, but differing from it in respect to certain components or structural makeup, which may have a similar or opposite action metabolically. An analog or derivative can also be a small molecule that differs in structure from the reference molecule, but retains the essential properties of the reference molecule. An analog or derivative may change its interaction with certain other molecules relative to the reference molecule. An analog or derivative molecule may also include a salt, an adduct, tautomer, isomer, or other variant of the reference molecule.

The term “tautomers” are constitutional isomers of organic compounds that readily interconvert by a chemical process (tautomerization).

The term “isomers” or “stereoisomers” refer to compounds, which have identical chemical constitution, but differ with regard to the arrangement of the atoms or groups in space.

The term “prodrug” refers to compounds that differ in structure from the reference molecule, but is chemically modified by a particular cellular process to ultimately become modified to retain the essential properties of the reference molecule or become the reference molecule.

As used herein, the term “alkyl,” by itself or as part of another substituent means, unless otherwise stated, a straight or branched chain hydrocarbon having the number of carbon atoms designated (i.e. C₁₋₆ means one to six carbon atoms) and includes straight, branched chain, or cyclic substituent groups. Examples include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. The term “alkyl,” unless otherwise noted, is also meant to include those derivatives of alkyl defined in more detail below, such as “heteroalkyl”, “haloalkyl” and “homoalkyl”.

As used herein, the term “substituted alkyl” means alkyl, as defined above, substituted by one, two or three substituents selected from the group consisting of halogen, —OH, alkoxy, —NH₂, —N(CH₃)₂, —C(═O)OH, trifluoromethyl, —C≡N, —C(═O)O(C₁-C₄)alkyl, —C(═O)NH₂, —SO₂NH₂, —C(═NH)NH₂, and —NO₂, preferably containing one or two substituents selected from halogen, —OH, alkoxy, —NH₂, trifluoromethyl, —N(CH₃)₂, and —C(═O)OH, more preferably selected from halogen, alkoxy and —OH. Examples of substituted alkyls include, but are not limited to, 2,2-difluoropropyl, 2-carboxycyclopentyl and 3-chloropropyl.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

As used herein, the term “cycloalkyl” or “carbocyclyl” refers to a mono cyclic or polycyclic non-aromatic radical, wherein each of the atoms forming the ring (i.e. skeletal atoms) is a carbon atom. In one embodiment, the cycloalkyl group is saturated or partially unsaturated. In another embodiment, the cycloalkyl group is fused with an aromatic ring. Cycloalkyl groups include groups having from 3 to 10 ring atoms. Illustrative examples of cycloalkyl groups include, but are not limited to, the following moieties:

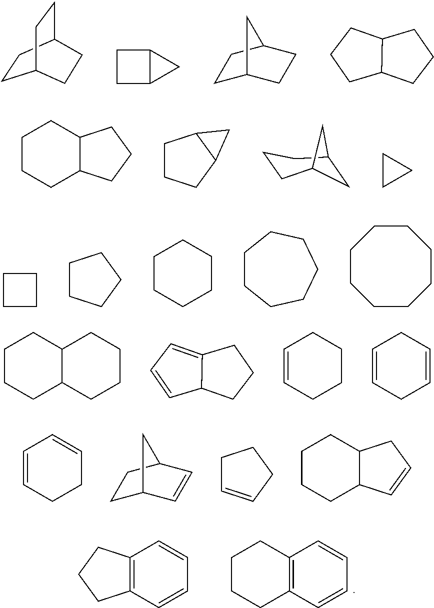

Monocyclic cycloalkyls include, but are not limited to, cyclopropyl, cyclobutyl, cyclopentyl, cyclohexyl, cycloheptyl, and cyclooctyl. Dicyclic cycloalkyls include, but are not limited to, tetrahydronaphthyl, indanyl, and tetrahydropentalene. Polycyclic cycloalkyls include adamantine and norbornane. The term cycloalkyl includes “unsaturated nonaromatic carbocyclyl” or “nonaromatic unsaturated carbocyclyl” groups, both of which refer to a nonaromatic carbocycle as defined herein, which contains at least one carbon carbon double bond or one carbon carbon triple bond.

As used herein, the term “heterocycle” or “heterocyclyl” or “heterocyclic” by itself or as part of another substituent means, unless otherwise stated, an unsubstituted or substituted, stable, mono- or multi-cyclic heterocyclic ring system that consists of carbon atoms and at least one heteroatom selected from the group consisting of N, O, and S, and wherein the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. An example of a 3-membered heterocycloalkyl group includes, and is not limited to, aziridine. Examples of 4-membered heterocycloalkyl groups include, and are not limited to, azetidine and a beta lactam. Examples of 5-membered heterocycloalkyl groups include, and are not limited to, pyrrolidine, oxazolidine and thiazolidinedione. Examples of 6-membered heterocycloalkyl groups include, and are not limited to, piperidine, morpholine and piperazine. Other non-limiting examples of heterocycloalkyl groups are:

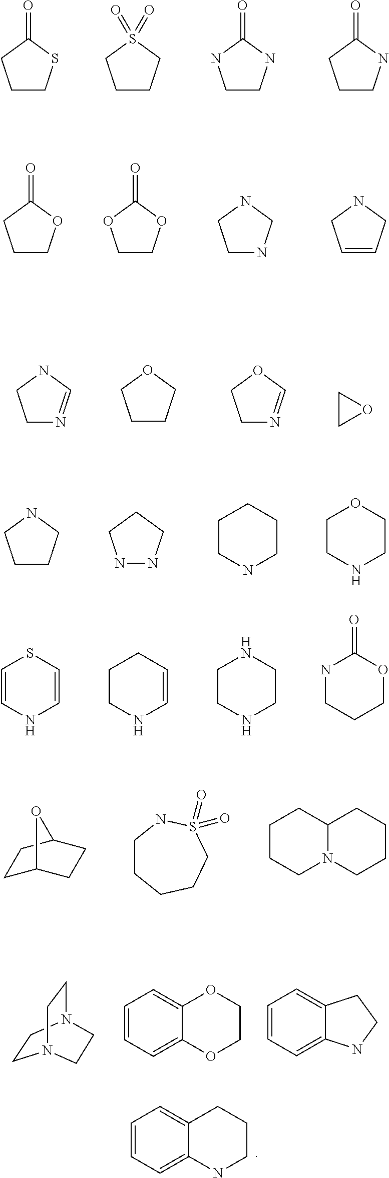

Examples of non-aromatic heterocycles include monocyclic groups such as aziridine, oxirane, thiirane, azetidine, oxetane, thietane, pyrrolidine, pyrroline, imidazoline, pyrazolidine, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin and hexamethyleneoxide.

As used herein, the term “aryl,” employed alone or in combination with other terms, means, unless otherwise stated, a carbocyclic aromatic system containing one or more rings (typically one, two or three rings) wherein such rings may be attached together in a pendent manner, such as a biphenyl, or may be fused, such as naphthalene. Examples include phenyl, anthracyl, and naphthyl. Preferred are phenyl and naphthyl, most preferred is phenyl.

Examples of heteroaryl groups include pyridyl, pyrazinyl, pyrimidinyl (particularly 2- and 4-pyrimidinyl), pyridazinyl, thienyl, furyl, pyrrolyl (particularly 2-pyrrolyl), imidazolyl, thiazolyl, oxazolyl, pyrazolyl (particularly 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles include indolyl (particularly 3-, 4-, 5-, 6- and 7-indolyl), indolinyl, quinolyl, tetrahydroquinolyl, isoquinolyl (particularly 1- and 5-isoquinolyl), 1,2,3,4-tetrahydroisoquinolyl, cinnolinyl, quinoxalinyl (particularly 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (particularly 3-, 4-, 5-, 6- and 7-benzofuryl), 2,3-dihydrobenzofuryl, 1,2-benzisoxazolyl, benzothienyl (particularly 3-, 4-, 5-, 6-, and 7-benzothienyl), benzoxazolyl, benzothiazolyl (particularly 2-benzothiazolyl and 5-benzothiazolyl), purinyl, benzimidazolyl (particularly 2-benzimidazolyl), benztriazolyl, thioxanthinyl, carbazolyl, carbolinyl, acridinyl, pyrrolizidinyl, and quinolizidinyl.

The aforementioned listing of heterocyclyl and heteroaryl moieties is intended to be representative and not limiting.

As used herein, the term “substituted” means that an atom or group of atoms has replaced hydrogen as the substituent attached to another group. For aryl, aryl-(C₁-C₄)alkyl and heterocyclyl groups, the term “substituted” as applied to the rings of these groups refers to any level of substitution, namely mono-, di-, tri-, tetra-, or penta-substitution, where such substitution is permitted. The substituents are independently selected, and substitution may be at any chemically accessible position. In one embodiment, the substituents vary in number between one and four. In another embodiment, the substituents vary in number between one and three. In yet another embodiment, the substituents vary in number between one and two. In yet another embodiment, the substituents are independently selected from the group consisting of C₁₋₆ alkyl, —OH, C₁₋₆ alkoxy, halo, amino, acetamido and nitro. In yet another embodiment, the substituents are independently selected from the group consisting of C₁₋₆ alkyl, C₁₋₆ alkoxy, halo, acetamido, and nitro. As used herein, where a substituent is an alkyl or alkoxy group, the carbon chain may be branched, straight or cyclic, with straight being preferred.

As used herein, the term “optionally substituted” means that the referenced group may be substituted or unsubstituted. In one embodiment, the referenced group is optionally substituted with zero substituents, i.e., the referenced group is unsubstituted. In another embodiment, the referenced group is optionally substituted with one or more additional group(s) individually and independently selected from groups described herein.

In one embodiment, the substituents are independently selected from the group consisting of oxo, halogen, —CN, —NH₂, —OH, —NH(CH₃), —N(CH₃)₂, alkyl (including straight chain, branched and/or unsaturated alkyl), substituted or unsubstituted cycloalkyl, substituted or unsubstituted heterocycloalkyl, fluoro alkyl, substituted or unsubstituted heteroalkyl, substituted or unsubstituted alkoxy, fluoroalkoxy, —S-alkyl, S(═O)₂alkyl, —C(═O)NH[substituted or unsubstituted alkyl, or substituted or unsubstituted phenyl], —C(═O)N[H or alkyl]2, —OC(═O)N[substituted or unsubstituted alkyl]2, —NHC(═O)NH[substituted or unsubstituted alkyl, or substituted or unsubstituted phenyl], —NHC(═O)alkyl, —N[substituted or unsubstituted alkyl]C(═O)[substituted or unsubstituted alkyl], —NHC(═O)[substituted or unsubstituted alkyl], —C(OH)[substituted or unsubstituted alkyl]2, and —C(NH₂)[substituted or unsubstituted alkyl]2. In another embodiment, by way of example, an optional substituent is selected from oxo, fluorine, chlorine, bromine, iodine, —CN, —NH₂, —OH, —NH(CH₃), —N(CH₃)₂, —CH₃, —CH₂C₃, —CH(CH₃)₂, —CF₃, —CH₂CF₃, —OCH₃, —OCH₂CH₃, —OCH(CH₃)₂, —OCF₃, —OCH₂CF₃, —S(═O)₂—CH₃, —C(═O)NH₂, —C(═O)—NHCH₃, —NHC(═O)NHCH₃, —C(═O)CH₃, —ON(O)₂, and —C(═O)OH. In yet one embodiment, the substituents are independently selected from the group consisting of C₁₋₆ alkyl, —OH, C₁₋₆ alkoxy, halo, amino, acetamido, oxo and nitro. In yet another embodiment, the substituents are independently selected from the group consisting of C₁₋₆ alkyl, C₁₋₆ alkoxy, halo, acetamido, and nitro. As used herein, where a substituent is an alkyl or alkoxy group, the carbon chain may be branched, straight or cyclic.

The term “antibody,” as used herein, refers to an immunoglobulin molecule, which specifically binds with an antigen. Antibodies can be intact immunoglobulins derived from natural sources or from recombinant sources and can be immunoreactive portions of intact immunoglobulins. The term “antibody” includes any antibody protein construct comprising at least one antibody variable domain comprising at least one antigen-binding site (ABS). Antibodies include, but are not limited to, immunoglobulins of types IgA, IgG, IgE, IgD, IgM (as well as subtypes thereof). The overall structure of Immunoglobulin G (IgG) antibodies assembled from two identical heavy (H)-chain and two identical light (L)-chain polypeptides is well established and highly conserved in mammals (Padlan et al., 1994, Mol. Immunol. 31:169-217). The antibody in the present invention may exist in a variety of forms where the antigen binding portion of the antibody is expressed as part of a contiguous polypeptide chain including, for example, a single domain antibody fragment (sdAb), a single chain antibody (scFv) and a humanized antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426).

As used herein, the language “pharmaceutically acceptable salt” refers to a salt of the administered compound prepared from pharmaceutically acceptable non-toxic acids, including inorganic acids, organic acids, solvates, hydrates, or clathrates thereof. Examples of such inorganic acids are hydrochloric, hydrobromic, hydroiodic, nitric, sulfuric, phosphoric, acetic, hexafluorophosphoric, citric, gluconic, benzoic, propionic, butyric, sulfosalicylic, maleic, lauric, malic, fumaric, succinic, tartaric, amsonic, pamoic, p-tolunenesulfonic, and mesylic. Appropriate organic acids may be selected, for example, from aliphatic, aromatic, carboxylic and sulfonic classes of organic acids, examples of which are formic, acetic, propionic, succinic, camphorsulfonic, citric, fumaric, gluconic, isethionic, lactic, malic, mucic, tartaric, para-toluenesulfonic, glycolic, glucuronic, maleic, furoic, glutamic, benzoic, anthranilic, salicylic, phenylacetic, mandelic, embonic (pamoic), methanesulfonic, ethanesulfonic, pantothenic, benzenesulfonic (besylate), stearic, sulfanilic, alginic, galacturonic, and the like. Furthermore, pharmaceutically acceptable salts include, by way of non-limiting example, alkaline earth metal salts (e.g., calcium or magnesium), alkali metal salts (e.g., sodium-dependent or potassium), and ammonium salts.

As used herein, the term “pharmaceutically acceptable” refers to a material, such as a carrier or diluent, which does not abrogate the biological activity or properties of the compound, and is relatively non-toxic, i.e., the material may be administered to an individual without causing an undesirable biological effect or interacting in a deleterious manner with any of the components of the composition in which it is contained.

As used herein, the terms “effective amount,” “pharmaceutically effective amount” and “therapeutically effective amount” refer to a sufficient amount of an agent to provide the desired biological or physiologic result. That result may be reduction and/or alleviation of a sign, a symptom, or a cause of a disease or disorder, or any other desired alteration of a biological system. An appropriate effective amount in any individual case may be determined by one of ordinary skill in the art using routine experimentation.

As used herein, the term “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary, and topical administration.

As used herein, the term “pharmaceutically acceptable carrier” means a pharmaceutically acceptable material, composition, or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, PA), which is incorporated herein by reference.

“Measuring” or “measurement,” or alternatively “detecting” or “detection,” means assessing the presence, absence, quantity, or amount (which can be an effective amount) of either a given substance within a sample, including the derivation of qualitative or quantitative concentration levels of such substances, or otherwise evaluating the values or categorization of the substance or the sample.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention is based, in part, on the unexpected discovery that inhibition of TET3 protein in the hypothalamic AgRP neurons induces hyperphagia. Thus, in one aspect, the present invention comprises methods of treating or preventing diseases or disorders associated with food intake, such as cancer-induced anorexia and anorexia nervosa, in a subject in need thereof by inhibiting one or more TET protein.

In another aspect, the present invention provides methods of treating or preventing a disease or disorder associated with the level of at least one TET protein, DAM, CAF, and/or agouti-related peptide-expressing neurons (AgRP neurons) in a subject in need thereof. In one embodiment, the disease or disorder is a disease or disorder associated with food intake, disease or disorder associated with reduced appetite level, including cancer-induced anorexia, anorexia nervosa, gynecological disease, including endometriosis and uterine fibroids, anxiety, stress-related disorder, depressive-like behavior, depression, cancer-induced depression, postpartum depression, major depression, non-alcoholic fatty liver disease (NAFLD) including non-alcoholic steatohepatitis (NASH) and liver fibrosis, and cancer, including liver cancer, ovarian cancer, leukemia, AML, breast cancer, lung cancer, glioma, and/or bladder cancer, disease or disorder associated with chronic inflammation, including NAFLD, cardiovascular disease, inflammatory bowel disease (IBD), Alzheimer's disease, Parkinson's disease, endometriosis, cancer, cancer-associated disease or disorder, or any combination thereof. In some embodiments, the method comprises administering a degrader of TET protein, inhibitor of TET protein, or a combination thereof, or a composition thereof to the subject. In one embodiment, the degrader of TET protein is 4-amino-1-[1,1′-biphenyl]-3-yl-5-chloro-2(1H)-pyrimidinone or a derivative, analog, pharmaceutically acceptable salt, hydrate, or prodrug thereof.

### Inhibitors of TET Protein

In some embodiments, the present invention provides a compound or composition thereof for modulating the level, activity, expression, stability, and/or degradation of at least one TET protein. Thus, in some embodiments, the present invention provides a compound or composition thereof for treating or preventing a disease or disorder associated with the level, activity, expression, stability, and/or degradation of at least one TET protein. In some embodiments, the present invention provides a compound or composition thereof for treating or preventing a disease or disorder associated with increased level, activity, expression, stability, and/or degradation of at least one TET protein.

In some embodiments, the compound or composition thereof decreases the activity of at least one TET protein, decreases the level of at least one TET protein, decreases the expression of at least one TET protein, decreases the function of at least one TET protein, decrease the stability of at least one TET protein, increases the degradation of at least one TET protein, or any combination thereof. In other embodiments, the compound or composition thereof inhibits the activity of at least one TET protein, inhibits the level of at least one TET protein, inhibits the expression of at least one TET protein, inhibits the function of at least one TET protein, inhibits the stability of at least one TET protein, induces the degradation of at least one TET protein, or any combination thereof.

In various embodiments, the at least one TET protein is in at least one AgRP cell (e.g., AgRP neuron), DAM (e.g., EAM or tumor-associated macrophage, lung cancer-associated macrophage, ovarian cancer-associated macrophage, leukemia-associated macrophage, AML-associated macrophage, breast cancer-associated macrophage, pancreatic cancer-associated macrophage, etc.), CAF (e.g., lung cancer-associated fibroblast, ovarian cancer-associated fibroblast, leukemia-associated fibroblast, AML-associated fibroblast, breast cancer-associated fibroblast, pancreatic cancer-associated fibroblast, etc.), cancer cell (e.g., ovarian cancer cell, leukemia cancer cell, AML cancer cell, breast cancer cell, lung cancer cell, pancreatic cancer cell, etc.), or any combination thereof. For example, in various embodiments, the at least one TET protein is in at least one AgRP neuron (e.g., hypothalamic AgRP neuron), DAM (e.g., EAM or tumor-associated macrophage, lung cancer-associated macrophage, ovarian cancer-associated macrophage, leukemia-associated macrophage, AML-associated macrophage, breast cancer-associated macrophage, pancreatic cancer-associated macrophage, etc.), CAF (e.g., lung cancer-associated fibroblast, ovarian cancer-associated fibroblast, leukemia-associated fibroblast, AML-associated fibroblast, breast cancer-associated fibroblast, pancreatic cancer-associated fibroblast, etc.), ovarian cancer cell, leukemia cancer cell, AML cancer cell, breast cancer cell, lung cancer cell, pancreatic cancer cell, or any combination thereof.

In one embodiment, the compound is a modulator of TET protein. In some embodiments, the modulator of TET protein is any compound, molecule, or agent that reduces, inhibits, or prevents the function of a TET protein. For example, an inhibitor of a TET protein is any compound, molecule, or agent that reduces expression of a TET protein, reduces function of a TET protein, reduces activity of a TET protein, induces degradation of a TET protein, or a combination thereof. In one embodiment, an inhibitor of a TET protein comprises a nucleic acid, a peptide, a small molecule, a siRNA, a ribozyme, an antibody, an antisense nucleic acid, an antagonist, an aptamer, a peptidomimetic, or any combination thereof. For example, in one embodiment, the modulator of TET protein is a degrader of TET protein, inhibitor of TET protein, or a combination thereof.

In one embodiment, the TET protein is at least one TET1 protein, TET2 protein, and/or TET3 protein.

Thus, in one embodiment, the composition comprises a modulator of TET1 protein, modulator of TET2 protein, modulator of TET3 protein, or any combination thereof.

In some embodiments, the modulator of TET protein inhibits an activity of a TET1 protein (e.g., oxidation of 5mC to 5hmC) by at least 10%, at least 20%, at least 30%, at least 40%, at least 50%, at least 60%, at least 70%, at least 80%, at least 90%, at least 99%, or at least 100%.

In some embodiments, the modulator of TET protein inhibits an activity of TET1 (e.g, oxidation of 5mC to 5hmC) by at least 10%, at least 20%, at least 30%, at least 40%, at least 50%, at least 60%, at least 70%, at least 80%, at least 90%, at least 99%, or at least 100% Thus, in one embodiment, the modulator of TET protein is a modulator of TET1 protein.

In some embodiments, the modulator of TET protein inhibits an activity of TET2 (e.g, oxidation of 5mC to 5hmC) by at least 10%, at least 20%, at least 30%, at least 40%, at least 50%, at least 60%, at least 70%, at least 80%, at least 90%, at least 99%, or at least 100%. Thus, in one embodiment, the modulator of TET protein is a modulator of TET2 protein.

In some embodiments, the modulator of TET protein inhibits an activity of TET3 (e.g, oxidation of 5mC to 5hmC) by at least 10%, at least 20%, at least 30%, at least 40%, at least 50%, at least 60%, at least 70%, at least 80%, at least 90%, at least 99%, or at least 100%. Thus, in one embodiment, the modulator of TET protein is a modulator of TET3 protein.

In some embodiments, the modulator of TET3 protein modulates the level or activity of TET3 protein. In some embodiments, the modulator of TET3 protein modulates the level or activity of TET3 protein and simultaneously does not affect the level or activity of TET1 protein and/or TET2 protein. For example, in some embodiments, the degrader of TET3 protein, inhibitor of TET3 protein, or a combination thereof decreases the level or activity of TET3 protein. In some embodiments, the degrader of TET3 protein, inhibitor of TET3 protein, or a combination thereof decreases the level or activity of TET3 protein and simultaneously does not affect the level or activity of TET1 protein and/or TET2 protein.

In one embodiment, the degrader of TET protein, inhibitor of TET protein, or a combination thereof is one or more small molecule.

In some embodiments, the degrader of TET protein, inhibitor of TET protein, or a combination thereof is a compound having the structure of Formula (I)

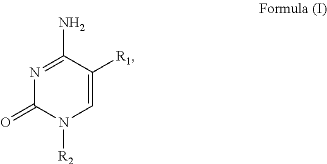

or a derivative, analog, pharmaceutically acceptable salt, hydrate, or prodrug thereof.

In some embodiments, R₁ is independently selected from the group consisting of hydrogen, halogen, optionally substituted alkyl, optionally substituted alkenyl, or optionally substituted alkynyl.

In one embodiment, R₁ is fluorine. In one embodiment, R₁ is chlorine. In one embodiment, R₁ is bromine. In one embodiment, R₁ is iodine.

In some embodiments, R₁ is substituted alkyl. In some embodiments, R₁ is unsubstituted alkyl. In some embodiments, R₁ is substituted C₁₋₆ alkyl. In some embodiments, R₁ is unsubstituted C₁₋₆ alkyl in one embodiment, R₁ is methyl. In one embodiments, R₁ is fluoromethyl. In one embodiment, R₁ is difluoromethyl. In one embodiment, R₁ is trifluoromethyl.

In some embodiments, R₁ is substituted alkenyl. In some embodiments, R₁ is unsubstituted alkenyl. In some embodiments, R₁ is vinyl. In one embodiment, R₁ is ethenyl.

In some embodiments, R₁ is substituted alkynyl. In some embodiments, R₁ is unsubstituted alkynyl. In one embodiment, R₁ is ethynyl. In one embodiment, R₁ is propargyl.

In some embodiments, R₂ is selected from selected from the group consisting of hydrogen, optionally substituted carbocyclyl, optionally substituted heterocyclyl, optionally substituted aryl, or optionally substituted heteroaryl.

In some embodiments, R₂ is substituted carbocyclyl. In some embodiments, R₂ is unsubstituted carbocyclyl. In some embodiments, R₂ is substituted heterocyclyl. In some embodiments, R₂ is unsubstituted heterocyclyl. In some embodiments, R₂ is substituted aryl. In some embodiments, R₂ is unsubstituted aryl. In some embodiments, R₂ is substituted heteroaryl. In some embodiments, R₂ is unsubstituted heteroaryl.

In some embodiments, R₂ is of formula —(CH₂)ₙC(═O)N(R^{A})₂, wherein n is 1, 2, or 3; and each instance of R^{A} is independently hydrogen, optionally substituted C₁₋₆ alkyl, or optionally substituted aryl. In some embodiments, n is 1. In some embodiments, n is 2. In some embodiments, n is 3. In some embodiments, R^{A} is hydrogen. In some embodiments, R^{A} is substituted C₁₋₆ alkyl. In some embodiments, R^{A} is unsubstituted C₁₋₆ alkyl. In some embodiments, R^{A} is substituted aryl. In some embodiments, R^{A} is unsubstituted aryl.

In some embodiments, R₂ is substituted phenyl. In some embodiments, R₂ is unsubstituted phenyl. In some embodiments, R₂ is phenyl substituted with halogen. In some embodiments, R₂ is phenyl substituted with chlorine. In some embodiments, R₂ is 4-chlorophenyl. In some embodiments, R₂ is 3-chlorophenyl. In some embodiments, R₂ is 2-chlorophenyl. In some embodiments, R₂ is phenyl substituted with C₁₋₆alkyl. In some embodiments, R₂ is 4-methylphenyl. In some embodiments, R₂ is 3-methylphenyl. In some embodiments, R₂ is 2-methylphenyl. In some embodiments, R₂ is 2-biphenyl. In some embodiments, R₂ is 3-biphenyl. In some embodiments, R₂ is 4-biphenyl.

In some embodiments, R₂ is substituted napthyl. In some embodiments, R₂ is unsubstituted napthyl. In some embodiments, R₂ is 1-napthyl. In some embodiments. R₂ is 2-napthyl.

In some embodiments, R₂ is substituted heteroaryl. In some embodiments, R₂ is unsubstituted heteroaryl. In some embodiments, R₂ is substituted pyridyl. In some embodiments, R₂ is unsubstituted pyridyl. In some embodiments, R₂ is substituted quinolyl. In some embodiments, R₂ is unsubstituted quinolyl. In some embodiments, R₂ is substituted dibenzofuranyl. In some embodiments, R₂ is unsubstituted dibenzofuranyl. In some embodiments of any one of the compositions or methods provided herein R₂ is substituted benzo[d]oxazolyl. In some embodiments, R₂ is unsubstituted benzo[d]oxazolyl.

In some embodiments, R₂ is nicotinonitrile. In some embodiments, R₂ is 5-methoxypyridin-2-yl. In some embodiments, R₂ is 4-dibenzofuranyl. In some embodiments, R₂ is unsubstituted 3-quinolinyl. In some embodiments, R₂ is 2-phenylbenzo[d]oxazol-6-yl. In some embodiments, R₂ is 2-phenylbenzo[d]oxazol-7-yl.

In some embodiments, R₂ is selected from the group consisting of

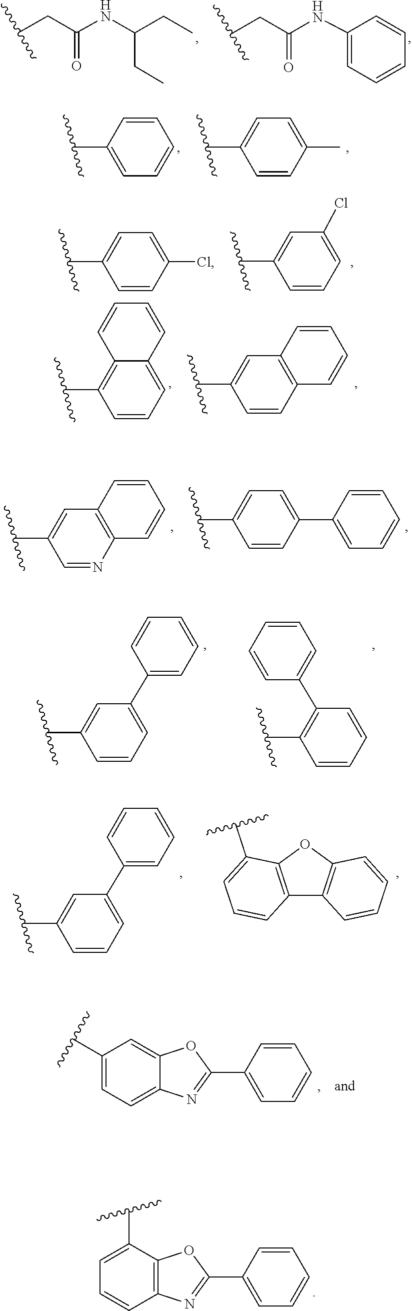

In some embodiments, the compound having the structure of Formula (I) is selected from the group consisting of

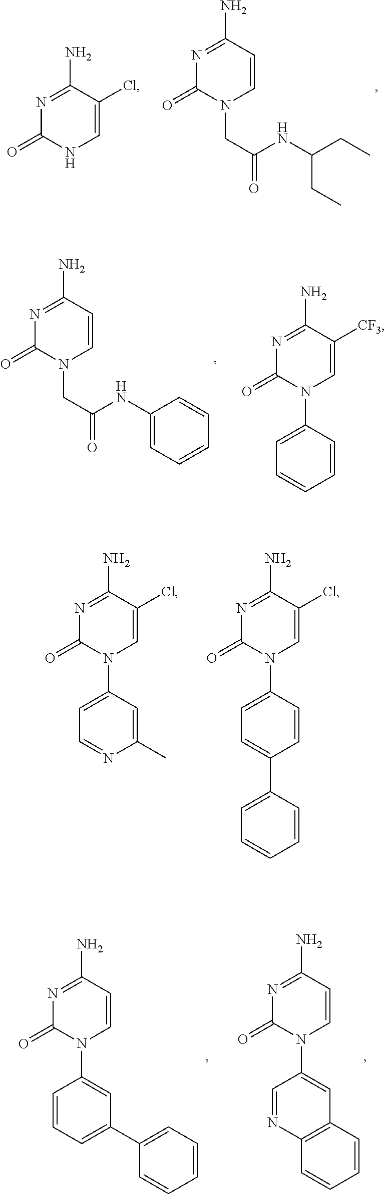

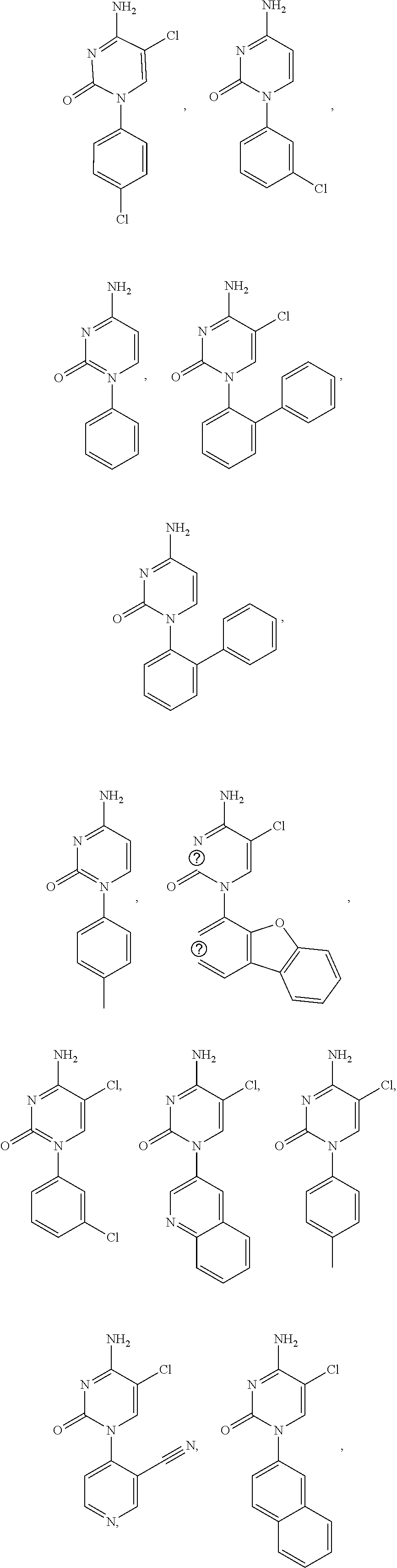

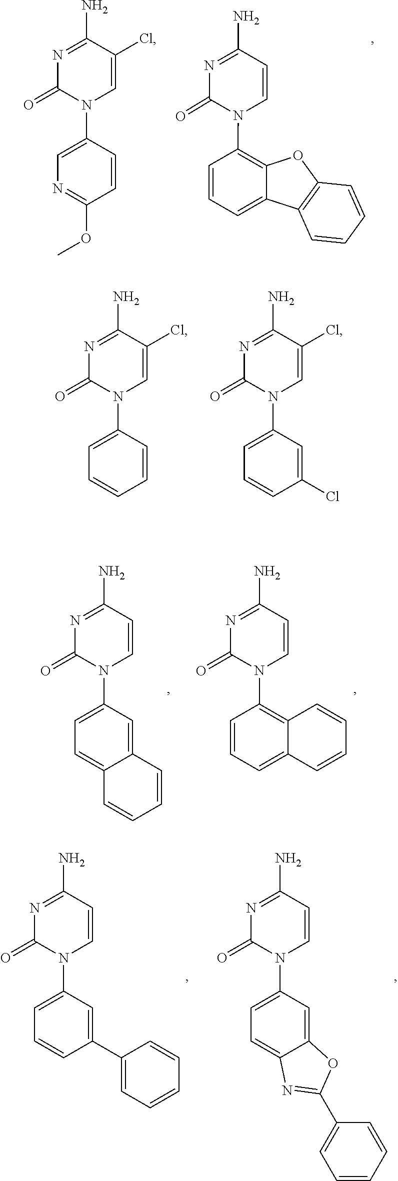

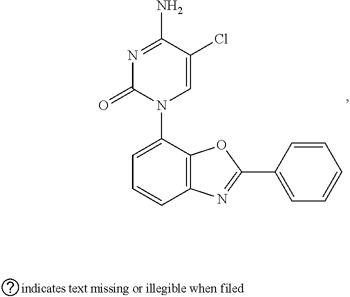 [SMILES: Nc1nc(=O)n(-c2cccc3nc(-c4ccccc4)oc23)cc1Cl]

or a derivative, analog, pharmaceutically acceptable salt, hydrate, or prodrug thereof.

For example, in some embodiments, the compound having the structure of Formula (1) is 4-amino-1-[1,1′-biphenyl]-3-yl-5-chloro-2(1H)-pyrimidinone or a derivative, analog, pharmaceutically acceptable salt, hydrate, or prodrug thereof.

### Small Molecule Inhibitors

In various embodiments, the inhibitor is a small molecule. When the inhibitor is a small molecule, a small molecule may be obtained using standard methods known to the skilled artisan. Such methods include chemical organic synthesis or biological means. Biological means include purification from a biological source, recombinant synthesis and in vitro translation systems, using methods well known in the art. In one embodiment, a small molecule inhibitor of the invention comprises an organic molecule, inorganic molecule, biomolecule, synthetic molecule, and the like.

Combinatorial libraries of molecularly diverse chemical compounds potentially useful in treating a variety of diseases and conditions are well known in the art as are method of making the libraries. The method may use a variety of techniques well-known to the skilled artisan including solid phase synthesis, solution methods, parallel synthesis of single compounds, synthesis of chemical mixtures, rigid core structures, flexible linear sequences, deconvolution strategies, tagging techniques, and generating unbiased molecular landscapes for lead discovery vs. biased structures for lead development.

In a general method for small library synthesis, an activated core molecule is condensed with a number of building blocks, resulting in a combinatorial library of covalently linked, core-building block ensembles. The shape and rigidity of the core determines the orientation of the building blocks in shape space. The libraries can be biased by changing the core, linkage, or building blocks to target a characterized biological structure (“focused libraries”) or synthesized with less structural bias using flexible cores.

The small molecule and small molecule compounds described herein may be present as salts even if salts are not depicted and it is understood that the invention embraces all salts and solvates of the inhibitors depicted here, as well as the non-salt and non-solvate form of the inhibitors, as is well understood by the skilled artisan. In some embodiments, the salts of the inhibitors of the invention are pharmaceutically acceptable salts.

Where tautomeric forms may be present for any of the inhibitors described herein, each and every tautomeric form is intended to be included in the present invention, even though only one or some of the tautomeric forms may be explicitly depicted. For example, when a 2-hydroxypyridyl moiety is depicted, the corresponding 2-pyridone tautomer is also intended.

The invention also includes any or all of the stereochemical forms, including any enantiomeric or diasteriomeric forms of the inhibitors described. The recitation of the structure or name herein is intended to embrace all possible stereoisomers of inhibitors depicted. All forms of the inhibitors are also embraced by the invention, such as crystalline or non-crystalline forms of the inhibitors. Compositions comprising an inhibitor of the invention are also intended, such as a composition of substantially pure inhibitor, including a specific stereochemical form thereof, or a composition comprising mixtures of inhibitors of the invention in any ratio, including two or more stereochemical forms, such as in a racemic or non-racemic mixture.

In one embodiment, the small molecule inhibitor of the invention comprises an analog or derivative of an inhibitor described herein.

In one embodiment, the small molecules described herein are candidates for derivatization. As such, in certain instances, the analogs of the small molecules described herein that have modulated potency, selectivity, and solubility are included herein and provide useful leads for drug discovery and drug development. Thus, in certain instances, during optimization new analogs are designed considering issues of drug delivery, metabolism, novelty, and safety.

In some instances, small molecule inhibitors described herein are derivatized/analoged as is well known in the art of combinatorial and medicinal chemistry. The analogs or derivatives can be prepared by adding and/or substituting functional groups at various locations. As such, the small molecules described herein can be converted into derivatives/analogs using well known chemical synthesis procedures. For example, all of the hydrogen atoms or substituents can be selectively modified to generate new analogs. Also, the linking atoms or groups can be modified into longer or shorter linkers with carbon backbones or hetero atoms. Also, the ring groups can be changed so as to have a different number of atoms in the ring and/or to include hetero atoms. Moreover, aromatics can be converted to cyclic rings, and vice versa. For example, the rings may be from 5-7 atoms, and may be homocycles or heterocycles.

In one embodiment, the small molecule inhibitors described herein can independently be derivatized/analoged by modifying hydrogen groups independently from each other into other substituents. That is, each atom on each molecule can be independently modified with respect to the other atoms on the same molecule. Any traditional modification for producing a derivative/analog can be used. For example, the atoms and substituents can be independently comprised of hydrogen, an alkyl, aliphatic, straight chain aliphatic, aliphatic having a chain hetero atom, branched aliphatic, substituted aliphatic, cyclic aliphatic, heterocyclic aliphatic having one or more hetero atoms, aromatic, heteroaromatic, polyaromatic, polyamino acids, peptides, polypeptides, combinations thereof, halogens, halo-substituted aliphatics, and the like. Additionally, any ring group on a compound can be derivatized to increase and/or decrease ring size as well as change the backbone atoms to carbon atoms or hetero atoms.

### Nucleic Acid Inhibitors

In other related aspects, the invention includes an isolated nucleic acid. In some instances, the inhibitor is an siRNA, shRNA, or antisense molecule, which inhibits a TET protein. In one embodiment, the nucleic acid comprises a promoter/regulatory sequence such that the nucleic acid is capable of directing expression of the nucleic acid. Thus, the invention encompasses expression vectors and methods for the introduction of exogenous DNA into cells with concomitant expression of the exogenous DNA in the cells such as those described, for example, in Sambrook et al. (2012, Molecular Cloning: A Laboratory Manual, Cold Spring Harbor Laboratory, New York), and in Ausubel et al. (1997, Current Protocols in Molecular Biology, John Wiley & Sons, New York) and as described elsewhere herein.

In another aspect of the invention, a TET protein can be inhibited by way of inactivating and/or sequestering a TET protein. As such, inhibiting the activity of a TET protein can be accomplished by using a transdominant negative mutant.

In one embodiment, siRNA or shRNA is used to decrease the level of a TET protein. RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. See, for example, U.S. Pat. No. 6,506,559; Fire et al., 1998, Nature 391(19):306-311; Timmons et al., 1998, Nature 395:854; Montgomery et al., 1998, TIG 14 (7):255-258; David R. Engelke, Ed., RNA Interference (RNAi) Nuts & Bolts of RNAi Technology, DNA Press, Eagleville, PA (2003); and Gregory J. Hannon, Ed., RNAi A Guide to Gene Silencing, Cold Spring Harbor Laboratory Press, Cold Spring Harbor, NY (2003). Soutschek et al. (2004, Nature 432:173-178) describe a chemical modification to siRNAs that aids in intravenous systemic delivery. Optimizing siRNAs involves consideration of overall G/C content, C/T content at the termini, Tm and the nucleotide content of the 3′ overhang. See, for instance, Schwartz et al., 2003, Cell, 115:199-208 and Khvorova et al., 2003, Cell 115:209-216. Therefore, the present invention also includes methods of decreasing levels of a TET protein using RNAi technology.

In another aspect, the invention includes a vector comprising an siRNA or antisense polynucleotide. In one embodiment, the siRNA or antisense polynucleotide is capable of inhibiting the expression of a target polypeptide, wherein the target polypeptide is selected from the group consisting of p21 and telomerase. The incorporation of a desired polynucleotide into a vector and the choice of vectors is well-known in the art as described in, for example, Sambrook et al. (2012), and in Ausubel et al. (1997), and elsewhere herein.

In certain embodiments, the expression vectors described herein encode a short hairpin RNA (shRNA) inhibitor. shRNA inhibitors are well known in the art and are directed against the mRNA of a target, thereby decreasing the expression of the target. In certain embodiments, the encoded shRNA is expressed by a cell, and is then processed into siRNA. For example, in certain instances, the cell possesses native enzymes (e.g., dicer) that cleaves the shRNA to form siRNA.

The siRNA, shRNA, or antisense polynucleotide can be cloned into a number of types of vectors as described elsewhere herein. For expression of the siRNA or antisense polynucleotide, at least one module in each promoter functions to position the start site for RNA synthesis.

In order to assess the expression of the siRNA, shRNA, or antisense polynucleotide, the expression vector to be introduced into a cell can also contain either a selectable marker gene or a reporter gene or both to facilitate identification and selection of expressing cells from the population of cells sought to be transfected or infected using a viral vector. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neomycin resistance and the like.

Therefore, in another aspect, the invention relates to a vector, comprising the nucleotide sequence of the invention or the construct of the invention. The choice of the vector will depend on the host cell in which it is to be subsequently introduced. In a particular embodiment, the vector of the invention is an expression vector. Suitable host cells include a wide variety of prokaryotic and eukaryotic host cells. In specific embodiments, the expression vector is selected from the group consisting of a viral vector, a bacterial vector and a mammalian cell vector. Prokaryote- and/or eukaryote-vector based systems can be employed for use with the present invention to produce polynucleotides, or their cognate polypeptides. Many such systems are commercially and widely available.

Further, the expression vector may be provided to a cell in the form of a viral vector. Viral vector technology is well known in the art and is described, for example, in Sambrook et al. (2012), and in Ausubel et al. (1997), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193.

By way of illustration, the vector in which the nucleic acid sequence is introduced can be a plasmid which is or is not integrated in the genome of a host cell when it is introduced in the cell. Illustrative, non-limiting examples of vectors in which the nucleotide sequence of the invention or the gene construct of the invention can be inserted include a tet-on inducible vector for expression in eukaryote cells.

The vector may be obtained by conventional methods known by persons skilled in the art (Sambrook et al., 2012). In a particular embodiment, the vector is a vector useful for transforming animal cells.

In one embodiment, the recombinant expression vectors may also contain nucleic acid molecules which encode a peptide or peptidomimetic inhibitor of invention, described elsewhere herein.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (U.S. Pat. Nos. 4,683,202, 5,928,906). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression, for example, see Sambrook et al. (2012). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

The recombinant expression vectors may also contain a selectable marker gene which facilitates the selection of transformed or transfected host cells. Suitable selectable marker genes are genes encoding proteins such as G418 and hygromycin which confer resistance to certain drugs, β-galactosidase, chloramphenicol acetyltransferase, firefly luciferase, or an immunoglobulin or portion thereof such as the Fc portion of an immunoglobulin, for example, IgG. The selectable markers may be introduced on a separate vector from the nucleic acid of interest.

Following the generation of the siRNA polynucleotide, a skilled artisan will understand that the siRNA polynucleotide will have certain characteristics that can be modified to improve the siRNA as a therapeutic compound. Therefore, the siRNA polynucleotide may be further designed to resist degradation by modifying it to include phosphorothioate, or other linkages, methylphosphonate, sulfone, sulfate, ketyl, phosphorodithioate, phosphoramidate, phosphate esters, and the like (see, e.g., Agrwal et al., 1987, Tetrahedron Lett. 28:3539-3542; Stec et al., 1985 Tetrahedron Lett. 26:2191-2194; Moody et al., 1989 Nucleic Acids Res. 12:4769-4782; Eckstein, 1989 Trends Biol. Sci. 14:97-100; Stein, In: Oligodeoxynucleotides. Antisense Inhibitors of Gene Expression, Cohen, ed., Macmillan Press, London, pp. 97-117 (1989)).

Any polynucleotide may be further modified to increase its stability in vivo. Possible modifications include, but are not limited to, the addition of flanking sequences at the 5′ and/or 3′ ends; the use of phosphorothioate or 2′ O-methyl rather than phosphodiester linkages in the backbone; and/or the inclusion of nontraditional bases such as inosine, queosine, and wybutosine and the like, as well as acetyl- methyl-, thio- and other modified forms of adenine, cytidine, guanine, thymine, and uridine.

In one embodiment of the invention, an antisense nucleic acid sequence which is expressed by a plasmid vector is used to inhibit a TET protein expression. The antisense vector is used to transfect a mammalian cell or the mammal itself, thereby causing reduced endogenous expression of a TET protein.

Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262:40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (1988, Anal. Biochem. 172:289). Such antisense molecules may be provided to the cell via genetic expression using DNA encoding the antisense molecule as taught by Inoue, 1993, U.S. Pat. No. 5,190,931.

Alternatively, antisense molecules of the invention may be made synthetically and then provided to the cell. In one embodiment, antisense oligomers may have between about 10 to about 30 nucleotides. In one embodiment, antisense oligomers may have about 15 nucleotides. In one embodiment, antisense oligomers having 10-30 nucleotides are easily synthesized and introduced into a target cell. Synthetic antisense molecules contemplated by the invention include oligonucleotide derivatives known in the art which have improved biological activity compared to unmodified oligonucleotides (see U.S. Pat. No. 5,023,243).

In one embodiment of the invention, a ribozyme is used to inhibit a TET protein expression. Ribozymes useful for inhibiting the expression of a target molecule may be designed by incorporating target sequences into the basic ribozyme structure which are complementary, for example, to the mRNA sequence encoding a TET protein. Ribozymes targeting a TET protein may be synthesized using commercially available reagents (Applied Biosystems, Inc., Foster City, CA) or they may be genetically expressed from DNA encoding them.

In one embodiment, the inhibitor of a TET protein comprises one or more components of a CRISPR-Cas system, where a guide RNA (gRNA) targeted to a gene encoding a TET protein, and a CRISPR-associated (Cas) peptide form a complex to induce mutations within the targeted gene. In one embodiment, the inhibitor comprises a gRNA or a nucleic acid molecule encoding a gRNA. In one embodiment, the inhibitor comprises a Cas peptide or a nucleic acid molecule encoding a Cas peptide.

### Polypeptide Inhibitors

In other related aspects, the invention includes an isolated peptide inhibitor that inhibits a TET protein. For example, in one embodiment, the peptide inhibitor of the invention inhibits a TET protein directly by binding to a TET protein, thereby preventing the normal functional activity of a TET protein. In another embodiment, the peptide inhibitor of the invention inhibits a TET protein by competing with an endogenous TET protein. In yet another embodiment, the peptide inhibitor of the invention inhibits activity of a TET protein by acting as a transdominant negative mutant.

The variants of the polypeptides according to the present invention may be (i) one in which one or more of the amino acid residues are substituted with a conserved or non-conserved amino acid residue and such substituted amino acid residue may or may not be one encoded by the genetic code, (ii) one in which there are one or more modified amino acid residues, e.g., residues that are modified by the attachment of substituent groups, (iii) one in which the polypeptide is an alternative splice variant of the polypeptide of the present invention, (iv) fragments of the polypeptides and/or (v) one in which the polypeptide is fused with another polypeptide, such as a leader or secretory sequence or a sequence which is employed for purification (for example, His-tag) or for detection (for example, Sv5 epitope tag). The fragments include polypeptides generated via proteolytic cleavage (including multi-site proteolysis) of an original sequence. Variants may be post-translationally, or chemically modified. Such variants are deemed to be within the scope of those skilled in the art from the teaching herein.

### Antibody Inhibitors

The invention also contemplates an inhibitor of a TET protein comprising an antibody, or antibody fragment, specific for a TET protein. That is, the antibody can inhibit a TET protein to provide a beneficial effect.

The antibodies may be intact monoclonal or polyclonal antibodies, and immunologically active fragments (e.g., a Fab or (Fab)₂ fragment), an antibody heavy chain, an antibody light chain, humanized antibodies, a genetically engineered single chain Fv molecule (Ladner et al., U.S. Pat. No. 4,946,778), or a chimeric antibody, for example, an antibody which contains the binding specificity of a murine antibody, but in which the remaining portions are of human origin. Antibodies including monoclonal and polyclonal antibodies, fragments and chimeras, may be prepared using methods known to those skilled in the art.

Antibodies can be prepared using intact polypeptides or fragments containing an immunizing antigen of interest. The polypeptide or oligopeptide used to immunize an animal may be obtained from the translation of RNA or synthesized chemically and can be conjugated to a carrier protein, if desired. Suitable carriers that may be chemically coupled to peptides include bovine serum albumin and thyroglobulin, keyhole limpet hemocyanin. The coupled polypeptide may then be used to immunize the animal (e.g., a mouse, a rat, or a rabbit).

In some embodiments, the compound further induces an apoptosis of at least one macrophage cell. In one embodiment, the compound further induces an apoptosis of at least one DAM, such as EAM, macrophages associated with a cancer, including, but not limiting to, lung cancer, ovarian cancer, leukemia, ALM, breast cancer, and/or pancreatic cancer. Thus, in some embodiments, the compound is further a modulator of a DAM.

In some embodiments, the compound further increases the level of at least one AgRP neuron, increases the activity of at least one AgRP neuron, increases the expression of at least one AgRP neuron, increases the function of at least one AgRP neuron, or any combination thereof. Thus, in some embodiments, the compound is further a modulator of an AgRP neuron.

In some embodiments, the compound further induces an apoptosis of at least one fibroblast cell. In one embodiment, the compound further induces an apoptosis of at least one CAF. Examples of such CAF include, but not limited to, lung cancer-associated fibroblast, ovarian cancer-associated fibroblast, leukemia-associated fibroblast, AML-associated fibroblast, breast cancer-associated fibroblast, pancreatic cancer-associated fibroblast, or any combination thereof.

### Methods

In one aspect, the present invention provides a method of modulating the level, activity, expression, stability, and/or degradation of at least one TET protein by administering the modulator of a TET protein. In various embodiments, the present invention provides a method of decreasing the level, activity, expression, and/or stability and/or increasing the degradation of at least one TET protein by administering the modulator of a TET protein.

The present invention also provides a method of treating or preventing a disease or disorder associated with the level of at least one TET protein. In one embodiment, the method comprises administering a modulator of a TET protein.

In various embodiments, the disease or disorder associated with the level of at least one TET protein is a disease or disorder associated with an increased level of at least one TET, disease or disorder associated with an increased activity of at least one TET, disease or disorder associated with an increased expression of at least one TET, disease or disorder associated with an increased function of at least one TET, or any combination thereof. Thus, in one embodiment, the method comprises administering an inhibitor of a TET protein.

In one embodiment, the disease or disorder associated with the level of a TET protein includes, but is not limited to, an eating disorder, disease or disorder associated with food intake, including cancer-induced anorexia and anorexia nervosa, disease or disorder associated with reduced appetite level, gynecological disease, including endometriosis and uterine fibroids, anxiety, stress-related disorder, depressive-like behavior, depression, postpartum depression, major depression, NAFLD, including NASH and liver fibrosis, cancer, including liver cancer, ovarian cancer, leukemia, AML, breast cancer, pancreatic cancer, lung cancer, glioma, and/or bladder cancer, disease or disorder associated with chronic inflammation, including NAFLD, cardiovascular disease, inflammatory bowel disease (IBD), Alzheimer's disease, Parkinson's disease, endometriosis, cancer, cancer-associated disease or disorder, such as cancer-induced depression, cancer-associated cachexia, or any combination thereof.

The present invention also provides a method of inducing apoptosis of at least one DAM (e.g., EAM, tumor-associated macrophage, etc.). In one embodiment, the invention provides a method of inducing apoptosis in DAM (e.g., EAM, tumor-associated macrophage, etc.) comprising administering a modulator of a TET protein. In various embodiments, the modulator of a TET is further a modulator of a DAM (e.g., EAM, tumor-associated macrophage, etc.).

The present invention also provides methods of treating or preventing a disease or disorder associated with the level of at least one DAM (e.g., EAM, tumor-associated macrophage, etc.). In one embodiment, the invention provides a method of treating or preventing a disease or disorder associated with the level of at least one DAM (e.g., EAM, tumor-associated macrophage, etc.) comprising administering a modulator of a TET protein.

In various embodiments, the disease or disorder associated with the level of at least one DAM (e.g., EAM, tumor-associated macrophage, etc.) is a disease or disorder associated with increased level of at least one DAM (e.g., EAM, tumor-associated macrophage, etc.), disease or disorder associated with increased activity of at least one DAM (e.g., EAM, tumor-associated macrophage, etc.), disease or disorder associated with increased expression of at least one DAM (e.g., EAM, tumor-associated macrophage, etc.), disease or disorder associated with increased function of at least one DAM (e.g., EAM, tumor-associated macrophage, etc.), or any combination thereof.

In one embodiment, the method of treating or preventing a disease or disorder associated with the level of at least one DAM (e.g., EAM, tumor-associated macrophage, etc.) comprises administering a composition that induces apoptosis of the DAM (e.g., EAM, tumor-associated macrophage, etc.). In one embodiment, the composition that induces apoptosis of DAM (e.g., EAM, tumor-associated macrophage, etc.) is an inhibitor of a TET protein. In one embodiment, diseases or disorders associated with the level of at least one DAM (e.g., EAM, tumor-associated macrophage, etc.) include, but are not limited to, endometriosis, NASH, cancer, and/or cancer-associated disease or disorder.

The present invention also provides a method of inducing apoptosis of at least one CAF. In one embodiment, the invention provides a method of inducing apoptosis in CAF comprising administering a modulator of a TET protein. In various embodiments, the modulator of a TET is further a modulator of a CAF.

The present invention also provides methods of treating or preventing a disease or disorder associated with the level of at least one CAF. In one embodiment, the invention provides a method of treating or preventing a disease or disorder associated with the level of at least one CAF comprising administering a modulator of a TET protein.

In various embodiments, the disease or disorder associated with the level of at least one CAF is a disease or disorder associated with increased level of at least one CAF, disease or disorder associated with increased activity of at least one CAF, disease or disorder associated with increased expression of at least one CAF, disease or disorder associated with increased function of at least one CAF, or any combination thereof.

In one embodiment, the method of treating or preventing a disease or disorder associated with the level of at least one CAF comprises administering a composition that induces apoptosis of the CAF. In one embodiment, the composition that induces apoptosis of CAF is an inhibitor of a TET protein. In one embodiment, diseases or disorders associated with the level of at least one CAF include, but are not limited to, endometriosis, NASH, cancer, and/or cancer-associated disease or disorder.

The present invention also provides a method of modulating the function or level of at least one AgRP neuron. In one embodiment, the invention provides a method of modulating the level of at least one TET protein in at least one AgRP neuron, activity of at least one TET protein in at least one AgRP neuron, expression of at least one TET protein in at least one AgRP neuron, stability of at least one TET protein in at least one AgRP neuron, degradation of at least one TET protein in at least one AgRP neuron, or any combination thereof.

In some embodiments, the modulator of TET protein increases the level of at least one AgRP neuron, activity of at least one AgRP neuron, expression of at least one AgRP neuron, stability of at least one AgRP neuron, degradation of at least one AgRP neuron, level of at least one AGRP peptide, activity of at least one AGRP peptide, expression of at least one AGRP peptide, stability of at least one AGRP peptide, degradation of at least one AGRP peptide, level of at least one NPY peptide, activity of at least one NPY peptide, expression of at least one NPY peptide, stability of at least one NPY peptide, degradation of at least one NPY peptide, level of at least one VGAT, activity of at least one VGAT, expression of at least one VGAT, stability of at least one VGAT, degradation of at least one VGAT, or any combination thereof.

Thus, in various embodiments, the modulator of TET protein is further a modulator of an AgRP neuron.

The present invention also provides a method of treating or preventing a disease or disorder associated with the level or function of at least one AgRP neuron. In one embodiment, the invention provides a method of treating or preventing a disease or disorder associated with the level or function of at least one AgRP neuron comprising administering a modulator of a TET protein.

In various embodiments, the disease or disorder associated with the level or function of at least one AgRP neuron is a disease or disorder associated with decreased level of at least one AgRP neuron, disease or disorder associated with decreased activity of at least one AgRP neuron, disease or disorder associated with decreased expression of at least one AgRP neuron, disease or disorder associated with decreased function of at least one AgRP neuron, or any combination thereof.

In one embodiment, the method of treating a disease or disorder associated with decreased level or function of AgRP neuron comprises administering a composition that activates AgRP neuron. In one embodiment, the composition that activates AgRP neuron is an inhibitor of a TET protein. In one embodiment, diseases or disorders associated with decreased with level or function of AgRP neuron include, but are not limited to an eating disorder, mood disorder, anxiety, anorexia, cancer-associated disease or disorder, such as cancer-associated cachexia, depression, depression-associated illnesses, and/or hypophagia.

In other aspects, the present invention also provides methods of treating or preventing cancer and/or cancer-associated disease or disorder in a subject in need thereof.

The following are non-limiting examples of cancers that can be treated by the disclosed methods and compositions: acute lymphoblastic leukemia; acute myeloid leukemia; adrenocortical carcinoma; adrenocortical carcinoma, childhood; appendiceal carcinoma; basal cell carcinoma; bile duct cancer, extrahepatic; bladder cancer; bone cancer; osteosarcoma and malignant fibrous histiocytoma; brain stem glioma, childhood; brain tumor, adult; brain tumor, brain stem glioma, childhood; brain tumor, central nervous system atypical teratoid/rhabdoid tumor, childhood; central nervous system embryonal tumors; cerebellar astrocytoma; cerebral astrocytotna/malignant glioma; craniopharyngioma; ependymoblastoma; ependymoma; medulloblastoma; medulloepithelioma; pineal parenchymal tumors of intermediate differentiation; supratentorial primitive neuroectodermal tumors and pineoblastoma; visual pathway and hypothalamic glioma; brain and spinal cord tumors; breast cancer; bronchial tumors; Burkitt's lymphoma; carcinoid tumor; carcinoid tumor, gastrointestinal; central nervous system atypical teratoid/rhabdoid tumor; central nervous system embryonal tumors; central nervous system lymphoma; cerebellar astrocytoma cerebral astrocytoma/malignant glioma, childhood; cervical cancer; chordoma, childhood; chronic lymphocytic leukemia; chronic myelogenous leukemia; chronic myeloproliferative disorders; colon cancer; colorectal cancer; craniopharyngioma; cutaneous T-cell lymphoma; esophageal cancer; Ewing family of tumors; extragonadal germ cell tumor; extrahepatic bile duct cancer; eye cancer, intraocular melanoma; eye cancer, retinoblastoma; gallbladder cancer; gastric (stomach) cancer; gastrointestinal carcinoid tumor; gastrointestinal stromal tumor (gist); germ cell tumor, extracranial; germ cell tumor, extragonadal; germ cell tumor, ovarian; gestational trophoblastic tumor; glioma; glioma, childhood brain stem; glioma, childhood cerebral astrocytoma; glioma, childhood visual pathway and hypothalamic; hairy cell leukemia; head and neck cancer; hepatocellular (liver) cancer; histiocytosis, langerhans cell; Hodgkin lymphoma; hypopharyngeal cancer; hypothalamic and visual pathway glioma; intraocular melanoma; islet cell tumors; kidney (renal cell) cancer; Langerhans cell histiocytosis; laryngeal cancer; leukemia, acute lymphoblastic; leukemia, acute myeloid; leukemia, chronic lymphocytic; leukemia, chronic myelogenous; leukemia, hairy cell; lip and oral cavity cancer; liver cancer; lung cancer, non-small cell; lung cancer, small cell; lymphoma, aids-related; lymphoma, Burkitt; lymphoma, cutaneous T-cell; lymphoma, non-Hodgkin lymphoma; lymphoma, primary central nervous system; macroglobulinemia, Waldenstrom; malignant fibrous histiocytoma of bone and osteosarcoma; medulloblastoma; melanoma; melanoma, intraocular (eye); Merkel cell carcinoma; mesothelioma; metastatic squamous neck cancer with occult primary; mouth cancer; multiple endocrine neoplasia syndrome, (childhood); multiple myeloma/plasma cell neoplasm; mycosis; fungoides; myelodysplastic syndromes; myelodysplastic/myeloproliferative diseases; myelogenous leukemia, chronic; myeloid leukemia, adult acute; myeloid leukemia, childhood acute; myeloma, multiple; myeloproliferative disorders, chronic; nasal cavity and paranasal sinus cancer; nasopharyngeal cancer; neuroblastoma; non-small cell lung cancer; oral cancer; oral cavity cancer; oropharyngeal cancer; osteosarcoma and malignant fibrous histiocytoma of bone; ovarian cancer; ovarian epithelial cancer; ovarian germ cell tumor; ovarian low malignant potential tumor; pancreatic cancer; pancreatic cancer, islet cell tumors; papillomatosis; parathyroid cancer; penile cancer; pharyngeal cancer; pheochromocytoma; pineal parenchymal tumors of intermediate differentiation; pineoblastoma and supratentorial primitive neuroectodermal tumors; pituitary tumor; plasma celt neoplasm/multiple myeloma; pleuropulmonary blastoma; primary central nervous system lymphoma; prostate cancer; rectal cancer; renal cell (kidney) cancer; renal pelvis and ureter, transitional cell cancer; respiratory tract carcinoma involving the nut gene on chromosome 15; retinoblastoma; rhabdomyosarcoma; salivary gland cancer; sarcoma, ewing family of tumors; sarcoma, Kaposi; sarcoma, soft tissue; sarcoma, uterine; sezary syndrome; skin cancer (nonmelanoma); skin cancer (melanoma); skin carcinoma, Merkel cell; small cell lung cancer; small intestine cancer; soft tissue sarcoma; squamous cell carcinoma, squamous neck cancer with occult primary, metastatic; stomach (gastric) cancer; supratentorial primitive neuroectodermal tumors; T-cell lymphoma, cutaneous; testicular cancer; throat cancer; thymoma and thymic carcinoma; thyroid cancer; transitional cell cancer of the renal pelvis and ureter; trophoblastic tumor, gestational; urethral cancer; uterine cancer, endometrial; uterine sarcoma; vaginal cancer; vulvar cancer; Waldenstrom macroglobulinemia; and Wilms tumor.

For example, in some embodiments, the cancer is a liver cancer, pancreatic cancer, breast cancer, lung cancer, and/or ovarian cancer.

In one aspect, the present invention also provides a method of modulating at least one pathway involved in transforming growth factor beta (TGF-β) signaling, metabolic reprogramming, pyroptosis, apoptosis, or any combination thereof in a subject in need thereof. In some embodiments, the method comprises inhibiting at least one pathway involved in TGF-βsignaling, metabolic reprogramming, or a combination thereof. In some embodiments, the method comprises activating at least one pathway involved in apoptosis. In some embodiments, the method comprises activating apoptosis.

In some embodiments, the method comprises administering to the subject a therapeutically effective amount of a degrader of TET protein, inhibitor of TET protein, or a combination thereof, or a composition thereof.

In various aspects, the present invention also provides a method of administering at least one modulator of a TET protein to a subject in need thereof. In some embodiments, the subject has a disease or disorder. In some embodiments, the disease or disorder includes, but is not limited to, an eating disorder, disease or disorder associated with food intake, including cancer-induced anorexia and anorexia nervosa, disease or disorder associated with reduced appetite level, gynecological disease, including endometriosis and uterine fibroids, anxiety, stress-related disorder, depressive-like behavior, depression, cancer-induced depression, postpartum depression, major depression, NAFLD, including NASH and liver fibrosis, cancer, including liver cancer, ovarian cancer, leukemia, AML, breast cancer, pancreatic cancer, lung cancer, glioma, and/or bladder cancer, disease or disorder associated with chronic inflammation, including NAFLD, cardiovascular disease, inflammatory bowel disease (IBD), Alzheimer's disease, Parkinson's disease, endometriosis, cancer, cancer-associated disease or disorder, or any combination thereof.

It will be appreciated by one of skill in the art, when armed with the present invention including the methods detailed herein, that the invention is not limited to treatment of a disease or disorder associated with increased level and/or activity of a TET protein that is already established. Particularly, the disease or disorder need not have manifested to the point of detriment to the subject; indeed, the disease or disorder need not be detected in a subject before treatment is administered. That is, significant signs or symptoms of the disease or disorder do not have to occur before the present invention may provide benefit. Therefore, the present invention includes a method for preventing a disease or disorder associated with increased level and/or activity of a TET protein, in that an inhibitor composition, as discussed previously elsewhere herein, can be administered to a subject prior to the onset of the disease or disorder, thereby preventing the disease or disorder. The preventive methods described herein also include the treatment of a subject that is in remission for the prevention of a recurrence a disease or disorder associated with abnormal immune cell activation.

One of skill in the art, when armed with the disclosure herein, would appreciate that the prevention of a disease or disorder associated with increased level and/or activity of a TET protein, encompasses administering to a subject an inhibitor composition as a preventative measure against the development of, or progression of a disease or disorder associated with abnormal increased level and/or activity of a TET protein. As more fully discussed elsewhere herein, methods of inhibiting the level or activity of a gene, or gene product, encompass a plethora of techniques for reducing not only the level and activity of polypeptide gene products, but also for modulating expression of a nucleic acid, including either transcription, translation, or both.

Additionally, as disclosed elsewhere herein, one skilled in the art would understand, once armed with the teaching provided herein, that the present invention encompasses methods of treating, or preventing, a wide variety of diseases, disorders and pathologies associated with increased level and/or activity of a TET protein, where reducing the level or activity of a gene, or gene product treats or prevents the disease or disorder. Various methods for assessing whether a disease is associated with increased level and/or activity of a TET protein are known in the art. Further, the invention encompasses treatment or prevention of such diseases discovered in the future.

The present invention also encompasses administration of an inhibitor of a TET protein, TGF-β, interleukin-1 beta (IL-1β), interleukin 6 (IL-6), or any combination thereof in a subject in need thereof. To practice the methods of the invention, the skilled artisan would understand, based on the disclosure provided herein, how to formulate and administer the appropriate inhibitor composition to a subject. The present invention is not limited to any particular method of administration or treatment regimen.

In one embodiment, the method comprises administering to the subject in need an effective amount of a composition that inhibits the level and/or activity of a TET protein, TGF-β, IL-1β, IL-6, or any combination thereof.

In one embodiment, the method of decreasing the level and/or activity of a TET protein, TGF-β, IL-1β, IL-6, or any combination thereof comprises administering to the subject an effective amount of a composition that reduces the level and/or activity of a TET protein, TGF-β, IL-1β, IL-6, or any combination thereof.

In one embodiment, the method of treating or preventing a disease or disorder associated with an increased level and/or activity of a TET protein comprises administering to the subject an effective amount of a composition that reduces the level and/or activity of a TET protein, TGF-β, IL-1β, IL-6, or any combination thereof.

One of skill in the art will appreciate that the inhibitors of the invention can be administered singly or in any combination, Further, the inhibitors of the invention can be administered singly or in any combination in a temporal sense, in that they may be administered concurrently, or before, and/or after each other. One of ordinary skill in the art will appreciate, based on the disclosure provided herein, that the inhibitor compositions of the invention can be used to prevent or to treat a disease or disorder associated with the level of at least one TET protein, and that an inhibitor composition can be used alone or in any combination with another inhibitor to achieve a therapeutic result. In various embodiments, any of the inhibitors of the invention described herein can be administered alone or in combination with other inhibitors of other molecules associated with a disease or disorder associated with the level of at least one TET protein.

### Pharmaceutical Compositions and Formulations

The invention also encompasses the use of pharmaceutical compositions of the invention or salts thereof to practice the methods of the invention. Such a pharmaceutical composition may consist of at least one inhibitor composition of the invention or a salt thereof in a form suitable for administration to a subject, or the pharmaceutical composition may comprise at least one inhibitor composition of the invention or a salt thereof, and one or more pharmaceutically acceptable carriers, one or more additional ingredients, or some combination of these. The compound or conjugate of the invention may be present in the pharmaceutical composition in the form of a physiologically acceptable salt, such as in combination with a physiologically acceptable cation or anion, as is well known in the art.

In an embodiment, the pharmaceutical compositions useful for practicing the methods of the invention may be administered to deliver a dose of between 1 ng/kg/day and 100 mg/kg/day. In another embodiment, the pharmaceutical compositions useful for practicing the invention may be administered to deliver a dose of between 1 ng/kg/day and 500 mg/kg/day.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

Pharmaceutical compositions that are useful in the methods of the invention may be suitably developed for oral, rectal, vaginal, parenteral, topical, pulmonary, intranasal, buccal, ophthalmic, or another route of administration. A composition useful within the methods of the invention may be directly administered to the skin, vagina or any other tissue of a mammal. Other contemplated formulations include liposomal preparations, resealed erythrocytes containing the active ingredient, and immunologically-based formulations. The route(s) of administration will be readily apparent to the skilled artisan and will depend upon any number of factors including the type and severity of the disease being treated, the type and age of the veterinary or human subject being treated, and the like.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

Although the descriptions of pharmaceutical compositions provided herein are principally directed to pharmaceutical compositions that are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist may design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs.

In one embodiment, the compositions of the invention are formulated using one or more pharmaceutically acceptable excipients or carriers. In one embodiment, the pharmaceutical compositions of the invention comprise a therapeutically effective amount of a compound or conjugate of the invention and a pharmaceutically acceptable carrier. Pharmaceutically acceptable carriers that are useful, include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In one embodiment isotonic agents, for example, sugars, sodium chloride, or polyalcohols such as mannitol and sorbitol, are included in the composition. Prolonged absorption of the injectable compositions may be brought about by including in the composition an agent that delays absorption, for example, aluminum monostearate or gelatin. In one embodiment, the pharmaceutically acceptable carrier is not DMSO alone.

Formulations may be employed in admixtures with conventional excipients, i.e., pharmaceutically acceptable organic or inorganic carrier substances suitable for oral, vaginal, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” that may be included in the pharmaceutical compositions of the invention are known in the art and described, for example in Genaro, ed. (1985, Remington's Pharmaceutical Sciences, Mack Publishing Co., Easton, PA), which is incorporated herein by reference.

The composition of the invention may comprise a preservative from about 0.005% to 2.0% by total weight of the composition. The preservative is used to prevent spoilage in the case of exposure to contaminants in the environment. Examples of preservatives useful in accordance with the invention included but are not limited to those selected from the group consisting of benzyl alcohol, sorbic acid, parabens, imidurea and combinations thereof. An exemplary preservative is a combination of about 0.5% to 2.0% benzyl alcohol and 0.05% to 0.5% sorbic acid.

In one embodiment, the composition includes an anti-oxidant and a chelating agent that inhibits the degradation of the compound. Exemplary antioxidants for some compounds are BHT, BHA, alpha-tocopherol and ascorbic acid in the range of about 0.01% to 0.3%. In one embodiment, the BHT is in the range of 0.03% to 0.1% by weight by total weight of the composition. In one embodiment, the chelating agent is present in an amount of from 0.01% to 0.5% by weight by total weight of the composition. Exemplary chelating agents include edetate salts (e.g. disodium edetate) and citric acid in the weight range of about 0.01% to 0.20%. In one embodiment, chelating agents may be in the range of 0.02% to 0.10% by weight by total weight of the composition. The chelating agent is useful for chelating metal ions in the composition that may be detrimental to the shelf life of the formulation. While BHT and disodium edetate are the exemplary antioxidant and chelating agent respectively for some compounds, other suitable and equivalent antioxidants and chelating agents may be substituted therefore as would be known to those skilled in the art.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

The regimen of administration may affect what constitutes an effective amount. The therapeutic formulations may be administered to the subject either prior to or after a diagnosis of disease. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a subject, such a mammal, including a human, may be carried out using known procedures, at dosages and for periods of time effective to prevent or treat disease. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the subject being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 1 and 5,000 mg/kg of body weight/per day. One of ordinary skill in the art would be able to study the relevant factors and make the determination regarding the effective amount of the therapeutic compound without undue experimentation.

The compound may be administered to a subject as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose will be readily apparent to the skilled artisan and will depend upon any number of factors, such as, but not limited to, the type and severity of the disease being treated, the type and age of the animal, etc.

Actual dosage levels of the active ingredients in the pharmaceutical compositions of this invention may be varied so as to obtain an amount of the active ingredient that is effective to achieve the desired therapeutic response for a particular subject, composition, and mode of administration, without being toxic to the subject.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a disease in a subject.

In certain embodiments, the composition of the present invention provides for a controlled release of a therapeutic agent, such as a inhibitor of a TET protein. In certain instances, controlled- or sustained-release formulations of a pharmaceutical composition of the invention may be made using conventional technology, using for example proteins equipped with pH sensitive domains or protease-cleavable fragments. In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, micro-particles, liposomes, or microspheres or a combination thereof to provide the desired release profile in varying proportions. Suitable controlled-release formulations known to those of ordinary skill in the art, including those described herein, can be readily selected for use with the pharmaceutical compositions of the invention. Thus, single unit dosage forms suitable for oral administration, such as tablets, capsules, gel-caps, lozenges, and caplets, which are adapted for controlled-release are encompassed by the present invention.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased subject compliance. In addition, controlled-release formulations can be used to affect the time of onset of action or other characteristics, such as blood level of the drug, and thus can affect the occurrence of side effects.

Most controlled-release formulations are designed to initially release an amount of drug that promptly produces the desired therapeutic effect, and gradually and continually release of other amounts of drug to maintain this level of therapeutic effect over an extended period of time. In certain embodiments, the controlled-release formulation of the composition described herein allows for release of a therapeutic agent precisely when the agent is most needed. In another embodiment, the controlled-release formulation of the composition described herein allows for release of a therapeutic agent precisely in conditions in which the therapeutic agent is most active. In order to maintain this constant level of drug in the body, the drug must be released from the dosage form at a rate that will replace the amount of drug being metabolized and excreted from the body.

In certain embodiment, the composition provides for an environment-dependent release, when and where the therapeutic agent is triggered for release. For example, in certain embodiments the composition invention releases at least one therapeutic agent when and where the at least one therapeutic agent is needed. The triggering of release may be accomplished by a variety of factors within the microenvironment of the treatment or prevention site, including, but not limited to, temperature, pH, the presence or activity of a specific molecule or biomolecule, and the like.

Controlled-release of an active ingredient can be stimulated by various inducers, for example pH, temperature, enzymes, water or other physiological conditions or compounds. The term “controlled-release component” in the context of the present invention is defined herein as a compound or compounds, including, but not limited to, polymers, polymer matrices, gels, permeable membranes, liposomes, or microspheres or a combination thereof that facilitates the controlled-release of the active ingredient.

In certain embodiments, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release that is longer that the same amount of agent administered in bolus form.

For sustained release, the compounds may be formulated with a suitable polymer or hydrophobic material that provides sustained release properties to the compounds. As such, the compounds for use the method of the invention may be administered in the form of microparticles, for example, by injection or in the form of wafers or discs by implantation.

In one embodiment of the invention, the compounds of the invention are administered to a subject, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

In one embodiment, the compositions of the invention are administered to the subject in dosages that range from one to five times per day or more. In another embodiment, the compositions of the invention are administered to the subject in range of dosages that include, but are not limited to, once every day, every two, days, every three days to once a week, and once every two weeks. It will be readily apparent to one skilled in the art that the frequency of administration of the various combination compositions of the invention will vary from subject to subject depending on many factors including, but not limited to, age, disease or disorder to be treated, gender, overall health, and other factors. Thus, the invention should not be construed to be limited to any particular dosage regime and the precise dosage and composition to be administered to any subject will be determined by the attending physical taking all other factors about the subject into account.

Compounds of the invention for administration may be in the range of from about 1 mg to about 10,000 mg, about 20 mg to about 9,500 mg, about 40 mg to about 9,000 mg, about 75 mg to about 8,500 mg, about 150 mg to about 7,500 mg, about 200 mg to about 7,000 mg, about 3050 mg to about 6,000 mg, about 500 mg to about 5,000 mg, about 750 mg to about 4,000 mg, about 1 mg to about 3,000 mg, about 10 mg to about 2,500 mg, about 20 mg to about 2,000 mg, about 25 mg to about 1,500 mg, about 50 mg to about 1,000 mg, about 75 mg to about 900 mg, about 100 mg to about 800 mg, about 250 mg to about 750 mg, about 300 mg to about 600 mg, about 400 mg to about 500 mg, and any and all whole or partial increments therebetween.

In some embodiments, the dose of a compound of the invention is from about 1 mg and about 2,500 mg. In some embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound (i.e., a drug used for treating the same or another disease as that treated by the compositions of the invention) as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In one embodiment, the present invention is directed to a packaged pharmaceutical composition comprising a container holding a therapeutically effective amount of a compound or conjugate of the invention, alone or in combination with a second pharmaceutical agent; and instructions for using the compound or conjugate to treat, prevent, or reduce one or more symptoms of a disease in a subject.

The term “container” includes any receptacle for holding the pharmaceutical composition. For example, in one embodiment, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating or preventing a disease in a subject, or delivering an imaging or diagnostic agent to a subject.

Routes of administration of any of the compositions of the invention include oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out certain embodiments of the present invention, and are not to be construed as limiting in any way the remainder of the disclosure.

### Example 1: TET3 Epigenetically Controlled Feeding and Stress Response Behaviors Via AgRP Neurons

The ten-eleven translocation (TET) family proteins (TET1, TET2 and TET3) oxidize 5-methylcytosine to 5-hydroxymethylcytosine and its derivatives to mediate DNA demethylation (Lio et al., 2020, J Biosci., 45). TET proteins can also regulate gene expression independently of their catalytic activities (Lio et al., 2020, J Biosci., 45). The catalytic domain of the three TET enzymes is highly conserved but not identical, and each of the members exhibits varying substrate preferences and catalytic activity (Tahiliani et al., 2009, Science, 324:930-935).

Bobcat339 is a synthetic cytosine derivative initially reported to inhibit the enzymatic activity of TET1 and TET2, but its effects in vivo and on TET3 were not defined (Chua et al., 2019, ACS Med Chem Lett., 10:180-185). It was later found that Bobcat339 on its own had a negligible inhibitory activity against TET1 and TET2 in the absence of contaminating copper (II) (Weirath et al., 2022, ACS Med Chem Lett., 13:792-798). Based on the crystal structure of TET2-DNA complex, Bobcat339 was predicted to bind to the catalytic pockets of all three TET enzymes, but the actual physical binding sites on these proteins have not been experimentally mapped (Chua et al., 2019, ACS Med Chem Lett., 10:180-185). Recently, it was unexpectedly found that Bobcat339 induced protein degradation of TET3 in the absence of contaminating copper(II) in human and mouse neuronal cells, a discovery not previously documented (Lv et al., 2023, Proc Natl Acad Sci USA, in press).

The TET family of dioxygenases (TET1/2/3) initiate DNA demethylation by converting 5-methylcytosines (5mC) to 5-hydroxymethylcytosines (5hmC), which they further oxidize into 5-formylcytosines (5fC) and 5-carboxylcytosines (ScaC), that are removed by thymine DNA glycosylase, completing the cytosine demethylation cycle (Wu X. et al., 2017, Nature Reviews Genetics, 18:517-534; Lio C. J. et al., 2020, Journal of Bioscience, 45:21; Yang J. et al., 2020, Development, 147:dev183129). 5hmC also serves as a stable epigenetic mark and functions to enhance or inhibit binding of regulatory protein factors in a context-dependent manner (Syed K. S. et al., 2016, Biochemistry, 55:6940-6948; Mellen M. et al., 2017, Proceedings of the National Academy of Sciences USA, 114:E7812-E7821). TETs can also regulate chromatin architecture and gene transcription independently of their catalytic activities (Williams K., 2011, Nature, 473:343-348; Kaas G. A. et al., 2013, Neuron, 79:1086-1093; Zhang Q. et al., 2015, Nature, 525:389-393; Xue S. et al., 2016, Cell Reports, 16:1096-1105; Guan W. et al., Proceedings of the National Academy of Sciences USA, 114:8229-8234; Montalban-Loro R. et al., 2019, Nature Communications, 10:1726; Tanaka S. et al., 2020, Nature Immunology, 21:950-961).

In the mouse brain, Tet genes are widely transcribed across different forebrain regions including cortex, hippocampus, cerebellum, and hypothalamus, with Tet3 being the most abundant (Szwagierczak A. et al., 2010, Nucleic Acids Research, 38:e181; Cisternas C. D. et al., 2020, Epigenetics, 15:72-84). TET1 and TET2 have been shown to play important roles in learning and memory processes in adult mice (Kaas G. A. et al., 2013, Neuron, 79:1086-1093; Zhang, R. R. et al., 2013, Cell Stem Cell, 13:237-245; Gontier G. et al., 2018, Cell Reports, 22:1974-1981; Rudenko A. et al., 2013, Neuron, 79:1109-1122; Kumar D. et al., 2015, Neuroepigenetics, 4:12-27). While Tet3 knockout in mice is neonatally lethal, Tet3 knockdown in the infralimbic prefrontal cortex or hippocampal neurons impairs fear extinction memory (Gu T. P. et al., 2011, Nature, 477:606-610; Li X. et al., 2014, Proceedings of the National Academy of Sciences USA, 111:7120-7125; Kremer E. A. et al., 2018, Scientific Reports, 8:1678). In adult mice, Tet3 ablation in forebrain neurons (particularly hippocampal neurons) results in increased anxiety and impaired spatial orientation (Antunes C. et al., 2021, Molecular Psychiatry, 26:1445-1457). These studies have relied on non-cell type-specific approaches and a clear mechanistic understanding linking cell type-specific epigenetic changes induced by TETs to specific behavioral phenotypes has not been accomplished. In addition, there is a paucity of information regarding the potential role of TETs in central control of energy metabolism, which is also addressed herein.

### TET Inhibition Elevated AGRP Expression in the ARC

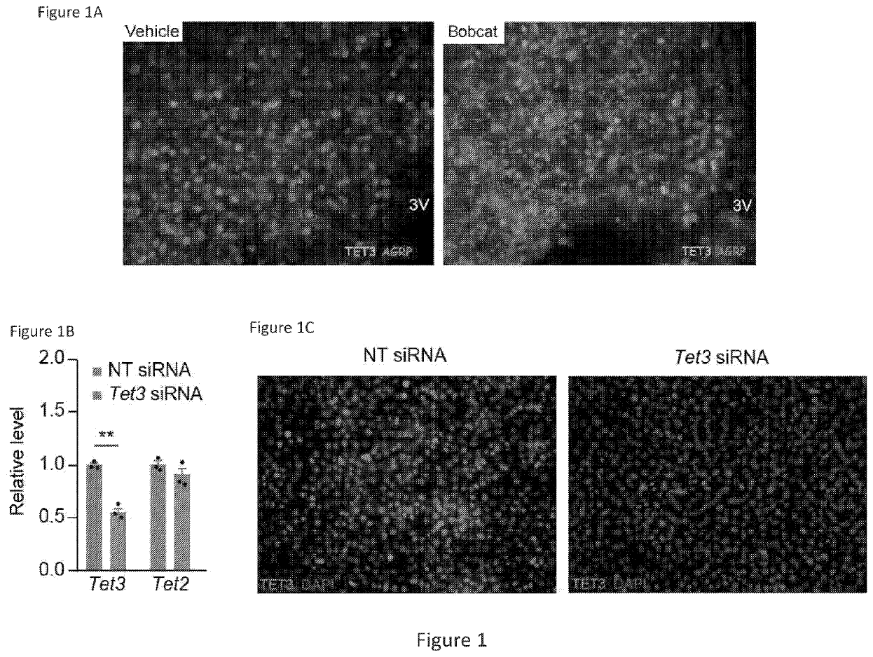

It has previously been shown that TET3 expression is aberrantly elevated in the livers of humans and mice with type 2 diabetes and that liver-specific, siRNA-mediated TET3 knockdown improves glucose homeostasis both in dietary and genetic mouse models of diabetes (Li D. et al., 2020, Nature Communications, 11:342). Bobcat339 (4-Amino-1-[1,1′-biphenyl]-3-yl-5-chloro-2(1H)-pyrimidinone) is a synthetic cytosine derivative capable of binding competitively with 5mC to the active sites of TETs and inhibits their enzymatic activity in cultured neuronal cells (Chua G. N. L. et al., 2019, ACS Medicinal Chemistry Letters, 10:180-185). Although not bound by any particular theory, it was hypothesized that Bobcat339 has an anti-diabetic effect owing to its ability to inhibit TETs. Thus, the present studies employed high fat diet (HFD)-induced diabetic mice that were treated with Bobcat339 in drinking water for two weeks. Unexpectedly, the Bobcat339-treated animal became hyperphagic, indicating that Bobcat339 had affected the brain. Thus, nondiabetic mice fed on regular chow were treated with Bobcat339 in drinking water. Four days later, ARCs were isolated from ad libitum-fed mice and analyzed. As shown in FIG. 1A, Bobcat339 treatment increased AGRP expression in the ARC, which at least in part explained why the mice became hyperphagic. In light of these observations, additional studies began to explore TETs in AgRP neurons for a potential role in the control of feeding and energy metabolism.

### Food Deprivation Downregulates TET3 in AgRP Neurons

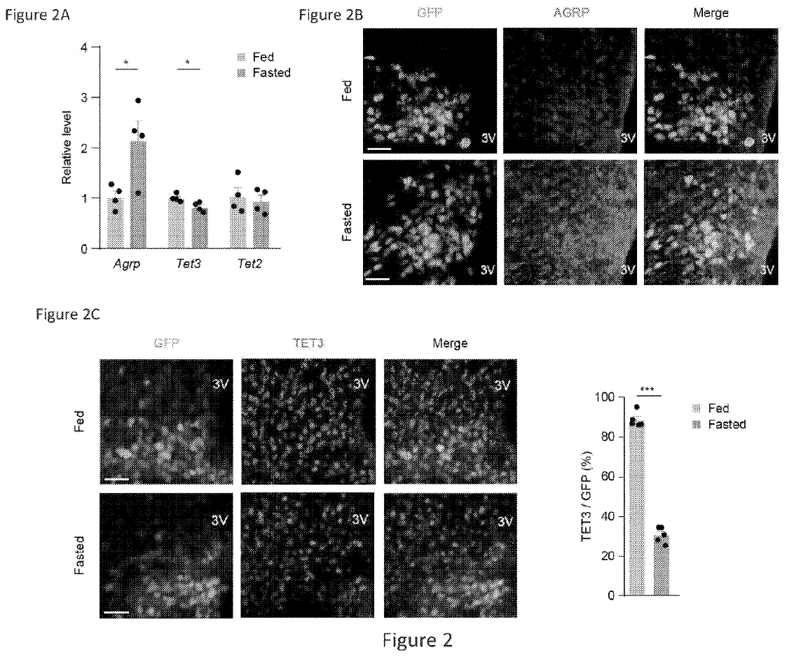

To determine whether fasting, which normally upregulates AGRP, affected TET expression in AgRP neurons, Agrp-IRES-Cre mice were mated with Cre-enabled Rosa26-LSL-Cas9-GFP-knockin mice to obtain Agrp-IRES-Cre:LSL-Cas9-GFP mice (hereinafter “Cas9+”), which co-expressed GFP and Cas9 endonuclease specifically in AgRP neurons. The Cas9+ mice were fasted overnight for 12 h and ARCs were isolated for RNA and protein analyses. Fasting increased Agrp mRNA by approximately 2-fold as compared to ad libitum-fed mice (FIG. 2A). Previous cell type-specific transcriptome sequencing using purified mouse AgRP neurons reported ~4-fold increase in Agrp mRNA following a 24 h fasting (Henry F. E. et al., 2015, eLife, 4:e09800). Fasting decreased Tet3 mRNA without affecting Tet2 mRNA in the ARC (FIG. 2A). Tet1 expression in the ARC was negligible.

Immunofluorescence analysis revealed a marked increase in AGRP in the ARC of fasted vs. fed mice (FIG. 2B). The diffuse, robust AgRP signal was consistent with AGRP being a secreted, stable peptide and AgRP neurons' broad projections to other areas in the brain (Deem J. D. et al., 2021, FEBS Journal, 289:2362-2381; Rosenfeld R. D. et al., 1998, Biochemistry, 37:16041-16052). While TET3 protein was readily detected in both AgRP and non-AgRP cells, fasting clearly decreased the number of TET3-positive AgRP neurons (FIG. 2C). The apparently modest decrease in Tet3 mRNA by fasting (FIG. 2A) vs. protein (FIG. 2C) was in part a result of using a mixed cell population of the ARC in the RT-qPCR assays. The specificity of the TET3 antibody was previously validated (Li, D. et al., 2020, Nature Communications, 11:342) and further confirmed using an siRNA specifically targeting mouse Tet3 (Tet3 siRNA) in a mouse hypothalamic neuronal cell line (FIG. 1). Taken together, these results demonstrated that fasting downregulates Tet3 expression in AgRP neurons.

### TET3 Knockdown Upregulated AGRP in AgRP Neurons

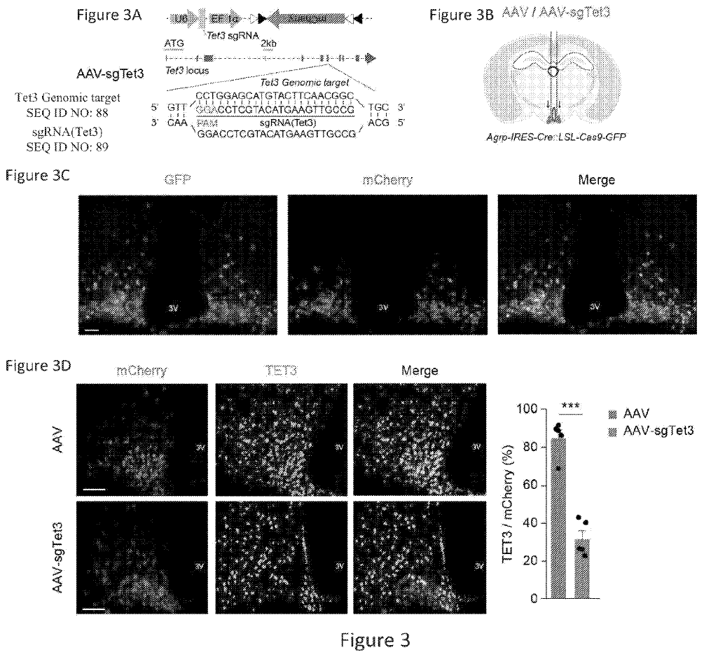

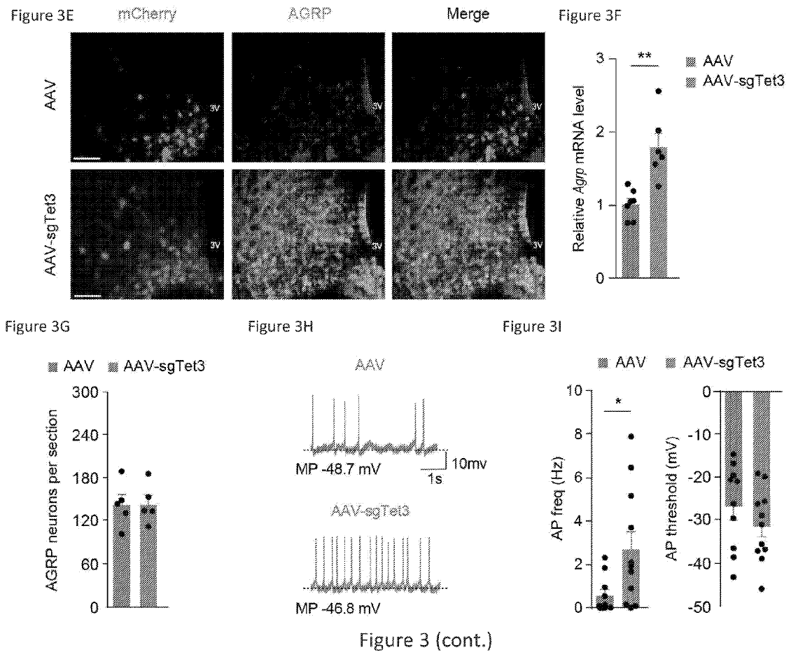

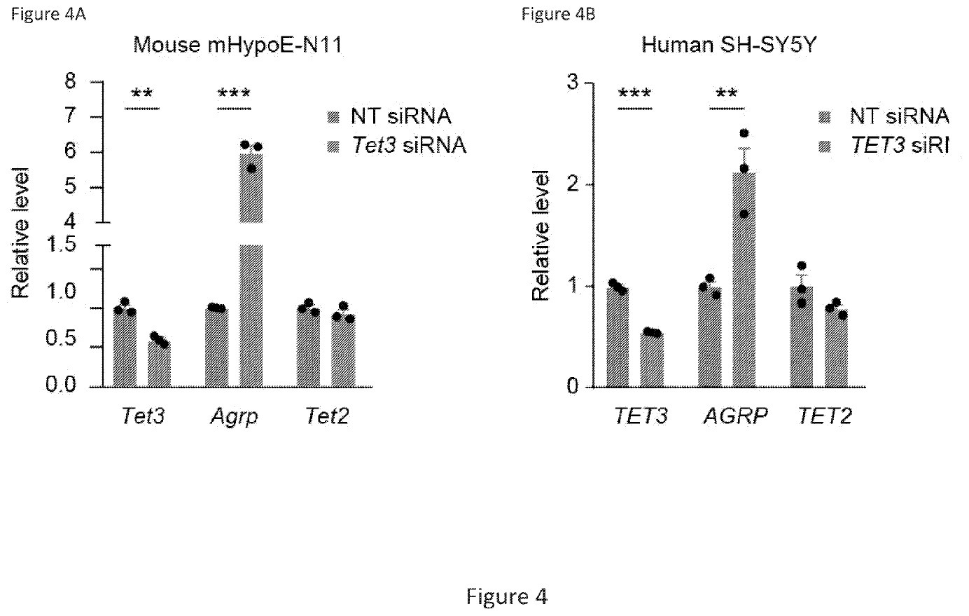

To directly evaluate the functional significance of TET3 in AgRP neurons, CRISPR gene-editing technology was utilized to downregulate TET3 specifically in AgRP neurons. An adeno-associated virus vector was prepared, containing a single-guide RNA targeting the mouse Tet3 locus (sgTet3) and a Cre-dependent mCherry reporter to indicate virus-transduced neurons (AAV-sgTet3, FIG. 3A). The sgTet3 sequence has been extensively validated for lack of CRISPR-mediated off-target mutagenesis (Sanjana N. E. et al., 2014, Nature Methods, 11:783-784). AAV-sgTet3 or a negative control AAV were injected bilaterally into the ARC of Cas9+ mice (FIG. 3B), and AgRP neuron-specific expression of sgTet3 was confirmed by the presence of GFP and mCherry double-positive cells (FIG. 3C) (Krashes, M. J. et al., 2011, Journal of Clinical Investigations, 121:1424-1428). To examine the effects of TET3 knockdown in AgRP neurons, ARCs were isolated (9:00 AM-10:00 AM) from fed mice injected with AAV-sgTet3 or AAV viruses. While the protein signal of TET3 in AAV-sgTet3-transduced AgRP neurons was significantly diminished as compared to AAV-transduced AgRP neurons (FIG. 3D), that of AGRP in the ARC was drastically increased (FIG. 3E). An increase in Agrp mRNA in the ARC of TET3 knockdown mice was also evident (FIG. 3F), and the level of increase was comparable to that seen in fasted animals (FIG. 2A). Importantly, AgRP neuron-specific TET3 knockdown did not affect AgRP neuronal viability (FIG. 3G). The negative regulation of Agrp expression by TET3 was further confirmed using neuronal cell lines. As seen in FIG. 4A, siRNA-mediated TET3 knockdown in a mouse embryonic hypothalamic cell line led to increased Agrp expression. Likewise, TET3 knockdown using an siRNA specifically targeting human TET3 (TET3 siRNA) in a human neuronal cell line upregulated AGRP expression (FIG. 4B). Collectively, these results demonstrated that TET3 negatively regulates Agrp expression in AgRP neurons and that this regulation was conserved between mouse and human.

### TET3 Knockdown Activated AgRP Neurons

TET3 knockdown led to an enhanced activity in AgRP neurons in brain slices isolated (9:00 AM-10:00 AM) from fed mice. The frequency of spontaneous action potentials (APs) was significantly higher (t=2.323, df=19, P<0.05, two-tailed t test) in ad libitum-fed knockdown mice (2.75±0.84 Hz, n=11 cells from 4 mice, FIG. 3H and FIG. 3I (red bar, left panel)) than in controls (0.62±0.28 Hz, n=10 cells from 3 mice, FIG. 3H and FIG. 3I (blue bar, left panel)). The AP threshold was −31.97±2.57 mV (n=11 cells from 4 mice, FIG. 3I, red bar, right panel) in knockdown animals and −27.17±3.21 (n=10 cells from 3 mice, FIG. 3I, blue bar, right panel) in controls, which was not significantly lowered (t=1.177, df=19, P=0.25) (FIG. 3I).

### Leptin Failed to Suppress Fasting-Induced Overeating in TET3 Knockdown Mice

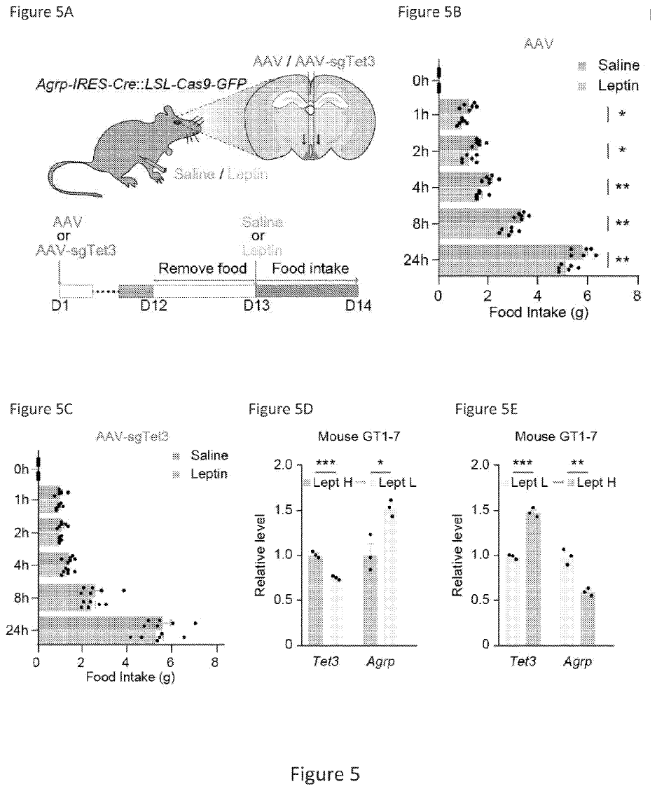

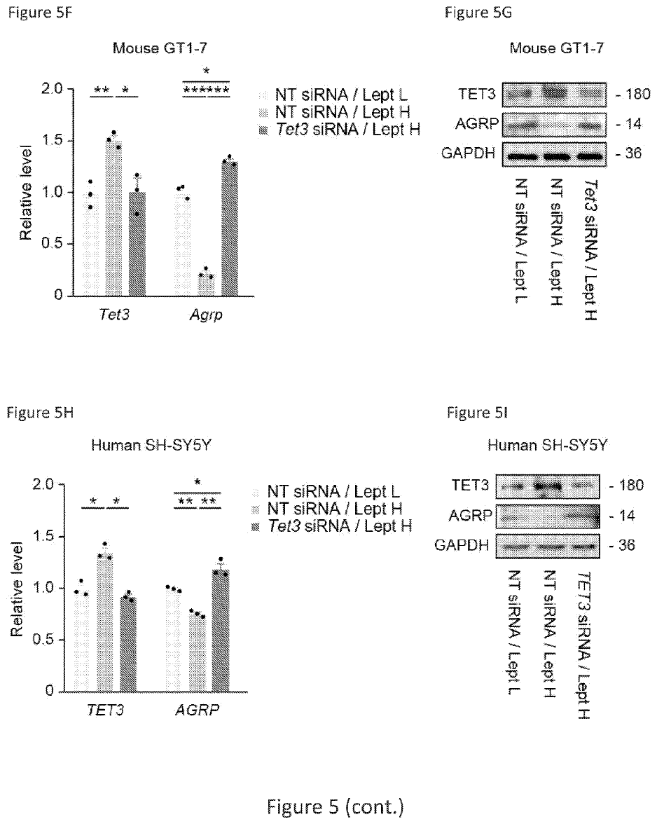

The activity of AgRP neurons is inhibited by the adipose hormone, leptin (Cowley, M. A. et al., 2001, Nature, 411:480-484). As CRISPR-mediated deletion of Lepr in adult AgRP neurons caused hyperphagia, obesity, and diabetes, and although not bound by any particular theory, it was hypothesized that TET3 might affect leptin signaling in AgRP neurons (Xu J. et al., 2019, Nature, 556:505-509). Thus, mice injected with AAV or AAV-sgTet3 were subjected to acute fasting followed by leptin or saline treatment and measurement of food intake (FIG. 5A). While leptin suppressed hunger-induced appetite in control mice (FIG. 5B), it failed to do so in TET3 knockdown animals (FIG. 5C), indicating that TET3 was necessary for leptin to inhibit hunger-induced overeating.

### TET3 Mediated Leptin-Induced Inhibition of Agrp Expression in Cell Lines

Circulating leptin levels fall during fasting or food deprivation (Ahima R. S. et al., 1996, Nature, 382:250-252; Burnett L. C. et al., 2017, International Journal of Obesity (London), 41:355-359; Buis D. T. P. et al., 2020, Thrombosis Research, 188:44-8). As food deprivation (i.e., low leptin signaling) downregulated TET3 in AgRP neurons (FIG. 2C), and although not bound by any particular theory, it was hypothesized that leptin might regulate TET3 expression, which was examined using neuronal cell lines. Thus, mouse GT1-7 hypothalamic cells maintained at a high leptin level were switched to a low leptin level (mimicking food deprivation), followed by gene expression analysis. While the expression of Agrp increased, that of Tet3 unexpectedly decreased in response to decreased leptin (FIG. 5D). Conversely, when cells maintained in low leptin levels were switched to high leptin levels (mimicking refeeding), opposite results were obtained (FIG. 5E). These results indicated that leptin had a positive effect on Tet3 expression.

Next, when GT1-7 cells maintained in low leptin levels were switched to high leptin levels in the presence of TET3 knockdown (FIG. 5F, left column), Agrp expression no longer decreased in response to increased leptin at both mRNA (FIG. 5F, right column) and protein (FIG. 5G) levels. Loss of leptin-induced inhibition of AGRP expression with TET3 knockdown was also observed in human neuronal cells (FIG. 5H and FIG. 5I). Collectively, these results showed that leptin upregulated TET3, which was required for leptin-induced inhibition of Agrp/AGRP expression both in mouse and human neuronal cells. Importantly, these results were in line with the in vivo findings that in fed mice, Agrp expression remained elevated in TET3 knockdown AgRP neurons (FIG. 3E and FIG. 3F).

### Mechanism of Leptin-Induced, TET3-Dependent Inhibition of Agrp Expression

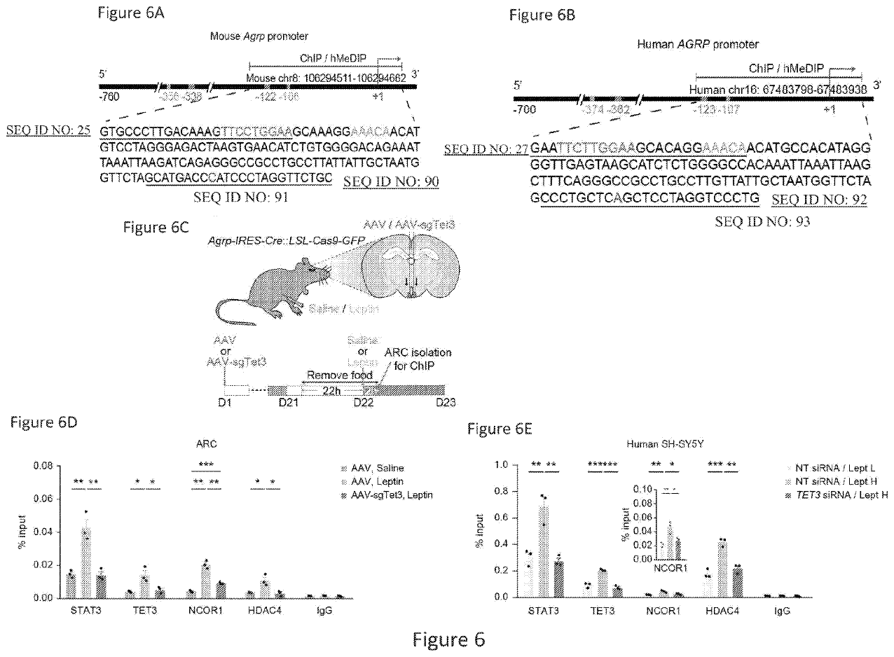

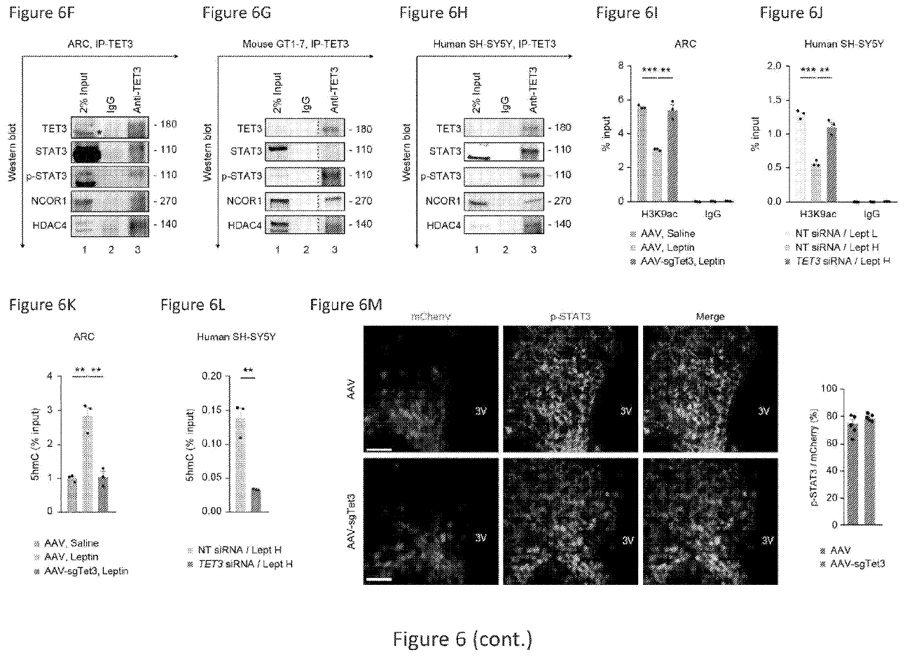

Binding of leptin to its receptor in AgRP neurons activates JAK2, which phosphorylates STAT3 at Tyr705 (p-STAT3); p-STAT3 then migrates as a dimer to the nucleus where it inhibits transcription of both Agrp and Npy (Wauman J. et al., 2017, Frontiers in Endocrinology (Lausanne), 8:30). However, the molecular mechanism underpinning this transcriptional regulation is complex and has remained incompletely understood. A large region of DNA (42.5-kb) upstream of the transcriptional start site of the mouse Agrp gene was identified to be both necessary and sufficient for the spatial expression and fasting response of AgRP in transgenic mice (Kaelin, C. B. et al., 2004, Endocrinology, 145:5798-5806). This regulatory region included an evolutionarily conserved proximal promoter of 760-bp (FIG. 6A) that overlapped with a minimal promoter of 700-bp of human AgRP (FIG. 6B) (Kaelin, C. B. et al., 2004, Endocrinology, 145:5798-5806; Brown, A. M. et al., 2001, Gene, 277:231-238). Using in vitro gel shift and luciferase reporter assays two STAT3 binding sites and two FOXO1 binding sites adjacent to each other were identified in the mouse Agrp promoter (Kitamura, T. et al., 2006, Nature Medicine, 12:534-540) (FIG. 6A). These studies also identified one STAT3 binding site adjacent to one FOXO1 binding site in the promoter of mouse Pomc, which is exclusively expressed in POMC neurons (Kitamura, T. et al., 2006, Nature Medicine, 12:534-540). Treating primary cells isolated from mouse hypothalamic, which contained mixed populations of AgRP and POMC neurons and other cell types, with leptin induced binding of STAT3 and inhibited binding of FOXO1 to these sequences (Kitamura, T. et al., 2006, Nature Medicine, 12:534-540). Using in vivo non-cell type-specific approaches (i.e., ARC injection of adenoviral expression vectors) STAT3 and FOXO1 were found to elicit opposing actions on the expression of Agrp and Pomc in mice, with STAT3 inhibiting and FOXO1 activating Agrp and FOXO1 inhibiting and STAT3 activating Pomc (Kitamura, T. et al., 2006, Nature Medicine, 12:534-540). Due to the non-cell type-specific nature of these studies, a clear mechanistic understanding of leptin induced, STAT3-mediated repression of Agrp expression in AgRP neurons was still lacking (Kitamura T. et al., 2006, Nature Medicine, 12:534-540).

Gene expression is strongly influenced by the accessibility of nucleosomal DNA and the state of chromatin compaction. Histone acetylation plays key roles in modulating chromatin structure and function. While acetylation is generally associated with an open chromatin state and active transcription, deacetylation is associated with transcriptional repression. Histone acetyltransferases (HATs) and histone deacetylases (HDACs) act antagonistically to control histone acetylation (Wang, Z. et al., 2009, Cell, 138:1019-1031). Transcriptional coregulators (including both coactivators and corepressors) act to bridge transcription factors and chromatin-modifying enzymes such as HATs and HDACs, determining the final transcriptional output. The same transcription factors can elicit opposing effects depending on which coregulators they interact with. NCOR1 is among the best-characterized corepressors shown to inhibit transcription by recruiting various HDACs in a context-specific manner (Mottis, A. et al., 2013, Genes & Development, 27:819-835).

Thus, these results indicated that in response to increased leptin levels, TET3, via interaction with STAT3, targeted a transcriptional corepressor complex containing NCOR1, and HDAC4 to Agrp to inhibit transcription. First, TET3 was required for leptin-induced repression of Agrp expression (FIG. 5F through FIG. 5I). Second, as a non-specific DNA-binding protein, TET3 was targeted to specific genomic loci via interaction with transcription factors (Lio C. J. et al., 2020, Journal of Bioscience, 45:21; Perera A. et al., 2015, Cell Reports, 11:283-294). Third, STAT3 was a transcription factor shown to physically interact with TET3 in human glioma cells (Herrmann A. et al., 2020, Oncogene, 29:2156-2169). Fourth, previous in vitro studies showed that STAT3 and FOXO1 competed for binding to the mouse Agrp promoter and that decreasing expression of FOXO1 induced binding of NCOR1 to the Agrp promoter (Kitamura T. et al., 2006, Nature Medicine, 12:534-540). Fifth, HDAC4 was found to be exclusively localized to the nuclei of mouse AgRP neurons by immunofluorescence (Takase K. et al., 2013, PLoS One, 8:e58473). Finally, mutations in the HDAC4/Hdac4 genes have been associated with eating disorders in both human and mouse (Lutter M. et al., 2017, Biological Psychiatry, 81:770-777; Cui H. et al., 2013, Journal of Clinical Investigations, 123:4706-4716).

As such, binding of STAT3, TET3, NCOR1, and HDAC4 to the mouse Agrp promoter at the basal level (in a fasted state when leptin level is low) was first detected and then evaluated to determine if the binding was affected by leptin treatment. Thus, mice injected with AAV or AAV-sgTet3 bilaterally into the ARC were fasted and treated with leptin or saline, followed by isolation of ARCs and ChIP-qPCR analysis (FIG. 6C). While leptin increased binding of STAT3, TET3, NCOR1, and HDAC4 to the Agrp promoter as compared to basal, it failed to do so in AAV-sgTet3 injected mice (FIG. 6D). Notably, in these mice the association of STAT3, NCOR1, and HDAC4 with the promoter remained at the basal level after leptin treatment (FIG. 6D), indicating that TET3 was required for leptin-induced association of these proteins with the promoter. Importantly, this leptin-induced TET3-dependent association of STAT3, NCOR1, and HDAC4 with the Agrp promoter in mice was recapitulated in human neuronal cells (FIG. 6E) indicating a conserved mechanism.

Further co-immunoprecipitation studies (co-IP) were then performed to examine protein-protein interactions in the presence of leptin. Thus, mice were fasted and then treated with leptin for 2 h, followed by ARC isolation and co-IP studies. When TET3 was pulled down (FIG. 6F, top blot, lane 3), STAT3/p-STAT3 (FIG. 6F, second and third blots from top, lane 3), NCOR1 (FIG. 6F, fourth blot from top, lane 3) and HDAC4 (FIG. 6F, bottom blot, lane 3) were detected in the immunoprecipitated complexes. Similar results were obtained using mouse and human neuronal cells (FIG. 6G and FIG. 6H). Both STAT3 and HDAC4 were known to undergo a variety of post-translational modifications including phosphorylation, acetylation and methylation in a context-dependent manner (Wang, Z. et al., 2014, Epigenomics, 6:139-150; Tesoriere, A. et al., 2021, Biomedicines, 9:956), which can affect protein mobility on western blot gels. The enrichment of p-STAT3 (phosphorylated at Tyr705) in TET3-containing complexes both in vivo (FIG. 6F) and in vitro (FIG. 6G and FIG. 6H) was consistent with a functional interaction between TET3 and p-STAT3. Taken together with the ChIP data (FIG. 6D and FIG. 6E), these results indicated that TET3, p-STAT3, NCOR1, and HDAC4 formed a multi-protein complex on the Agrp/AGRP promoters.

To determine whether the leptin-induced, TET3-depedent association of the corepressor complex affected histone acetylation, ChIP-qPCR analysis was performed using an antibody specific for H3K9ac, a histone mark for active transcription (Wang, Z. et al., 2009, Cell, 138:1019-1031). Thus, mice were treated as in FIG. 6C, and ChIP experiments were performed using anti-H3K9ac. A leptin-induced reduction was observed in histone acetylation at the Agrp promoter, which was abolished in TET3 knockdown mice (FIG. 6I). This leptin-induced, TET3-dependent reduction in histone acetylation at the AGRP promoter was also observed in human neuronal cells (FIG. 6J). Collectively, these results indicated that leptin induced formation of a transcriptional corepressor complex on the Agrp/AGRP promoters in a TET3-dependent manner, leading to histone deacetylation and inhibition of transcription.

### TET3 Induced 5hmC Modification of the Agrp/AGRP Promoters

TETs initiate DNA demethylation by oxidizing 5mC to 5hmC; 5hmC also serves as a stable epigenetic mark. In postmitotic neurons 5hmC accumulates at ~10 times the level present in peripheral cell types (Kriaucionis S et al., 2009, Science, 324:929-930; Globisch D et al., 2010, PLoS One, 5:e15367; Munzel M et al., 2010, Angew Chem Int Ed Engl. 49:5375-5377). 5mC and 5hmC have been detected at both CpG and non-CpG (CpH, where H=A/C/T) dinucleotides and can function to enhance or inhibit binding of regulatory protein factors in a context-dependent manner (Syed, K. S. et al., 2016, Biochemistry, 55:6940-6948; Mellen, M. et al., 2017, Proceedings of the National Academy of Sciences USA, 114:343-348; DeNizio, J. E. et al., 2021, Journal of Molecular Biology, 433:166877; Takamura, N. et al., 2021, Genes to Cells, 26:121-135). In postmitotic neurons, non-CpG 5hmC occurs predominantly in CpA dinucleotides (Mellen, M. et al., 2017, Proceedings of the National Academy of Sciences USA, 114:343-348).

An enrichment of CpA dinucleotides was observed in both the mouse and human Agrp/AGRP promoters (FIG. 6A and FIG. 6B). Binding of TET3 to the Agrp/AGRP promoters induced 5hmC modification, enabling a stable association of STAT3 and the corepressor complex with the promoters. Thus, mice were treated as in FIG. 6C, and genomic DNA was isolated from the ARCs and subjected to hydroxymethylated DNA immunoprecipitation (hMeDIP)-qPCR analysis as previously described (Li, D. et al., 2020, Nature Communications, 11:342). As seen in FIG. 6K, an increase in 5hmC in the Agrp promoter in leptin- vs. saline-treated mice was evident, while in TET3 knockdown animals no increase in 5hmC was detected after leptin treatment. Thus, there exists a positive correlation between TET3 binding (FIG. 6D) and 5hmC modification (FIG. 6K) of the Agrp promoter in vivo. Importantly, the TET3-dependent increase in 5hmC modification of the AGRP promoter was recapitulated in human cells (FIG. 6L). Further, TET3 knockdown did not affect STAT3 phosphorylation in AgRP neurons in mice (FIG. 6M), indicating that the reduced association of STAT3 with the Agrp promoter seen in TET3 knockdown AgRP neurons (FIG. 6D) was not a result of decreased STAT3 phosphorylation. Given the positive connection between 5hmC (FIG. 6K and FIG. 6L) and binding of STAT3 and the corepressor complex to the Agrp/AGRP promoters (FIG. 6D and FIG. 6E), TET3-induced 5hmC modification in the Agrp/AGRP promoters enabled a stable association of STAT3 and the corepressor complex with the promoters.

### TET3 Negatively Affected the Expression of Npy and Slc32a1

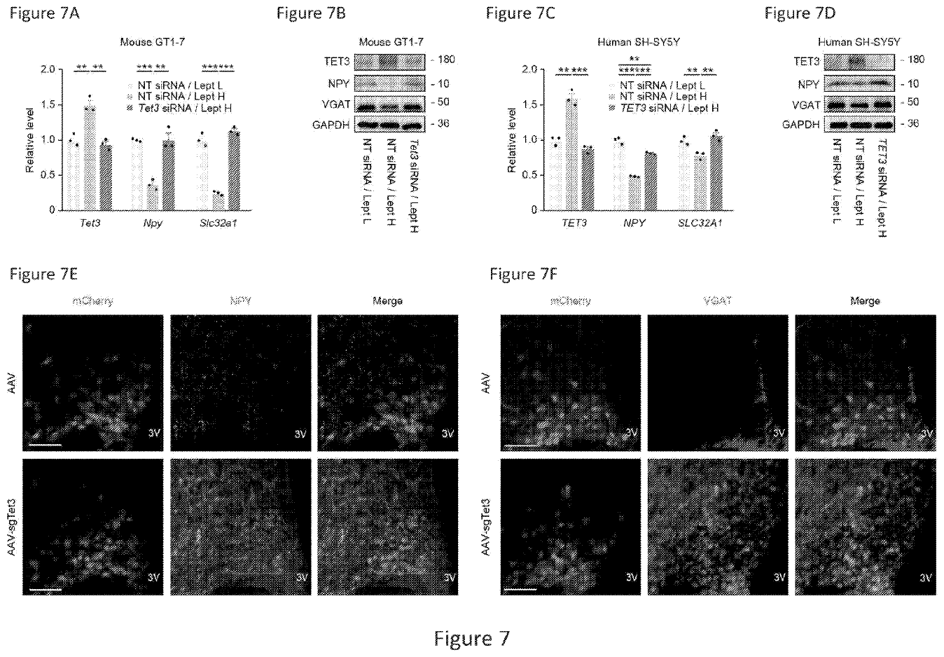

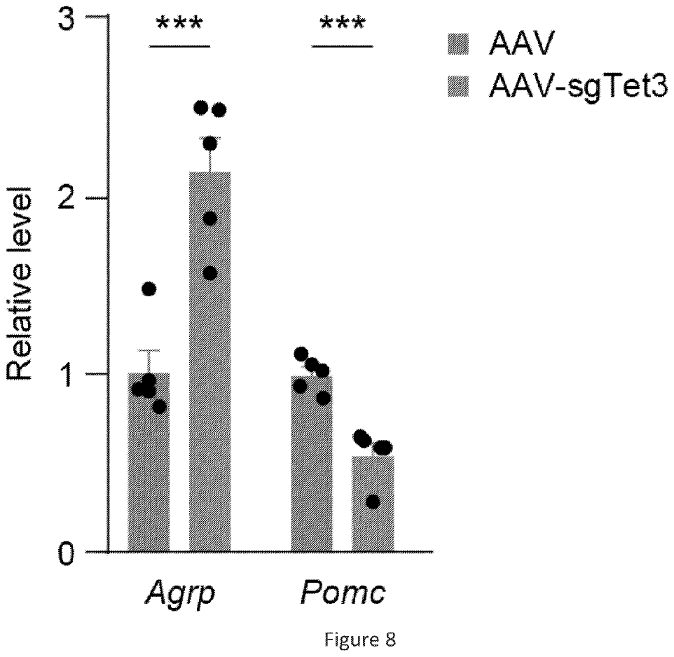

Activated AgRP neurons release AGRP, NPY, and GABA, which act in concert to stimulate food intake and reduce energy expenditure, which act in concert to stimulate food intake and reduce energy expenditure. As such, additional studies sought to test whether the expression of Npy and Slc32a1 (encoding VGAT, which is required for loading GABA into synaptic vesicles) was also regulated by TET3. Unlike AGRP, which was expressed exclusively in AgRP neurons, both NPY and VGAT were also expressed in other neurons. Similar to what was seen for AGRP (FIG. 5F through FIG. 5I), leptin negatively affected the expression of NPY and VGAT in a TET3-dependent manner, both at the mRNA and protein levels (FIG. 7A and FIG. 7B). This regulation was also observed in human cells (FIG. 7C and FIG. 7D). Further, there was a significant increase in the protein signals of NPY and VGAT in the ARCs of TET3 knockdown vs. control mice (FIG. 7E and FIG. 7F), consistent with TET3-dependent inhibition of expression of NPY and VGAT in AgRP neurons. The diffuse signals of NPY and VGAT reflect NPY being a secreted peptide and VGAT being in synaptic vesicles of neuronal projections. Further, an increased expression of Agrp mRNA and decreased expression of Pomc mRNA were observed in the ARCs of ad libitum-fed mice injected with AAV-sgTet3 vs. AAV (FIG. 8), consistent with the notion that activated AgRP neurons inhibit POMC neurons through the release of GABA (Deem, J. D. et al., 2021, FEBS Journal, 289:2362-2381).

### AgRP Neuron-Specific TET3 Knockdown Caused Hyperphagia, Obesity, and Diabetes

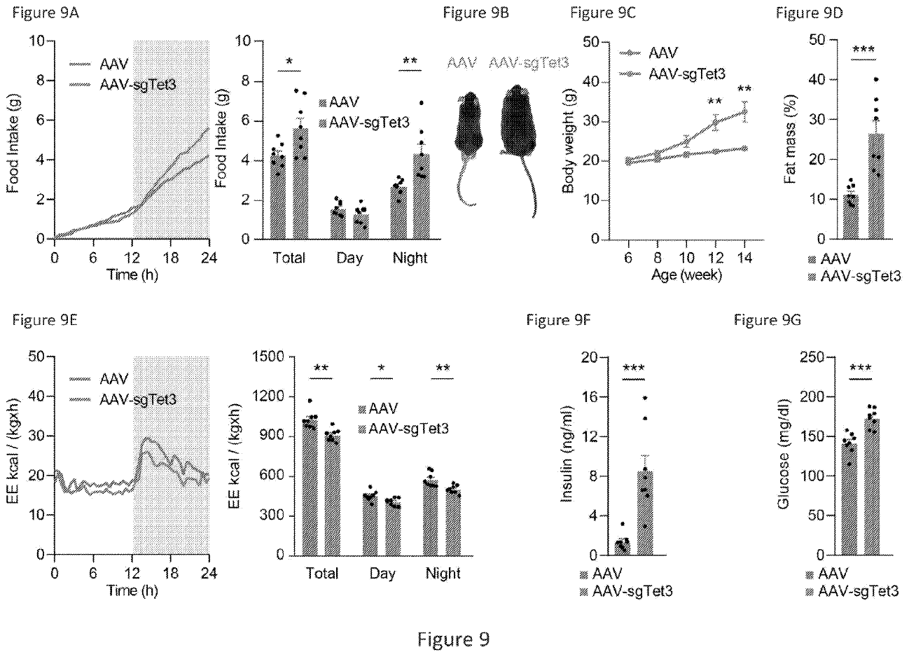

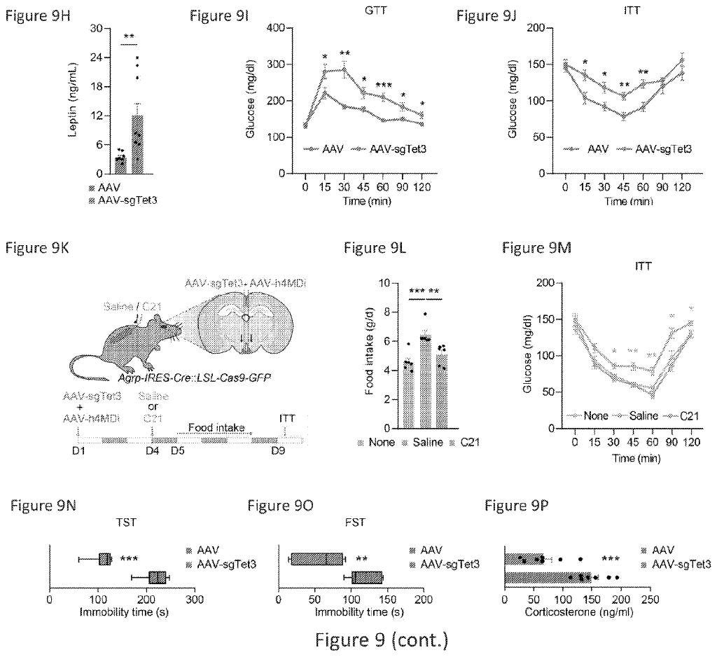

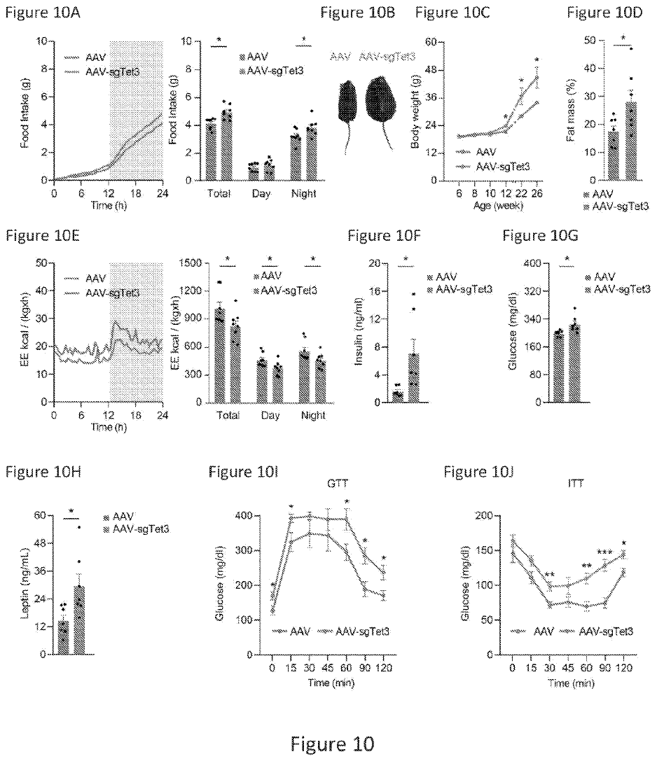

Activated AgRP neurons can release AGRP, NPY, GABA, and augmentor a, all of which individually and in concert can potently affect feeding and systemic glucose metabolism (Horvath T. L. et al., 1997, Brain Research, 756:283-286; Pu S. et al., 1999, Endocrinology, 140:933-940; Steculorum S. M. et al., 2016, Cell, 165:125-138; Engstrom R. L. et al., 2020, Nature Communications, 11:442; Ahmed M. et al., 2022, Proceedings of the National Academy of Sciences USA, 119:e2200476119). Notably, deletion of VGAT, which is required for vesicular loading of GABA, led to complete loss of synaptic GABA release from AgRP neurons (Wojcik, S. M. et al., 2006, Neuron, 50:575-587; Tong, Q. et al., 2008, Nature Neuroscience, 11:998-1000). In line with these observations, CRISPR-mediated TET3 knockdown in AgRP neurons induced hyperphagia, obesity and diabetes, as determined by increased food intake (FIG. 9A), increased body weight and fat mass (FIG. 9B through FIG. 9D), decreased energy expenditure (FIG. 9E), elevated blood insulin, glucose, and leptin levels (FIG. 9F through FIG. 9H), and decreased glucose tolerance and insulin sensitivity (FIG. 9I and FIG. 9J), both in female (FIG. 9A through FIG. 9J) and male (FIG. 10) mice. Notably, the increases in food intake (FIG. 9A and FIG. 10A) and energy expenditure (FIG. 9E and FIG. 10E) in AAV-sgTet3 injected mice were observed at 2-3 weeks post-injection, before significant increases in body weight/fat mass were detected (FIG. 9C and FIG. 10C) indicating direct and body weight/fat mass-independent effects of TET3 knockdown in AgRP neurons.

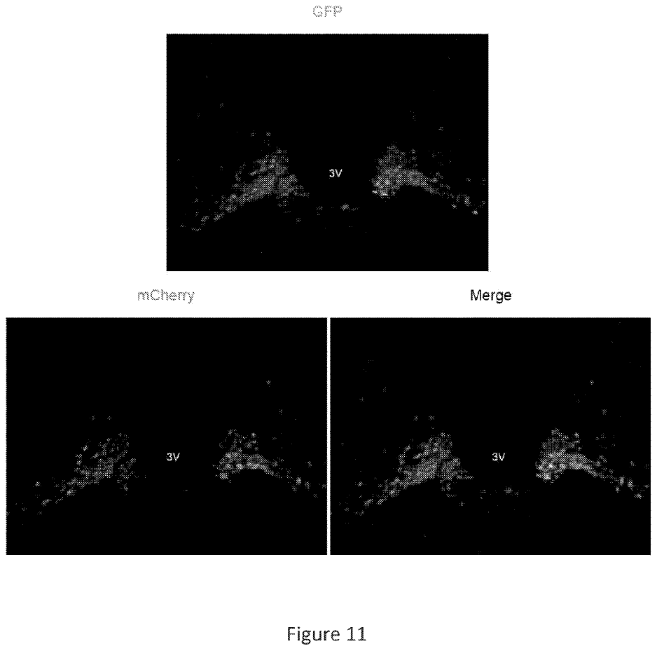

To determine the relevance of activated AgRP neurons from TET3 knockdown, the chemogenetic tool of designer receptors exclusively activated by designer drugs (DREADDs) was used. AAV-sgTet3 was co-injected with an AAV containing a Cre-dependent hM4Di-mCherry transgene (AAV-hM4Di) (Krashes, M. J. et al., 2011, Journal of Clinical Investigations, 121:1424-1428) bilaterally into the ARC of Cas9+ mice, followed by implantation of an osmotic pump to infuse DREADD agonist 21 (C21) (Thompson, K. J. et al., 2018, ACS Pharmacology & Translational Science, 1:61-72) or saline (FIG. 9K). AgRP neuron-specific expression of hM4Di was confirmed by immunofluorescence (FIG. 11). Stimulation of hM4Di with C21, thereby inhibiting AgRP neurons, suppressed hyperphagia and reversed systemic insulin resistance induced by TET3 knockdown in ad libitum-fed mice as early as one-week post-injection (FIG. 9L and FIG. 9M). These results showed that activation of AgRP neurons as a result of TET3 knockdown contributes to both hyperphagia and systemic insulin resistance.

### AgRP Neuron-Specific TET3 Knockdown Reduced Stress-Like Behaviors

It has previously shown that activation of AgRP neurons affects many complex behaviors beyond feeding (Dietrich M. O. et al., 2012, Nature Neuroscience, 15:1108-1110; Dietrich M. O. et al., 2015, Cell, 160:1222-1232; Miletta M. C. et al., 2020, Nature Metabolism, 2:1204-1211; Copperi F. et al., 2021, Biological Psychiatry, 91:879-887). The melanocortin system, to which AgRP belongs, has also been tied to stress and depression (Copperi F. et al., 2021, Biological Psychiatry, 91:879-887; Bruschetta G. et al., 2020, Cell Reports, 33:108267). Because TET3 knockdown in AgRP neurons activated these cells, the stress-like behaviors were evaluated in these animals using a tail suspension test and forced swim test. The TET3 knockdown animals spent less immobility time than the control mice both in the tail suspension test (FIG. 9N) and in the forced swim test (FIG. 9O), indicating decreased stress-like states. Further, compared to control animals the TET3 knockdown mice had reduced plasma cortisol levels (FIG. 9P). Collectively, these data indicated that TET3 knockdown in AgRP neurons produced anti-stress effects.

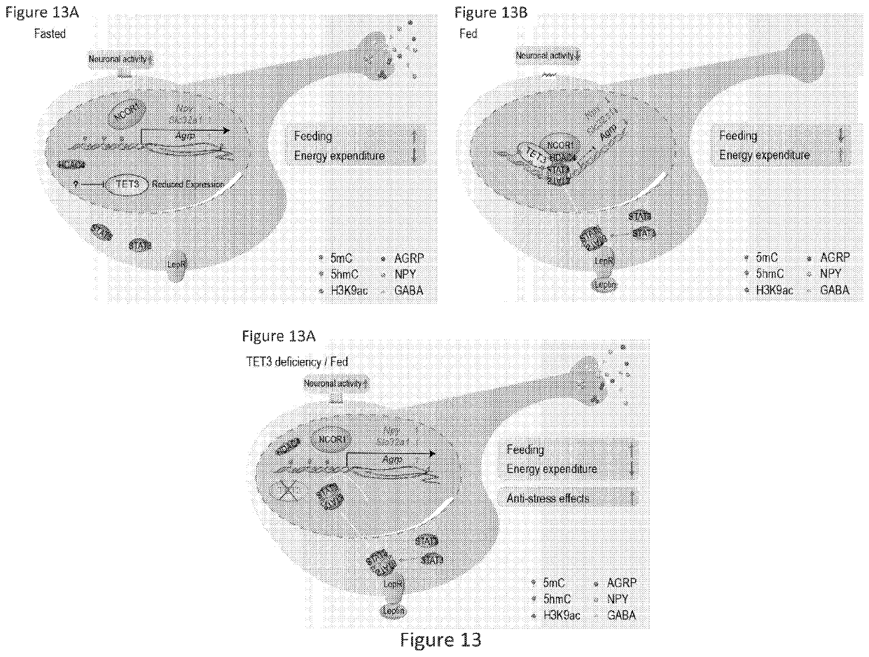

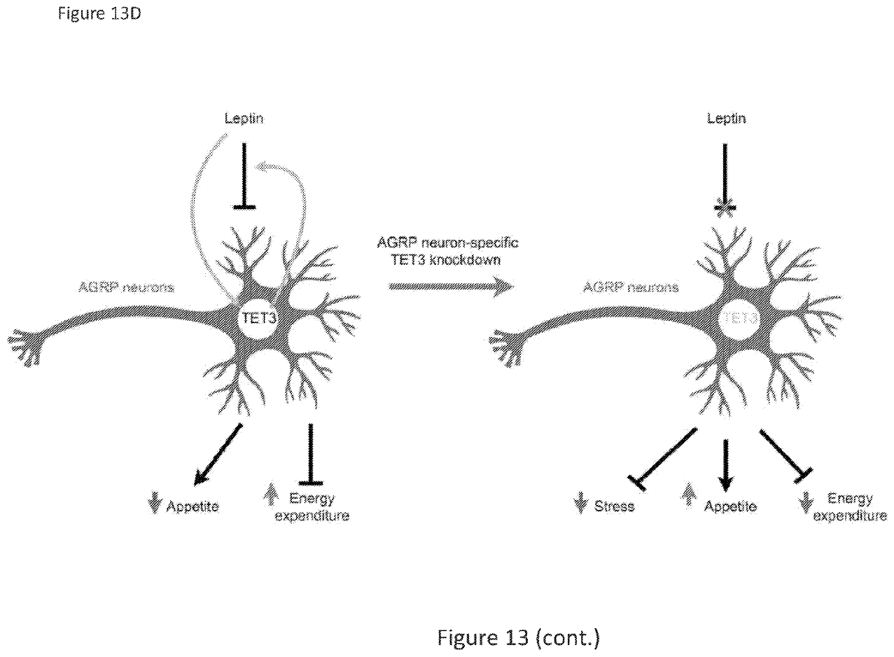

In the present study, it was found that food deprivation downregulated TET3 in AgRP neurons and that CRISPR-mediated cell-specific ablation of TET3 in AgRP neurons activated these neurons and upregulated expression of Agrp, Npy and Slc32a1 in adult mice. It was also found that AgRP neuron-specific TET3 knockdown caused hyperphagia, obesity, and diabetes both in female and male mice, highlighting a central role of TET3 in regulation of feeding, body weight, and glucose metabolism by AgRP neurons. Using both mouse models and human and mouse neuronal cell lines, the present studies demonstrated that TET3 knockdown in AgRP neurons dysregulated neuronal activity and impaired leptin signaling. In particular, the herein described studies revealed that TET3 was required for leptin-induced inhibition of Agrp/AGRP expression both by promoting 5hmC modification and by recruiting a novel chromatin-modifying complex to the promoters of Agrp/AGRP and that this mechanism of dual action of TET3 was conserved between mouse and human. Furthermore, the studies described herein showed that TET3 knockdown in AgRP neurons induced anti-stress effects (FIG. 13).

In summary, the studies have uncovered an important aspect of TET3 regulation as a critical epigenetic component of the neural circuits in control of satiety, energy metabolism, and non-feeding behaviors. Furthermore, while the requirement of STAT3 in leptin signaling has been well-established, the present data showing that STAT3, TET3, NCOR1, and HDAC4 formed a multi-protein complex on the Agrp/AGRP promoters indicated that NCOR1, and HDAC4 are important for leptin signaling. Second, FOXO1 has been indicated to compete with STAT3 for binding to the Agrp promoter, stimulating transcription (Kitamura T et al., 2006, Nat Med., 12:534-540). Thus, additional studies also focus on determining what role FOXO1 plays in the TET3-dependent regulation of Agrp expression in AgRP neurons. Furthermore, based on the robust metabolic phenotype of TET3 knockdown in AgRP neurons, Agrp, Npy and, Slc32a1 genes are regulated by TET3. Indeed, TET3 deficiency caused chronic activation of AgRP neurons independent of food/energy status indicated other yet unidentified genes affected by TET3.

It has been previously reported that leptin failed to elicit acute suppression of hunger-induced overeating in mice with AgRP neuron-specific disruption of GABA_{A} receptors. This phenotype was recapitulated by DMH neuron-specific deletion of Lepr due, at least in part, to the loss of GABAergic afferents on AgRP neurons that are necessary for AgRP neurons to inhibit appetite. Under normal conditions leptin acted on its receptor in DMH neurons to promote release of GABA which in turn acts on AgRP neurons enabling AgRP neurons to suppress food intake (Xu J et al., 2018, Nature, 556:505-509). The studies described herein demonstrated that AgRP neuron-specific TET3 knockdown abolished leptin's ability to suppress fasting-induced overeating, indicating that TET3 directly or indirectly regulated expression of genes encoding GABA_{A} receptors.

Further, in cultured hippocampal neurons, TET3 acted as a synaptic sensor in regulation of neuronal activity: increased synaptic activity upregulated TET3 expression, whereas TET3 inhibition elevated excitatory glutamatergic synaptic transmission (Yu H et al., 2015, Nat Neurosci., 18:836-843). Likewise, CRISPR-mediated TET3 deletion in young mice increased excitatory and decreases inhibitory synaptic transmission in cerebral cortex neurons (Wang L et al., 2017, Cell Res., 27:815-829). Future studies are aimed at identification of other TET3 targets as well as a more in-depth dissection of protein-protein interactions of TET3, STAT3, NCOR1, HDAC4 and FOXO1 to obtain a more comprehensive mechanistic understanding of TET3 in central regulation of feeding and energy metabolism, including its involvement with the known role of synaptic plasticity in these circuits (Pinto S et al., 2004, Science, 304:110-115; Horvath T L et al., 2004, Nat Rev Neurosci., 5:662-667).

The essential role of AgRP neurons in regulation of food intake, body weight, and energy metabolism has been unambiguously established (Luquet S et al., 2005, Science, 310:683-685; Gropp E et al., 2005, Nat Neurosci., 8:1289-1291). The herein described discovery of TET3 as an essential mediator of leptin-induced suppression of Agrp expression in AgRP neurons is conceptually novel. First, none of the TET family proteins has been previously documented to play a role in central control of feeding, obesity and glucose metabolism. Second, the current understanding has been focusing on the notion that leptin signaling activates STAT3, which binds to the Agrp promoter and inhibits transcription. However, how inhibition of transcription is accomplished has not been well-defined. The herein described studies demonstrated that leptin-induced STAT3 binding enabled TET3-dependent recruitment of the transcriptional corepressor NCOR1, and HDAC4 to the Agrp/AGRP promoters, which in turn promoted histone deacetylation leading to inhibition of transcription. Third, as 5hmC modification of DNA is known to affect protein binding (Syed K S et al., 2016, Biochemistry, 55:6940-6948; Mellen M et al., 2017, Proc Natl Acad Sci USA, 114:E7812-E7821), the results indicated that TET3-induced 5hmC modification of the Agrp/AGRP promoters enabled a stable association of STAT3 and a chromatin-modifying complex with the promoters and that TET3 knockdown did not alter STAT3 phosphorylation. As dysregulation of leptin signaling is tightly associated with human obesity and diabetes (Friedman J M et al., 2019, Nat Metab., 1:754-764), the herein described discovery of dual action of TET3 (5hmC modification and recruitment of chromatin-modifiers) in regulation of Agrp/AGRP expression in both human and mouse cells offered new opportunities for development of therapeutic interventions for metabolic disorders and related psychiatric conditions.

The materials and methods employed in Example 1 are now described.

Immunofluorescence Postfixed sections were cut into 40 μm-thick sections. After being washed 5 times for 10 minutes in washing buffer (0.1 M PB, 0.4% Triton x-100, 1% BSA, 0.1 L-Lysine, pH 7.3-7.5), the sections were incubated in blocking solution (1:50 normal donkey serum in washing buffer) for 20 minutes at room temperature. Sections were incubated with anti-TET3 (dilution 1:2000; ABE290, Millipore Sigma) (FIG. 1), anti-AGRP (dilution 1:2000; PA5-78739, Invitrogen) (validated by the vendor), anti-Phospho-Stat3 (Tyr705) (dilution 1:2000; Cell Signaling, 9145S) (Liang et al., 2019, Mol Ther Nucleic Acids, 18:183-193), anti-NPY (dilution 1:800, Cell Signaling, 11976S) (Zhang et al., 2021, Nat Commun, 12:5740), or anti-VGAT (dilution 1:200, Abcam, Ab235952) (validated by the vendor) overnight at 4 °C (Li, D. et al., 2020, Nature Communications, 11:342; Liang, C. et al., 2019, Molecular Therapy—Nucleic Acids, 18:183-193; Zhang, X. et al., 2021, Nature Communications, 12:5740). Negative controls were performed by omitting the respective primary antibodies. The next day, sections were washed 5 times (15 minutes each) in phosphate buffered saline (PBS) and incubated in 0.4% Triton x-100 PBS with the respective secondary antibodies for 1 h at room temperature: donkey anti-Rabbit IgG Fluor 350 (dilution 1:500, A10039, Invitrogen); donkey anti-Rabbit IgG Fluor 594 (dilution 1:500, A-21207, Invitrogen). The sections were coverslipped and visualized using a Keyence BZ-X700 fluorescence microscope. The fluorescence signals from GFP and mCherry in AgRP neurons were detected without immunostaining.

### Neuronal Cell Culture and Treatments

Mouse GT1-7 hypothalamic neuronal cell line (Sigma-Aldrich, SCC116), human SH-SY5Y neuronal blastoma cell line (ATCC, CRL-2266), and embryonic mouse hypothalamus cell line N11 (mHypoE-N11) (Cedarlane, CLU107) were purchased and cultured according to the manufacturers' instructions. For siRNA transfection in a 24-well plate scale, cells were seeded at a density of 2×10⁵ cells/well the day before transfection. To prepare siRNA transfection solution for each well of cells, 5 pmol of NT siRNA (non-targeting control siRNA, AM4636, Ambion), Tet3 siRNA (siRNA specifically targeting mouse Tet3, 4390815/s101483, Ambion), or TET3 siRNA (siRNA specifically targeting human TET3, 4392420/s47238, Ambion) (Cao et al., 2019, Oncogene, 38:5356-5366; Xu et al., 2020, Cell Rep., 30:1310-1318) were mixed with 25 μl of OPTI-MEM (Gibco, 31985-070) by gentle pipetting (Cao, T. et al., 2019, Oncogene, 38:5356-5366; Xu, Y. et al., 2020, Cell Reports, 30:1310-1318 e5). In parallel, 1.5 μl of Lipofectamine RNAiMAX (Invitrogen, 13778-150) was mixed with 25 μl of OPTI-MEM by gentle pipetting. Following 5 min of incubation at room temperature, the resulting 50 μl of transfection solution was added to one well of cells containing 1 ml culture media. For GT1-7 transfection shown in FIG. 1B and FIG. 1C, and mHypoE-N11 transfection shown in FIG. 4A, media were changed the next day, followed by RNA extraction or immunofluorescence at 48 h posttransfection. In the experiments shown in FIG. 1B and FIG. 1C and FIG. 4A, no leptin was present in the culture media. For leptin treatments, cells were incubated with leptin (mouse leptin L3772-1MG, Sigma Aldrich, for GT1-7; human leptin L4146-1MG, Sigma Aldrich, for SH-SY5Y) at concentrations of 1×10⁻⁸M (Lept H) or 1×10⁻¹⁰ M (Lept L) in culture media. The durations of Lept H and Lept L treatments are indicated in the figure legends.

### Chromatin Immunoprecipitation-Quantitative PCR (ChIP-qPCR)

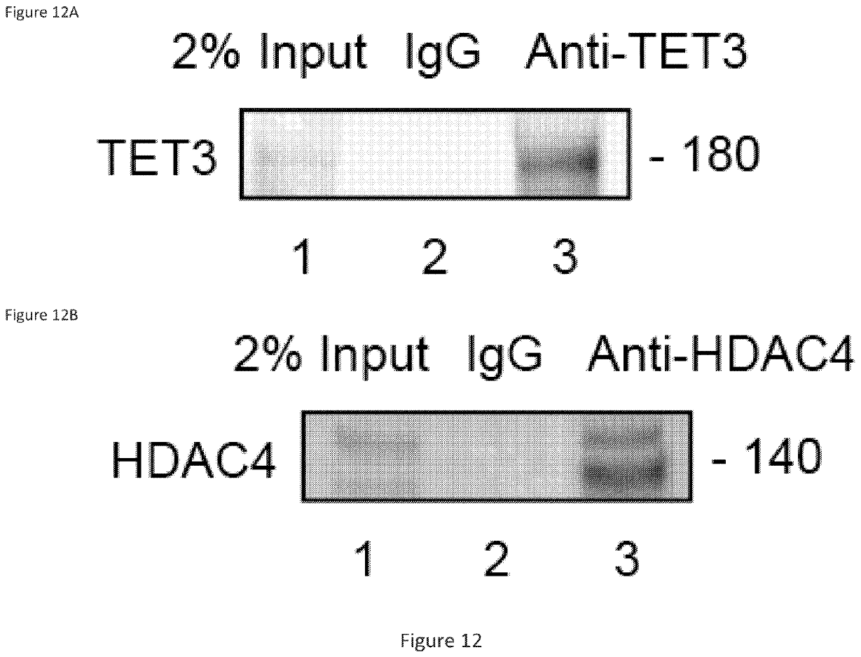

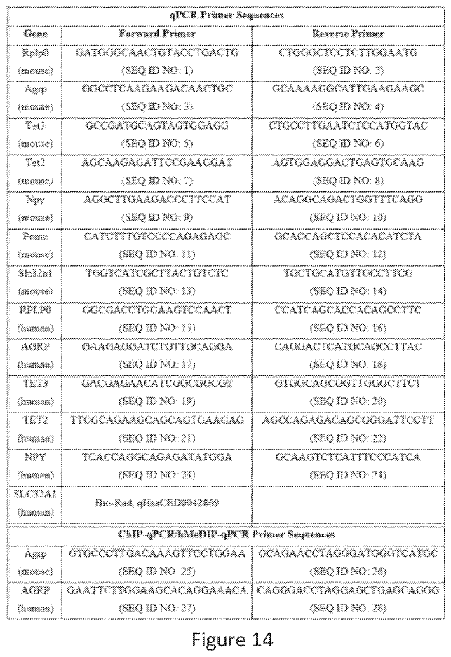

To prepare antibodies, 5 μl (packed volume) of ChIP grade Dynabeads Protein G (Invitrogen, Thermo Scientific, 10004D) were washed twice with 1 ml of binding buffer (0.2% Tween-20 in PBS), followed by incubation on a rotator with 10 μg of rabbit polyclonal anti-TET3 (Active Motif, 61395) (FIG. 12A), anti-STAT3 (Proteintech, 10253-2-AP), anti-NCOR1 (Cell Signaling, 5948S) (Hainberger et al., 2020, Front Immunol., 11:579), anti-HDAC4 (Active Motif, 40969) (FIG. 12B), anti-H3K9ac (Active Motif, 39137) (validated by the vendor), or preimmune rabbit IgG (as a negative control) in 350 μl of binding buffer at 4 °C overnight (Gao, Y. et al., 2020, Advanced Science, 7:2002518; Hainberger, D. et al., 2020, Frontiers in Immunology, 11:579). Antibody bound beads were washed twice with 1 ml of binding buffer, resuspended in 50 μl of dilution buffer (0.01% SDS, 1.1% Triton X-100, 1.2 mM EDTA, 16.7 mM Tris-HCl at PH 8.0, 167 mM NaCl), and kept on ice until use. To prepare chromatin, freshly isolated ARCs (2 ARCs from one mouse per ChIP, FIG. 6D and FIG. 6I) were washed twice with 1 ml of cold-PBS, followed by cross-linking in 1% paraformaldehyde/PBS on a rotator at room temperature for 15 min. Glycine buffer (150 mM final concentration) was added and incubated in rotation at room temperature for 10 min to quench cross-linking. Cross-linked ARCs were washed twice with PBS and homogenized (5-10 strokes) using a disposable pellet pestle (Fisher Scientific, 12-141-368) in 300 μl of cold cell lysis buffer (50 mM Tris-HCl at PH 8.0, 140 mM NaCl, 1 mM EDTA, 10% glycerol, 0.5% NP-40, 0.25% Triton X-100), followed by incubation at 4 °C for 20 min to lyse the plasma membrane. For cell ChIP (FIG. 6E and FIG. 6J), SH-SY5Y cells seeded at a density of 6×10⁶ cells/plate in a 100 mm plate the night before were transfected with NT siRNA in Lept L (NT siRNA/Lept L) or Lept H (NT siRNA/Lept H), or with TET3 siRNA in Lept H. After 48 h, 360 μl of 32% paraformaldehyde (final concentration 1%) was added to the plate to cross-link cells at room temperature for 10 min, followed by addition of glycine buffer (150 mM final concentration) to quench cross-linking for 5 min. Cross-linked cells were washed twice with PBS and harvested in 1000 μl of cold cell lysis buffer, followed by incubation at 4 °C for 20 min to lyse the plasma membrane. Nuclei from ARCs or SH-SY5Y cells were pelleted by centrifugation at 2000×g for 5 min at 4 °C and resuspended in 300 μl of cold nuclear lysis buffer (10 mM Tris-HCl at PH 8.0, 0.5 mM EGTA, 1 mM EDTA, 0.2% SDS), followed by rotation at 4 °C for 20 min. Chromatin was sheared to produce 200-500 bp DNA fragments using a sonifier (Branson 150), with a setting of 15 pulses of 10 sec each at 35% amplitude followed by a 40 sec rest period on ice between each pulse. Samples were centrifuged at 16000×g for 10 min at 4 °C to remove insoluble materials, and the resulting supernatant was subjected to a 2-fold dilution using nuclear lysis buffer. 5%-10% of diluted chromatin were saved as input samples and stored at 4 °C until use. To perform ChIP, 500 μl of diluted chromatin was added to each tube containing antibody-bound beads and incubation on a rotator was carried out overnight at 4 °C. Beads were washed eight times with 1 ml of cold wash buffer (100 mM Tris-HCl at PH 8.0, 500 mM NaCl, 1% deoxycholic acid, 1% NP-40) by rotating at 4 °C for 5 min each. Beads were eluted with 85 μl of elution buffer (50 mM Tris-HCl at PH 8.0, 10 mM EDTA, 1% SDS) by agitation in a thermomixer at 65 °C for 10 min. Elution was repeated once and the two eluants were combined. The eluants and the input samples were incubated at 65 °C for 4 h to reverse the cross-links. 10 μg of RNase A was added to each sample and incubation was carried out for 1 h at 37 CC. 200 μg of proteinase K dissolved in 120 μl of TE buffer (50 mM Tris-HCl at PH 8.0, 10 mM EDTA) was added to each sample and incubation was carried out for 2 h at 65 °C. Samples were purified using the QIAquick PCR purification Kit (QIAGEN, 28104) and eluted in 30 μl of ddH₂O. Levels of ChIP-purified DNA were determined by qPCR. For ARC ChIP-qPCR, a previously reported primer set was used (Loganathan, N. et al., 2021, Neuroendocrinology, 111:678-695). For SH-SY5Y cell ChIP-qPCR, a pair of house-designed primers was used. The sequences of both primer sets are listed in FIG. 14 and Table 1. The relative enrichments of the DNA regions were calculated using the Percent Input Method and are presented as % input as previously described (Li, D. et al., 2020, Nature Communications, 11:342).

TABLE 1
Primer Sequences

| | | |
|---|---|---|
| Gene | Forward Primer | Reverse Primer |

qPCR Primer Sequences

| | | |
|---|---|---|
| Rplp0 | GATGGGCAACTGTACCTGACTG | CTGGGCTCCTCTTGGAATG |
| (mouse) | (SEQ ID NO: 1) | (SEQ ID NO: 2) |
| Agrp | GGCCTCAAGAAGACAACTGC | GCAAAAGGCATTGAAGAAGC |
| (mouse) | (SEQ ID NO: 3) | (SEQ ID NO: 4) |
| Tet3 | GCCGATGCAGTAGTGGAGG | CTGCCTTGAATCTCCATGGTAC |
| (mouse) | (SEQ ID NO: 5) | (SEQ ID NO: 6) |
| Tet2 | AGCAAGAGATTCCGAAGGAT | AGTGGAGGACTGAGTGCAAG |
| (mouse) | (SEQ ID NO: 7) | (SEQ ID NO: 8) |
| Npy | AGGCTTGAAGACCCTTCCAT | ACAGGCAGACTGGTTTCAGG |
| (mouse) | (SEQ ID NO: 9) | (SEQ ID NO: 10) |
| Pomc | CATCTTTGTCCCCAGAGAGC | GCACCAGCTCCACACATCTA |
| (mouse) | (SEQ ID NO: 11) | (SEQ ID NO: 12) |
| Slc32a1 | TGGTCATCGCTTACTGTCTC | TGCTGCATGTTGCCTTCG |
| (mouse) | (SEQ ID NO: 13) | (SEQ ID NO: 14) |
| RPLPO | GGCGACCTGGAAGTCCAACT | CCATCAGCACCACAGCCTTC |
| (human) | (SEQ ID NO: 15) | (SEQ ID NO: 16) |
| AGRP | GAAGAGGATCTGTTGCAGGA | CAGGACTCATGCAGCCTTAC |
| (human) | (SEQ ID NO: 17) | (SEQ ID NO: 18) |
| TET3 | GACGAGAACATCGGCGGCGT | GTGGCAGCGGTTGGGCTTCT |
| (human) | (SEQ ID NO: 19) | (SEQ ID NO: 20) |
| TET2 | TTCGCAGAAGCAGCAGTGAAGAG | AGCCAGAGACAGCGGGATTCCTT |
| (human) | (SEQ ID NO: 21) | (SEQ ID NO: 22) |
| NPY | TCACCAGGCAGAGATATGGA | GCAAGTCTCATTTCCCATCA |
| (human) | (SEQ ID NO: 23) | (SEQ ID NO: 24) |
| SLC32A1 | Bio-Rad, qHsaCED0042869 | |
| (human) | | |

ChIP-qPCR/hMeDIP-qPCR Primer Sequences

| | | |
|---|---|---|
| Agrp | GTGCCCTTGACAAAGTTCCTGGAA | GCAGAACCTAGGGATGGGTCATGC |
| (mouse) | (SEQ ID NO: 25) | (SEQ ID NO: 26) |
| AGRP | GAATTCTTGGAAGCACAGGAAACA | CAGGGACCTAGGAGCTGAGCAGGG |
| (human) | (SEQ ID NO: 27) | (SEQ ID NO: 28) |

### Immunoprecipitation

To prepare antibodies, 5 μl (packed volume) of ChIP grade Dynabeads Protein G (Invitrogen, Thermo Scientific, 10004D) were washed twice with 1 ml of IP buffer (0.5% Triton X-100, 150 mM NaCl, 10 mM Tris-HCl at pH 7.5, and 10 mM EDTA), followed by incubation with 5 μg of rabbit polyclonal anti-TET3 (Active Motif, 61395) (FIG. 12A) or preimmune rabbit IgG in 300 μl of IP buffer at 4 °C overnight. Antibody bound beads were pelleted and kept on ice until use. To prepare lysate from ARCs (FIG. 6F), PBS washed ARCs (2 ARCs from one mouse per IP) freshly isolated from mice were homogenized (5-10 strokes) using a disposable pellet pestle in 500 μl of freshly prepared gentle lysis buffer (GLB, 0.5% Triton X-100, 10 mM NaCl, 10 mM Tris-HCl at pH 7.5, 10 mM EDTA, and 1× protease inhibitor cocktail) and incubated on ice for 20 min with occasional inversion. To prepare lysate from GT1-7 (FIG. 6G) and SH-SY5Y (FIG. 6H), cells at a density of 6×10⁶ cells/well in a 100 mm plate were treated with Lept H (1×10⁻⁸ M) for 2 h. Cells were then rinsed with cold PBS three times, collected by manual scraping in cold PBS, and pelleted by gentle centrifugation. The cell pellet was resuspended in 1 ml of cold freshly prepared Glo Lysis Buffer (GLB) and incubated on ice for 20 min with occasional inversion. For IP of ARCs or cells, after centrifugation at 12,000×g at 4 °C for 15 min to remove insoluble materials, 5 M of NaCl was added to a final concentration of 200 mM, and the lysate was transferred to a tube containing antibody/preimmune IgG-coated beads (400 μl of lysate per IP). IP was carried out at 4 °C for 4 h. Following IP, beads were quickly washed twice with 1 ml of cold IP buffer and washed additional three times with IP buffer by rotating at 4 °C for 5 min each time. After the final wash, residual liquid was completely removed and the beads were eluted with 16 μl of 2×SDS buffer (containing 1× phosphatase inhibitor cocktail and 1× protease inhibitor cocktail) at 100 °C for 5 min. 7 μl per gel well of eluant was loaded onto a 4-15% gradient SDS gel (Bio-rad, 456-8086). For Western blot analysis, anti-TET3 (Genetex, GTX121453) (Xu et al., 2020, Cell Rep., 30:1310-1318), anti-STAT3 (Proteintech, 10253-2-AP), anti-p-STAT3 (Cell Signaling, 9145S Y705) (Liang et al., 2019, Mol Ther Nucleic Acids., 18:183-193), anti-NCOR1 (Cell Signaling, 5948S) (Du et al., 2020, J Am Heart Assoc., 9:e015862), and anti-HDAC4 (Active Motif, 40969) (FIG. 12B) were diluted at 1:1000 (Liang, C. et al., 2019, Molecular Therapy—Nucleic Acids, 18:183-193; Xu, Y. et al., 2020, Cell Reports, 30:1310-1318 e5; Gao, Y. et al., 2020, Advanced Science, 7:2002518; Du, L. J. et al., 2020, Journal of the American Heart Association, 9:e015862). The secondary antibodies used were Rabbit IgG TrueBlot® (1:1000, Rockland, 18-8816-33). These unique HRP-conjugated monoclonal secondary antibodies enable detection of immunoblotted target proteins without hindrance by interfering immunoprecipitating immunoglobulin heavy and light chains.

### Mice

Both male and female mice were used for experiments. Mice were housed at 22 °C-24 °C with a 12 h light/12 h dark cycle with regular chow (Harlan Teklad no. 2018, 18% calories from fat) and water provided ad libitum. C57BL/6J (Jax, 000664), Agrp-IRES-Cre (Jax, 012899), and Rosa26-LSL-Cas9-GFP (Jax, 026175) were purchased from the Jackson Laboratory (Tong, Q. et al., 2008, Nature Neuroscience, 11:998-1000; Platt, R. J., 2014, Cell, 159:440-455). Following stereotaxic injection to express AAVs, mice were individually housed with ad libitum access to regular chow and water. Littermates of the same sex were randomly assigned to either control or experimental groups. For all experiments, age- and sex-matched animals were used. For information on animal numbers, refer to figure legends.

### Viruses

The AAV-sgTet3 (pAAV-sgRNA-Tet3-pEF1a-DIO mCherry) was constructed based on pAAV-pEF1α-DIO EYFP plasmid, a gift from Karl Deisseroth (Addgene #27056). NHE I and BSRG I (New England Biolabs) were used to replace the EYFP with an mCherry fragment using the same sites. This vector was then linearized with MLU I for a later ligation. The guide RNA was made by phosphorylating and annealing overlapping oligos from Integrated DNA Technologies and cloned into the bbs1 digested pSpCas9(BB)-2A-Puro (PX459V2) plasmid, a gift from Feng Zhang (Addgene #48139) (Ran, F. A. et al., 2013, Nature Protocols, 8:2281-2308). The U6 promoter, guide RNA and gRNA scaffold were PCR amplified and cloned into the MLU I site in the above digested vector. The resulting AAV-sgTet3 was packaged at Vigene Biosciences, Inc. The pAAV-hSyn-DIO-mCherry (AAV) (Addgene viral prep #50459-AAV9) and pAAV-hSyn-DIO-hM4D(Gi)-mCherry (AAV-h4MDi) (Addgne viral prep #44362-AAV9) were gifts from Bryan Roth (Krashes, M. J. et al., 2011, Journal of Clinical Investigations, 121:1424-1428). Viral particles were resuspended in calcium/magnesium-free DPBS (Gibco, catalog #14190144) at 2×1013 GC/ml and viral aliquots were stored at −80 OC before stereotaxic injection. The viruses were freshly diluted using DPBS before stereotaxic injection.

### Leptin Treatment of Mice

To assess leptin effects on suppression of hunger-induced appetite, female Cas9+ mice injected with AAV or AAV-sgTet3 bilaterally into the ARC were fasted overnight for 22 h. On the second day, mouse leptin (L3772-1MG, Sigma Aldrich) was administrated at 5 mg/kg intraperitoneally at 10:00 and pre-weighed food was placed in the cage and monitored for the following 24 h. For ChIP studies, Cas9+ mice injected with AAV or AAVsgTet3 bilaterally into the ARC were fasted overnight for 22 h. On the second day, saline or leptin (5 mg/kg) was administrated intraperitoneally at 10:00. Two hours later, ARCs were isolated for ChIP-qPCR analysis. To examine leptin-induced protein-protein interactions, Cas9+ mice were fasted overnight for 22 h. On the second day, leptin was administrated at 5 mg/kg intraperitoneally at 10:00, and ARCs were isolated 2 h later, followed by co-IP studies.

### Stereotaxic Injection

Injections were made into the ARC of anesthetized 6-week-old Cas9+ mice, placed in a stereotaxic apparatus (model 902; Kopf Instruments). Viruses (500 nL, 5×1012 GC/ml per site of injection) were applied into each hemisphere (coordinates: bregma, anterior-posterior: −1.45 mm, dorsal-ventral: −5.8 mm, lateral: +/−0.27 mm) by using an air pressure system (injection time: 5 minutes). After surgery, mice were allowed to recover for 2 weeks before electrophysiological recording. Stereotaxic injection sites were verified by double fluorescence labeling for GFP and mCherry, which could be detected without immunostaining. Mice with “missed” or “partial” hits were excluded from data analyses.

### Osmotic Pump Installation

Three days after bilateral ARC co-injection with AAV-sgTet3 and AAV-h4MDi, a mini-osmotic pump (model 1007D, Alzet) was implanted subcutaneously. The osmotic pump was filled with either sterile saline solution or DREADD agonist compound 21 (C21) dihydrochloride (0.5 mg/kg, HB6124-25 mg, Hello Bio). Food intake measurement and ITT were performed at day 5 and day 9 post-injection, respectively. For food intake assays, food pellets were weighed at 10:00 each day for 3 continuous days and an average of three-day food intake was calculated.

### Body Weight, Body Composition, and Food Intake Measurement

Mice were singly housed after surgery. Body weight was measured every other week, and body composition was assessed using EchoMRI analysis. Food intake and energy expenditure were measured using an indirect calorimetry chamber (TSE Systems, Germany).

### Electrophysiology

Coronal hypothalamic slices containing the ARC were prepared from virus injected mice as previously reported (Varela, L. et al., 2021, Journal of Clinical Investigations, 131:e144239). In brief, mice were anesthetized with isoflurane and decapitated. The brain was rapidly removed and immersed in cold (4 °C) and oxygenated cutting solution containing (in mM): sucrose 220, KCl 2.5, NaH2PO4 1.23, NaHCO3 26, CaCl2 1, MgCl2 6, and glucose 10 (pH 7.3 with NaOH). Coronal slices (300 μm thick) were prepared with a Leica vibratome after the brain was trimmed to a small tissue block containing the hypothalamus. After preparation, slices were maintained at room temperature (23 °C-25 °C) in a storage chamber in artificial cerebrospinal fluid (ACSF) (bubbled with 5% CO2 and 95% O2) containing (in mM): NaCl 124, KCl 3, CaCl₂) 2, MgCl2 2, NaH2PO4 1.23, NaHCO3 26, glucose 10 (pH 7.4 with NaOH) for recovery and storage. After recovery at room temperature for at least 1 hour, slices were transferred to a recording chamber constantly perfused at a rate of 2 mL/min with ACSF containing 2.5 mM glucose at a temperature of 33 °C for electrophysiological experiments. To identify virus infected AgRP neurons, mCherry and GFP fluorescence were detected using LED illumination (CoolLED pE-300). Whole-cell patch clamp recordings were obtained from AgRP neurons visualized using infrared differential interference contrast (IR-DIC) imaging. Spontaneous membrane and action potentials (MP) were recorded under current clamp as previously reported (Tan, Y. et al., 2020, Journal of Clinical Investigations, 130:4985-4998; Liu, Z. W. et al., 2011, Journal of Physiology, 589:4157-4166). The micropipettes (4-6 M2) were made of borosilicate glass (World Precision Instruments) with a micropipette puller (Sutter P-97) and backfilled with a pipette solution containing (in mM): K-gluconate 108, KCl 27, MgCl2 2, HEPES 10, EGTA 1.1, Mg-ATP 2.5, Na2-GTP 0.3, and Na2-phosphocreatine 10, pH 7.3 with KOH. Both input resistance and series resistance were monitored throughout the experiments, and the former was partially compensated. Only recordings with stable series resistance and input resistance were accepted. All data were sampled at 3 kHz, filtered at 3 kHz, and analyzed with an Apple Macintosh computer using AxoGraph X. t test was used to examine the statistical significance of the difference in AP frequency and threshold in the recorded AgRP neurons.

### Bobcat339 Treatment of Mice

Chow-fed C57BL/6J mice at the age of 12 weeks were treated with Bobcat339 (100 mg/kg per day) or vehicle (DMSO) in drinking water for 4 days. ARCs were isolated at 10:00 from ad libitum-fed mice and subjected to immunofluorescence analysis. Bobcat339 was dissolved in DMSO at a concentration of 100 mg/ml and stored at −20 °C in aliquots. Working solution (1 mg/ml) was freshly prepared every other day by dilution using tap water.

### GTT and ITT

Glucose tolerance tests (GTT) were performed following 16 h overnight fasting. Each animal received an intraperitoneal injection of 2 g/kg glucose (Sigma-Aldrich, G5767) in sterile saline. Insulin tolerance tests (ITT) were performed following a 3 h morning-fasting. Each animal received an intraperitoneal injection of 1 U/kg insulin (Novolin R Regular U-100 insulin) in sterile saline. Blood glucose concentrations were measured using Contour next blood glucose meter (Ascensia Diabetes Care) via tail vein bleeding at the indicated time points after injection.

### Western Blot Analysis

GT1-7 and SH-SY5Y cells in 24-well plates (2.5×10⁵ cells/well) were rinsed with cold PBS three times and collected by manual scraping in 150 μl of 2×SDS-sample buffer containing 1× Phosphatase inhibitor cocktail (Thermo, 78427) and 1× Protease inhibitor cocktail (Thermo, 78438), followed by heating at 100 °C for 5 min with occasional vortexing. The lysate was then centrifuged at 12,000 g for 5 min to remove insoluble materials before loading onto a 4-15% gradient SDS gel (Bio-rad, 456-8086) (10 μl/well), followed by Western blot analysis. The antibodies used were anti-TET3 (diluted at 1:1000; mouse/human, GeneTex, GTX121453) (FIG. 12A), anti-AGRP (mouse) (diluted at 1:500; MilliporeSigma, AB3402P) (validated by the vendor), anti-AGRP (human) (diluted at 1:500; Abcam, Ab113481), anti-NPY (diluted at 1:1000; mouse/human, Cell Signaling 11976S), anti-VGAT (diluted at 1:4000; mouse/human, Abcam, Ab235952) (validated by the vendor), and HRP-conjugated anti-GAPDH (diluted at 1:5000; Proteintech, HRP-60004) (Xu, Y. et al., 2020, Cell Reports, 30:1310-1318 e5; Lopez, R. et al., 2013, PLoS One, 8:e79708; Imbernon, M. et al., 2014, Molecular Metabolism, 3:441-451; Glaser, J. et al., 2022, eLife, 11:65641). The secondary antibody was HRP-linked Anti-rabbit IgG (Cell Signaling, 7074).

Hydroxymethylated DNA Immunoprecipitation Coupled with qPCR (hMeDIP-qPCR)

The experiments were carried out using the EpiQuik hMeDIP Kit (P-1038-48, Epigentek) according to the manufacturer's instructions. Briefly, for ARC hMeDIP, freshly isolated ARCs (2 ARCs from one mouse per IP, FIG. 6K) were washed twice with 1 ml of cold PBS and homogenized (5-10 strokes) using a disposable pellet pestle (Fisher Scientific, 12-141-368) in 500 μl of Genomic Lysis Buffer. For SH-SY5Y hMeDIP (FIG. 6L), cells seeded in 6-well plates at 1×106 cells/well the night before were transfected with NT siRNA or TET3 siRNA under Lept H conditions, and genomic DNAs were isolated at 48 h following transfection using Quick gDNA MicroPrep Kit (D3021, Zymo Research Corporation) and sheared using a sonifier (Branson 150), with a setting of 9 pulses of 10 sec each at 35% amplitude followed by a 40 sec rest period on ice between each pulse. Sheared DNA fragments (ranged in size from 200-600 bps as assessed by agarose gel electrophoresis) were immunoprecipitated using the 5hmC rabbit polyclonal antibody from the kit. qPCR was performed in a 25 μl reaction containing 2.5 μl of the eluted DNA using iTAC SYBGreen in a Bio-Rad iCycler. The relative enrichments (after normalization against control IgG) of the indicated DNA regions were calculated using the Percent Input Method according to the manufacturer's instructions.

### Plasma Insulin, Leptin, and Corticosterone

For insulin and leptin, blood samples were collected in EDTA tubes (Microtainer with K2EDTA, BD, 365974) by cardiac puncture of terminally anesthetized animals between 9:00 and 11:00. For corticosterone, blood samples were obtained via retroorbital bleeding between 19:00 and 20:00. The tubes were centrifuged at 2,000×g at 4 °C for 20 min, and plasma was collected and stored at −80 °C until use. Plasma insulin, leptin, and corticosterone levels were measured using Mouse Insulin ELISA kit (Crystal Chem, 90080), Mouse Leptin ELISA kit (Crystal Chem, 90030), and Corticosterone ELISA kit (Enzo, ADI-900-097), respectively, according to the manufacturer's instructions.

### Behavioral Tests

For all behavioral tests, mice were transferred to the testing room 1 h prior to testing for acclimation to the environment. All behavioral apparatus was wiped with 70% ethanol prior to each trial and between trials. The tail suspension test (TST) and the forced swim test (FST) lasted for 6 min and the total amount of immobility time was measured for each animal and considered as an index of “depressive-like” behavior (Steru, L. et al., 1985, Psychopharmacology, 85:367-370; Yankelevitch-Yahav, R. et al., 2015, Journal of Visualized Experiments; 97:52587). For the TST, cylindrical plastic tubes were placed at the base of the tail to prevent tail climbing.

### Statistical Analysis

All statistical analyses were performed using GraphPad Prism version 8 for Windows (GraphPad Software, La Jolla California USA, www.graphpad.com) and are presented as mean±SEM. Two-tailed Student's t tests (or as otherwise indicated) were used to compare means between groups. P<0.05 was considered significant.

### Example 2: Targeting TET3 for the Treatment of Anorexia and Stress-Related Disorder

Anorexia nervosa (AN) is a psychiatric illness with the highest mortality. Current treatment options have been limited to psychotherapy and nutritional support, with low efficacy and high relapse rates (Scharner et al., 2020, Front Hum Neurosci., 14:596381; van Eeden et al., 2021, Curr Opin Psychiatry, 34:515-524). Hypothalamic AgRP neurons that co-express agouti-related peptide (AGRP) and neuropeptide Y (NPY) play a critical role in driving feeding while also modulating other complex behaviors. Genetic ablation of Tet3 specifically in AgRP neurons in mice activated these neurons and increased the expression of AGRP, NPY, and the vesicular GABA transporter (VGAT), leading to hyperphagia and anxiolytic effects (Xie et al., 2022, J Clin Invest., 132).

Bobcat339 was effective in mitigating AN and anxiety/depressive-like behaviors using a mouse model of activity-based anorexia (ABA) (Lv H et al., 2023, Proc Natl Acad Sci USA 2023; in press). It was shown that treating mice with Bobcat339 decreased TET3 protein in AgRP neurons and activated these neurons leading to increased feeding, decreased compulsive running, and diminished lethality in the ABA model. Mechanistically, Bobcat339 induced TET3 protein degradation while simultaneously stimulating the expression of AGRP, NPY, and VGAT in a TET3-dependent manner both in mouse and human neuronal cells, demonstrating a conserved, previously unsuspected mode of action of Bobcat339. These findings indicated that Bobcat339 is likely a new therapeutic for anorexia nervosa and cancer-induced anorexia and its associated mood disorders, such as anxiety and depression.

### Bobcat339 Destabilized TET3 Protein in Neuronal Cells

The catalytic domain of the three TET enzymes is highly conserved but not identical, and each of the members exhibits varying substrate preferences and catalytic activity (Tahiliani et al., 2009, Science, 324:930-935). Bobcat339 is a synthetic cytosine derivative initially reported to inhibit the enzymatic activity of TET1 and TET2, but its effects in vivo and on TET3 were not defined (Chua et al., 2019, ACS Med Chem Lett, 10:180-185). It was later found that Bobcat339 on its own had a negligible inhibitory activity against TET1 and TET2 in the absence of contaminating copper (II) (Weirath et al., 2022, ACS Med Chem Lett, 13:792-798).

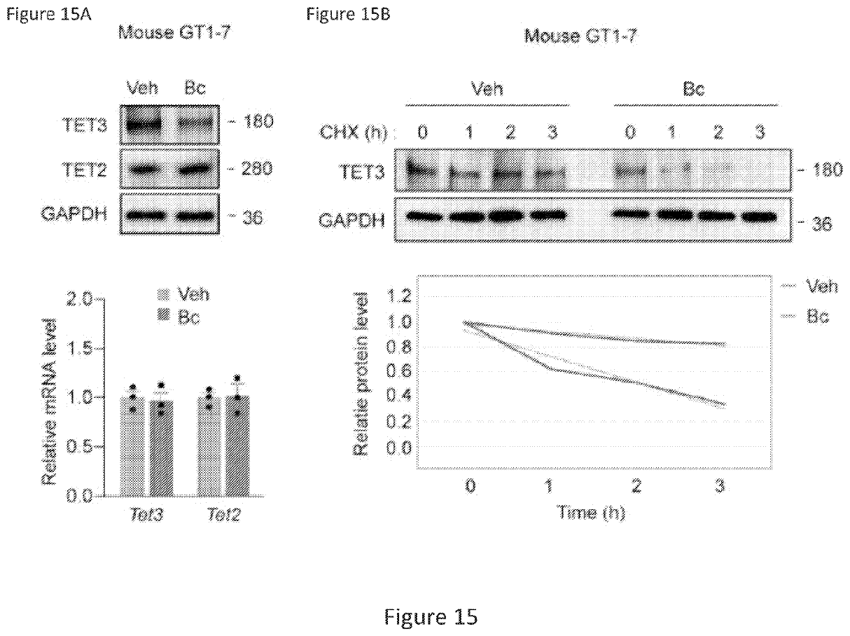

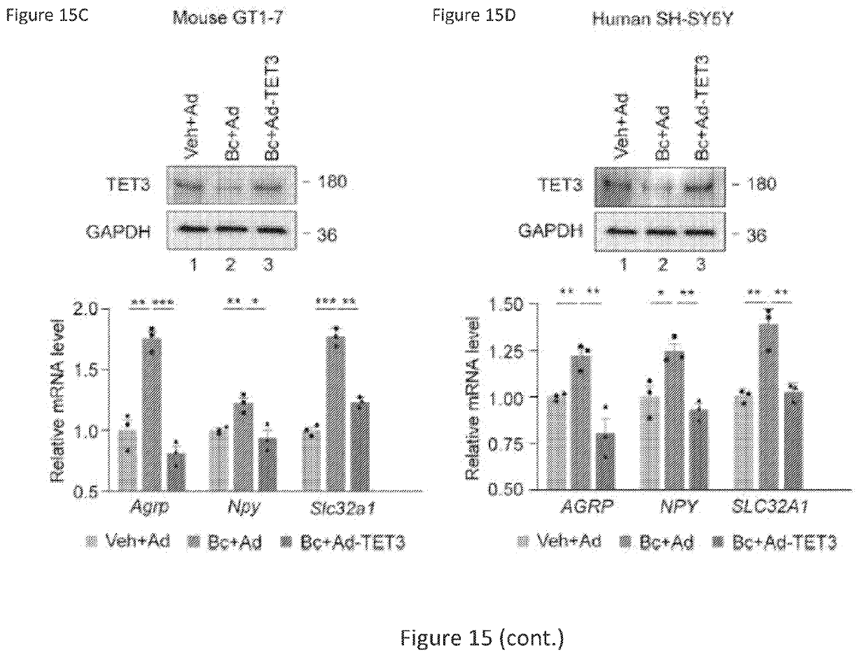

Nonetheless, as Bobcat339 was predicted to bind to the catalytic sites of all three TET enzymes based on the crystal structure of TET2-DNA complex (Chua et al., 2019, ACS Med Chem Lett, 10:180-185). Thus, GT1-7, an immortalized mature mouse hypothalamic GnRH neuronal cell line, was incubated with Bobcat339 at 10 μM for 6 h and a decrease in the protein level of TET3 was unexpectedly found (FIG. 15A, top panel) without affecting its mRNA abundance (FIG. 15A, bottom panel). Bobcat339 did not affect TET2 expression (FIG. 15A). No expression of TET1 was detected in GT1-7 cells, consistent with previous studies reporting negligible TET1 expression in the adult mouse hypothalamus (Xie et al., 2022, J Clin Invest, 132).

It is not unprecedented that compounds initially developed as protein function modulators are later serendipitously found to promote protein degradation (Dauvois et al., 1992, Proc Natl Acad Sci USA, 89:4037-4041; Bekes et al., 2022, Nat Rev Drug Discov, 21:181-200). To test whether Bobcat339 affect TET3 protein stability, time course experiments were performed in the presence of cycloheximide, a protein synthesis inhibitor. It was found that TET3 was less stable in Bobcat339-treated vs. vehicle treated cells (FIG. 15B, upper panel). While TET3 remained stable in vehicle-treated cells, it became dramatically unstable in Bobcat339-treated cells with a half-life of ~2 h (FIG. 15B, bottom panel). Since the studies used Bobcat339 purchased from Sigma-Aldrich that was shown to be free from Cu (II) contamination (Weirath et al., 2022, ACS Med Chem Lett, 13:792-798), Bobcat339 induced TET3 protein degradation without affecting its enzymatic activity, a discovery not previously documented.

### Bobcat339 Destabilized TET3 Protein and Upregulated AGRP and NPY Expression in a TET3-Dependent Manner

As exposing mice to Bobcat339 increased AGRP production and induced hyperphagia, phenocopying AgRP neuron-specific TET3 knockdown in AgRP neurons, and although not bound by any particular theory, it was tested whether Bobcat339 inhibit TET3 expression in AgRP neurons. In light of a recent report that Bobcat339 on its own had negligible inhibitory activity against TET1 and TET2 in the absence of contaminating copper (II), copper-free Bobcat339 was purchased from Sigma-Aldrich (Weirath, N. A. et al., 2022, ACS Medicinal Chemistry Letters, 13:792-798). It was observed that incubation of mouse hypothalamic cell line GT1-7 and Bobcat339 rapidly decreased the level of TET3 protein without altering its mRNA abundance (FIG. 15A). When TET3 was analyzed in the presence of cycloheximide, a protein synthesis inhibitor, TET3 was less stable in Bobcat339-treated compared to vehicle-treated cells (FIG. 15B, Upper Panel). The half-life of TET3 was 2 hours in Bobcat339-treated cells, while in vehicle treated cells the half-life was over 3 hours (FIG. 15B, Bottom Panel), demonstrating that Bobcat339 induced TET3 protein degradation in neuronal cell lines. Similar observations were made in SH-SY5Y human neuronal cells.

Given that TET3 inhibited the transcription of Agrp and Npy and that Bobcat339 destabilized TET3 protein (FIG. 15A and FIG. 15B), it was tested whether exposing cells to Bobcat339 increase the mRNA level of AGRP. Indeed, GT1-7 cells were incubated with Bobcat339 in the presence or absence of exogenous TET3 expression from an adenoviral vector (Ad-TET3). While Bobcat339 expectedly decreased the level of TET3 (FIG. 15C, top panels, compare lane 2 to lane 1), exogenous TET3 expression restored it to the control level (top panels, compare lane 3 to lane 1). When mRNAs were examined, increased in the mRNA levels of both Agrp and Npy were observed, which were not seen when the level of TET3 protein was restored by exogenous TET3 expression (FIG. 15C). Similar observations were made in human SH-SY5Y neuronal cells (FIG. 15D), demonstrating that Bobcat339 promoted TET3 protein destabilization, which in turn upregulated expression of AGRP in both mouse and neuronal cell lines.

### Bobcat339 Affected the Expression of Agrp/AGRP, Npy/NPY, and Slc32a1/SLC32A1

Given that TET3 inhibited the expression of Agrp/AGRP, Npy/NPY, and Slc32a1/SLC32A1 (Xie et al., 2022, J Clin Invest, 132) and that Bobcat339 destabilized TET3 protein (FIG. 15A and FIG. 15B), additional studies were conducted to test whether exposing cells to Bobcat339 would increase the expression of Agrp/AGRP, Npy/NPY, and Slc32a1/SLC32A1 in a TET3-dependent manner. Thus, GT1-7 cells were incubated with Bobcat339 in the presence or absence of exogenous TET3 expression from an adenoviral vector (Ad-TET3). While Bobcat339 expectedly decreased the level of TET3 protein (FIG. 15C, top panels, compare lane 2 to lane 1), exogenous TET3 expression restored it to the level of control (top panels, compare lane 3 to lane 1). When mRNAs were examined, increases in the mRNA levels of Agrp, Npy and Slc32a1 were observed (FIG. 15C, bottom panel, compare red bars to blue bars), which were not seen when the level of TET3 protein was restored by exogenously expressed TET3 (compare purple bars to blue bars). Similar observations were made in SH-SY5Y human neuronal cells (FIG. 15D). Taken together, the results showed that Bobcat339 stimulated the expression of Agrp/AGRP, Npy/NPY, and Slc32a1/SLC32A1 in a TET3-dependent manner and that this regulation was conserved in human and mouse cells.

### Bobcat339 Downregulated TET3 Expression in AgRP Neurons

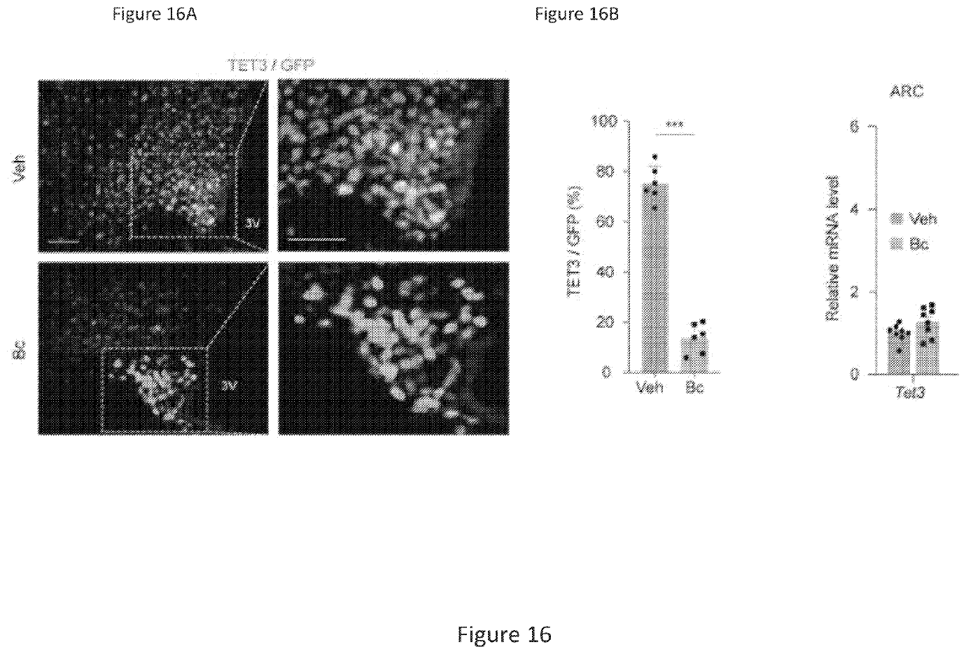

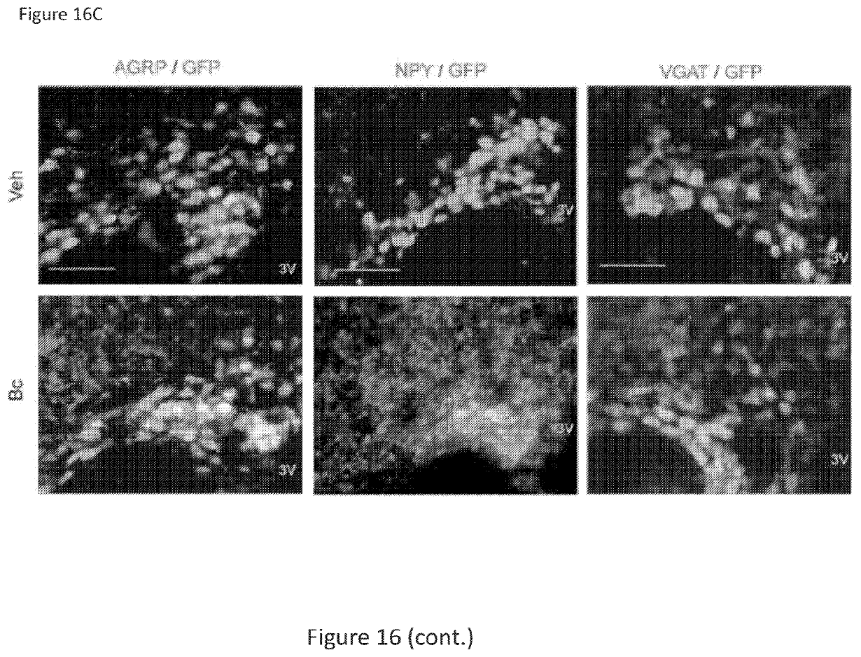

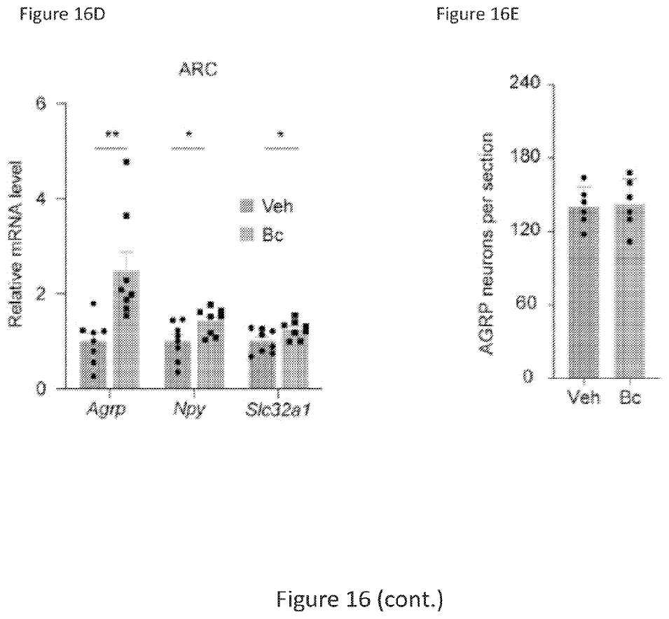

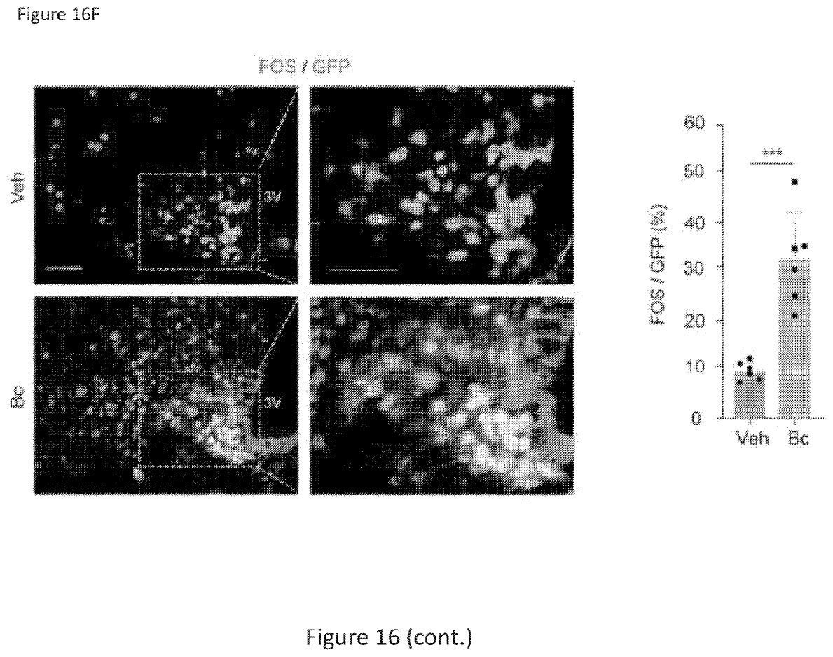

To test whether Bobcat339 affects TET3 protein expression in AgRP neurons, Agrp-IRES-Cre::LSL-Cas9-GFP mice with GFP expression specifically in AgRP neurons were intraperitoneally injected with vehicle or Bobcat339 at 2.5 mg/kg, followed by isolation of ARCs under fed conditions (9:00-11:00) two days later. This dose was chosen based on the dose response studies. TET2 expression was widespread but was negligible in AgRP neurons. Importantly, Bobcat339 did not significantly affect TET2 expression, consistent with in vitro findings (FIG. 15A). In contrast, TET3 was readily detected both in AgRP and non-AgRP cells, with a clear decrease in the number of TET3-positive AgRP neurons in Bobcat339-treated animals (FIG. 16A), demonstrating decreased expression of TET3 protein in AgRP neurons. Given that Bobcat339 did not alter the mRNA abundance of Tet3 in the ARC (FIG. 16B) and that Bobcat339 destabilized TET3 protein in vitro (FIG. 15A and FIG. 15B), Bobcat339 destabilized TET3 protein in AgRP neurons.

Notably, Bobcat339 treatment also decreased TET3 protein in non-AgRP cells (FIG. 16A, left panels), but the decrease appeared to be limited to the tip regions of the ARC even at a higher dose (4 mg/kg). The ARC neurons resided in the areas where the blood-brain barrier is modified to be more permeable to facilitate the access of blood-borne nutrients, hormones, and metabolites (Haddad-Tovolli et al., 2017, Front Neurosci 11, 224). Thus, additional studies also investigated minimizing off-target effects of Bobcat339 through dose optimization.

### Bobcat339 Upregulated the Expression of AGRP, NPY and VGAT in the ARC

Next, increased productions of AGRP, NPY, and VGAT were detected in Bobcat339-exposed animals (FIG. 16C). The GFP construct used for tagging AgRP neurons labels almost exclusively the perikaryon. The presence of AGRP, NPY, and VGAT outside the perikarya reflected the fact that these fast-firing neurons transport their neuromodulatory products rabidly to the neuronal processes. Increases in the mRNA levels of Agrp, Npy and Slc32a1 were also observed in the ARCs of animals treated with Bobcat339 (FIG. 16D). Further, Bobcat339 treatment did not affect AgRP neuronal viability (FIG. 16E). These results showed that Bobcat339 increased the expression of AGRP, NPY, and VGAT, likely through inhibiting TET3 protein expression in AgRP neurons, consistent with the in vitro findings (FIG. 15C and FIG. 15D).

### Bobcat339 Increased the Number of FOS-Positive AgRP Neurons

As genetic TET3 knockdown specifically in AgRP neurons activated these neurons (Xie et al., 2022, J Clin Invest, 132), additional studies tested whether Bobcat339 treatment can activate AgRP neurons using FOS (a marker for neuronal activation) as a readout. Thus, ARCs were isolated under fed conditions (9:00-11:00) from mice treated with Bobcat339 or vehicle as above (FIG. 16A through FIG. 16D). Immunofluorescence analysis revealed increased FOS expression in AgRP neurons in Bobcat339 vs. vehicle treated mice (FIG. 16F). These results indicated that Bobcat339 downregulated TET3 protein in AgRP neurons, leading to neuronal activation.

### Bobcat339 Mitigated Anorexia Nervosa

Anorexia nervosa is a life-threatening illness with poorly understood pathophysiological mechanisms. A number of animal models have been developed to study anorexia; the most widely used is the activity-based anorexia (ABA) (Spadini, S. et al., 2021, Journal of Eating Disorders, 9:123). In the ABA model, adolescent rodents are subjected to time-restricted feeding with unlimited access to a running wheel. The combination of these two factors leads to low caloric intake, significant weight loss, and excessive physical activity, recapitulating key features of the human condition (Miletta, M. C. et al., 2020, Nature Metabolism, 2:1204-1211; Scharner, S. et al., 2020, Frontiers in Human Neuroscience, 14:596381).

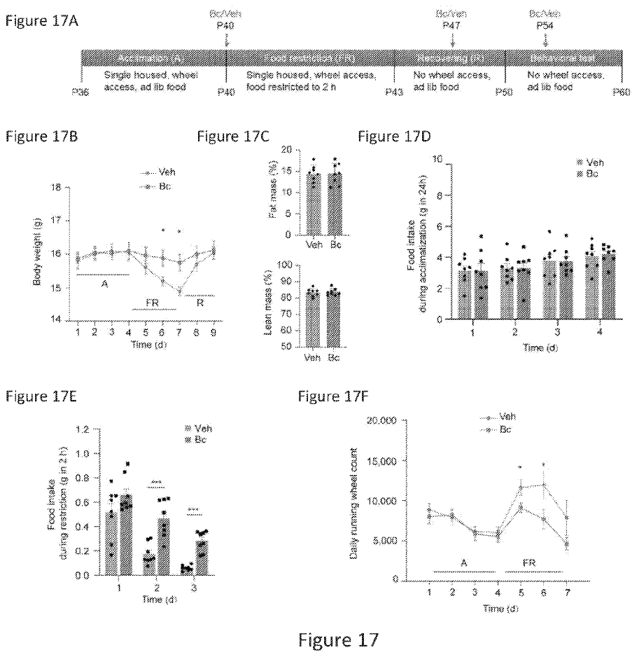

Using this protocol, it was demonstrated that mice with AgRP neuron ablation and food restriction died within 72 h of compulsive running whereas daily activation of AgRP neurons by way of chemogenetic tools prevented the mortality (Miletta, M. C. et al., 2020, Nature Metabolism, 2:1204-1211). The importance of AgRP neurons in psychiatric conditions such as anorexia was further underscored by a recent report showing that chemogenetic activation of AgRP neurons in mice during ABA attenuated bodyweight loss with reduction in excessive physical activity (Hickey et al., 2022, Mol Psychiatry). Because treating mice with Bobcat339 activated AgRP neurons (as assessed by increased expression of FOS, AGRP, and NPY) (FIG. 16) and induced hyperphagia, it was examined whether Bobcat339 could be used to treat anorexia. Thus, peripubertal female mice were exposed to the ABA paradigm as previously described (Miletta, M. C. et al., 2020, Nature Metabolism, 2:1204-1211). Mice were singly housed at postnatal day 36 (P36) with ad libitum access to food, water, and a running wheel. After 4 days of acclimation at P40, mice were food restricted with free access to food for only 2 h daily for 3 days. At P40 and before the onset of food restriction, mice received first intraperitoneal (i.p.) injection of Bobcat339 or vehicle, with second and third injections performed at P47 and P54, respectively (FIG. 17A).

There were no differences in body weight (FIG. 17B), daily food intake (FIG. 17D), and running wheel account (FIG. 17F) between Bobcat339 and control groups during acclimation. Similar to what was reported before, animals in the control group exhibited a progressive decline in body weight during food restriction; however, Bobcat339 treated animals were able to maintain their body weight throughout the experiment (FIG. 17B) (Miletta, M. C. et al., 2020, Nature Metabolism, 2:1204-1211). In addition, animals in the Bobcat339 group showed a significant increase in food intake during food restriction (FIG. 17E), with a parallel decline in compulsive wheel running (FIG. 17F). Given the hallmarks of anorexia being low caloric intake, progressive weight loss, and hyperactivity, the herein described results demonstrated that Bobcat339 was effective in mitigating anorexia by increasing food intake, preventing weight loss, and attenuating compulsive running (Scharner, S. et al., 2020, Frontiers in Human Neuroscience, 14:596381; Spadini, S. et al., 2021, Journal of Eating Disorders, 9:123).

### Bobcat339 Elicited Anxiolytic Effects

A growing body of evidence showed that exposing rodents in adolescence to the ABA paradigm activates the hypothalamic-pituitary-adrenal (HPA) axis and produces anxiogenic long-term effects in adulthood (Spadini, S. et al., 2021, Journal of Eating Disorders, 9:123). For example, mice subjected to the ABA protocol displayed increased anxiety following the recovery period when their body weight had been restored (Chen, Y. W. et al., 2017, Cerebral Cortex, 27:3980-3993). Importantly, anorexia patients showed dysregulation of the HPA axis at both the neuroendocrine (increased corticotropic-releasing hormone) and the endocrine level (increased cortisol) (Hotta et al., 1986, J Clin Endocrinol Metab, 62:319-324; Kaye et al., 1987, J Clin Endocrinol Metab, 64:203-208; Lawson et al., 2013, Eur J Endocrinol, 169:639-647).

It has been reported that AgRP neuron-specific TET3 knockdown reduces stress-like behaviors with decreased levels of circulating corticosterone. Given that Bobcat339 downregulated TET3 expression in AgRP neurons (FIG. 16A), mice were subjected to behavioral tests after the recovery period. Given that Bobcat339 downregulated TET3 protein in AgRP neurons (FIG. 16A), mice were subjected to behavioral tests after the recovery period (FIG. 17A). The open field test (OFT) has been used to assess general locomotor activity and anxiety; the tail suspension test (TST) and forced swim test (FST) have been used to evaluate behavioral despair and also to test the efficacy of new antidepressant compounds. All three tests (OFT, TST, and FST) have been used in previous studies to assess behavioral impacts of AgRP neurons in mice (Miletta, M. C. et al., 2020, Nature Metabolism, 2:1204-1211).

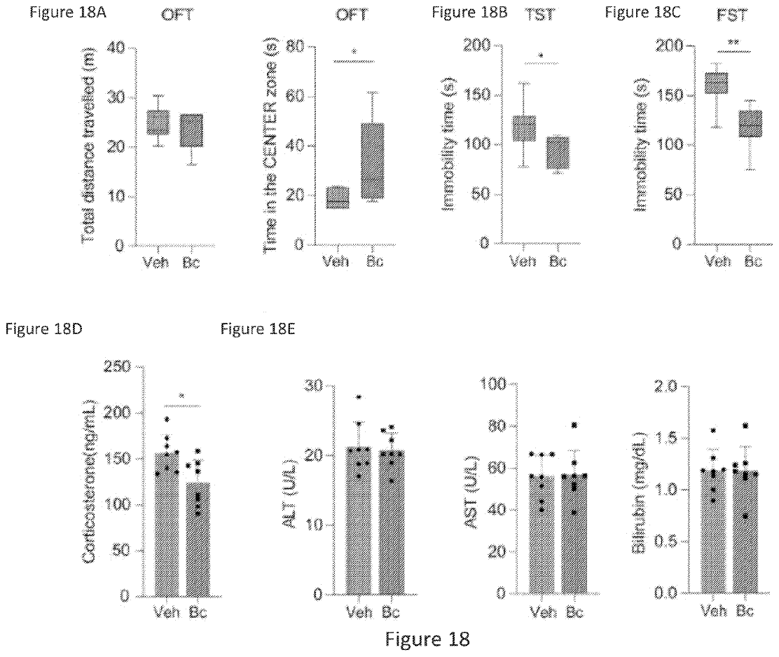

During the recovery period body weight of the animals was quickly restored within three days (FIG. 17B). No differences in body composition were observed between the two groups at the end of the recovery period (FIG. 17C). In the OFT, there were no differences in the total distance travelled between Bobcat339 and vehicle-treated groups (FIG. 18A, Left Panel), indicating that Bobcat339 did not affect general locomotor activity. However, Bobcat339-treated animals showed an increased time spent in the center zone (FIG. 18A, Right Panel), indicating reduced anxiety. In addition, Bobcat339-treated animals spent less immobility time than the controls both in TST (FIG. 18B) and FST (FIG. 18C), indicating decreased in depressive-like states. Further, compared to control animals Bobcat339-treated animals had reduced plasma cortisol levels (FIG. 18D). Finally, there was no evidence of liver toxicity following three weeks of once-a-week Bobcat339 treatment (FIG. 18E). Taken together, these results demonstrated that Bobcat339 was effective in reducing anxiety/depressive-like states.

The present studies used activity-based paradigm shown to recapitulate characteristics of individuals with anorexia nervosa and demonstrated the potential of Bobcat339 as a novel therapeutic for this disorder and associated anxiety/depressive behaviors. Specifically, it was shown that Bobcat339 acted to destabilize TET3 protein, though the exact mechanism remains to be determined. It was also shown that Bobcat339 phenocopies AgRP neuron-specific TET3-knockdown in that it activated AgRP neurons, simultaneously increased the expression of AGRP, NPY, and VGAT, and induced hyperphagia and anxiolytic effects. Thus, the results identified TET3 as a molecular, and AgRP neurons as a cellular, target of Bobcat339 action.

Notably, Bobcat339 was able to stimulate appetite via multiple routes of administration, including oral and intraperitoneal. Further, Bobcat339 exerted its appetite-stimulating and anti-stress effects within 24 h and its effects lasted for at least one week following a single i.p. injection. This was in contrast to many current antidepressants, which elicited antidepressant effects after 1 to 2 weeks of taking the medication. Moreover, Bobcat339 appeared to be well tolerated and no liver toxicity has been detected after 3-weeks of once-a-week i.p. injection.

Finally, as a proof of principle the studies set the stage for more in-depth investigations including pharmacokinetic and toxicology studies before establishing Bobcat339 as a novel therapeutic for anorexia nervosa and perhaps cancer-induced anorexia and associated mood disorders.

In summary, anorexia nervosa is a psychiatric illness with the highest mortality. Current treatment options have been limited to psychotherapy and nutritional support, with low efficacy and high relapse rates. The hypothalamic agouti-related peptide (AGRP)-expressing neurons are essential for driving feeding while also modulating other complex behaviors. Previous studies have recently reported that genetically reducing the expression of TET3, a member of the TET family dioxygenases, specifically in AgRP neurons in mice activates these neurons and increases expression of both AGRP and neuropeptide Y (NPY), leading to hyperphagia and anti-stress effects. Bobcat339 is a synthetic cytosine derivative originally reported to inhibit the enzymatic activity of TET1 and TET2 in vitro, but its effects in vivo and on TET3 were not previously documented. The present studies demonstrated that Bobcat339 was effective in mitigating AN and anxiety/depressive-like behaviors using a well-established mouse model of activity-based anorexia. The present studies showed that treating mice with Bobcat339 decreased TET3 expression in AgRP neurons and activated these neurons. These effects were detected as early as three days following exposure to Bobcat339. Mechanistically, Bobcat339 induced TET3 protein degradation while stimulating expression of AGRP and NPY in a TET3-dependent manner both in mouse and human neuronal cells, representing a conserved, previously unsuspected mode of action of Bobcat339. The present findings indicated that Bobcat339 is a new therapeutic for anorexia and stress-related disorders, such as anxiety and depression.

The materials and methods employed in Example 2 are now described.

### Animals

Female C57BL/6J mice were purchased. The Agrp-IRES-Cre::LSL-Cas9-GFP mice with GFP expression specifically in AgRP neurons were generated as previously described (Xie et al., 2022, J Clin Invest, 132). Mice were housed at 22 °C-24 °C with a 12 h light/12 h dark cycle with regular chow (Harlan Teklad no. 2018, 18% calories from fat) and water provided ad libitum. For all experiments, age-matched female animals were used. For experiments shown in FIG. 16 through FIG. 18, 6-8 animals per group were used.

### Bobcat339 Treatment of Mice

Bobcat339 powder (Sigma-Aldrich, SML2611) was freshly dissolved in DMSO (vehicle) at a concentration of 50 mg/mL and filtered through a 0.22 micron. It was further diluted with 1×PBS to a final concentration of 0.5 mg/mL before injections. Mice were injected intraperitoneally with Bobcat339 at 1 mg/kg, 2.5 mg/kg, or 4 mg/kg.

### Cell Lines and Adenoviruses

Mouse GT1-7 hypothalamic neuronal cell line (Sigma-Aldrich, SCC116) and human SH-SY5Y neuroblastoma cell line (Sigma Aldrich, 94030304) were purchased and cultured according to the manufacturers' instructions. Purified Ad-TET3 adenovirus (Ad-FLAG.h-TET3, ADV-225322, Vector Biolabs) expressing human TET3 from a CMV promoter and Ad-GFP control adenovirus (1060, Vector Biolabs) were purchased.

### Cell Culture and Treatments

For Bobcat339 treatment (FIG. 15A and FIG. 15B), GT1-7 cells grown in 24-well plates at 2×10⁵ cells/well were incubated with vehicle or Bobcat339 at a final concentration of 10 μM for 6 h, followed by RNA and protein extractions. For TET3 protein stability assay (FIG. 15B), GT1-7 cells in 24-well plates at 2×10⁵ cells/well were incubated with vehicle or Bobcat339 at a final concentration of 10 μM for 3 hours, followed by addition of cycloheximide (CHX, Cell Signaling, 2112) at a final concentration of 50 g/ml in the presence of 10 μM of Bobcat339. Proteins were harvested at 0, 1, 2, and 3 hours after addition of CHX. For TET3 expression restoration experiments (FIG. 15C and FIG. 15D), GT1-7 or SH-SY5Y cells seeded in 24-well plates at 2×10⁵ cells/well were infected with Ad-GFP or Ad-TET3 at 4000 gc/cell. Following 16 h of infection, vehicle or Bobcat339 were added at a final concentration of 10 μM. Protein and RNA were isolated 48 h later and analyzed.

RNA extraction and RT-qPCR

Total RNAs were extracted from neuronal cells or homogenized hypothalamic arcuate nucleus tissue samples using PureLink RNA Mini Kit (Ambion, 12183025). cDNA was synthesized using PrimeScript RT Reagent Kit in a 20 μl reaction containing 0.5-1 μg of total RNA. Real-time quantitative PCR was performed in a 15 μl reaction containing 0.5-1 μl of cDNA using SsoAdvanced Universal SYBR Green Supermix in a Bio-Rad iCycler. Specificity was verified by melting curve analysis. The Ct values of each sample were used in the post-PCR data analysis. Gene expression levels were normalized against RPLP0.

### Western Blot Analysis

GT1-7 and SH-SY5Y cells in 24-well plates were collected by manual scraping in 2×SDS-sample buffer containing 1× Phosphatase inhibitor cocktail (Thermo, 78427) and 1× Protease inhibitor cocktail (Thermo, 78438), followed by heating at 100 °C for 5 min with occasional vortexing. The lysate was then centrifuged at 12,000 g for 5 min at room temperature (RT) to remove insoluble materials before loading onto 4-15% gradient SDS gels (Bio-rad, 456-8086), followed by Western blot analysis. The antibodies used were anti-TET3 (diluted at 1:1000, Active motif, 61395), anti-TET2 (dilution 1:500; Cell Signaling Technology, 18950), and HRP-conjugated anti-GAPDH (dilution 1:5000; Proteintech, HRP-60004). The secondary antibody was HRP-linked Anti-rabbit IgG (dilution 1:10,000; Rockland, 611-1322).

### Immunofluorescence

The immunofluorescence for brain slices was conducted using previous methods (18). In brief, postfixed sections were cut into 40-μm-thick sections, followed by 5 times washing. Then the sections were incubated in blocking solution for 20 minutes and incubated with anti-TET3 (dilution 1:2000; Millipore Sigma, ABE290), anti-TET2 (dilution 1:500; Proteintech, 21207-1-AP), anti-AGRP (dilution 1:400; H-003-57, Phoenix Pharmaceuticals), anti-NPY (dilution 1:800, Cell Signaling Technology, 11976S), anti-VGAT (dilution 1:200; Abcam, Ab23592), or anti-FOS (dilution 1:1000; Biosensis, R-1751-050) overnight at 4 °C. Negative controls were performed by omitting the respective primary antibodies. The next day, sections were washed 5 times and incubated in 0.4% Triton x-100 PBS with the secondary antibody donkey anti-Rabbit IgG Fluor 594 (dilution 1:500; A-21207, Invitrogen) for 2 h at RT. The sections were coverslipped and scoped using a Keyence BZ-X700 fluorescence microscope. The fluorescence signals from GFP in AgRP neurons were detected without immunostaining.

### Activity-Based Anorexia Model

On P36, animals were single housed with free access to food and water and 24 h access to a running wheel. After 4 days of acclimation, on P40, all food was removed from the cage and returned only for 2 h daily (free food access from 19:00 to 21:00) and 24 h access to a running wheel for 3 days. On P43, 24 h ad libitum access to food was returned and the running wheel access was blocked to allow the animals to recover. The animals were allowed to recover for at least 1 week before undergoing behavioral testing. Continuous multiday analysis of running wheel activity was recorded using VitalView Data Acquisition System software version 5.1 (Starr Life Science). Food intake was measured after measurement of the food pellets before and after the 2 h food restriction. Body weight was monitored in the morning. Body composition was assessed using EchoMRI analysis.

### Behavioral Tests

For all behavioral tests, mice were transferred to the testing room 1 h prior to testing for acclimation to the environment. All behavioral tests were performed in the afternoon (14:00-16:00). All behavioral apparatus was wiped with 70% ethanol prior to each trial and between trials. The open field (OF) apparatus consisted of a 56×56 cm open arena with 30 cm high walls. The mouse was placed into the center of the arena and allowed to move freely for 10 min with the activity being recorded and tracked by LimeLight 3 software (Actimetrics, Coulbourn Instruments). The software recorded and analyzed the distance and time traveled in the central (28×28 cm central area of the OF) and outer areas of the arena. The tail suspension test (TST) and the forced swim test (FST) lasted for 6 min and the total amount of immobility time during the final 4 min was measured for each animal.

### Blood Chemistry

For corticosterone, blood samples were obtained via retroorbital bleeding between 19:00 and 20:00. For alanine transaminase, aspartate transaminase and bilirubin, blood samples were collected by cardiac puncture of terminally anesthetized animals. All blood samples were collected in EDTA tubes (Microtainer with K2EDTA, BD, 365974). The tubes were centrifuged at 2,000×g at 4 °C for 20 min, and plasma was collected and stored at −80 °C until use. Plasma corticosterone levels were measured using Corticosterone ELISA kit (Enzo, ADI-900-097) according to the manufacturer's instructions. Kits used to measure alanine transaminase (EALT-100) and aspartate transaminase (EASTR-100) were purchased from Bioassay Systems. The bilirubin assay kit (MAK126) was purchased from Sigma Aldrich.

### Example 3: TET3 Regulated Disease-Associated Macrophages

Tissue-resident macrophages are comprised of embryonically derived cells as well as infiltrated monocytes (Park M. D. et al., 2022, Cell, 185:4259-4279). These macrophages play essential roles in tissue repair and maintenance, but under disease conditions, they can be “educated” to become molecularly, phenotypically, and functionally distinct disease-associated macrophages (DAMs), often accelerating disease progression (Park M. D. et al., 2022, Cell, 185:4259-4279).

Lung cancer accounts for ⅕ of all cancer deaths worldwide, with non-small cell lung cancer (NSCLC) representing the most common histological subtype (Saito A. et al., 2018, Int. J. Mol. Sci. 19). Lung tumor-associated macrophages (TAMs) of both embryonic and monocyte-derived origins promote immunosuppression and support tumor growth, metastasis and therapeutic resistance, in part by secreting inflammatory cytokines such as TGF-β1, IL-1b and IL-6 (Saito A. et al., 2018, Int. J. Mol. Sci., 19; Loyher P. L. et al., 2018, J. Exp. Med., 215:2536-2553; Garon E. B. et al., 2020, JTO Clin Res Rep, 1:100001; Xu F. et al., 2020, Mol. Med. Rep., 22:4107-4115; Mittal P. et al., 2020, Cancers (Basel), 12). Comprising up to 50% of the tumor mass, these TAMs have been identified as predominantly CD163-positive cells (Xu F. et al., 2020, Mol. Med. Rep., 22:4107-4115; Larionova I. et al., 2020, Front. Oncol., 10:566511; Larroquette M. et al., 2022, J. Immunother. Cancer, 10).

Endometriosis is defined as the growth of endometrial-like tissue outside of the uterus. It is a chronic inflammatory disease that affects approximately 190 million women worldwide causing pain and infertility (Zondervan K. T. et al., 2020, New England Journal of Medicine, 382:1244-1256; Hogg C. et al., 2020, Frontiers in Endocrinology, 11:00007). It causes pain and infertility and is associated with an increased risk of ovarian cancer (Pearce C. L. et al., 2012, Lancet. Oncol., 13:385-394; Lu Y. et al., 2015, Hum. Mol. Genet., 24:5955-5964: Lee A. W. et al., 2016, Fertil. Steril., 105:35-43; Sainz de la Cuesta R. et al., 1996, Gynecol. Oncol., 60:238-244). Current treatment consisting of hormonal mediation and surgical removal of lesions has been ineffective and associated with complications and morbidity, owing to the limited mechanistic understanding of the disease (Zondervan, K. T. et al., 2020, New England Journal of Medicine, 382:1244-1256; Hogg, C. et al., 2020, Frontiers in Endocrinology, 11:00007). Many theories have been proposed on the pathogenesis of endometriosis, including immune dysregulation.

Macrophages, the most abundant immune cells present in endometriosis lesions, play a central role in the growth, development, vascularization, and innervation of lesions as well as generation of pain symptoms (Zondervan K. T. et al., 2020, New England Journal of Medicine, 382:1244-1256; Hogg C. et al., 2020, Frontiers in Endocrinology, 11:00007). Endometriosis-associated macrophages (EAMs) are derived from recruited monocytes, macrophages and granulocytes and are phenotypically and functionally distinct from tissue resident macrophages. Given the critical role of EAMs in endometriosis pathology, it is important to identify and characterize factors responsible for the establishment and maintenance of EAMs. TET3 presents an interesting candidate.

First, Tanaka et al. reported that double deletion of Tet2 and Tet3 in B cells led to B cell hyperactivation and autoimmune disease in mice (Tanaka S. et al., 2020, Nature Immunology, 21:950-961). Second, in macrophages TET3 was found to inhibit virus-induced INF-b production (Xue S. et al., 2016, Cell Reports, 16:1096-1105). Third, TET3 expression in hepatic stellate cells (HSCs) was reported thereby promoting liver fibrosis both in human and mouse (Xu Y. et al., 2020, Cell Reports, 30:1310-1318). HSCs play a major role in regulation of various forms of liver inflammation (Fujita T. et al., 2016, Inflammation and Regeneration, 36:1). TET3-mediated regulation of extracellular matrix-remodeling genes in human uterine fibroids were also documented (Cao T. et al., 2019, Oncogene, 38:5356-5366). Collectively, these studies point to an important role of TET3 in regulation of inflammation, raising the possibility that TET3 may regulate EAMs contributing to the pathogenesis of endometriosis.

### TET3 was Expressed in CD163-Positive Monocytes/Macrophages in Endometriosis Lesions

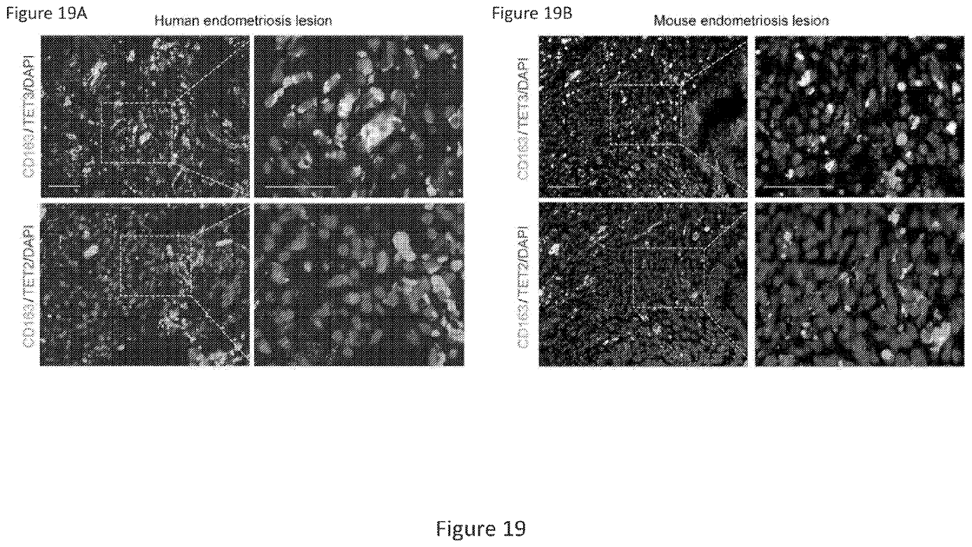

The predominant TET family isoforms expressed in macrophages are TET2 and TET3 (Tanaka, S. et al., 2020, Nature Immunology, 21:950-961; Xue, S. et al., 2016, Cell Reports, 16:1096-1105). The transmembrane scavenger receptor CD163 is expressed exclusively in monocytes and macrophages (Kristiansesn, M. et al., 2001, Nature, 409:198-201). Accumulation of CD163-expressing macrophages at the site of inflammation have been reported in a number of inflammatory diseases, including cutaneous arteritis, carotid atherosclerosis, and multiple sclerosis (Skytthe, M. K. et al., 2020, International Journal of Molecular Sciences, 21:5497). Extensive co-expression of TET3 with CD163 in human endometriosis lesions was observed, whereas co-expression of TET2 with CD163 was less prominent (FIG. 19A). Similar observations were made in mouse endometriosis lesions (FIG. 19B). These results indicated that TET3 may have an important function in CD163⁺ EAMs.

### TET3 Knockdown in Macrophages Cells Induced Apoptosis

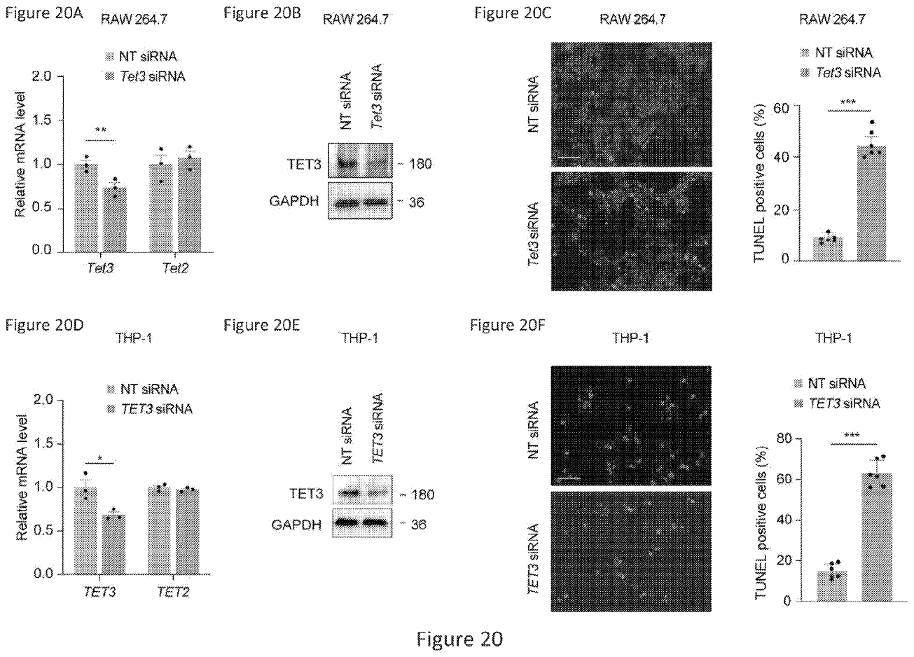

Macrophage cell lines were used to explore the functional significance of TET3 expression in CD163⁺ cells. TET3 expression was reduced in Raw 264.7 (hereafter called RAW), a mouse macrophage cell line, using siRNAs specifically targeting mouse Tet3 (Tet3 siRNA). Transfection of Tet3 siRNA led to decreased expression of Tet3 both at the mRNA (FIG. 20A) and protein (FIG. 20B) levels without affecting that of Tet2. Importantly, TET3 knockdown induced apoptosis in these cells (FIG. 20C). Likewise, when TET3 was downregulated in human THP-1 derived macrophages using an siRNA specifically targeting human TET3 (FIG. 20D and FIG. 20E), an increase in cell apoptosis was also observed (FIG. 20F). These results indicated that TET3 expression was required for the maintenance of macrophage cell viability.

### Bobcat339 Destabilized TET3 Protein and Induced Apoptosis in Macrophage Cells

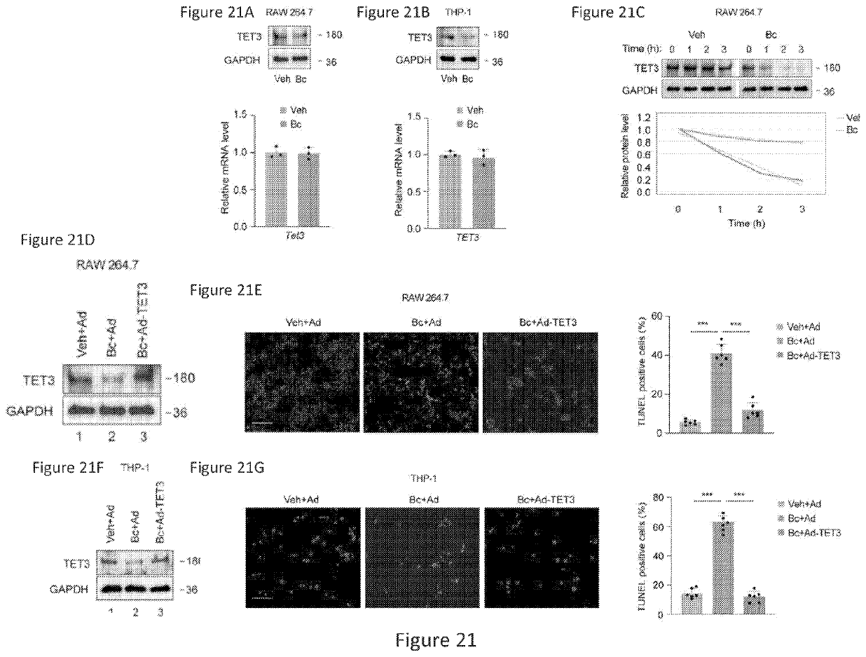

As incubation of HT-22 mouse hippocampal neuronal cells with Bobcat339 at 10 μM for 24 h decreased DNA 5hmC levels, indicating TET inhibition, RAW cells were incubated with 10 μM Bobcat339 for 24 h and decreased the levels of TET3 protein were observed without altering its mRNA abundance (FIG. 21A). Similar results were obtained in THP-1 cells (FIG. 21B). It was not unprecedented that compounds initially developed as protein function inhibitors were later serendipitously found to be protein degraders (Bekes, M. et al., 2022, Nature Reviews Drug Discovery, 21(3):181-200). For example, the estrogen antagonist ICI 164,384 binds to the hormone-binding domain of the estrogen receptor and induces its degradation without affecting its mRNA expression (Dauvois, S. et al., 1992, Proceedings of the National Academy of Sciences, 89:4037-4041).

To test whether Bobcat339 affect TET3 protein turnover, time course experiments were performed in the presence of cycloheximide, a protein synthesis inhibitor. TET3 was less stable in Bobcat339-treated vs. vehicle treated cells (FIG. 21C, upper panel). The half-life of TET3 was ~80 minutes in Bobcat339-treated cells, while that in vehicle treated cells was greater than 3 hours (FIG. 21C, bottom panel). It is important to note that while Bobcat339 was originally reported as an inhibitor or TET1 and TET2 activity, it was recently reported that Bobcat339 on its own had negligible inhibitory activity against TET1 and TET2 in the absence of contaminating coper (II) (Weirath, N. A. et al., 2022, ACS Medicinal Chemistry Letters, 13(5):792-798). Thus, Bobcat339 used in the present studies were shown to be free from Cu(II) contamination (Weirath, N. A. et al., 2022, ACS Medicinal Chemistry Letters, 13(5):792-798). Accordingly, the treatment of macrophage cells with Bobcat339 induced TET3 protein degradation that did not involve inhibition of TET3 enzymatic activity.

### Bobcat339 Promoted Apoptosis of Macrophage Cells

Because Bobcat339 decreased TET3 protein abundance (FIG. 21A through FIG. 21C) and because TET3 knockdown using siRNAs promoted apoptotic cell death (FIG. 20), it was tested whether exposing cells to Bobcat339 would induce apoptosis and if so, whether it would dependent on TET3 expression. Thus, RAW 246.7 cells were incubated with Bobcat339 in the presence or absence of exogenous TET3 expression from an adenoviral vector (Ad-TET3). While Bobcat339 expectedly decreased the level of TET3 (FIG. 3D, top blot, compare lane 2 to lane 1), exogenous TET3 expression restored it to the control level (compare lane 3 to lane 1). Consistent with earlier findings that TET3 knockdown led to apoptosis of macrophage cells (FIG. 20), treatment of RAW cells with Bobcat339 induced apoptosis, which was reversed by overexpression of TET3 (FIG. 21D and FIG. 21E). Similar observations were made in THP-1 microphages (FIG. 21F and FIG. 21G). Taken together, the results demonstrated that Bobcat339 promoted apoptosis of macrophage cells in a TET3-dependent manner.

### Bobcat339 Reduced Disease Burden in a Murine Model of Endometriosis

Given the preceding results and although not bound by any particular theory, it was hypothesized that TET3-expressing CD163⁺ EAM contributes critically to the pathogenesis of endometriosis and that depleting these cells using Bobcat339 produces therapeutic effects. To test this, a well-established murine model of endometriosis was employed (Rosa, E. S. A. et al., 2019, Reproductive Sciences, 26(10):1395-1400).

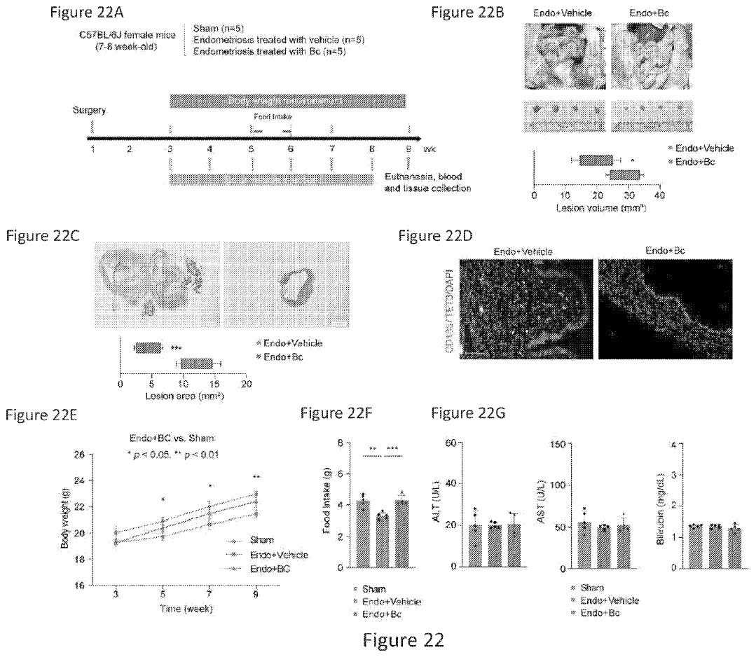

Female mice were randomly divided into three groups (sham, endometriosis treated with vehicle, and endometriosis treated with Bobcat) and subjected to surgery (week 1) to induce endometriosis or sham (FIG. 22A). The first once-a-week intraperitoneal (i.p.) injection of Bobcat339 (or vehicle) at a dose of 2.5 mg/kg was performed on week 3, followed by euthanasia and blood and tissue collection at week 9. Bobcat339 treatment significantly reduced lesion volume both macroscopically (FIG. 22B) and histologically (FIG. 22C).

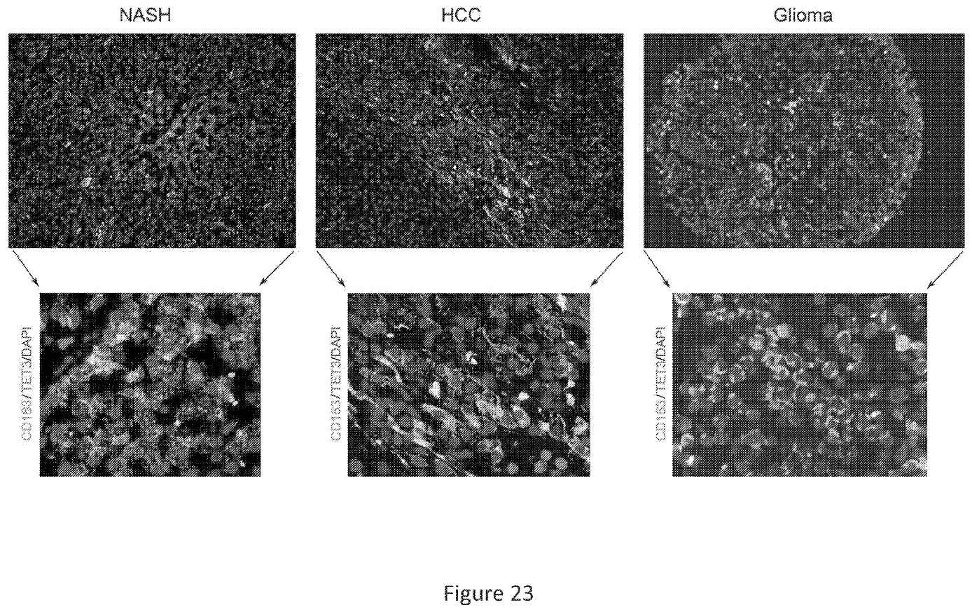

Immunofluorescence analysis revealed depletion of TET3/CD163-double positive cells in the lesions of Bobcat339-treated mice (FIG. 22D). While no statistically significant differences were observed in body weight between Sham and Endo+Bobcat339 and between Endo+Veh and Endo+Bobcat, body weight in Endo+Veh was significantly lower compared to that of Sham group (FIG. 22E). A decrease in food intake was observed in Endo+Veh mice vs. Sham mice but the decrease was not observed in Endo+Bobcat339 mice (FIG. 22F). Finally, there was no evidence of liver toxicity following 6 weeks of once-a-week Bobcat339 treatment (FIG. 22G). These results demonstrated that Bobcat339 was effective in treating endometriosis, in part, by depleting the disease-promoting TET3-positive macrophages. TET3/CD163-double positive macrophages in cells were present in tissue samples from human patients with NASH, cancers, HCC, and glioma (FIG. 23). For this reason, and because TET3 was highly expressed in human tissue macrophages associated with NAFLD and cancers, additional studies focus on immunofluorescence images of TET3/CD163 double positive macrophages in NASH, cancers, and CVD.

### Example 4: TET3 Controlled Survival and Key Functions of Disease-Associated Macrophages

Macrophages are tissue-resident or infiltrated immune cells that play critical roles in the development and progression of chronic inflammatory diseases including non-alcoholic fatty liver disease (NAFLD) and endometriosis (Ardura et al., 2019, Front Pharmacol, 10:1255; Watanabe et al., 2019, J Clin Invest, 129:2619-2628; Hogg et al., 2020, Front Endocrinol, 11:7; Fonseca et al., 2023, Nat Genet). However, targeting these DAMs for therapy has remained extremely challenging, largely owing to their high heterogeneity both molecularly and phenotypically (Ardura et al., 2019, Front Pharmacol, 10:1255; Skytthe et al., 2020, Int J Mol Sci 21, 5). Historically, macrophages have been classified into an “M1” or “M2” phenotype, with the former being pro-inflammatory and the latter being anti-inflammatory. However, this M1/M2 polarization paradigm is over-simplistic because these extreme polarization states only exist in vitro and do not recapitulate the remarkable heterogeneity and plasticity of macrophages in vivo. In fact, macrophages are able to adopt intermediate phenotypes that present mixed M1 and M2 characteristics and modulate their transcriptomes, phenotypes (e.g., surface markers), and functions in response to microenvironment in a tissue- and disease stage-dependent manner (Ardura et al., 2019, Front Pharmacol, 10:1255; Watanabe et al., 2019, J Clin Invest, 129:2619-2628). Thus, identification of specific subsets of macrophages and factors/mechanisms that drive disease progression is of paramount importance for the development of effective intervention strategies for the diseases.

NAFLD is an emerging health issue affecting nearly 25% of the world adult population (Younossi et al., 2018, Nat Rev Gastroenterol Hepatol, 15:11-20). NAFLD encompasses multiple disease states from simple steatosis to nonalcoholic steatohepatitis (NASH) and cirrhosis. NASH is characterized by inflammation of the liver that causes fibrosis and predisposes to cirrhosis and hepatocellular carcinoma (HCC) (Younossi et al., 2018, Nat Rev Gastroenterol Hepatol, 15:11-206; Barreby et al., 2022, Nat Rev Endocrinol, 18:461-472). Liver macrophages have been shown to drive NASH progression (Barreby et al., 2022, Nat Rev Endocrinol, 18:461-472; Cai et al., 2020, Cell Metab, 31:406-421).

Endometriosis is defined as the growth of endometrial-like tissue outside of the uterus (Hogg et al., 2020, Front Endocrinol, 11:7). It causes pain and infertility and is associated with an increased risk of ovarian cancer (Pearce et al., 2012, Lancet Oncol, 13:385-394; Lu et al., 2015, Hum Mol Genet, 24:5955-5964; Lee et al., 2016, Fertil Steril, 105:35-43; Sainz de la Cuesta et al., 1996, Gynecol Oncol, 60:238-244). Endometriosis occurs in approximately 10% of reproductive-aged females, impacting ~175 million women worldwide (Hogg et al., 2020, Front Endocrinol, 11:7; Zondervan et al., 2020, N Engl J Med, 382:1244-1256; Taylor et al., 2021, Lancet, 397:839-852; Donnez et al., 2022, Lancet, 400:896-907). As the most abundant immune cells present in endometriosis lesions, macrophages have been implicated in playing a central role in the growth, development, vascularization, and innervation of lesions as well as generation of pain symptoms (Hogg et al., 2020, Front Endocrinol, 11:7; Zondervan et al., 2020, N Engl J Med, 382:1244-1256). Clearly, DAMs are critical drivers of both endometriosis and NASH, which represent two distinct types of inflammatory diseases. Despite high unmet medical needs, there are no effective treatments for both diseases (Hogg et al., 2020, Front Endocrinol, 11:7; Younossi et al., 2018, Nat Rev Gastroenterol Hepatol, 15:11-20; Zondervan et al., 2020, N Engl J Med, 382:1244-1256).

The TET family proteins (TET1, TET2 and TET3) oxidize 5-methylcytosines (5mC) to 5-hydroxymethylcytosines (5hmC) and its derivatives to mediate DNA demethylation (Wu et al., 2017, Nat Rev Genet, 18:517-534; Lio et al., 2020, J Biosci, 45; Yang et al., 2020, Development, 147). TET proteins can also regulate gene expression independently of their catalytic activities (Williams et al., 2011, Nature, 473:343-348; Kaas et al., 2013, Neuron, 79:1086-1093; Zhang et al., 2015, Nature, 525:389-393; Xue et al., 2016, Cell Rep, 16, 1096-1105; Guan et al., 2017, Proc Natl Acad Sci USA, 114:8229-8234; Montalban-Loro et al., 2019, Nat Commun, 10:1726; Tanaka et al., 2020, Nat Immunol, 21:950-961). TET loss-of-function has been associated with hematopoietic malignancies, myeloid malignancies, and solid cancers (Lio et al., 2020, J Biosci, 45). However, myeloid-specific Tet2 ablation inhibits melanoma growth in mice (Pan et al., 2017, Immunity, 47:284-297), highlighting cell-specific function of TET proteins. While a low level of TET3 protein has been detected in hepatocytes and hepatic stellate cells (HSCs) in healthy livers, abnormally increased expressions of TET3 in these cells promote hepatic fibrogenesis (Xu et al., 2020, Cell Rep, 30:1310-1318). The current work reported identification of TET3-expressing macrophages in tissue samples from patients with fibrotic NASH and intraperitoneal endometriosis. These cells represented a unifying pathological subset of DAMs which may be exploited as a new therapeutic target for their associated diseases.

The present studies identified TET3-expressing DAMs in patients with NASH and endometriosis. The present studies also demonstrated that depleting these DAMs through myeloid-specific Tet3 genetic ablation or administration of Bobcat339 ameliorated both diseases in murine models. Mechanistically, it was shown that TET3 targeted the activation of multiple regulatory pathways in macrophages crucial for disease pathology and progression and that this TET3-dependent mechanism was conserved between human and mouse macrophages. It was further shown that TET3 suppressed apoptosis, rendering DAMs vulnerable to TET3 inhibitors, such as Bobcat339.

TET3 was Expressed in a Subset of Macrophages Associated with NASH, Endometriosis, and NSCLC

Liver resident macrophages (Kupffer cells or KCs) comprise approximately 15% of total liver cells and are the largest population (80-90%) of tissue-resident macrophages in the body (Williams et al., 2002, Toxicol Pathol, 30:41-53). In the human endometrium, macrophages constitute up to 15% of all cells (Salamonsen et al., 2002, J Reprod Immunol, 57:95-108; Cominelli et al., 2014, Mol Hum Reprod, 20:767-775). The transmembrane scavenger receptor CD163 is exclusively expressed in monocytes and macrophages, with expression level increasing upon maturation from monocytes into macrophages (Kristiansen et al., 2011, Nature, 409:198-201). CD163 is highly expressed in KCs (Eichendorff et al., 2015, Mol Imaging Biol, 17:87-93; Svendsen et al., 2017, Mol Ther Methods Clin Dev, 4:50-61) and endometrial macrophages (Cominelli et al., 2014, Mol Hum Reprod, 20:767-775), with co-expressions of other surface markers. For example, single-cell RNA-sequencing studies have identified the human KC population as CD163+MARCO+CD5L+TIMD4+.

Notably, accumulation of CD163+ macrophages at sites of inflammation has also been documented including NASH, NSCLC, endometriosis, atherosclerosis, lupus nephritis, and neurodegenerative diseases (Hogg et al., 2020, Front Endocrinol (Lausanne), 11:7; Skytthe et al., 2020, Int J Mol Sci, 21; De Vito et al., 2012, Int J Mol Med, 30:49-56; Kazankov et al., 2015, J Gastroenterol Hepatol, 30:1293-1300). In children with NASH, increases in the numbers of CD163+ macrophages in the liver significantly correlated with severity of the disease (De Vito et al., 2012, Int J Mol Med, 30:49-56; Kazankov et al., 2015, J Gastroenterol Hepatol, 30:1293-1300).

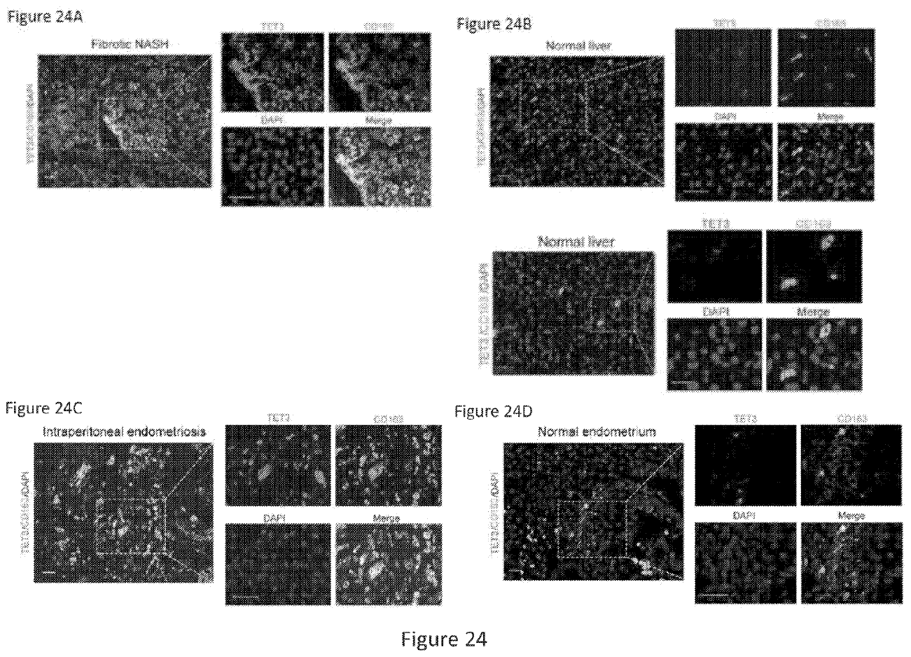

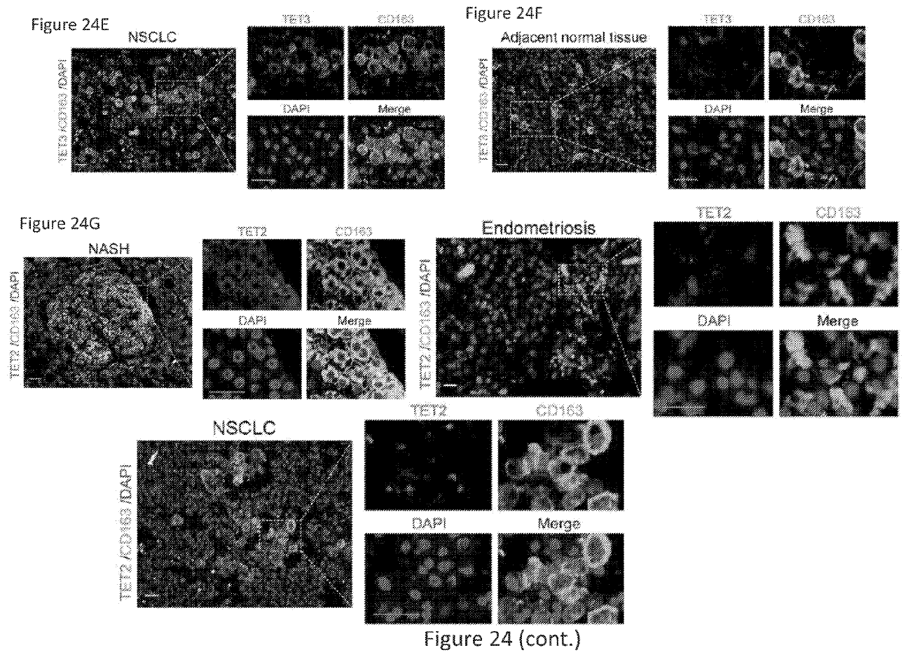

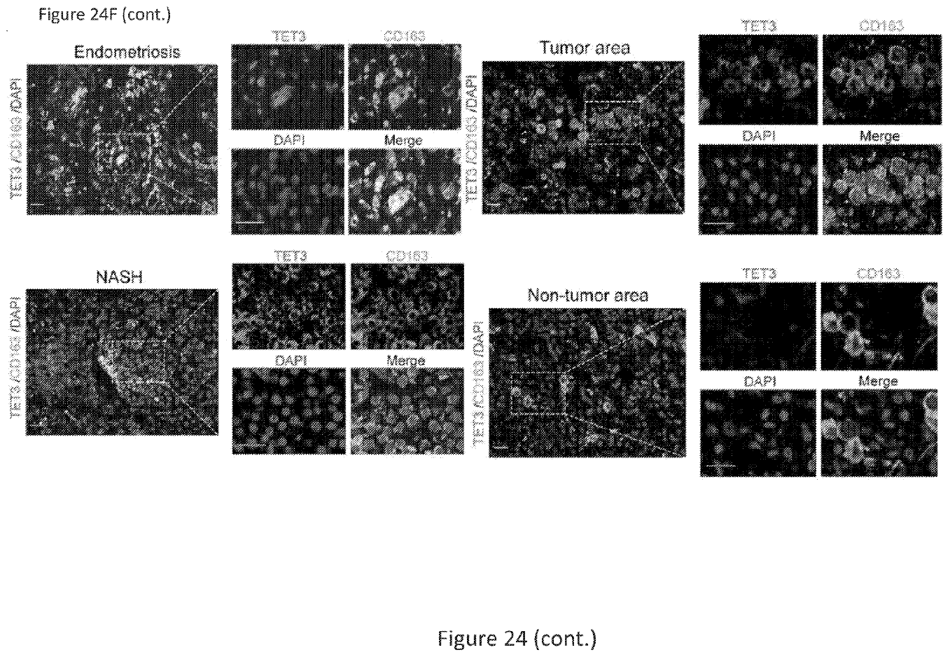

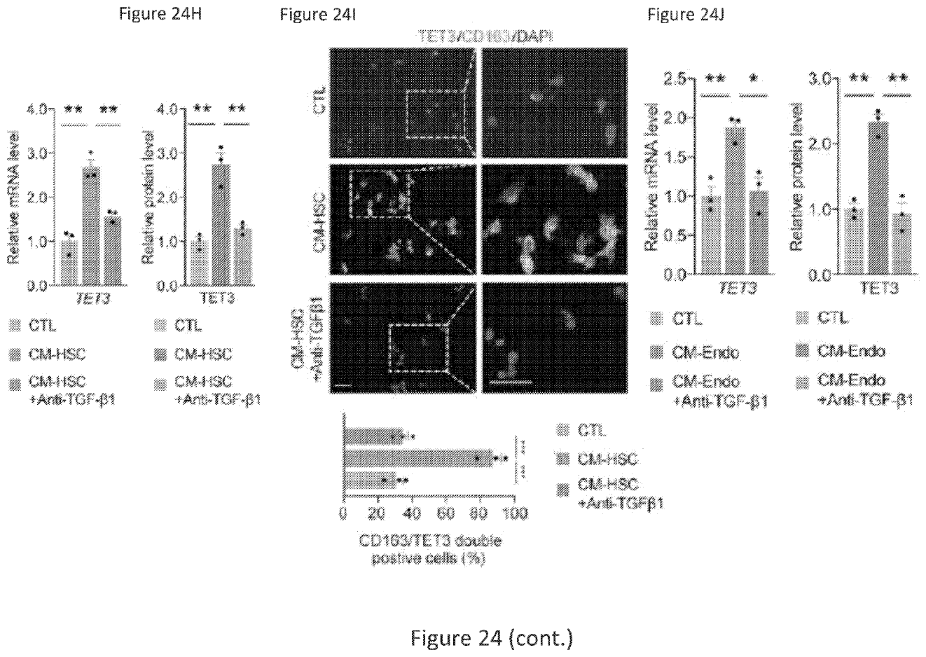

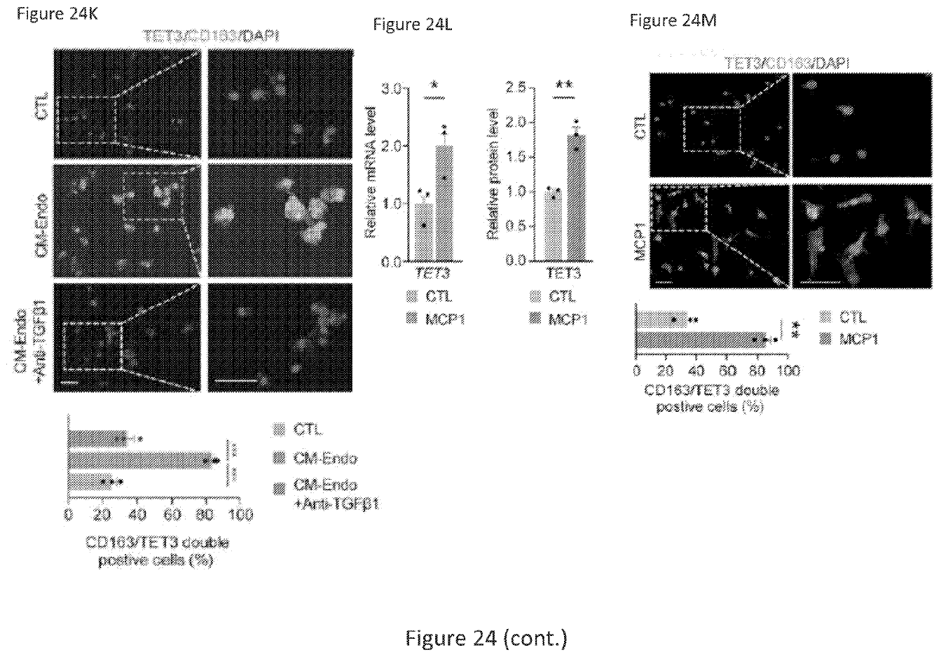

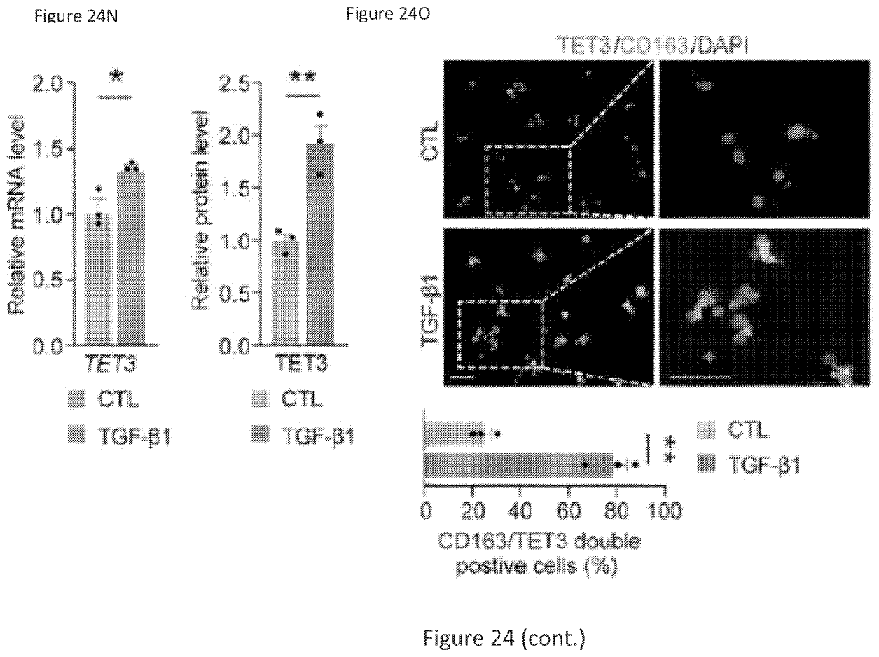

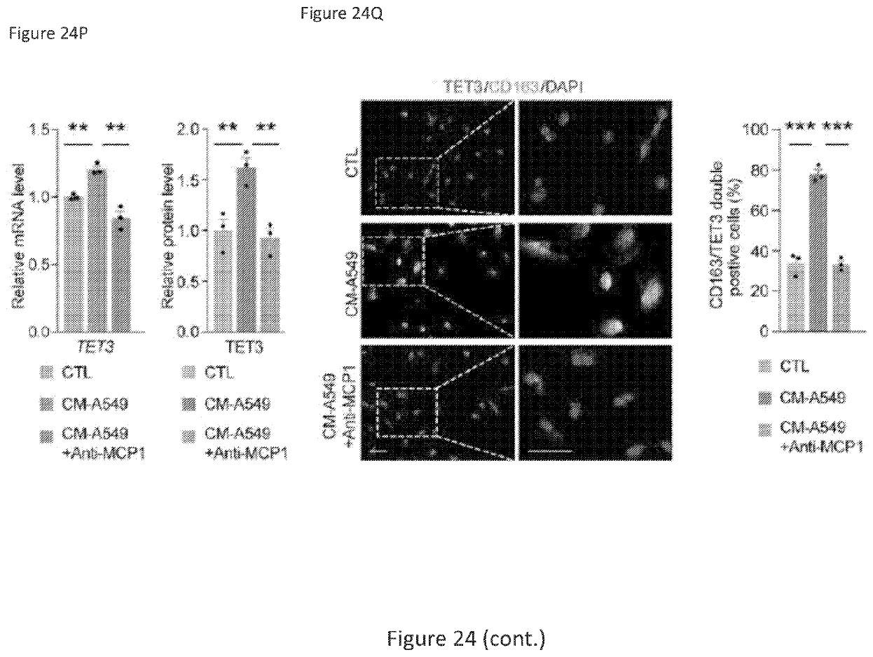

Though similar numbers of CD163+ macrophages were found in the livers of adults with and without NASH, CD163+ macrophage aggregates were seen only in livers of patients with NASH (Kazankov et al., 2015, J Gastroenterol Hepatol, 30:1293-1300), indicating a distinct subset of CD163+ macrophages in NASH. In support of this view, TET3 expression was detected in aggregates of CD163+ macrophages in the livers of adult patients with NASH (FIG. 24A), while no TET3/CD163 double-positive macrophages were detected in healthy livers (FIG. 24B). Note, normal human hepatocytes expressed a low level of TET3 in the cytoplasm (FIG. 24B) (Xu et al., 2020, Cell Rep, 30:1310-1318). Likewise, an extensive co-localization of TET3 with CD163+ macrophages was evident in intraperitoneal endometriosis lesions (FIG. 24C) but not in the endometrium of non-endometriosis controls (FIG. 24D).

The present experiments defined TET3-overexpressing CD163⁺ macrophages as “TET3/CD163-double positive macrophages”. While TET3/CD163 double-positive macrophages were found in NSCLC (FIG. 24E), these cells were absent in adjacent normal tissues (FIG. 24F). The specificity of the TET3 antibody was confirmed in TET3 knockdown cells (FIG. 1C).

Heterogeneous subcellular localization of TET3 in CD163⁺ macrophages was also observed (FIG. 24). Heterogeneous subcellular localization of TET proteins has been previously reported (Xu et al., 2020, Cell Rep, 30:1310-1318; 27, 37-40; Arioka et al., 2012, PLoS One, 7:e45031; Zhang et al., 2014, J Biol Chem, 289:5986-5996; Mi et al., 2015, Int J Mol Sci, 16:21846-21857; Huang et al., 2016, Clin Epigenetics, 8:9). As TET3 and TET2 are the predominant TET family isoforms expressed in macrophages (Xue et al., 2016, Cell Rep, 16:1096-1105; Cull et al., 2017, Exp Hematol., 55:56-70), TET2 in tissue samples was also examined. While TET2 expression was readily detectable in CD163⁺ aggregates in NASH, it was negligible in CD163⁺ macrophages in endometriosis and NSCLC (FIG. 24G). Further studies focused on studying TET3/CD163 double-positive macrophages as they may represent a molecularly and functionally distinct subset of macrophages associated with these diseases.

Thus, while TET3 showed a predominantly cytoplasmic staining in CD163+ cells in NASH (FIG. 24A), it was prominently nuclear in the endometriosis lesions (FIG. 24C). These results indicated that the TET3/CD163 double-positive macrophages represent a molecularly and functionally distinct subset of macrophages associated with NASH and endometriosis.

TET3 Expression was Altered by Signals from Tissue Microenvironment

The cross-talk between tissue microenvironment and macrophages reprograms macrophages transcriptomically and phenotypically, contributing to disease progression (Ardura et al., 2019, Front Pharmacol, 10:1255; Watanabe M et al., 2019, J Clin Invest, 129:2619-2628). Thus, human peripheral blood monocyte-derived macrophages (MDMs) were incubated with conditioned media from activated, fibrogenic human hepatic stellate cells (Xu et al., 2005, Gut, 54:142-151) (CM-HSC) or from human endometriosis stromal cells (CM-Endo). Increased expression of TET3 were observed (FIG. 24H and FIG. 24J, compare blue bars to orange bars) and increases in the number of TET3/CD163-double positive cells (FIG. 24I and FIG. 24K, compare blue bars to orange bars). It was previously reported that TGF-β1 stimulated TET3 expression in hepatocytes and HSCs during hepatic fibrosis progression (Xu et al., 2020, Cell Rep, 30:1310-1318). Likewise, levels of TGF-β1 were found to be increased in the serum, peritoneal fluid, ectopic endometrium, and peritoneum of women with endometriosis as compared to women without endometriosis and Tgfb1 deletion in mice reduced the growth of endometriosis lesions (Young et al., 2017, Hum Reprod Update, 23:548-559). In light of these prior studies, additional studies tested whether TGF-β1 might be a factor present in the conditioned media that contributed to the upregulation of TET3 and the TET3/CD163 double-positive cells in MDMs. Indeed, a TGF-β1-specific antibody was able to block the conditioned media-induced upregulation of TET3 and TET3/CD163 double-positive cells (FIG. 24H through FIG. 24K, compare gray bars to blue bars), while exposing macrophages to purified TGF-β1 increased TET3 expression and the number of TET3/CD163 double-positive cells (FIG. 24L and FIG. 24M). Collectively, these results indicated that TET3 expression can be modulated by tissue microenvironments and that TET3 may play an important role in DAMs.

### TET3 Expression in Macrophage was Upregulated by Inflammatory Mediators

Macrophages are remarkably plastic cells which can be modified molecularly and functionally in response to microenvironmental cues (Park et al., 2022, Cell, 185:4259-4279; Ardura et al., 2019, Front Pharmacol, 10:1255). TGF-β1 upregulated TET3 in hepatic stellate cells (HSCs) and hepatocytes during liver fibrosis (Xu et al., 2020, Cell Rep, 30:1310-1318). Levels of TGF-β1 were found to increase in the peritoneal cavity of women with endometriosis and Tgtb1 deletion reduced the growth of endometriosis lesions in mice (Young et al., 2017, Hum Reprod Update, 23:548-559).

MCP1, also known as CCL2, secreted from lung tumor cells and associated stromal compartment altered macrophages to facilitate tumor growth and metastasis (Yoshimura et al., 2015, Front Immunol, 6:332; Wang et al., 2022, Oncol Lett, 23:26). MCP1 blockage using neutralizing antibodies elicited anti-tumor effects in multiple NSCLC mouse models (Fridlender et al., 2011, Am J Respir Cell Mol Biol, 44:230-237).

To address whether TGF-β1 and MCP1 might affect macrophage TET3 expression, the studies exposed human MDMs to these factors. TGF-β1 increased TET3 mRNA (FIG. 24L) with a concomitant increase in the number of TET3/CD163 double-positive cells (FIG. 24M). Incubation of MDMs with MCP1 resulted in similar effects (FIG. 24N and FIG. 24O). Next, MDMs were exposed to conditioned media from human HSCs (CM-HSC). An increased expression of TET3 mRNA (FIG. 24H) accompanied by increased numbers of TET3/CD163-double positive cells was observed (FIG. 24J). However, when cells were incubated with the conditioned media in the presence of a TGF-β1-specific antibody, these effects were abolished (FIG. 24H and FIG. 24J).

Similar observations were made when cells were exposed to conditioned media from human endometriosis stromal cells (CM-Endo; Chen et al., 2021, Reprod Sci, 28:426-434; FIG. 24J and FIG. 24K). A549 is the most commonly used human NSCLC cell line. Exposing MDMs to conditioned media from A549 increased TET3 expression and numbers of TET3/CD163-double positive cells, and the effects were blocked by an MCP1-specific antibody (FIG. 24P and FIG. 24Q). Collectively, these results indicated that TET3 overexpression in macrophages is induced by inflammatory cytokines present in disease microenvironments and that these macrophages may be molecularly and functionally distinct from those not overexpressing TET3.

### TET3 Knockdown Increased Apoptosis of Macrophages

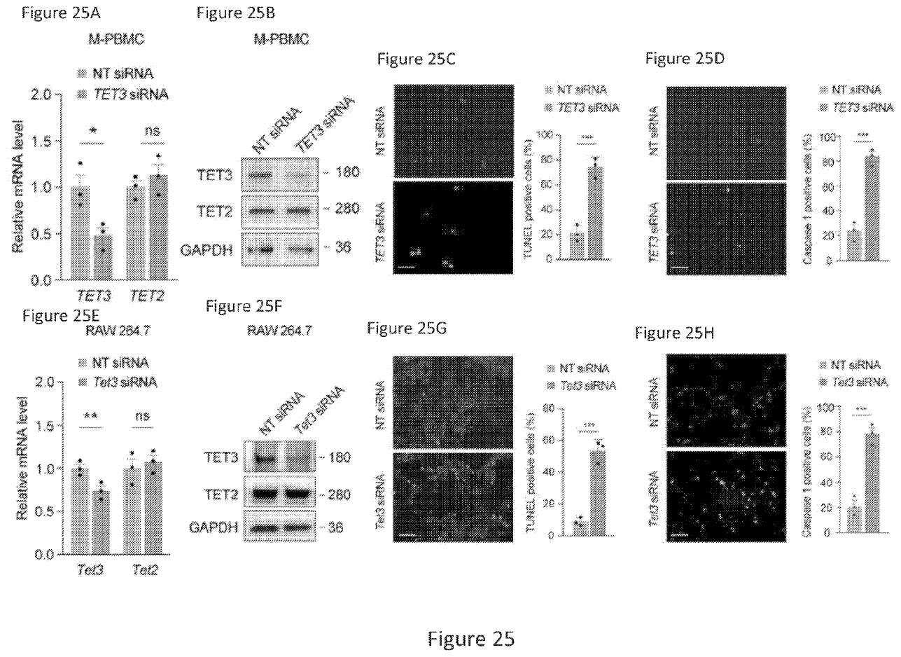

To explore the functional significance of TET3 in DAMs, an in vitro approach was first investigated. TET3 expression was reduced in M-PMBCs using an siRNA specifically targeting human TET3 (TET3 siRNA) (Xie et al., 2022, J Clin Invest 132) (FIG. 24J and FIG. 24K). Transfection of TET3 siRNA resulted in decreased expression of TET3 mRNA (FIG. 25A) and protein (FIG. 25B) without affecting TET2 mRNA expression (FIG. 25A). TET1 expression was not detected in MDMs, consistent with precious reports that the predominant TET family isoforms expressed in macrophages are TET2 and TET3 (Xue et al., 2016, Cell Rep, 16:1096-1105; Tanaka et al., 2016, Nat Immunol, 21:950-961).

Importantly, TET3 knockdown induced programmed cell death, as assessed by the Terminal deoxynucleotidyl transferase dUTP nick end labeling (TUNEL) assay which detects DNA damage during programmed cell death (FIG. 25C). Consistently, there was a significant increase in caspase-1 activity in TET3 knockdown macrophages (FIG. 25D). Likewise, TET3 knockdown using an siRNA specifically targeting mouse Tet3 (Tet3 siRNA) (Xie et al., 2022, J Clin Invest 132) in mouse Raw 264.7 macrophages increased apoptosis (FIG. 25E through FIG. 25H). These results indicated that TET3 expression is essential for maintaining the viability of both human and mouse macrophages.

### Bobcat339 Destabilized TET3 Protein in Macrophages

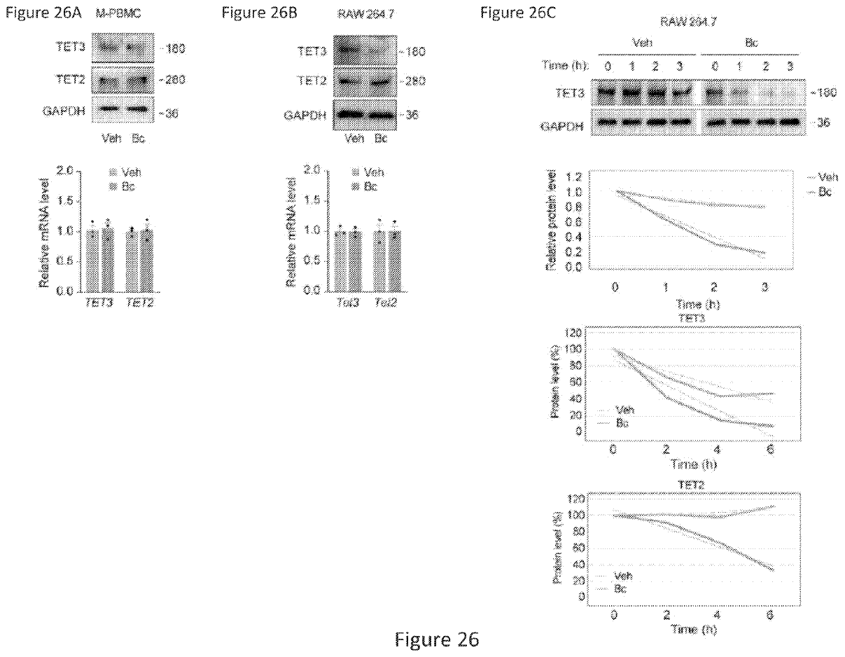

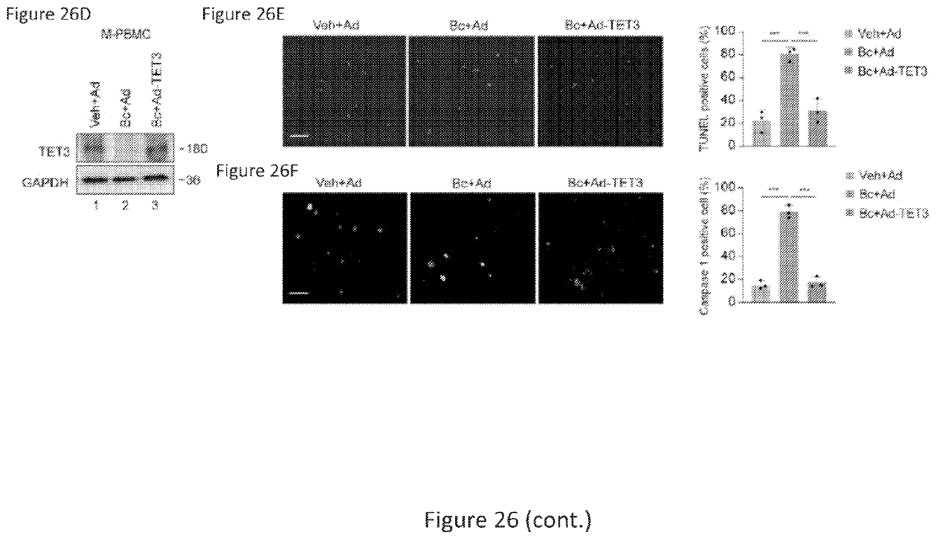

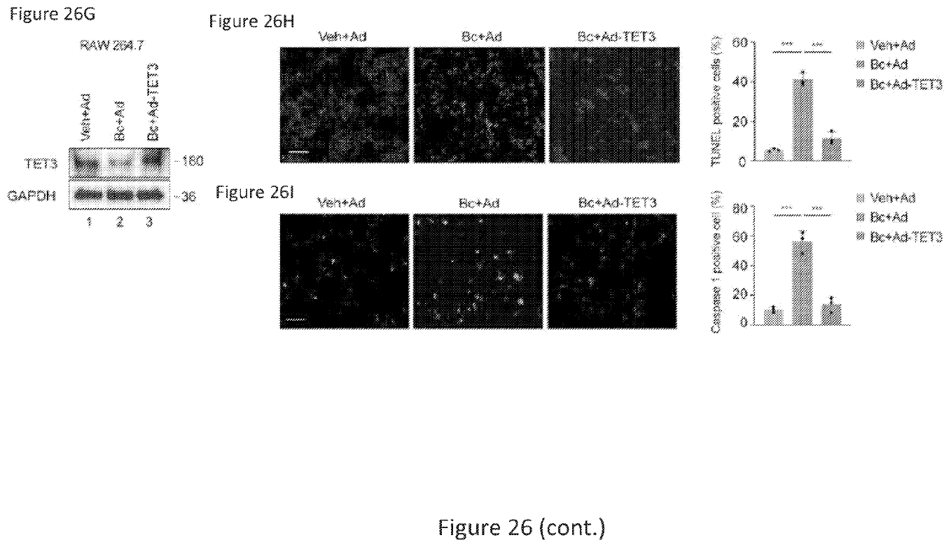

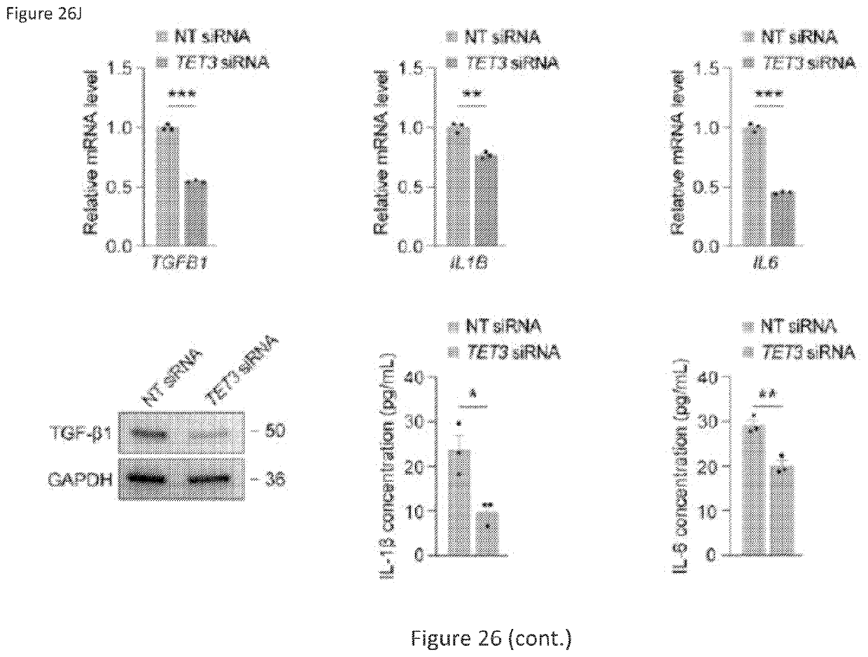

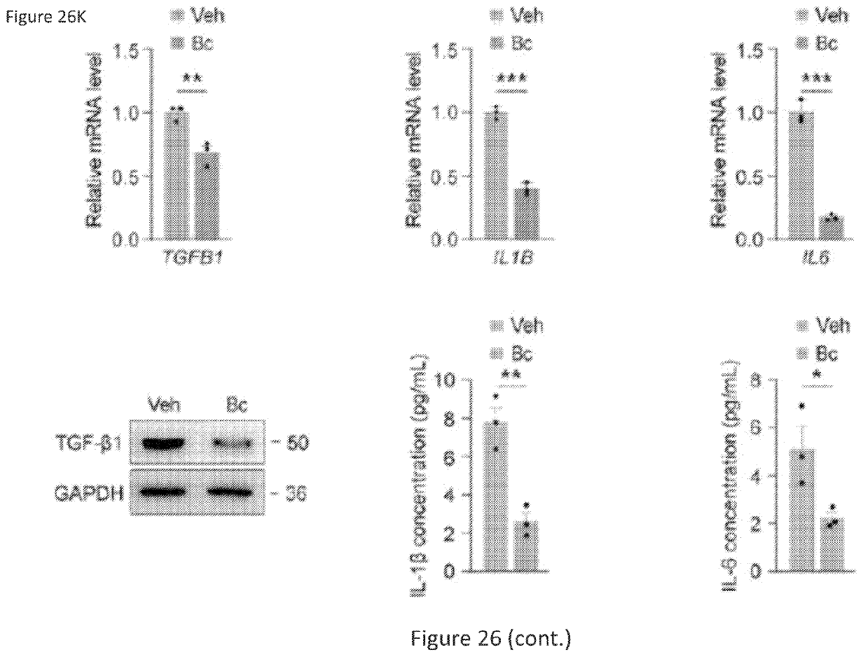

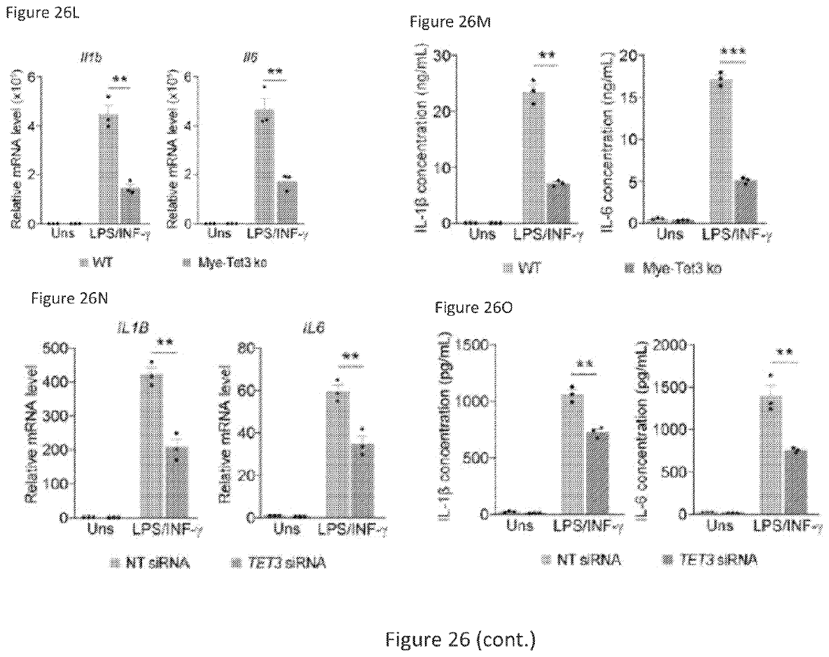

It was recently reported that Bobcat339, a synthetic cytosine derivative (Chua et al., 2019, ACS Med Chem Lett, 10:180-185), induced TET3 protein degradation in human and mouse neuronal cells (Lv et al., 2023, Proc Natl Acad Sci USA, In press). When MDMs was incubated with Bobcat339 at 10 μM for 24 h, a decrease in TET3 expression was observed at the protein (FIG. 26A, upper panel) but not the mRNA level (FIG. 26A, bottom panel), while the expression of TET2 was not affected (FIG. 26A). Bobcat339 treatment did not alter the mRNA levels of TET3 and TET2 (FIG. 26A). Similar results were obtained with Raw 264.7 cells (FIG. 26B). The 10 μM concentration was chosen based on the previous studies showing that it decreased the steady-state protein level of TET3 (but not TET2) in human and mouse neuronal cells (Lv et al., 2023, Proc Natl Acad Sci USA, In press).

To determine whether Bobcat339 might affect TET3 protein turnover in macrophages, time course experiments were performed in the presence of cycloheximide, a protein synthesis inhibitor. It was found that TET3 was less stable in Bobcat339-treated vs. vehicle treated macrophages (FIG. 26C, upper panel). While the half-life of TET3 in vehicle-treated cells remained stable, that of TET3 was dramatically shortened (~80 min) in Bobcat339-treated cells (FIG. 26C, bottom panel). It was concluded that Bobcat339 promoted TET3 protein degradation in macrophages.

### Bobcat339 Promoted Macrophage Apoptosis in a TET3-Dependent Manner

Because Bobcat339 destabilized TET3 (FIG. 26A through FIG. 26C) and because siRNA-mediated TET3 knockdown induced apoptosis of macrophages (FIG. 25), studies were to performed to determine whether exposing cells to Bobcat339 would increase apoptosis, and if so, whether it would occur in a TET3-dependent manner. 10 μM, the minimal effective concentration of Bobcat339 (Lv et al., 2023, Proc Natl Acad Sci USA, In press) were used in the following rescue experiments. Thus, MDMs were incubated with Bobcat339 in the presence or absence of exogenous TET3 expression from an adenoviral vector (Ad-TET3) (Lv et al., 2023, Proc Natl Acad Sci USA, In press).

While Bobcat339 expectedly decreased the level of TET3 (FIG. 26D, top blot, compare lane 2 to lane 1), exogenous TET3 expression restored it to the control level (compare lane 3 to lane 1). Consistent with the earlier observation that siRNA-mediated TET3 knockdown induces apoptosis (FIG. 25), treatment of these cells with Bobcat339 increased apoptosis, as determined by TUNEL (FIG. 26E) and Caspase-1 (FIG. 26F) assays, which was abolished when TET3 protein was restored to control levels (FIG. 26D through FIG. 26F). Similar observations were made in RAW 246.7 cells (FIG. 26G through FIG. 26J). Thus, Bobcat339 induced apoptosis in a TET3-dependent manner, which was conserved in human and mouse macrophages, and promoted macrophage apoptosis with TET3 being a critical effector.

### TET3 Affects Pathways Involved in TGF-β Signaling, Metabolic Reprogramming, and Apoptosis—TET3 Regulated Expression of Inflammatory Cytokines

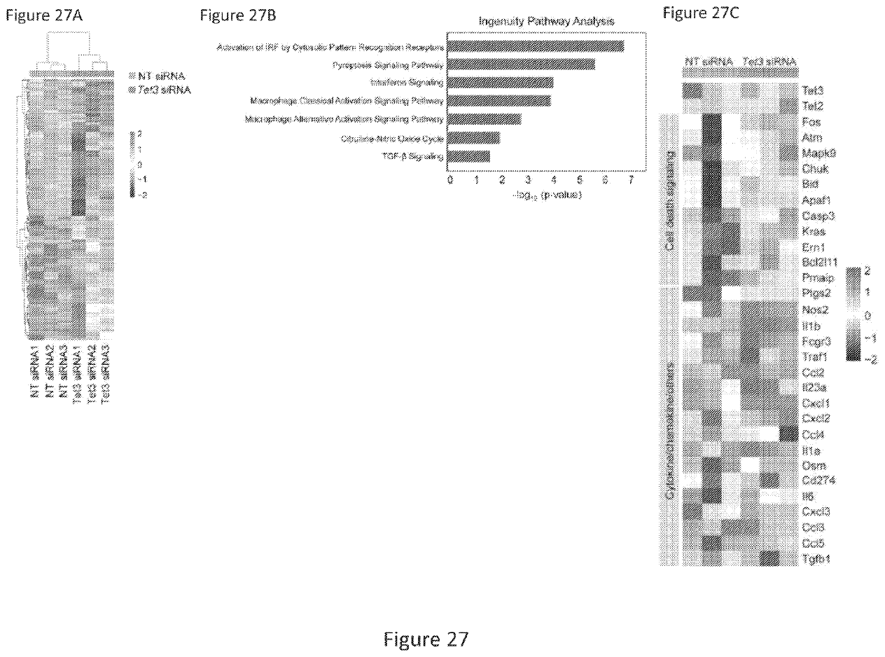

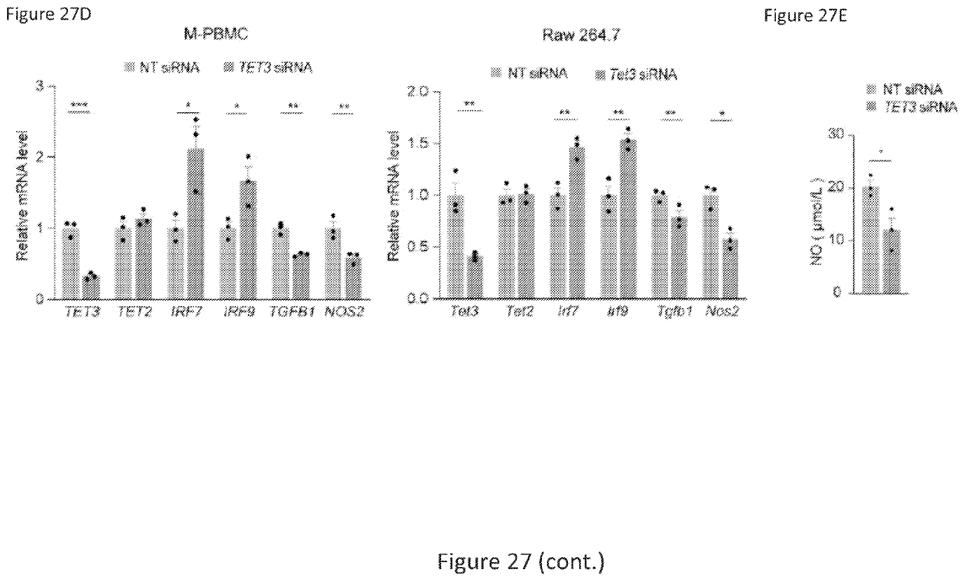

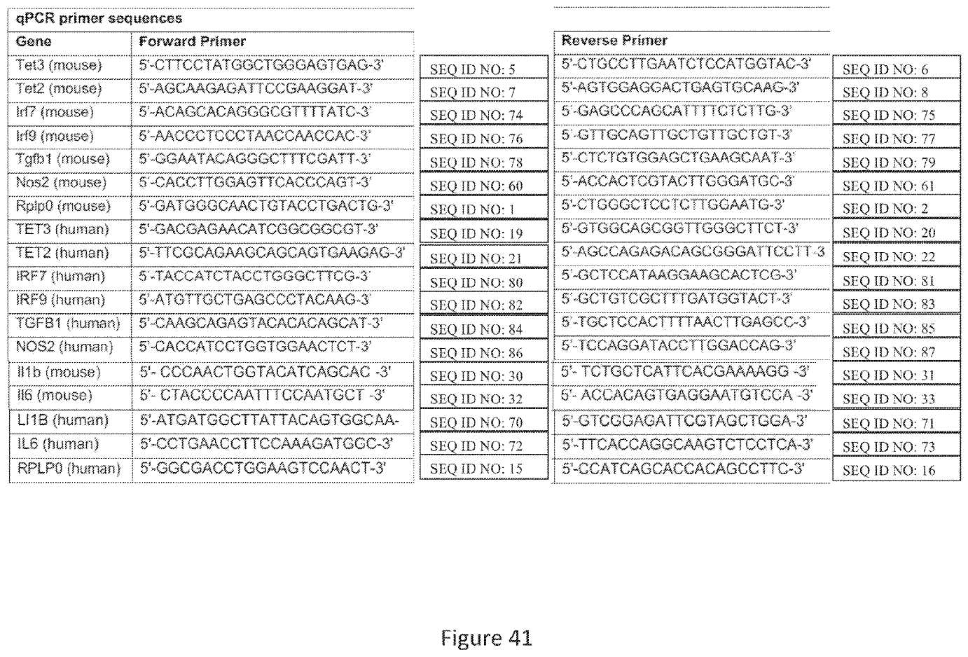

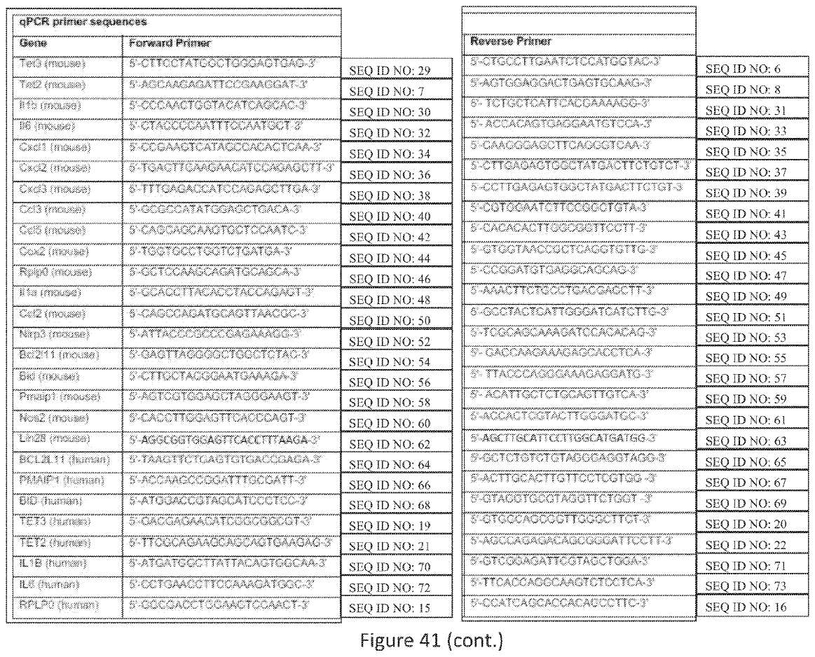

To gain a deeper mechanistic understanding into how TET3 regulates DAMs to affect disease progression, genome-wide expression profiling (RNA-seq) were performed on RNAs isolated from Raw 264.7 cells at 48 h after transfection with NT siRNA or Tet3 siRNA. TET3 knockdown induced profound gene expression changes (FIG. 27A, FIG. 27C, and FIG. 41). While Tet3 expression was significantly downregulated in Tet3 siRNA when compared to NT siRNA treated cells, that of Tet2 was unaltered (FIG. 27C). Importantly, TET3 knockdown decreased proinflammatory signaling and fibrosis-related gene signatures (FIG. 27C).

Ingenuity Pathway Analysis (IPA) highlighted several pathways that were significantly affected by TET3, and studies here focused on elaborating those associated with TGF-β signaling, citrulline-nitric oxide cycle, and apoptosis as well as the study of TET3-mediated regulation of TGF-β1, IL-1β and IL-6, as these have been shown to play important roles in the development and progression of NSCLC, endometriosis and NASH (FIG. 27B).

TGF-β1: In NSCLC, besides its direct effect on tumor cells by inducing epithelial-to-mesenchymal transition, TGF-β1 orchestrates the tumor microenvironment by promoting fibroblast activation, extracellular matrix remodeling, immune evasion and angiogenesis, thereby facilitating tumor progression (Saito et al., 2018, Int J Mol Sci, 19; Baba et al., 2022, Front Pharmacol, 13:791272). Aberrant TGF-β signaling also underlies endometriosis and NASH. In mice, Tgfb1 ablation reduced endometriosis lesions (Young et al., 2017, Hum Reprod Update, 23:548-559); inhibition of TGF-β1 production from liver macrophages decreased NASH and associated fibrosis (Cai et al, 2020, Cell Metab, 31:406-421).

IL-1β: The importance of IL-1β in NSCLC has been demonstrated. IL-1β activates signaling pathways including those mediated by NF-kB, cyclooxygenase, and MAPK, contributing to the establishment and maintenance of TAM functions (Garon et al., 2020, JTO Clin Res Rep, 1:100001). Consistently, in a recent phase III clinical trial, treatment with canakinumab, a human monoclonal antibody specific for IL-1β, substantially reduced incident NSCLC and lung cancer mortality in a dose-dependent manner (Garon et al., 2020, JTO Clin Res Rep, 1:100001). IL-1β was found to increase in peritoneal fluids and endometriosis lesions from women with endometriosis and pelvic pain (Peng et al., 2020, Hum Reprod, 35:901-912; Akoum et al., 2008, Fertil Steril, 89:1618-1624). Notably, IL-1β promotes local neurogenesis thereby increasing nerve density around endometriosis and endometriosis-associated pain (Peng et al., 2020, Hum Reprod, 35:901-912). Further, IL-1β has a well-established role in all the stages of NAFLD by promoting steatosis, inflammation, and fibrosis (Mirea et al., 2018, Trends Mol Med, 24:458-471; Tilg et al., 2020, Expert Opin Investig Drugs, 29:103-106).

IL-6: IL-6 acts as an important downstream effector of IL-1β-mediated regulation of TAMs in NSCLC (Garon et al., 2020, JTO Clin Res Rep, 1:100001). The role of IL-6 in endometriosis is supported by several lines of evidence. First, IL-6 is elevated in the peritoneal fluid and the serum of patients with endometriosis. Second, IL-6 released from activated macrophages stimulates the migration of endometriotic epithelial cells during formation of extra-pelvic endometriosis (Woo et al., 2017, Biol Reprod, 97:660-670). Third, treating rats with tocilizumab, an anti-IL-6 receptor monoclonal antibody, leads to significantly reduced endometriosis burden (El-Zayadi et al., 2020, Immunol Res, 68:389-397). Finally, an elevated production of IL-6 from Tet2-deficient macrophages was implicated in NASH progression (Wong et al., 2023, Nature, 616:747-754).

The RNA-seq analysis revealed altered TGF-β signaling following TET3 knockdown (FIG. 27B, FIG. 27C, and FIG. 41). RT-qPCR analysis confirmed decreased expression of Tgfb1/TGFB1 in TET3 knockdown macrophages (FIG. 27D). It has been well established that aberrant TGF-β signaling underlies various forms of chronic inflammation, including hepatic fibrosis and endometriosis. For example, suppressing TGF-β1 production specifically from liver macrophages decreases fibrotic NASH in mice (Cai et al., 2020, Cell Metab, 31:406-421). Likewise, it was previously reported a positive feedback regulation between TGF-β1 and TET3 in both hepatocytes and HSCs where TGF-β1 stimulates TET3 expression which in turn upregulates Tgfb1/TGFB1 expression, promoting hepatic fibrogenesis (Xu et al., 2020, Cell Rep, 30:1310-1318). Furthermore, Tgfb1 ablation in mice decreases the growth of endometriosis lesions and levels of TGF-β1 increase in the ectopic endometrium of women with endometriosis (Young et al., 2017, Hum Reprod Update, 23:548-559). Taking into account of these prior studies, the results demonstrated the notion that TET3-induced upregulation of TGF-β1 expression in DAMs promoted fibrotic NASH and endometriosis. As such, the present results indicated a mechanistic contribution of TET3-induced upregulation of TGF-β1, IL-1β, and IL-6 in macrophages to the pathogenesis of NSCLC, endometriosis, and NASH.

Next, the studies examined effects of TET3 knockdown on expression of TGF-β1, IL-1β, and IL-6 in human MDMs. TGF-β1 has been mechanistically linked to the pathogenesis NSCLC, endometriosis, and NASH (Saito et al., 2018, Int J Mol Sci, 19; Young et al., 2017, Hum Reprod Update, 23:548-559; Cai et al., 2020, Cell Metab, 31:406-421; Baba et al., 2022, Front Pharmacol, 13:791272). It is also an important factor in the conditioned media responsible for inducing TET3 overexpression (FIG. 24H, FIG. 24J, and FIG. 24K through FIG. 24M). Therefore, MDMs were treated with TGF-β1 to mimic microenvironment-induced TET3 overexpression, followed by siRNA transfection and gene expression analyses. Decreased expressions of TGF-β1, IL-1β, and IL-6 were observed at both mRNA and protein levels in TET3 siRNA- vs. NT siRNA-transfected cells (FIG. 26J). ELISA was not used to measure TGF-β1 because the transfection experiments were performed in the presence of exogenous TGF-β1. Remarkably, Bobcat339 was able to recapitulate these effects on all three factors (FIG. 26K), consistent with its ability to decrease the steady-state level of TET3 protein (FIG. 26A and FIG. 26B).

Nitric oxide (NO) is synthesized by the NO synthase (NOS) enzymes via the citrulline-nitric oxide cycle. There are three isoforms of the enzymes: neuronal (nNOS encoded by Nos1), inducible (iNOS, encoded by Nos2), and endothelial (eNOS, encoded by Nos3). In immune cells, the high production of NO (at micromolar levels) is sustained by iNOS (Palmieri et al., 2020, Metabolites, 10). In macrophages, NO orchestrates metabolic rewiring in part by inhibiting mitochondrial oxidative phosphorylation (OXPHOS) leading to glycolytic commitment (Palmieri et al., 2020, Metabolites, 10; Poderoso et al., 2019, Nitric Oxide, 88:61-72; Palmieri et al., 2020, Nat Commun, 11:698). Macrophage-derived NO can also act on neighboring cells (both immune and non-immune cells) inducing metabolic reprogramming of these cells (Palmieri et al., 2020, Metabolites, 10). Both hepatocytes in NAFLD and endometriotic cells favor glycolytic metabolism over mitochondrial OXPHOS (Wegiel et al., 2018, Front Oncol, 8:284; Kobayashi et al., 2021, Reprod Fertil, 2:C42-C57; Lu et al., 2021, Front Physiol, 12:710420). The RNA-seq data revealed inhibition of the “Citrulline-Nitric Oxide Cycle” (FIG. 27B and FIG. 41) and a decrease in Nos2 expression (FIG. 27C) in TET3 knockdown Raw 264.7 cells. Downregulation of Nos2/NOS2 expression in response to TET3 knockdown was further validated by RT-qPCR of RNAs from human and mouse macrophages (FIG. 27D). Consistently, NO production was significantly decreased in TET3 knockdown MDMs (FIG. 274E). Therefore, TET3-induced upregulation of NO production from DAMs in NASH and endometriosis promoted disease progression.

Apoptosis usually occurs in macrophages upon pathogen infection but also can occur in nonimmune cells (Man et al., 2017, Immunol Rev, 277:61-75). Interferon (IFN) signaling and interferon regulatory factors (IRFs, a family of transcription factors) are critical regulators of apoptosis with various IRFs shown to facilitate pyroptotic death of macrophages (Wu et al., 2016, Shock, 46:329-338; Kayagaki et al., 2019, Sci Signal, 12; Karki et al., 2018, Cell, 173:920-933; Bhatt et al., 2022, FEBS J, 289:1536-1551). “Activation of IRF by Cytosolic Pattern Recognition Reception”, “Apoptosis Signaling Pathway” and “Interferon Signaling” were identified to be activated in response to TET3 knockdown in macrophages (FIG. 27B and FIG. 41). Increased expressions of Irf7 and Irf9 in TET3 knockdown Raw 264.7 cells (FIG. 27C) were validated by RT-qPCR of RNAs from human and mouse macrophages (FIG. 27D). Collectively, the RNA-seq data were in line with the notion that TET3 knockdown induced apoptosis in macrophages (FIG. 25).

Overall, the genome-wide studies revealed that TET3 activated pathways involving TGF-β signaling and metabolic reprogramming known to play important roles in driving the pathology and progression of NASH and endometriosis. Most critically, the studies enabled to harness the mechanism of TET3-dependent suppression of apoptosis to eliminate TET3-expressing DAMs thereby inhibiting disease progression.

### Myeloid-Specific Tet3 Ablation Mitigated Endometriosis

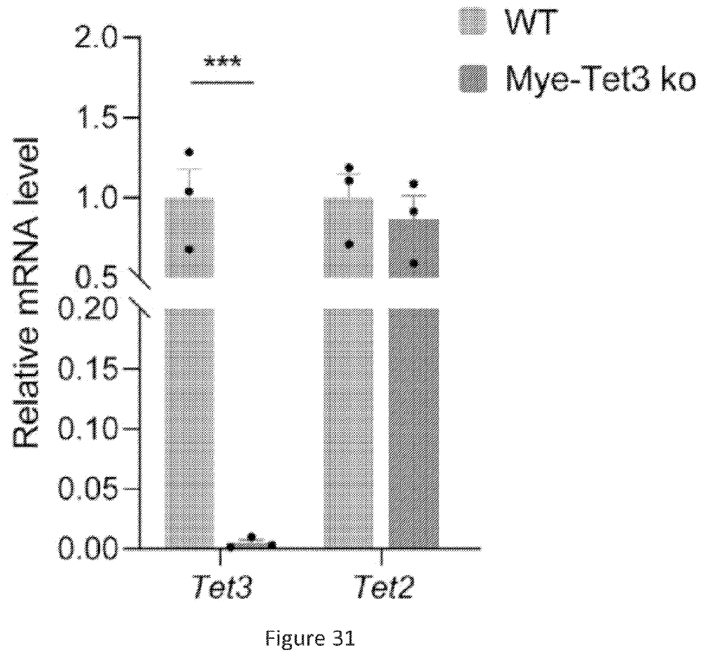

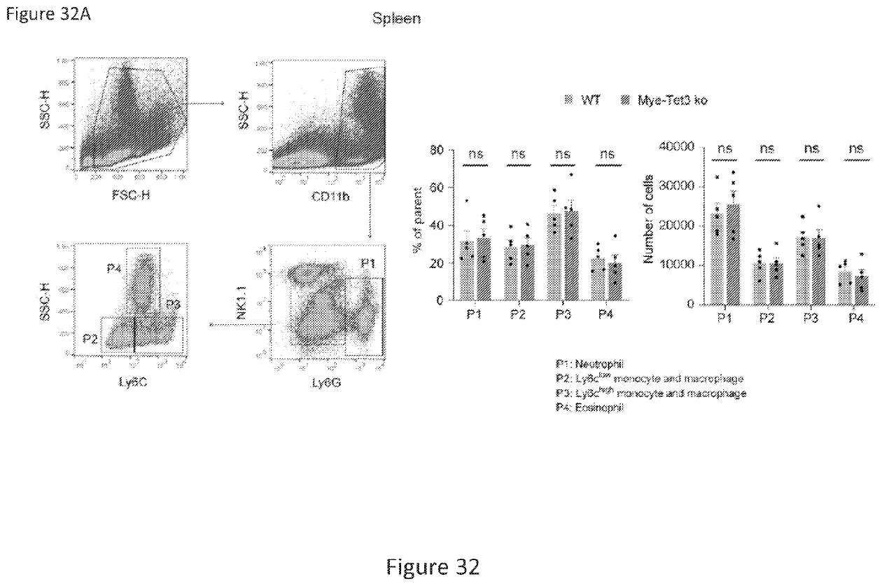

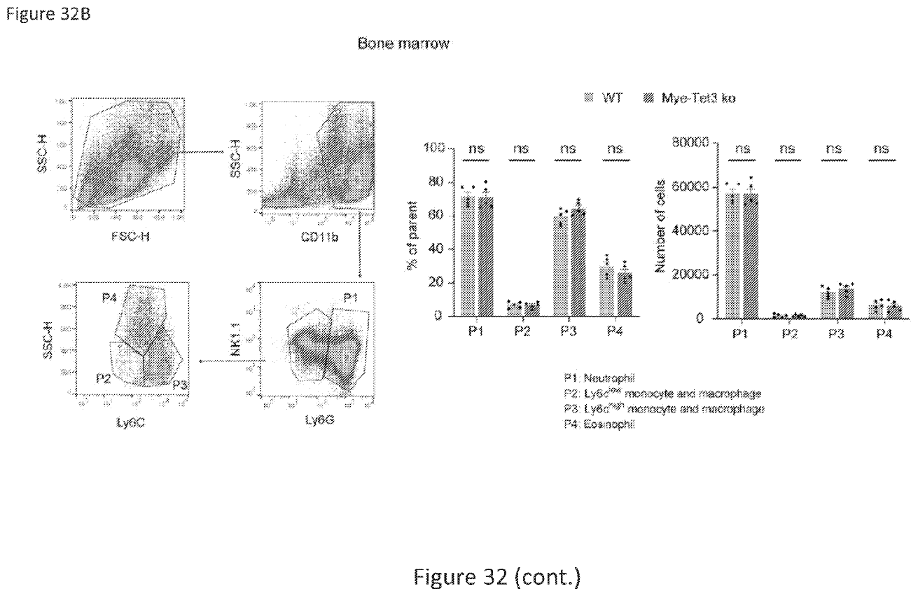

Additional studies investigated whether TET3-expressing DAMs contribute critically to disease progression. The lysozyme gene Lyz2 which encodes lysozyme M (LysM) is exclusively expressed in myelomonocytic cells including monocytes, macrophages, and granulocytes (Shi et al., 2018, Methods Mol Biol, 1784:263-275). Tet3fl/fl mice were mated with LysM-cre mice to knockout Tet3 in the myelomonocytic compartment, including macrophages (Lysm+/wt Tet3fl/fl, herein referred to as “Mye-Tet3 ko”). Peritoneal macrophages from Mye-Tet3 ko mice showed ~90% decrease in the expression of Tet3 (but not Tet2 and with limited effects (~40%) on tissue-resident macrophages) (FIG. 31), consistent with the documented efficiency in the LysM-cre model (Shi et al., 2018, Methods Mol Biol, 1784:263-275). Compared to wildtype control (Lysmwt/wt Tet3fl/fl, herein WT), the Mye-Tet3 ko mice showed similar numbers and percentages of myeloid populations including macrophage and monocytic cells in spleen and bone marrow (FIG. 32), demonstrating no alteration in steady-state myeloid cell lineage distribution by Tet3 ablation.

Studies utilized mated Tet3fl/fl mice with LysM-cre mice to knockout Tet3 in the myelomonocytic compartment, including macrophages (Lysm+/wt Tet3fl/fl, hereinafter referred to as “Mye-Tet3 ko”). Peritoneal macrophages from Mye-Tet3 ko mice showed ~90% decrease in the expression of Tet3 without affecting Tet2 (FIG. 31). Compared to wildtype control (Lysmwt/wt Tet3fl/fl, herein WT), the Mye-Tet3 ko mice showed similar numbers and percentages of myeloid populations including macrophage and monocytic cells in spleen and bone marrow (FIG. 32), demonstrating no alteration in steady-state myeloid cell lineage distribution by Tet3 ablation. These results were consistent with the notion that TET3 expression is not required for monocyte development and differentiation (Gerecke et al., 2022, Front Immunol, 13:861351). Further, myeloid-specific Tet3 ablation did not alter body weight, body composition, and fasting glucose levels (FIG. 33).

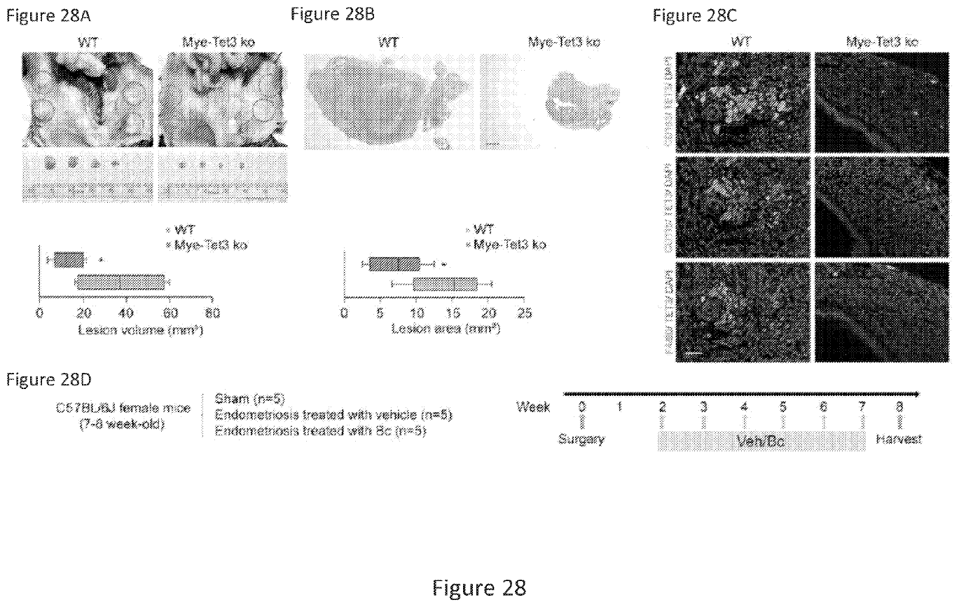

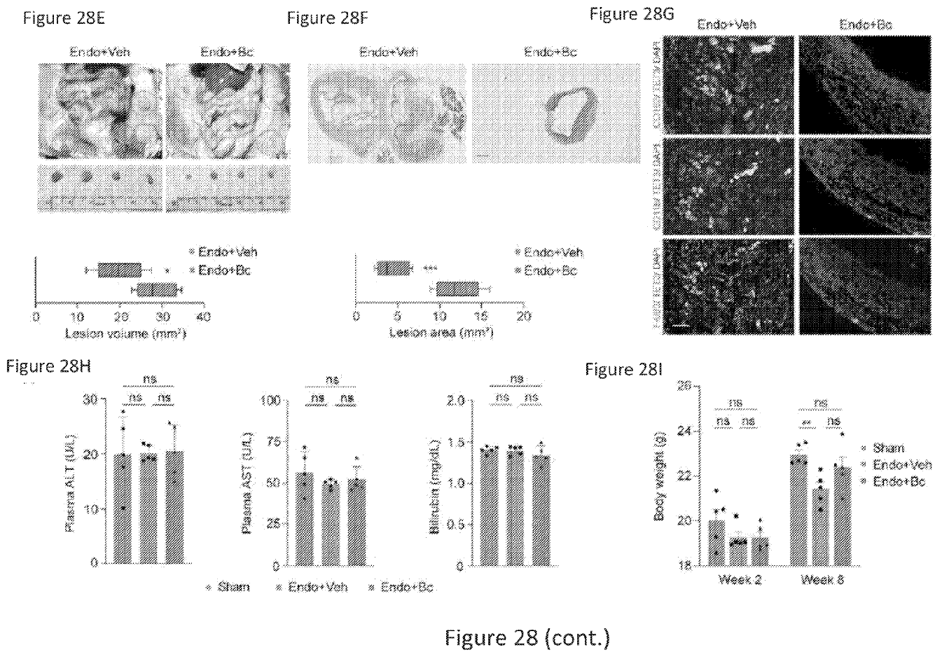

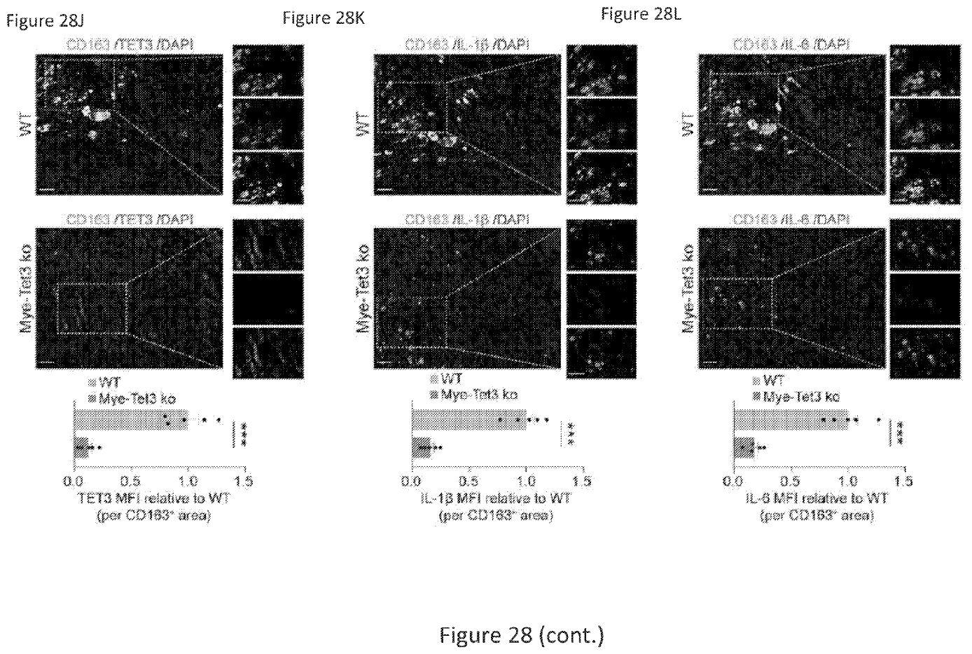

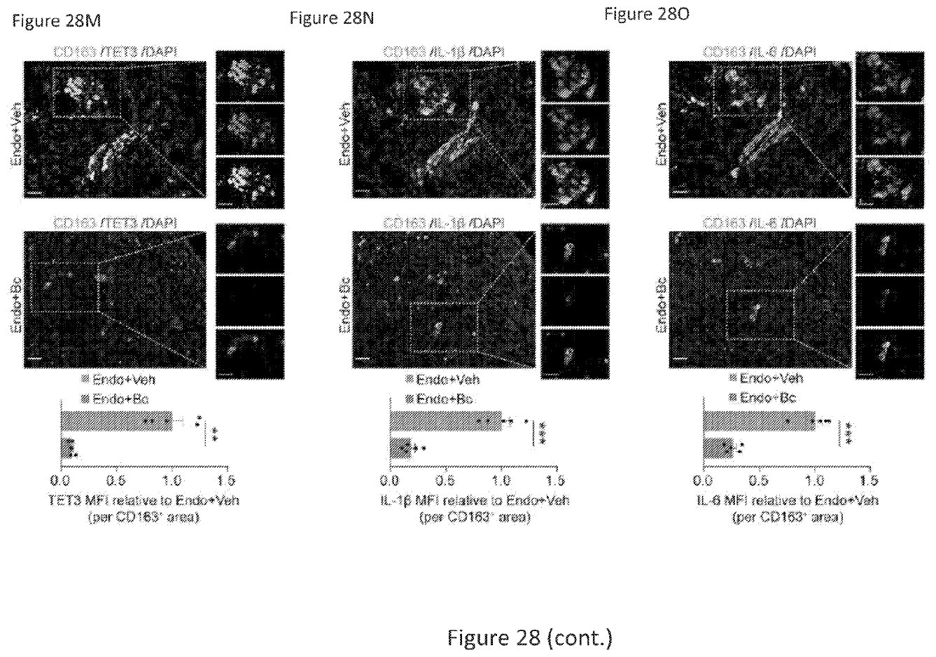

Further studies then investigated whether myeloid-specific Tet3 ablation would affect endometriosis progression using a well-established, surgically induced murine model of endometriosis (Rosa et al., 2019, Reprod Sci, 26:1395-1400; Mamillapalli et al., 2022, Reprod Sci, 29:243-249; Pluchino et al., 2020, J Cell Mol Med, 24:2464-2474; Miller et al., 2020, Front Immunol, 11:108). Thus, female mice of WT and Mye-Tet3 ko of 6-7 weeks old were subjected to surgery (week 0) to induce intraperitoneal endometriosis, followed by euthanasia and blood and tissue collection 6 weeks later (week 6). While no body weight difference between the groups was observed (FIG. 33), compared to the WT mice, the Mye-Tet3 ko mice showed a significant reduction in the lesion volume both macroscopically (FIG. 28A) and histologically (FIG. 28B). IHC analysis revealed depletion of TET3/CD163 double-positive cells in the lesions of the ko mice (FIG. 28C, top panels). These cells were largely also positive for CD11b (a marker for monocyte/macrophages, granulocytes and natural killer cells) (middle panels) and F4/80 (a unique marker of murine macrophages) (bottom panels), consistent with the notion that CD163 often co-expresses with other surface markers (Barreby et al., 2022, Nat Rev Endocrinol, 18:461-472; Cominelli et al., 2014, Mol Hum Reprod, 20:767-775; Ramachandran et al., 2020, Nat Rev Gastroenterol Hepatol, 17:457-472). Thus, Tet3 ablation in the myeloid compartment leads to depletion of TET3-positive macrophages and suppression of endometriosis.

### Bobcat339 Treatment Reduced Endometriosis Burden

Given that Bobcat339 degrades TET3 inducing apoptosis (FIG. 26) and that myeloid-specific Tet3 ablation attenuates endometriosis (FIG. 28A through FIG. 28C), studies were performed to test whether Bobcat339 would elicit therapeutic effects on endometriosis. Thus, female mice were randomly divided into three groups (sham, endometriosis treated with vehicle, and endometriosis treated with Bobcat339) and subjected to surgery (week 0) to induce endometriosis or sham (FIG. 28D). The first once-a-week intraperitoneal (i.p.) injection of Bobcat339 (or vehicle) at a dose of 2.5 mg/kg body weight was performed on week 2, followed by euthanasia and blood and tissue collection on week 8. The dose of 2.5 mg/kg was chosen based on the previous studies showing no discernible liver toxicity in mice (Lv et al., 2023, Proc Natl Acad Sci USA, In press). Bobcat339 treatment significantly reduced the lesion volume both macroscopically (FIG. 28E) and histologically (FIG. 28F). IHC analysis showed depletion of TET3-expressing macrophages (FIG. 28G). Finally, there was no evidence of liver toxicity following 6 weeks of once-a-week Bobcat339 treatment (FIG. 28H), nor was there a significant difference in body weight between the Endo+Veh and Endo+Bobcat339 groups (FIG. 28I). The results demonstrated that Bobcat339 was effective in treating endometriosis at least in part by depleting TET3-positive macrophages.

### Bobcat339 Elicited Therapeutic Effects in a Murine Model of NASH

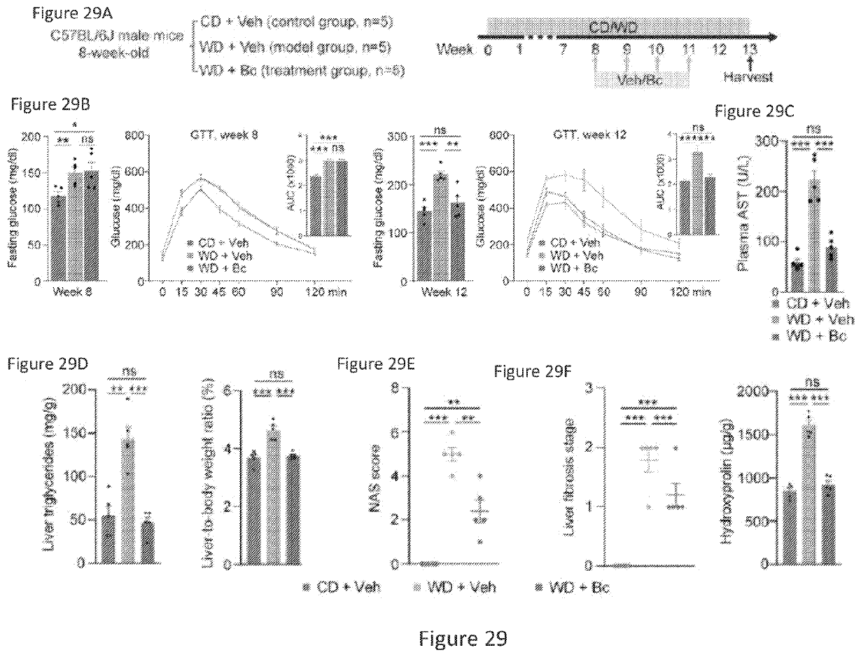

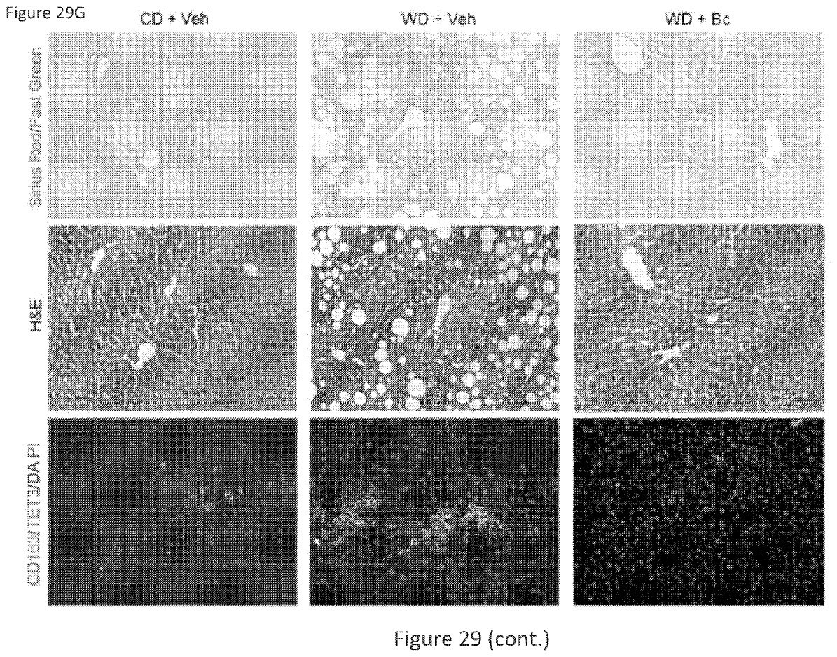

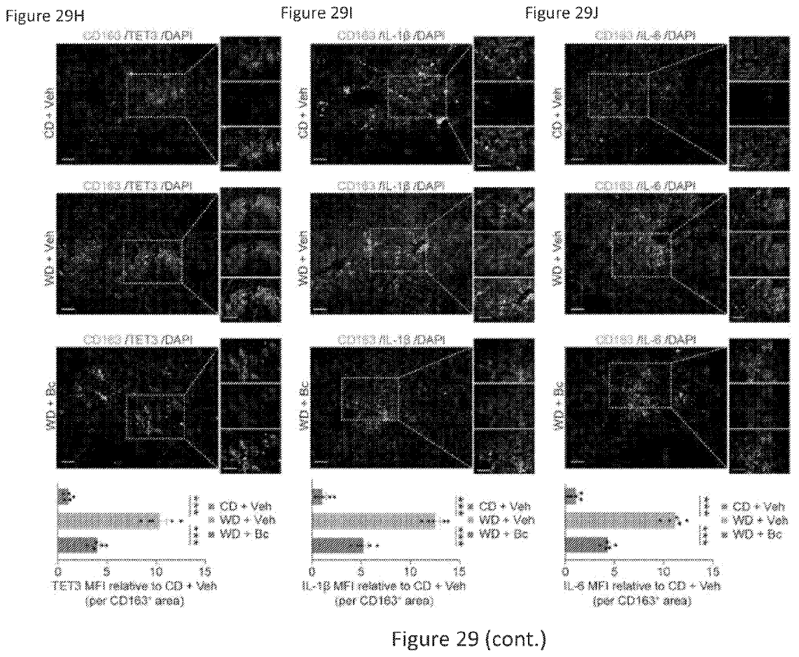

As TET3-positive macrophages were also detected in human NASH (FIG. 24B), studies were performed to investigate whether depleting these macrophages would reduce NASH progression. Despite the high efficiency (up to 90%) of the LysM-cre strain in manipulating macrophages originated from myeloid progenitors including those in the peritoneal cavity, peripheral blood and bone marrow, only ~40% of tissue resident macrophages derived from yolk sac (e.g., those in the liver and the spleen red pulp) were affected (Shi et al., 2018, Methods Mol Biol, 1784:263-275). While the inefficacy of the LysM-cre strain in depleting tissue resident macrophages has precluded using the Mye-Tet3 ko mice, potential effects of Bobcat339 in steatosis, inflammation, and fibrosis were assessed using a high fat high sugar diet-induced murine model of NASH (Ishimoto et al., 2013, Hepatology, 58:1632-1643; Kroh et al., 2020, Gastroenterol Res Pract, 7347068; Baiges-Gaya et al., 2021, J Nutr Biochem, 89:108559). As shown schematically in FIG. 29A, mice were placed on a standard chow diet (CD) (control group) or a western diet with 10% sucrose in drinking water (WD) for 8 weeks to induce an early NASH phenotype (Kroh et al., 2020, Gastroenterol Res Pract, 7347068). Then, these mice were therapeutically dosed intravenously once a week from week 8 through week 11 with either 2.5 mg/kg Bobcat339 (treatment group) or vehicle (model group) for a total of 13 weeks on diets. Mice were euthanized following a 12 h overnight fasting, and plasma and liver were harvested for analyses.

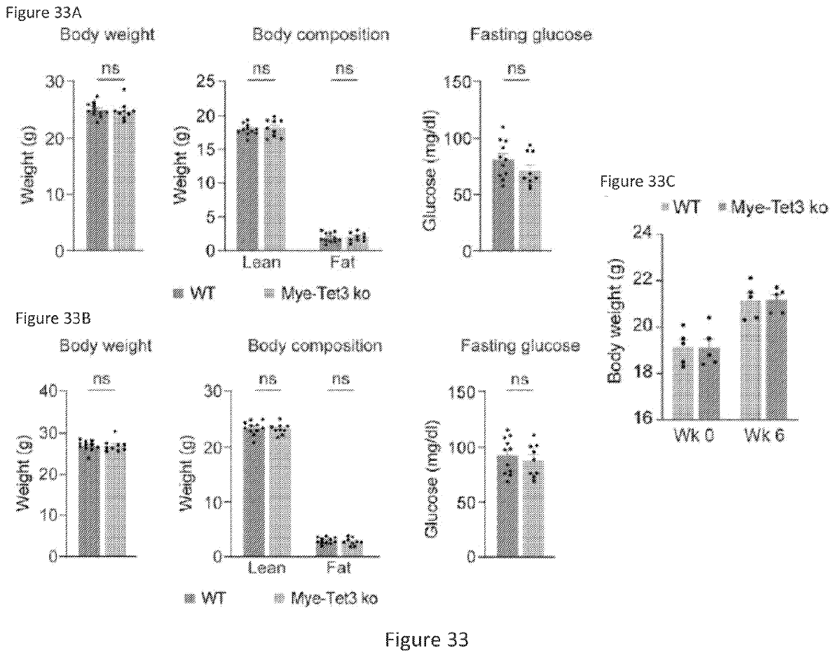

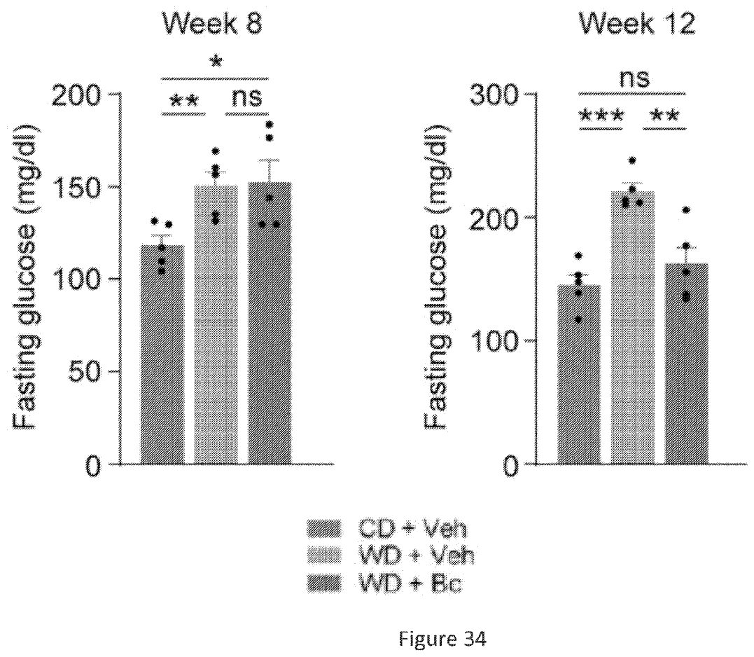

8 weeks of WD significantly increased fasting glucose (FIG. 34) and decreased glucose tolerance in both the model and treatment groups as compared to control group (FIG. 29B, left panel), indicating impaired glucose metabolism. While fasting glucose (FIG. 34) and glucose tolerance in the model group remained abnormal at week 12, those in the treatment group showed a significant improvement following Bobcat339 exposure (FIG. 29B, right panel). At week 13, in contrast to the control animals, the model group animals showed an elevated plasma aspartate aminotransferase (AST), indicating hepatocellular injury, and Bobcat339 normalized this marker (FIG. 29C).

The animals in the control group had a normal liver histology. The animals in the model group exhibited a general disturbance of hepatic architecture, with moderate/severe steatosis and lobular inflammation, and no/mild hepatocyte ballooning. These histopathological alterations were accompanied with significant increases in liver triglycerides and liver-to-body weight ratio (FIG. 29D). This group of animals had a mean NAS score of 5 (FIG. 29E), indicating the presence of NASH. Bobcat339 normalized liver triglycerides, decreased liver-to-body weight ratio, and reduced NAS scores (FIG. 29D and FIG. 29E).

Hepatic fibrosis stage was assessed using Sirius Red/Fast Green stain and the measurement of liver hydroxyproline, which is a unique amino acid in collagen molecules and which serves as an important biomarker of liver fibrosis (Xu et al., 2020, Cell Rep, 30:1310-1318). The model group animals developed more fibrosis than the control group animals, which was further supported by the significant increase in hepatic hydroxyproline content (FIG. 29F). Bobcat339 treatment led to a significant reduction in both the fibrosis stage and the hydroxyproline content (FIG. 29F). Representative images of Sirius Red/Fast Green and H&E liver sections depict severe steatosis and definitive fibrosis in the model group, and Bobcat339 significantly reduced the severity of both steatosis and fibrosis (FIG. 29G). Finally, IHC analysis revealed accumulation of TET3-expressing macrophages in the liver of the model group mice, which was depleted after Bobcat339 treatment. Taken together, the results show that Bobcat339 treatment ameliorates NASH (NAS score and fibrosis) at least in part via depleting TET3-expressing macrophages.

### Bobcat339 Exhibited Therapeutic Effects in a Syngeneic Murine Model of Lung Cancer

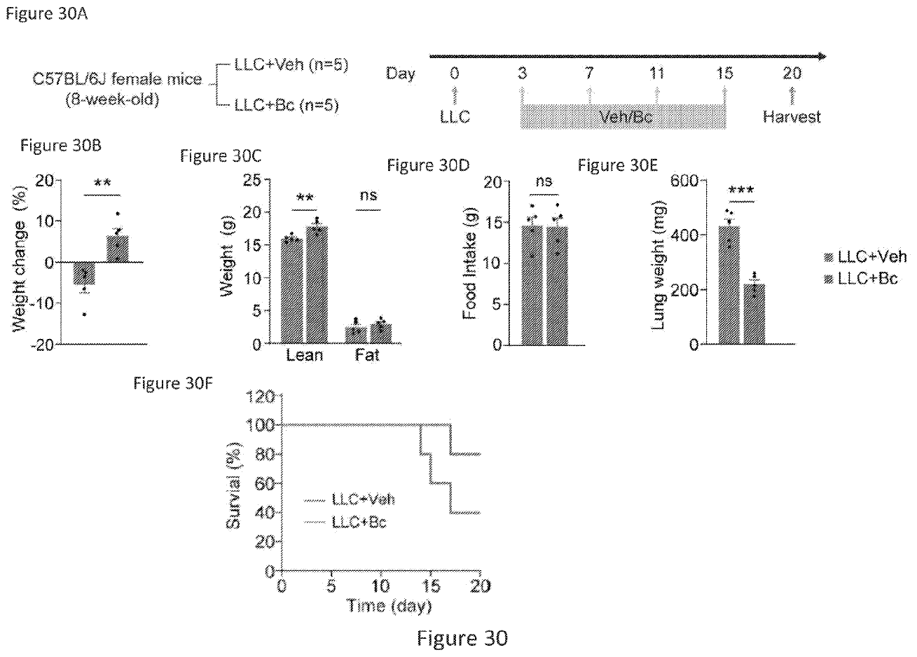

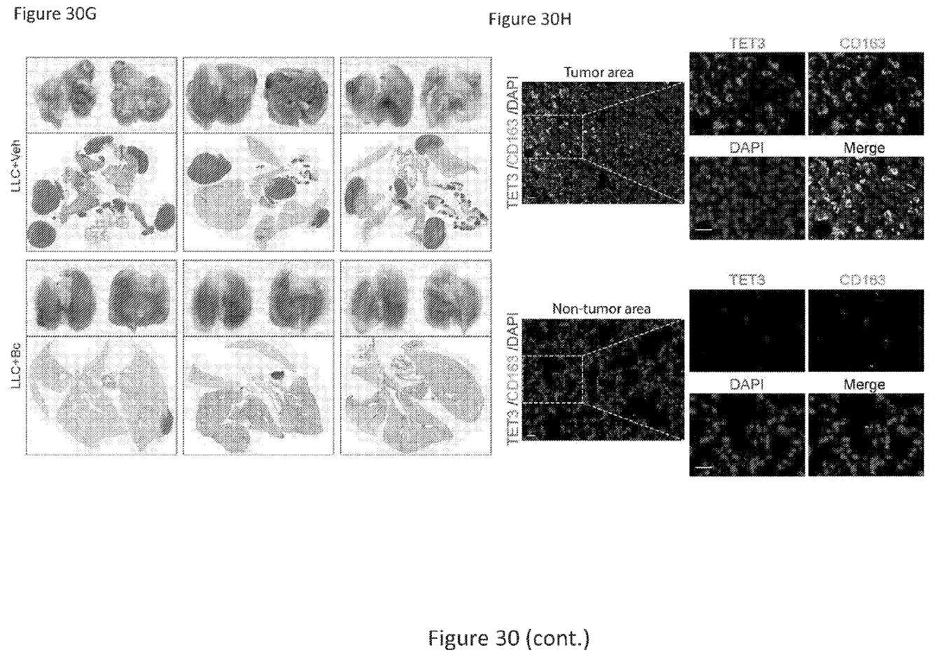

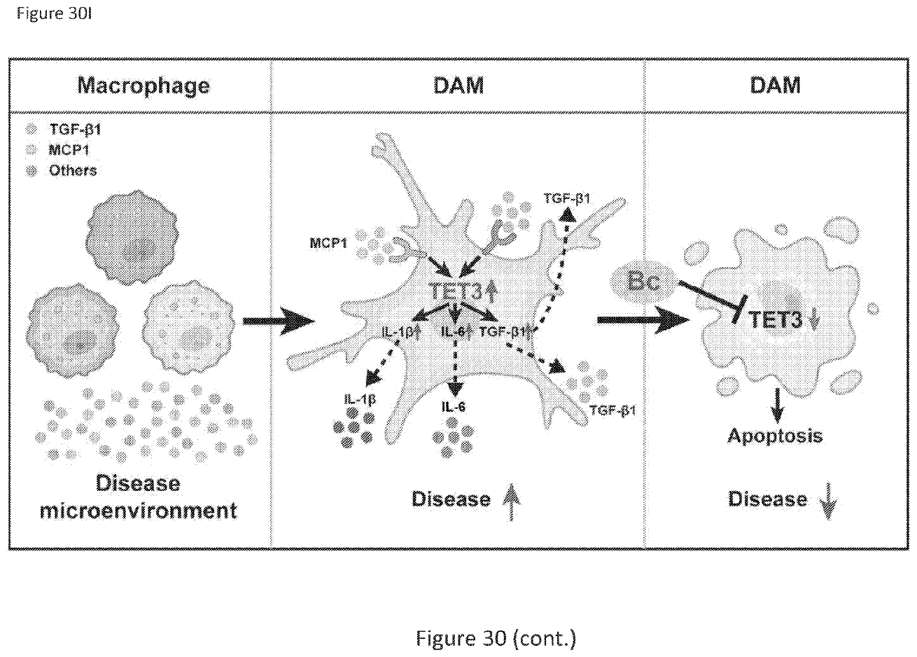

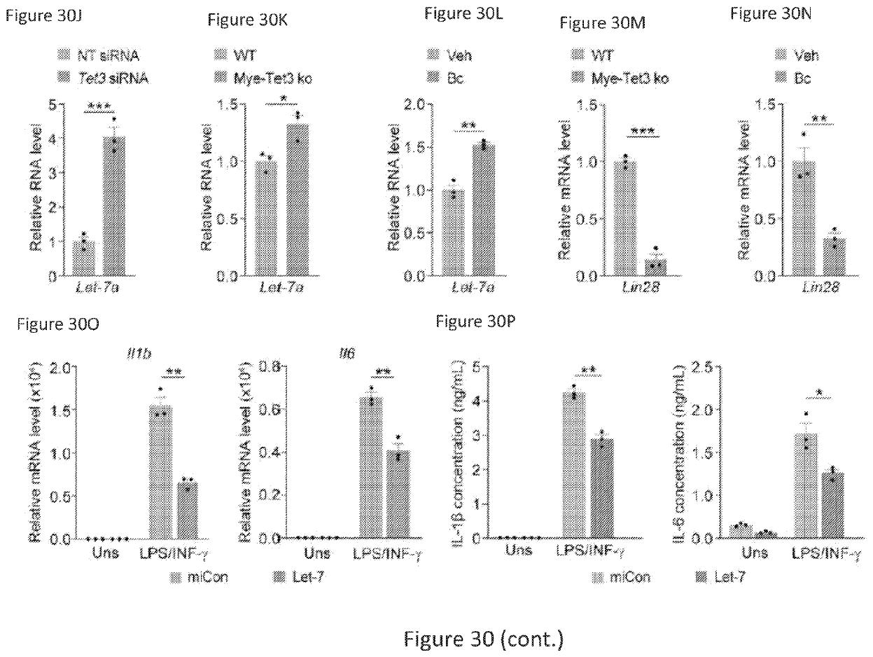

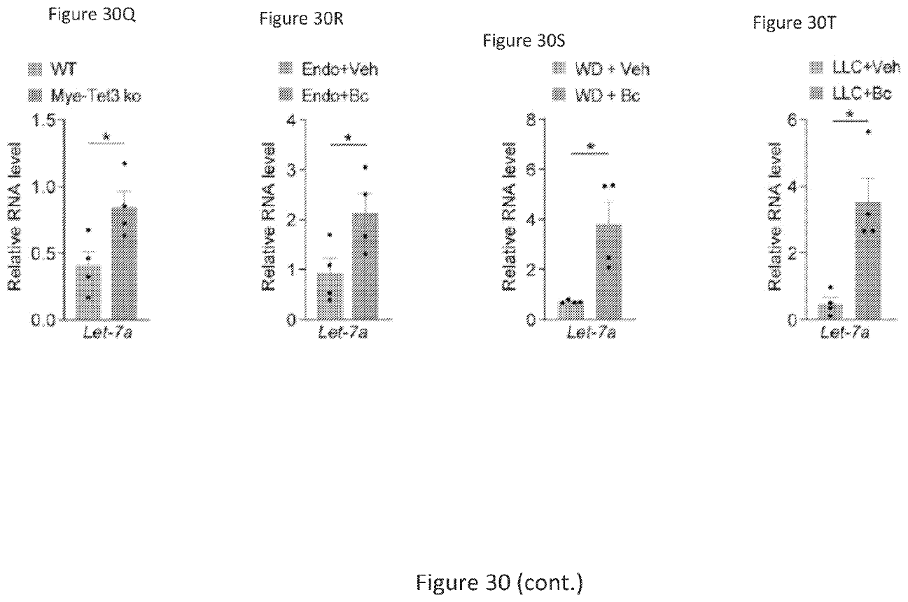

Metastatic lung cancer was established by tail vein injection of Lewis lung carcinoma (LLC) (a mouse NSCLC cell line, Bertram et al., 1980, Cancer Lett, 11:63-73) cells into immunocompetent mice (FIG. 30A). Intravenous dosing of Bobcat339 (or vehicle) at 2.5 mg/kg every four days was initiated three days after tumor cell injection. The lungs were collected at day 20 from sacrificed mice. On day 16 as compared to day 0, mice in the Veh group lost ~5% of body weight, whereas those in the Bobcat339 group gained ~6% of body weight, which would be expected for a normal female mouse during this age period (FIG. 30B). The body weight loss in the Veh group was attributed mainly to the loss of lean body mass (FIG. 30C), though no difference in food intake was found between the groups (FIG. 30D). Severe weight loss is frequently seen in patients with NSCLC, which is also associated with systemic inflammation and cancer cachexia (Staal-van den Brekel et al., 1995, J Clin Oncol, 13:2600-2605; Fredrix et al., 1991, Cancer, 68:1616-1621; Jatoi et al., 2001, Ann Thorac Surg, 72:348-351; Ulmann et al., 2019, Ann Nutr Metab, 75:223-230; Jouinot et al., 2020, Clin Nutr, 39:1893-1899).

The weight of the lungs in Bobcat339 treated animals was significantly less than that of the lungs in Veh-treated animals (FIG. 30E). Fewer LLC metastases were found in Bobcat339 treated lungs than in the lungs of Veh-treated animals (FIG. 30G). These results indicated that lung metastasis was strongly inhibited by treatment with Bobcat339. Importantly, TET3/CD163 double-positive cells were detected in tumors but not in paired non-cancerous lung tissues (FIG. 30H), reminiscent of the observations in human lung adenocarcinoma (FIG. 24E and FIG. 24F).

In this study, the identification of a novel subset of macrophages that express TET3 and which are associated with human lung cancer, NASH, and endometriosis was described. It was shown that TET3 overexpression in these cells was induced by inflammatory disease microenvironments and that TGF-β1 was an important factor responsible for the induction. TET3 activated pathway gene expression involving TGF-β signaling and metabolic reprogramming known to contribute critically to disease progression, therefore underscoring the importance of TET3 in DAMs. TET3 promoted the expression of inflammatory cytokines TGF-β1, IL-1β, and IL-6 known to contribute pivotally to the development and progression of these diseases. An additional critical finding was that TET3 suppressed apoptosis, rendering DAMs vulnerable to TET3 inhibitors, such as Bobcat339, a small molecule degrader of TET3 described in this and the previous studies (Lv et al., 2023, Proc Natl Acad Sci USA, In press). Importantly, it was shown that this TET3-dependent mechanism was conserved in human and mouse macrophages. A working model was also presented (FIG. 30I).

Despite the notion that the majority of CD163/TET3 double-positive cells also seemed to express other monocyte markers such as CD11b and F4/80 (FIG. 28C and FIG. 28G), it is possible that some TET3-expressing DAMs may not be CD163 positive. As TET3-positive macrophages were seen in major inflammatory diseases of two distinct types, NASH and endometriosis, these macrophages represent a unifying pathological subset of macrophages in chronic inflammatory diseases. Indeed, the data presented in FIG. 28 and FIG. 29 have demonstrated that inhibiting TET3, either genetically or pharmacologically, leads to depletion of TET3-positive macrophages and significant attenuation of NASH (NAS score and fibrosis) and endometriosis.

As such, these studies are the first to describe the molecular and cellular function of TET3 in macrophages with its significance in lung cancer and major chronic inflammatory diseases, and the usage of a first-in-class small molecule degrader of TET3 as a potential new therapeutic agent for these diseases and other cancers and chronic inflammatory diseases without discernible side effects.

In summary, macrophages in the disease microenvironment contribute critically to the development and progression of chronic inflammatory diseases. Yet, targeting the DAMs for therapy has remained extremely challenging due to their high heterogeneity both molecularly and phenotypically and which is also dynamically modified in response to disease microenvironments. Here, identification of TET3-expressing DAMs in nonalcoholic steatohepatitis (NASH) and endometriosis was reported. It was shown that TET3 expression was induced by disease microenvironments and was required to maintain the viability of these cells. TET3 activated multiple regulatory pathways crucial for disease pathology and this TET3-dependent mechanism was conserved between human and mouse macrophages. Further, depleting TET3-positive macrophages through myeloid-specific Tet3 ablation or using a synthetic small-molecule degrader of TET3 ameliorated both diseases in mice. Although DAMs exhibited extensive heterogeneity in major chronic inflammatory diseases, the TET3-expressing macrophages constituted a unifying pathological subset and that targeting TET3 are exploited as a potential therapeutic for them.

### Example 5: TET3 Rendered Pathogenic Macrophages Vulnerable to Targeted Elimination

Tissue macrophages are comprised of both embryonic-derived and monocyte-derived phagocytes. These macrophages play essential roles in tissue maintenance as well as acting as the first line of defense against pathogens. During disease, tissue macrophages are “educated” to become phenotypically and functionally distinct disease-associated macrophages (DAMs), some of which accelerate disease progression (Park et al., 2022, Cell, 185:4259-4279).

IL-1b and IL-6 are proinflammatory cytokines shown to play important roles in the development and progression of NSCLC, endometriosis, and NASH. The importance of IL-1b in NSCLC has been well demonstrated. IL-1b activated signaling pathways including those mediated by NF-kB, Cox2, and MAPK, contributing to the establishment and maintenance of TAM functions (Zhang et al., 2022, Biomark Res, 10:5). Consistently, in a recent phase III clinical trial, treatment with canakinumab, a human monoclonal antibody specific for IL-1b, substantially reduced incident NSCLC and lung cancer mortality in a dose-dependent manner (Garon et al., 2020, JTO Clin Res Rep, 1:100001). Further, increased IL-1b was found in peritoneal fluids and lesions from women with endometriosis and pelvic pain (Peng et al., 2020, Hum Reprod, 35:901-912; Akoum et al., 2008, Fertil Steril, 89:1618-1624). Notably, IL-1b promoted local neurogenesis and increased nerve density around endometriosis and endometriosis-associated pain (Peng et al., 2020, Hum Reprod, 35:901-912). IL-1b also has a well-established role in all stages of NAFLD by promoting steatosis, inflammation, and fibrosis (Mirea et al., 2018, Trends Mol Med, 24:458-471; Tilg et al., 2020, Expert Opin Investig Drugs, 29:103-106).

In NSCLC, IL-6 promoted disease progression in part by upregulating expression of immune checkpoint protein PD-L1 and by recruiting myeloid-derived suppressor cells to the tumor microenvironment (TME). Importantly, antibody-mediated blockade of IL-6 strongly inhibited tumor growth in murine models of NSCLC (Song et al., 2014, J Thorac Oncol, 9:974-982; Liu et al., 2022, BMC Med, 20:187). The role of IL-6 in endometriosis was supported by several lines of evidence. First, IL-6 was elevated in the peritoneal fluid and the serum of patients with endometriosis (Nematian et al., 2018, J Clin Endocrinol Metab, 103:64-74). Second, IL-6 released from activated macrophages stimulated the migration of endometriotic epithelial cells during formation of extra-pelvic endometriosis (Woo et al., 2017, Biol Reprod, 97:660-670). Third, treating rats with tocilizumab, an anti-IL-6 receptor monoclonal antibody, lead to significantly reduced endometriosis burden (El-Zayadi et al., 2020, Immunol Res, 68:389-397). Finally, an elevated production of IL-6 from Tet2-deficient mouse macrophages has been linked to NASH progression (Wong et al., 2023, Nature, 616:747-754).

Despite the clear contributions of pathogenic DAMs and their secreted proinflammatory cytokines to the pathophysiology of lung cancer, endometriosis, and NASH, targeting pathogenic DAMs for therapy has remained extremely challenging due to their phenotypic and functional heterogeneity (Ardura et al., 2019, Front Pharmacol, 10:1255; Skytthe et al., 2020, Int J Mol Sci, 21). Indeed, recent single-cell RNA sequencing (scRNA-seq) studies have revealed dynamic shifts of DAMs in molecular programs, surface marker composition and functional properties in a disease/disease stage-dependent manner (Cochain et al., 2018, Circ Res, 122:1661-1674; Xiong et al., 2019, Mol Cell, 75:644-660; Ramachandran et al., 2019, Nature, 575:512-518; Krenkel et al., 2020, Gut, 69:551-563). The complexity of DAMs was further highlighted by the notion that DAMs can play protective or pathogenic roles. For example, in mouse models of NASH, accumulation of Tim3/Tim4-overexpressing macrophages was shown to be protective against liver injury (Du et al., 2019, Cell Mol Immunol, 16:878-886; Liu et al., 2019, J Immunol, 203:990-1000; Daemen et al., 2021, Cell Rep, 34:108626). On the other hand, TREM2⁻ macrophages identified in multiple diseases (including NASH, Alzheimer's disease, and cancers) and across different tissues exhibited both pathogenic and protective potential in a tissue-specific manner (Park et al., 2022, Cell, 185:4259-4279; Xiong et al., 2019, Mol Cell, 75:644-660; Perugorria et al., 2019, Gut, 68:533-546; Wang et al., 2023, Immunity, 56:58-77; Liebold et al., 2023, Cells, 12). Thus, identification of specific classes of macrophages that contribute critically to disease progression is essential to the development of effective therapeutic strategies.

The TET family proteins (TET1, TET2 and TET3) oxidize 5-methylcytosine to 5-hydroxymethylcytosine and its derivatives to facilitate DNA demethylation. TETs also regulate gene expression independently of their catalytic activities (Lio et al., 2020, J Biosci, 45). Altered TET expression has been shown to elicit various pathophysiological effects in a tissue/cell-dependent fashion (Wong et al., 2023, Nature, 616:747-754; Pan et al., 2017, Immunity, 47:284-297; Fuster et al., 2017, Science, 355:842-847; Jiang et al., 2019, Proc Natl Acad Sci, 116:12416-12421; Li et al., 2020, Nat Commun, 11; Damal Villivalam et al., 2020, Nat Commun, 11:4313; Qin et al., 2021, Cells, 11; Rui et al., 2021, Nat Commun, 12:5074; Xu et al., 2022, Proc Natl Acad Sci, 119; Xie et al., 2022, Proc Natl Acad Sci, 119:e2122217119; Xie et al., 2022, J Clin Invest, 132; Byun et al., 2022, Proc Natl Acad Sci, 119:e2205626119; Liu et al., 2023, Nat Cardiovasc Res, 2:572-586; Yeaton et al., 2022, Cancer Discov, 12:2392-2413). The present example reports identification of TET3-overexpressing macrophages that were CD163-positive in tissue samples from patients with endometriosis, NASH, and NSCLC. The present example also demonstrates that these TET3/CD163 double-positive cells were proinflammatory and can be targeted for therapy.

### TET3 was Overexpressed in a Subset of CD163⁺ Macrophages in Endometriosis, NASH, and NSCLC

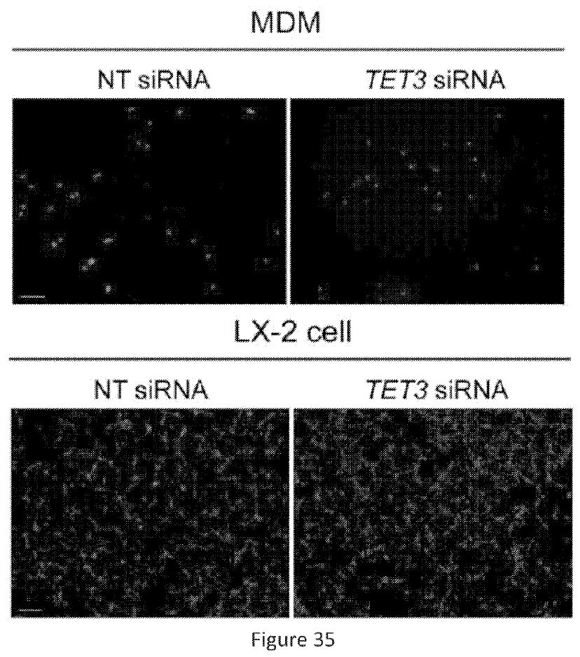

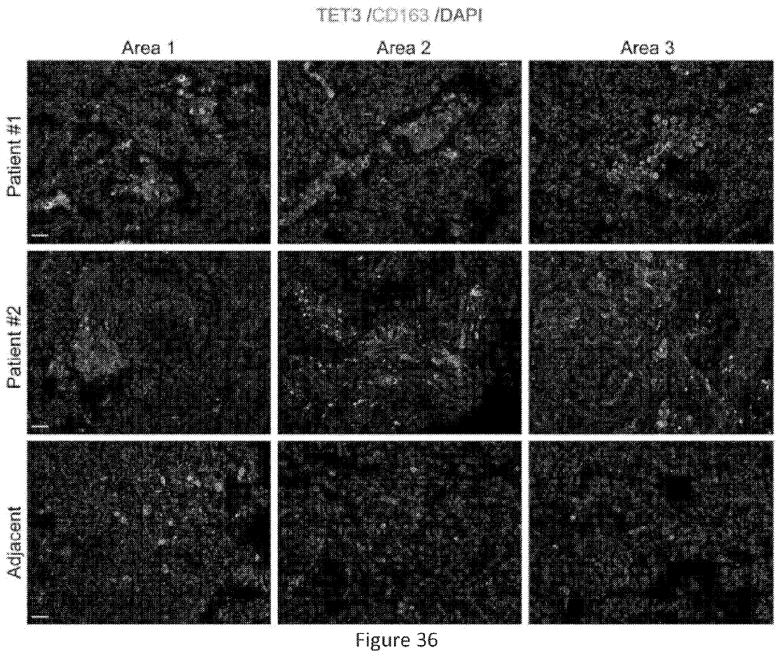

Aggregates of CD163⁺ macrophages were observed in endometriosis lesions and in livers of patients with NASH (De Vito et al., 2012, Int J Mol Med, 30:49-56; Cominelli et al., 2014, Mol Hum Reprod, 20:767-775; Kazankov et al., 2015, J Gastroenterol Hepatol, 30:1293-1300). Also, TAMs in NSCLC have been identified as predominantly CD163⁺ cells (Xu et al., 2020, Mol Med Rep, 22:4107-4115; Larionova et al., 2020, Front Oncol, 10:566511; Larroquette et al., 2022, J Immunother Cancer, 10). TET3 expression were examined in CD163⁺ cells in human tissue samples using fluorescent immunohistochemistry (IHC). The specificity of the TET3 antibody was confirmed in TET3 knockdown cells (FIG. 35). While CD163⁺ cells expressing low or undetectable levels of TET3 (herein referred to as TET3 negative CD163 macrophages) were found in all tissue samples examined, those strongly expressing TET3 (herein referred to as TET3/CD163 double-positive macrophages) appeared to be specifically associated with disease conditions, albeit not uniformly distributed and with varying abundance across disease tissue areas and across patients (for example, FIG. 36). As such, TET3/CD163 double-positive macrophages were found in lesions of peritoneal endometriosis (the most common form of endometriosis) but not in normal endometrium (FIG. 24).

TET3/CD163 double-positive cells were also evident in livers of NASH but not in healthy livers (FIG. 24). Further, TET3/CD163 double-positive macrophages were detected in tumor areas of NSCLC but not in adjacent non-tumor tissue (FIG. 24). In addition, there was a heterogeneous subcellular localization of TET3 in CD163⁺ macrophages (FIG. 24). Heterogeneous subcellular localization of TET proteins has been documented in other cell types (Arioka et al., 2012, PLoS One, 7:e45031; Zhang et al., 2014, J Biol Chem, 289:5986-5996; Lin et al., 2022, Leukemia, 36:1150-1159).

As TET3 and TET2 were the predominant TET family isoforms expressed in macrophages (Xue et al., 2016, Cell Rep, 16:1096-1105; Cull et al., 2017, Exp Hematol, 55:56-70), TET2 was also examined. While TET2 expression was readily detectable in a subset of CD163⁺ macrophages in NASH, it was negligible in CD163⁻ macrophages in endometriosis and NSCLC (FIG. 24). Further studies therefore focused on studying TET3/CD163 double-positive macrophages as these were specifically associated with disease conditions.

### Macrophage TET3 Expression was Upregulated by Disease-Associated Factors

Macrophages are remarkably plastic cells, which can be modified molecularly and functionally in response to microenvironmental cues (Park et al., 2022, Cell, 185:4259-4279; Ardura et al., 2019, Front Pharmacol, 10:1255). TGF-b1 upregulated TET3 in hepatic stellate cells (HSCs) and hepatocytes during liver fibrosis (Xu et al., 2020, Cell Rep, 30:1310-1318 e1315). Levels of TGF-b1 were found to increase in the peritoneal cavity of women with endometriosis and Tgtb1 deletion reduced the growth of endometriosis lesions in mice (Young et al., 2017, Hum Reprod Update, 23:548-559). MCP1 (also known as CCL2) secreted from lung tumor cells and associated stromal compartment altered macrophages to facilitate tumor growth and metastasis (Yoshimura et al., 2015, Front Immunol, 6:332; Wang et al., 2022, Oncol Lett, 23:26). MCP1 blockage using neutralizing antibodies elicited anti-tumor effects in multiple NSCLC mouse models (Fridlender et al., 2011, Am J Respir Cell Mol Biol, 44:230-237).

Although not bound by any particular theory, it was hypothesized that disease-associated factors might alter TET3 expression in macrophages. First, TGF-b1 was tested in human primary peripheral blood monocyte-derived macrophages (MDMs). TGF-b1 increased TET3 expression both at mRNA and protein levels with a concomitant increase in the number of TET3/CD163 double-positive cells (FIG. 24). Incubation of MDMs with MCP1 resulted in similar effects (FIG. 24).

Next, MDMs were exposed to conditioned media from human HSCs (CM-HSC). Increased TET3 expression accompanied by increased numbers of TET3/CD163-double positive cells were observed (FIG. 24). However, when cells were incubated with the conditioned media in the presence of a TGF-b1-specific antibody, these effects were abolished (FIG. 24). Similar observations were made when cells were exposed to conditioned media from human endometriosis stromal cells (Chen et al., 2021, Reprod Sci, 28:426-434) (CM-Endo) (FIG. 24).

A549 is the most commonly used human NSCLC cell line. Exposing MDMs to conditioned media from A549 increased TET3 expression as well as the number of TET3/CD163 double-positive cells, and the effects were blocked by an MCP1-specific antibody (FIG. 24). Further, when peritoneal macrophages isolated from wild-type mice were tested, TGF-b1 and MCP1 were able to increase TET3 expression and TET3/CD163 double-positive cells (FIG. 24N through FIG. 24Q). In aggregate, the results showed that disease-associated soluble factors upregulated expression of both TET3 and CD163 in macrophages.

### TET3 Affected Macrophage Viability

To explore the functional significance of TET3 overexpression, TET3 expression was downregulated using an siRNA specifically targeting human TET3 (TET3 siRNA) (Xie et al., 2022, J Clin Invest, 132) in MDMs exposed to conditioned media shown to induce TET3 overexpression (FIG. 24). Transfection of TET3 siRNA resulted in decreased expression of TET3 mRNA and protein without affecting TET2 expression (FIG. 25). Importantly, TET3 knockdown induced apoptosis, as assessed by the Terminal deoxynucleotidyl transferase dUTP nick end labeling (TUNEL) assay, which detects DNA damage during programmed cell death (FIG. 25). Likewise, TET3 knockdown, using an siRNA specifically targeting mouse Tet3 (Tet3 siRNA) (Xie et al., 2022, J Clin Invest, 132) in mouse Raw 264.7 macrophages, increased apoptosis (FIG. 25). Of note, unstimulated Raw 264.7 macrophages expressed a high basal level of TET3 and also allowed for a high efficiency of siRNA-mediated knockdown of gene expression. These results indicated that maintaining a high level of TET3 expression was required for preventing TET3-overexpressing macrophages from apoptosis.

### Bobcat339 Decreased TET3 Protein Level in Macrophages

The catalytic domains of the three TET enzymes were highly conserved, although each of the members exhibited varying catalytic activity and substrate preferences (Tahiliani et al., 2009, Science, 324:930-935). Bobcat339 was designed to bind specifically to the catalytic sites of all three TETs enzymes, and was a synthetic cytosine derivative originally reported to inhibit the enzymatic activity of TETs in vitro (Chua et al., 2019, ACS Med Chem Lett, 10:180-185). It was later reported that Bobcat339 by itself had no inhibitory activity against TETs in the absence of contaminating copper(II) (Weirath et al., 2022, ACS Med Chem Lett, 13:792-798). The present examples, however, demonstrated that Bobcat339 induced TET3 protein degradation in the absence of contaminating copper(II) in human and mouse neuronal cells (Lv et al., 2023, Proc Natl Acad Sci, 120). Thus, a small molecule initially designed as protein function modulators was shown to induce protein degradation (Dauvois et al., 1992, Proc Natl Acad Sci, 89:4037-4041; Bekes et al., 2022, Nat Rev Drug Discov, 21:181-200).

When MDMs were incubated with Bobcat339 at 10 mM for 24 h, a decrease in the steady-state level of TET3 protein was observed while that of TET2 was not significantly affected under the same conditions (FIG. 25). Bobcat339 treatment did not alter the mRNA levels of TET3 and TET2 (FIG. 25). Similar observations were made with Raw 264.7 cells (FIG. 25). The 10 mM concentration was used based on the studies that showed that it decreased the steady-state protein level of TET3 (but not TET2) in human and mouse neuronal cells (Lv et al., 2023, Proc Natl Acad Sci, 120).

To determine whether Bobcat339 affect TET protein stability in macrophages, time course experiments were performed on MDMs in the presence of cycloheximide, a protein synthesis inhibitor. In the absence of Bobcat339, TET3 appeared to be much less stable than TET2 in these cells (FIG. 25). While Bobcat339 destabilized both proteins, it reduced the steady-state level of TET3 much more dramatically than that of TET2 under the same conditions (FIG. 25).

### Bobcat339 Induced Macrophage Apoptosis in a TET3-Dependent Manner

Given that Bobcat339 decreased the steady-state level of TET3 (FIG. 25) and that siRNA-mediated TET3 knockdown induced macrophage apoptosis (FIG. 25), studies investigated whether exposing cells to Bobcat339 promotes apoptosis, and if so, whether this effect could be overcome by overexpression of exogenous TET3.

Thus, MDMs were incubated with Bobcat339 in the presence or absence of exogenous TET3 expression from an adenoviral vector (Ad-TET3) (Lv et al., 2023, Proc Natl Acad Sci, 120). While Bobcat339 decreased the level of TET3 protein, exogenous TET3 expression was able to restore it to the control level (FIG. 25). Treatment of these cells with Bobcat339 induced apoptosis, which was abolished when TET3 protein was restored to control levels through exogenous expression (FIG. 25). Similar observations were made in RAW 246.7 cells (FIG. 25). Thus, Bobcat339 promoted macrophage apoptosis and TET3 was a critical mediator of this effect.

### TET3 Acted as a Positive Regulator of Proinflammatory Genes

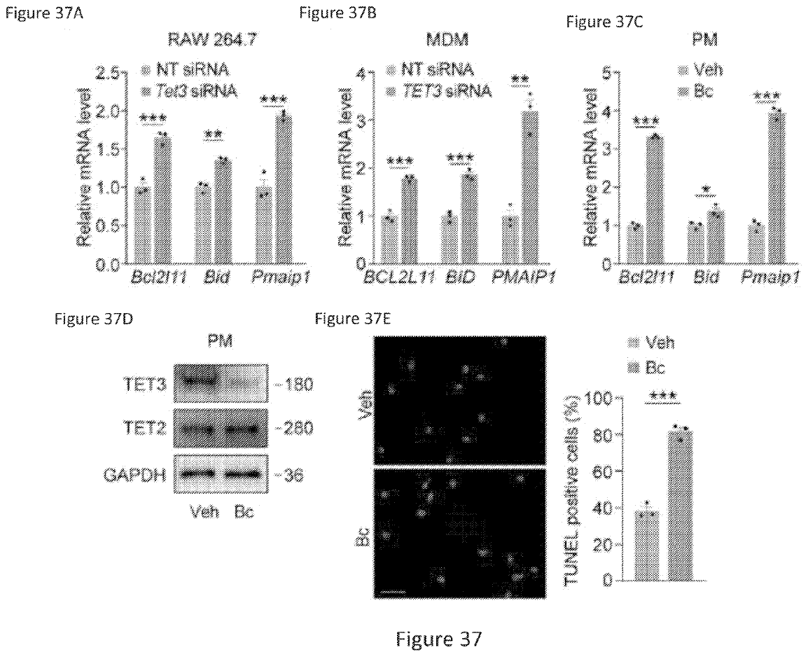

To gain a deeper molecular understanding of the function of TET3 in macrophages, siRNA knockdown of TET3 was performed in unstimulated Raw 264.7 cells, followed by RNA deep-sequencing (RNA-seq). TET3 knockdown led to altered expression of 342 genes (P<0.05) when compared with control siRNA transfected cells (FIG. 37).

Ingenuity Pathway Analysis (IPA) identified “Cytokine/Chemokine”, “Pattern recognition receptor” and “Cell death signaling” to be among the top classes affected by TET3 (FIG. 37). In TET3 knockdown cells, genes in the “Cell death signaling” class with known pro-apoptosis functions were mostly upregulated and genes in the “Cytokine/Chemokine” class with known proinflammatory actions were mostly downregulated (FIG. 37).

Bcl2l11 (encoding BIM), Bid (encoding BID) and Pmaip1 (encoding NOXA) were key pro-apoptotic genes of the Bcl-2 family (Roufayel et al., 2022, Life (Basel), 12). Consistent with TET3 knockdown inducing apoptosis, qRT-PCR analysis revealed significantly increased expression of Bcl2l11, Bid and Pmaip1 in unstimulated Raw 264.7 macrophages as well as TGF-b1 primed human MDMs transfected with TET3 siRNAs vs. control siRNAs (FIG. 25 and FIG. 37).

When TGF-b1 primed mouse peritoneal macrophages were tested, an upregulation of all three pro-apoptosis genes in Bobcat339- vs. Veh-treated cells was observed, consistent with Bobcat339-induced apoptosis of macrophages (FIG. 25 and FIG. 37). Thus, TET3 protects macrophages from apoptosis at least in part through inhibition of expression of key pro-apoptosis genes.

The lysozyme gene Lyz2, which encodes lysozyme M (LysM), was exclusively expressed in myelomonocytic cells including monocytes, macrophages and granulocytes (Shi et al., 2018, Methods Mol Biol, 1784:263-275). The LysM-cre mice allowed for a highly efficient (~90%) manipulation of peritoneal macrophages (Shi et al., 2018, Methods Mol Biol, 1784:263-275). Thus, Tet3^{fl/fl} mice were mated with LysM-cre mice to knockout Tet3 in the myelomonocytic compartment (Lysm^{+/wt} Tet3^{fl/fl}, herein referred to as “Mye-Tet3 ko”). Peritoneal macrophages from Mye-Tet3 ko mice showed ~90% decrease in the expression of Tet3 without affecting Tet2 (FIG. 31). Compared to wild-type control (Lysm^{wt/wt} Tet3^{fl/fl}, herein WT), the Mye-Tet3 ko mice showed similar numbers and percentages of myeloid populations including macrophage and monocytic cells in spleen and bone marrow, demonstrating no alteration in steady-state myeloid cell lineage distribution by Tet3 ablation (FIG. 32). This was consistent with the notion that TET3 expression was not required for monocyte development and differentiation (Gerecke et al., 2022, Front Immunol, 13:861351). Further, myeloid-specific Tet3 ablation did not alter body weight, body composition, and fasting glucose levels (FIG. 33).

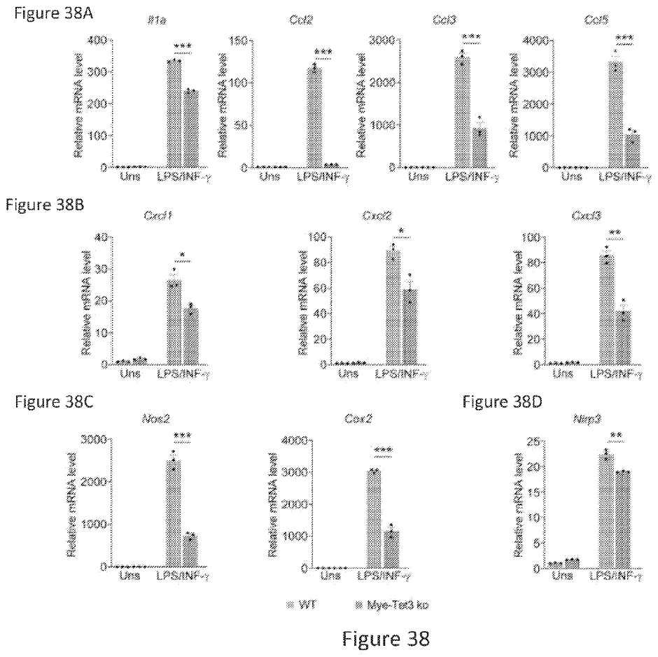

Next, TET3 effects on the expression of proinflammatory response genes was evaluated in peritoneal macrophages isolated from WT and Mye-Tet3 ko mice and stimulated with a combination of lipopolysaccharide (LPS) and IFN-g. qRT-PCR analysis showed markedly decreased expression of proinflammatory cytokines, chemokines, enzymes, and the main NLRP3 inflammasome component in peritoneal macrophages isolated from Mye-Tet3 ko mice as compared to WT mice (FIG. 25 and FIG. 38). TET3-deficiency also led to decreased production of IL-1b and IL-6 proteins (FIG. 25). Significantly decreased expression of IL-1b and IL-6 were also observed in TET3 knockdown human MDMs (FIG. 25). Overall, the data demonstrated that TET3 positively regulated inflammation by upregulating the expression of proinflammatory genes.

### Myeloid-Specific Tet3 Ablation Mitigated Endometriosis

Given that chronic inflammation contributed critically to endometriosis, NASH, and lung cancer and that TET3 positively regulated inflammation, additional experiments focused on the evaluation of whether TET3-overexpressing macrophages promote disease progression. The establishment and maintenance of endometriosis involved mainly peritoneal macrophages (Hogg et al., 2020, Front Endocrinol (Lausanne), 11:7). Indeed, depletion of peritoneal macrophages profoundly inhibited growth and vascularization of lesions and attenuated pain in mice (Bacci et al., 2009, Am J Pathol, 175:547-556; Forster et al., 2019, FASEB J, 33:11210-11222). The Mye-Tet3 ko mice (achieving ~90% efficiency of TET3 knockdown in peritoneal macrophages) offered the opportunity to perform these experiments using endometriosis (FIG. 31).

To address whether myeloid-specific Tet3 ablation affect endometriosis, a well-established, surgically induced murine model of endometriosis was used (Hogg et al., 2020, Front Endocrinol (Lausanne), 11:7; Sahin et al., 2018, J Cell Mol Med, 22:5346-5353). Thus, female mice of WT and Mye-Tet3 ko were subjected to surgery (week 0) to induce intraperitoneal endometriosis, followed by euthanasia and blood and tissue collection on week 6. While no body weight difference between the groups was observed, compared to the WT mice, the ko mice showed a significant reduction in the lesion volume both macroscopically and histologically (FIG. 26 and FIG. 33). IHC analysis revealed a marked decrease in the number of TET3-overexpressing CD163⁺ macrophages, which was accompanied by a dramatic reduction in the expression of IL-1b and IL-6 in CD163⁺ macrophage-enriched areas in the lesions of the ko mice as compared to the WT mice (FIG. 26). Thus, Tet3 ablation in the myeloid compartment significantly reduced lesioned TET3-overexpressing CD163⁺ macrophages, pro-inflammatory cytokine production, and endometriosis burden.

### Bobcat339 Recapitulated the Myeloid-Specific Tet3 Ko Effects on Endometriosis

As Bobcat339 destabilized TET3, thus promoting macrophage apoptosis, additional studies tested whether Bobcat339 inhibits endometriosis (FIG. 25). Female mice were randomly divided into three groups (sham, endometriosis treated with vehicle, and endometriosis treated with Bobcat339) and subjected to surgery (week 0) to induce endometriosis or sham (FIG. 26). The first once-a-week intraperitoneal (i.p.) injection of Bobcat339 (or vehicle) at a dose of 3 mg/kg body weight was performed on week 2, followed by euthanasia and sample collection on week 8.

Bobcat339 treatment significantly reduced the lesion volume both macroscopically and histologically (FIG. 28). Importantly, there was a marked decrease in the number of TET3-overexpressing CD163⁺ macrophages, which was paralleled by a dramatic reduction in the expression of IL-1b and IL-6 in CD163⁺ macrophage-enriched areas in the lesions of Bobcat339- vs. Veh-treated mice (FIG. 26). There was no evidence of liver toxicity following 6 weeks of once-a-week Bobcat339 treatment, nor was there a significant difference in body weight between the Endo+Veh and Endo+Bobcat339 groups (FIG. 28). These results indicated that Bobcat339 attenuated endometriosis at least in part by depleting TET3/CD163 double-positive macrophages.

### Bobcat339 Elicited Therapeutic Effects in a Murine Model of NASH

As Bobcat339 mimicked the therapeutic effects of myeloid-specific Tet3 ko in endometriosis without discernible toxicity, additional studies investigated whether Bobcat339 treats NASH using a high fat high sugar diet-induced murine model (Kroh et al., 2020, Gastroenterol Res Pract, 7347068). As shown in FIG. 28, mice were placed on a chow diet (CD) (control group) or a western diet with 10% sucrose in drinking water (WD) for 8 weeks to induce an early NASH phenotype (Kroh et al., 2020, Gastroenterol Res Pract, 7347068). Then, these mice were therapeutically dosed intravenously once a week from week 8 through week 11 with either 3 mg/kg Bobcat339 (treatment group) or vehicle (model group) for a total of 13 weeks on diets. At the end of the study, mice were euthanized following a 12 h overnight fasting, and plasma and liver were harvested for analyses.

8 weeks of WD significantly increased fasting glucose and decreased glucose tolerance in both model and treatment groups as compared to control group, indicating impaired glucose metabolism (FIG. 29). While fasting glucose and glucose tolerance in the model group remained abnormal at week 12, those in the treatment group showed a significant improvement following Bobcat339 exposure (FIG. 29). At week 13, in contrast to the control animals, the model group animals showed an elevated plasma aspartate aminotransferase (AST), indicating hepatocellular injury, and Bobcat339 normalized this marker (FIG. 28).

The animals in the control group had normal liver histology. The animals in the model group exhibited a general disturbance of hepatic architecture, with moderate/severe steatosis and lobular inflammation, and no/mild hepatocyte ballooning. These histopathological alterations were accompanied with significant increases in liver triglycerides and liver-to-body weight ratio (FIG. 28). This group of animals had a mean NAS score of 5, indicating the presence of NASH. Bobcat339 normalized liver triglycerides, decreased liver-to-body weight ratio, and reduced NAS scores (FIG. 28).

Hepatic fibrosis stage was assessed using Sirius Red/Fast Green stain and the measurement of liver hydroxyproline, which is a unique amino acid in collagen and which serves as an important biomarker of liver fibrosis (Xu et al., 2020, Cell Rep, 30:1310-1318). The model group animals developed more fibrosis than the control group animals, which was further supported by a significant increase in hepatic hydroxyproline content (FIG. 28). Bobcat339 treatment led to a significant reduction in both the fibrosis stage and hydroxyproline content (FIG. 28).

Representative images of Sirius Red/Fast Green and H&E liver sections depicted severe steatosis and definitive fibrosis in the model group, and Bobcat339 significantly reduced the severity of both steatosis and fibrosis (FIG. 29). Importantly, IHC analysis revealed accumulation of TET3-overexpressing CD163⁺ macrophages in the liver of the model group mice, which was significantly reduced after Bobcat339 treatment (FIG. 28). Bobcat339 treatment also reduced the expression of IL-1b and IL-6 in CD163⁺ macrophage-enriched areas in the liver tissue sections (FIG. 28). Taken together, the results indicated that Bobcat339 attenuated NASH (NAS score and fibrosis) at least in part by removing TET3-overexpressing CD163⁺ macrophages.

### Bobcat339 Exhibited Therapeutic Effects in a Syngeneic Murine Model of Lung Cancer

Metastatic lung cancer was established by tail vein injection of Lewis lung carcinoma (LLC) (a mouse NSCLC cell line) (Bertram et al., 1980, Cancer Lett, 11:63-73) cells into immunocompetent mice (FIG. 29). Intravenous dosing of Bobcat339 (or vehicle) at 3 mg/kg every four days was initiated three days after tumor cell injection. The lungs were collected at day 20 from sacrificed mice.

The Bobcat339-treated group showed an increased survival rate (FIG. 29). The weight of the lungs in Bobcat339 treated animals was significantly less than that of the lungs in Veh-treated animals (FIG. 29). Also, fewer LLC metastases were found in Bobcat339 treated lungs than in the lungs of Veh-treated animals, consistent with a decreased tumor burden (FIG. 29).

Further, on day 16, as compared to day 0, mice in the Veh group lost ~5% of body weight, whereas those in the Bobcat339 group gained ~6% of body weight (FIG. 29). The body weight loss in the Veh group could be attributed mainly to the loss of lean body mass, although no difference in food intake was found between the groups (FIG. 29). Severe weight loss was frequently seen in patients with NSCLC, which was also associated with systemic inflammation and cancer cachexia (Staal-van den Brekel et al., 1995, J Clin Oncol, 13:2600-2605; Ulmann et al., 2019, Ann Nutr Metab, 75:223-230).

At homeostasis mouse lung macrophages do not express CD163 whereas human lung macrophages do (Bain et al., 2022, Mucosal Immunol, 15, 223-234). Therefore, an antibody specific for Mac2 (also called Galectin 3) was used to identify macrophages (Liu et al., 2023, Nat Cardiovasc Res, 2:572-586) in mouse lung tissues where CD163⁺ macrophages were not found using CD163 antibodies. However, CD163⁺ macrophages that also overexpressed TET3 were detected in lung tumors of both vehicle- and Bobcat339-treated animals (FIG. 29). These macrophages also expressed high levels of IL-1b and IL-6 (FIG. 29).

In contrast, in non-tumor areas of the lungs of vehicle- or Bobcat339-treated animals, no CD163⁺ macrophages except for Mac2⁺ macrophages that were TET3 negative were detected (FIG. 29). This was reminiscent of human NSCLC where TET3-overexpressing CD163⁺ macrophages were detected in tumor areas but not in non-tumor areas (FIG. 24). Further, the Mac2⁺ macrophages expressed negligible levels of IL-1b or IL-6 (FIG. 29). Thus, metastatic lung cancer was strongly inhibited by treatment with Bobcat339, likely through depletion of the inflammatory TET3/CD163 double-positive macrophages.

The interaction between the immune checkpoint proteins PD1 (programmed cell death protein 1) and its primary ligand PD-L1 plays a vital role in cancer immune evasion. Immune checkpoint inhibitors (ICIs) have revolutionized the treatment of NSCLC, yet only a small subset of patients respond to ICIs. Currently, tumor tissue PD-L1 expression using IHC is the only approved biomarker for patient selection (Sahin et al., 2018, J Cell Mol Med, 22:5346-5353). Studies have documented high expression of PD-L1 in TAMs in tumor tissues of NSCLC, ovarian cancer and gastric cancer, which has been associated with poor clinical outcomes (Gottlieb et al., 2017, Gynecol Oncol, 144:607-612; Yamashita et al., 2020, Gastric Cancer, 23:95-104; Shinchi et al., 2022, Cancer Immunol Immunother, 71:2645-2661). The binding of PD-L1 on macrophages to PD-1 on T cells induced T-cell exhaustion (Ma et al., 2023, J Cancer Res Clin Oncol 149, 8143-8152). Intriguingly, a recent study reported a strong positive association between high levels of IL-6 in tumor tissues and poor response to ICIs in NSCLC (Liu et al., 2022, BMC Med, 20:187).

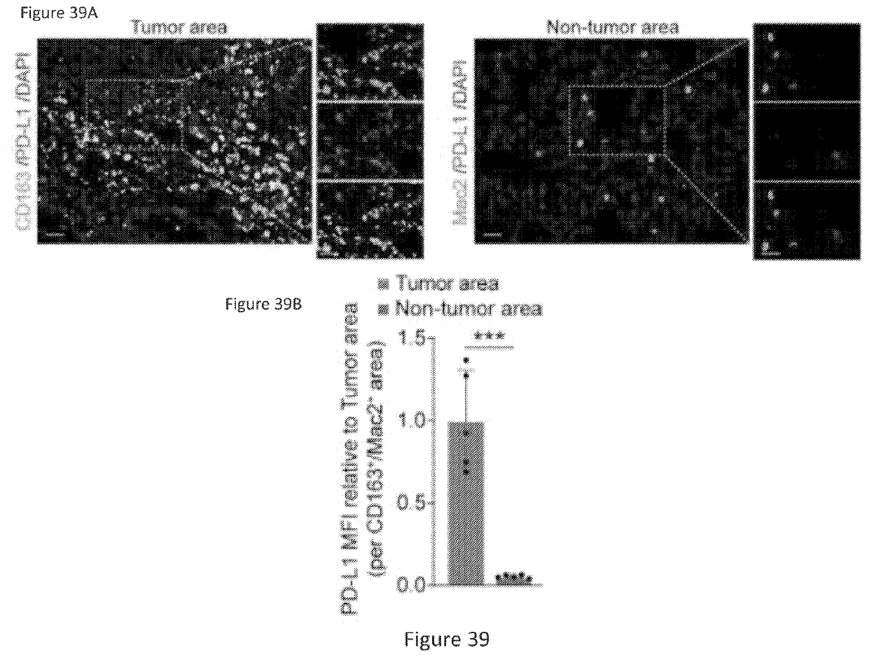

It was also shown that IL-6 promoted PD-L1 expression and that dual blockage of IL-6 and PD-L1 elicited synergistic antitumor effects in murine models (Liu et al., 2022, BMC Med, 20:187). High levels of PD-L1 expression were detected in CD163⁺ macrophages in tumor areas as compared to non-tumor areas (FIG. 39). As ~80% of CD163⁺ macrophages overexpressed TET3, this indicated that the TET3/CD163 double-positive macrophages promoted TME in part by increasing PD-L1 expression and also indicated that targeting these double-positive macrophages significantly enhanced the beneficial effects of ICIs (FIG. 29).

TET3 Enhanced Expression of IL-1b and IL-6 by Decreasing Let-7 miRNA Levels

The transcription factor NF-kB induced expression of proinflammatory cytokines including IL-1b and IL-6, both of which also activated NF-kB, forming a positive feedback loop (Tak et al., 2001, J Clin Invest, 107:7-11). Let-7 miRNAs post-transcriptionally suppressed IL-6 expression by directly targeting the 3′ UTR of its mRNA, thereby indirectly inhibiting NF-kB signaling (Iliopoulos et al., 2009, Cell, 139:693-706). The let-7 family contained 12 members, which were made as precursors and were then processed to become mature miRNAs (Roush et al., 2008, Trends Cell Biol, 18:505-516). Notably, let-7 miRNA levels were significantly reduced in human endometriosis tissue (Grechukhina et al., 2012, EMBO Mol Med, 4:206-217; Cho et al., 2016, Fertil Steril, 106:673-680), NSCLC tissue (Takamizawa et al., 2004, Cancer Res, 64:3753-3756), and NASH/fibrosis tissue (Zhang et al., 2019, Exp Ther Med, 17:3935-3942; Hong et al., 2020, Front Pharmacol, 11:529553).

Further, intraperitoneal injection of let-7 led to decreased endometriosis lesion size (Sahin et al., 2018, J Cell Mol Med, 22:5346-5353) and let-7 overexpression from lentiviral vectors reduced growth of NSCLC in tumor xenograft mouse models (Kumar et al., 2008, Proc Natl Acad Sci, 105:3903-3908). Although not bound by any particular theory, it was hypothesized that TET3 affects let-7 expression in macrophages. Because of the redundancy between let-7 family members, let-7a was tested.

Let-7a levels increased in Raw 264.7 macrophages following treatment with Tet3 siRNA (FIG. 30). Likewise, let-7a levels were higher in Tet3 ko macrophages than in WT macrophages (FIG. 30). Further, in Bobcat339-treated macrophages where TET3 protein was shown to be downregulated, higher levels of let-7a were detected as compared to vehicle treated macrophages (FIG. 30 and FIG. 37). Taken together, these results indicated that TET3 negatively regulates let-7 miRNA expression.

Further, the expression of let-7 miRNAs was regulated by Lin28, which selectively blocked let-7 biogenesis through repression of let-7 processing (Piskounova et al., 2011, Cell, 147:1066-1079). Consistent with this mechanism, Lin28 expression was decreased in Tet3 ko vs. WT macrophages as well as in Bobcat339- vs. vehicle-treated macrophages (FIG. 30). These results indicated that TET3 reduced let-7 miRNA levels by upregulating Lin28.

Next, WT mouse macrophages were transfected with let-7a (or control miRNA), followed by stimulation with LPS/IFN-g. A significant decrease in expression of IL-1b and IL-6 both at mRNA and protein levels in let-7a vs. control transfected macrophages was observed (FIG. 30). Finally, let-7a miRNA levels were increased by Tet3 ko or Bobcat339 treatment in all three disease tissues (FIG. 30). Collectively, the results indicated a mechanism by which macrophage TET3 promoted IL-1b and IL-6 expression at least in part through modulating let-7 miRNA levels.

Overall, this example described the identification of a novel class of CD163⁺ macrophages that overexpressed TET3 and which were associated with lung cancer, endometriosis, and NASH. The studies provided evidence that TET3 overexpression in these cells was induced by disease microenvironments. TET3 overexpression altered gene expression genome-wide, transforming these macrophages into pathogenic DAMs characterized by TET3/CD163 double-positivity. TET3 positively regulated the expression of proinflammatory genes, including IL-1b and IL-6, both of which have well-established roles in promoting these diseases.

Mechanistically, the studies showed that TET3 negatively regulated the expression of let-7 miRNAs which in turn inhibited IL-6 expression and hence the activity of NF-kB. The studies also showed that TET3 upregulated expression of Lin28, known to function to specifically block the processing and accumulation of let-7 miRNAs. Owing to its activity as a DNA demethylase, it is likely that Lin28 expression was stimulated by TET3 at an epigenetic level.

An additional critical finding was that TET3 overexpression was required to suppress apoptosis of these macrophages, rendering them vulnerable to TET3 knockdown, such as by the small molecule TET3 degrader, Bobcat339. As TET3 negative CD163⁺ macrophages were also detected in disease tissues, some of these may be pathogenic or protective (FIG. 24 and FIG. 36). The studies also do not imply that the TET3/CD163 double-positive macrophages were the only class of pathogenic macrophages or that they were identical in other surface markers/molecular features in all three diseases. Nonetheless, because TET3/CD163 double-positive macrophages were found in all three diseases, were proinflammatory both in vitro and in vivo, and eliminating them mitigated all three diseases, the working model is illustrated in FIG. 30.

In an in vitro study, macrophage TET3 was reported to inhibit Infb gene transcription (Xue et al., 2016, Cell Rep, 16:1096-1105). The results demonstrated that macrophage TET3 positively regulated inflammation both in vitro and in vivo. This is in striking contrast to reports that macrophage TET2 functioned in an opposite manner, to restrain expression of proinflammatory cytokines and chemokines, including IL-1b and IL-6 (Wong et al., 2023, Nature, 616:747-754; Pan et al., 2017, Immunity, 47:284-297; Fuster et al., 2017, Science, 355:842-847; Jiang et al., 2019, Proc Natl Acad Sci, 116:12416-12421; Qin et al., 2021, Cells, 11; Liu et al., 2023, Nat Cardiovasc Res, 2:572-586; Yeaton et al., 2022, Cancer Discov, 12:2392-2413), highlighting non-overlapping functions of TET family proteins.

Bobcat339 was originally designed to specifically interact with the catalytic domains of all three TET proteins (Chua et al., 2019, ACS Med Chem Lett, 10:180-185). Like LysM-cre and other cre drivers, which have non-specific targets (Shi et al., 2018, Methods Mol Biol, 1784:263-275; Sahasrabuddhe et al., 2022, Cell Rep, 38:110252; Rumianek et al., 2022, Front Immunol, 13:918636), off-target effects of Bobcat339 were of potential concern. While Bobcat339 likely affect all cells which express TETs, more specific targeted TET3 degradation/inhibition in macrophages is likely to be even more therapeutically efficacious. However, the dosage and administration regimes have revealed minimal adverse pathology (Lv et al., 2023, Proc Natl Acad Sci, 120).

First, Bobcat339 recapitulated the therapeutic effects of myeloid-specific Tet3 ko on endometriosis without discernible side effects. Second, in both the NASH and lung cancer models, Bobcat339 not only mitigated diseases but also elicited other beneficial effects, including decreasing liver injury, maintaining body weight, and increasing survival. Third, in all three disease models, Bobcat339 reduced TET3/CD163 double-positive macrophages as well as proinflammatory cytokine production, corroborating the in vitro observation that Bobcat339 promoted apoptosis of TET3-overexpressing macrophages via targeting TET3. Finally, as increased TET3 expression in hepatocytes and hepatic stellate cells promoted liver fibrosis (Xu et al., 2020, Cell Rep, 30:1310-1318), it was possible that Bobcat339 targeted TET3 in these cells as well as macrophages. Therefore, the beneficial effects of Bobcat339 in the NASH model involve a combination of effects on all three types of cells.

Significant therapeutic effects of myeloid-specific Tet3 ko was observed on endometriosis, which was correlated with significantly diminished expression of TET3/CD163 double-positive macrophages and inflammatory cytokines in endometriosis lesions (FIG. 26). This was consistent with the notion that peritoneal macrophages play a key role in endometriosis and that peritoneal macrophages from Mye-Tet3 ko showed 90% TET3 ko efficiency (FIG. 31).

Unlike endometriosis, NASH and lung cancer appeared to involve the contribution of a more complex collection of macrophages. In lung cancer, both alveolar macrophages and interstitial macrophages were involved (Loyher et al., 2018, J Exp Med, 215:2536-2553; Hogg et al., 2020, Front Endocrinol (Lausanne), 11:7, Bain et al., 2022, Mucosal Immunol, 15:223-234). In NASH, both Kupffer cells and monocyte-derived macrophages played important roles (Barreby et al., 2022, Nat Rev Endocrinol, 18:461-472; Krenkel et al., 2018, Hepatology, 67:1270-1283; Cai et al., 2020, Cell Metab, 31:406-421 e407; Li et al., 2020, Front Immunol, 11:1169). Although LysM-cre was functional in both embryonic and recruited macrophages in the liver and other cre drivers have been used to target macrophage lineages in part or whole in other tissues, a high efficiency (at least 90%) TET3 ko simultaneously in all macrophage lineages may be necessary to elicit therapeutic effects in NASH and lung cancer, given that TET3 controlled the viability of pathogenic macrophages. Indeed, no therapeutic effects on NASH and LLC lung cancer was observed using Mye-Tet3 ko mice. A significant decrease in expression of TET3/CD163 double-positive macrophages in disease tissues in Mye-Tet3 ko mice as compared to WT controls was also not observed, indicating limited efficacy in myeloid-specific Tet3 ablation in these models.

Nonetheless, Bobcat339 was tested in NASH and NSCLC, as this compound appeared to act in all TET3 overexpressing macrophages (and perhaps all macrophage lineages), eliciting impressive therapeutic effects with no discernible toxicity. Thus, despite caveats associated with Mye-Tet3 ko (potential off-target effects and inefficient Tet3 ko in NASH and LLC models) and Bobcat339 (potential off-target effects), the combination of the two complementary approaches and their therapeutic effects in three distinct disease settings offered strong support for the herein described model.

In summary, the present studies have identified a novel class of pathogenic DAMs characterized by TET3 overexpression and CD163-positivity in lung cancer, endometriosis, and NASH. These TET3/CD163 double-positive cells exerted their disease-promoting action at least in part through IL-1b and IL-6 and these cells can be eliminated via targeting TET3. Additional studies, aimed at deeper understanding of the molecular mechanisms, by which macrophage TET3 regulated its target gene expression, aid in the potential usage of TET3 degraders/inhibitors as new therapeutic agents for these diseases and other cancers and chronic inflammatory diseases.

In conclusion, macrophages were essential for innate immunity and tissue homeostasis. Signals from disease microenvironments programed macrophages into DAMs that were phenotypically and functionally distinct from tissue macrophages and which played either pathogenic or protective roles in disease progression (Park et al., 2022, Cell, 185:4259-4279). Targeting DAMs for therapy has remained unsuccessful due to their high heterogeneity (Ardura et al., 2019, Front Pharmacol, 10:1255; Skytthe et al., 2020, Int J Mol Sci, 21).

Here, TET3 overexpression was reported in a subset of CD163⁺ macrophages in human lung cancer and two major chronic inflammatory diseases of distinct types: endometriosis and nonalcoholic steatohepatitis. The studies showed that soluble disease-associated factors upregulated the expression of both TET3 and CD163 in human and mouse macrophages. These macrophages exhibited increased expression of proinflammatory genes. Specifically, TET3 stimulated IL-1b and IL-6 expression via a feedback mechanism involving inhibition of let-7 miRNA expression.

Further, TET3 overexpression was required to maintain the viability of these macrophages, rendering them vulnerable to TET3 inhibitors. Depleting these TET3/CD163 double-positive macrophages through myeloid-specific Tet3 ablation or pharmacologically using Bobcat339, a synthetic small-molecule degrader of TET3, was strongly associated with amelioration of all three diseases in murine models. Therefore, despite DAMs exhibiting extensive heterogeneity, the TET3/CD163 double-positive macrophages identified in this work serve as targets for therapy for lung cancer and major inflammatory diseases.

The materials and methods employed in Example 3 through Example 5 are now described.

### Human Samples

Human peripheral blood mononuclear cells (PBMCs) were collected and enriched by density gradient centrifugation. Macrophage differentiation was induced by treating PBMCs with recombinant human macrophage colony-stimulating factor (M-CSF, Gibco, PHC9504). Briefly, 15 mL of blood samples were taken from voluntary donors in sterile EDTA (K2) tubes (BD #366643) and diluted 1:1 in Phosphate-Buffered Saline (PBS). Diluted samples (15 mL) were laid onto 15 mL of Ficoll-Paque PLUS density gradient medium (Cytiva, 17144003) and centrifuged without excel and brake at 400×g at room temperature (RT) for 20 min. PBMCs were harvested from the mononuclear layer and washed twice with PBS by centrifugation at 300× g for 8 min at 4 °C each time. To induce differentiation to macrophages (MDMs), PBMCs were resuspended in growth media (RPMI 1640 supplemented with 10% heat inactivated fetal bovine serum [FBS, Gibco, 16140-017], 1% Anti-Anti [Gibco, 15240-062], and 50 ng/mL of M-CSF), seeded in 24-well plates at the density of 1×10⁶/well and maintained at 37 °C in a 5% humidified CO₂ tissue culture incubator. Seven days later, nonadherent cells were removed and media were replaced with new growth media every 3 days. Formalin-fixed paraffin embedded (FFPE) normal human endometrium (proliferating phase, n=3 patients) and endometriosis (intraperitoneal, stage I-III, n=5 patients) tissue blocks and human normal liver (n=4 patients) and fibrotic NASH liver (fibrosis stage 1-III, n=4 patients) tissue blocks were obtained from the Yale Pathology Tissue Services.

### Mouse

Mice were housed at 22 °C-24 °C with a 12 h light/12 h dark cycle with a standard chow diet (Harlan Teklad no. 2018, 18% calories from fat) and water provided ad libitum. Female C57BL/6J (JAX, 000664) mice (for endometriosis model) were purchased from the Jackson Laboratory. Male C57BL/6J mice (for NASH model) were purchased from Beijing Vital River Laboratory Animal Technology. Tet3fl/fl mice were provided by Anjana Rao from La Jolla Institute for Immunology. Mye-Tet3 ko mice (Lysm+/wtTet3fl/fl) were generated by crossing LysM-cre (JAX, 004781, Jackson Laboratory) and Tet3fl/fl mice. Littermate Lysmwt/wtTet3fl/fl mice were used as control (WT). For all experiments, age-matched animals were used. For information on animal numbers, refer to figure legends.

### Immunofluorescence of Tissue Sections

Slides were deparaffinized by heating at 65 °C for 60 min, followed by xylene wash 3 times 5 min each. The slides were then rehydrated in 100%, 90%, 80% and 70% ethanol for 5 min each, followed by ddH2O wash 2 times 5 min each. For antigen retrieval, slides were heated to near boiling in sodium citrate solution (freshly prepared, pH 6.0) for 2 min, followed by incubation in a steamer (BELLA Food Steamer, Model #XJ-10102A) for 15 min. The slides were cooled at RT to below 40 °C, followed by sequential wash in PBS and PBS-T (0.05% Tween 20/PBS) for 5 min each. After permeabilization in 1% Triton X-100/PBS for 15 min, slides were blocked in 5% donkey serum/PBS-T at RT for 1 h and quickly washed once with PBS-T before antibody incubation. For TET3/CD163 double staining, slides were incubated with antibodies diluted in 2% donkey serum/PBS-T. Antibodies used are anti-TET3 (GeneTex, GTX121453, diluted at 1:400 for human tissue; Millipore Sigma, ABE290, diluted at 1:2000 for mouse tissue), anti-CD163 (Abcam, ab156769, diluted at 1:400), anti-F4/80 (Invitrogen, 17-4801-82, diluted at 1:50), and anti-CD11b (Biolegend, 101208, diluted at 1:50) overnight at 4 °C. Negative controls were performed by omitting the respective primary antibodies. The next day, slides were washed 3 times and incubated in 0.4% Triton X-100/PBS with the secondary antibody donkey anti-Rabbit IgG Fluor 594 (Invitrogen, A-21207, diluted at 1:500), goat anti-Mouse lgG Fluor 488 (Invitrogen, A-11029, diluted at 1:500), donkey anti-Rabbit IgG Flour 488 (Invitrogen, A-21206, diluted at 1:500), and goat anti-Rat IgG Flour 488 (Abcam, ab150157, diluted at 1:500), for 1 hour at RT. The slides were covered with antifade mounting medium with DAPI (Vector laboratories, H-2000), coverslipped and scoped using a Keyence BZ-X700 fluorescence microscope.

### Immunocytochemistry of MDMs

After wash in cold PBS, adherent cells were fixed with 4% paraformaldehyde in PBS for 15 min and permeabilized with 0.4% TritonX-100 for 15 min. Cells were blocked in 5% donkey serum/PBS-T at RT for 30 min and quickly washed once with PBS-T before antibody incubation. For TET3/CD163 double staining, cells were incubated with anti-TET3 (GeneTex, GTX121453, diluted at 1:400 in 2% donkey serum/PBS-T) and anti-CD163 (Abcam, ab156769, diluted at 1:400) overnight at 4 °C. The next day, cells were washed 3 times with PBS-T and incubated in 0.4% Triton X-100/PBS with donkey anti-Rabbit IgG Fluor 594 (Invitrogen, A-21207, diluted at 1:500) and goat anti-Mouse lgG Fluor 488 (Invitrogen, A-11029, diluted at 1:500) for 1 hour at RT. Cells were covered with antifade mounting medium with DAPI (Vector laboratories, H-2000), coverslipped and scoped using a Keyence BZ-X700 fluorescence microscope.

### Bobcat339 Treatment of Mice

Bobcat339 powder was freshly dissolved in DMSO at a concentration of 50 mg/mL and filtered through a 0.22 micron filter. It was further diluted with 1×PBS to a final concentration of 0.5 mg/mL before injections. Mice were injected intraperitoneally with Bobcat339 at 2.5 mg/kg body weight.

### Induction of Endometriosis

Endometriosis in mice was surgically induced using the previously described methods (Rosa et al., 2019, Reprod Sci, 26:1395-1400; Mamillapalli et al., 2022, Reprod Sci, 29:243-249; Pluchino et al., 2020, J Cell Mol Med, 24:2464-2474). Briefly, uterus horns were removed from wild-type female donor mice, opened longitudinally and cut into fragments of 3 mm. Two uterine segments were sutured to each right and left parietal peritoneum of recipient mice (6-7 weeks old) with absorbable suture. Sham surgeries were performed for sham group, using the same surgical procedure without the introduction of extraneous uterine tissue. The experimental model was allowed to develop for more than 4 weeks. Development of the model was confirmed by opening the abdominal cavity and measuring the size of endometriotic lesions both macroscopically and histologically.

### NASH Induction and Treatment

Male C57BL/6J mice of 6-weeks old were randomly divided into three groups, with each containing 5 animals. The control group were fed a standard chow diet (CD). The model group and the treatment group were fed a western diet (WD) (40 kcal % fat, 20 kcal % fructose, and 2 kcal % cholesterol, Research Diets, D09100301) supplemented with 10% sucrose in drinking water provided ad libitum. In the treatment group, Bobcat339 administration was initiated following 8 weeks of WD by tail vein injection at 2.5 mg/kg body weight once a week for 4 weeks. The control group and the model group received vehicle (DMSO) tail vein injections. As Bobcat339 was shown to temporally (3 days) increase food intake in a mouse model of activity-based anorexia (Lv et al., 2023, Proc Natl Acad Sci, 120), pair-feeding was performed from week 8 through week 12 such that the amount of food provided to the treatment group was matched to that consumed by the model group. After a total of 13 weeks on diets, mice were sacrificed following a 12 h overnight fasting, and plasma and liver were harvested for analyses.

### Glucose Tolerance Test

GTT were performed following 16 h overnight fasting. Each animal received an i.p. injection of 2 g/kg of glucose (Sigma-Aldrich, G5767) in sterile saline. Blood glucose concentrations were measured using Contour next blood glucose meter (Ascensia Diabetes Care) via tail vein bleeding at the indicated time points after injection.

### Measurements of Liver Tissue Triglycerides and Hydroxyproline Contents

These were assessed using Triglyceride Content Assay Kit (Solarbio, BC0625) and Hydroxyproline Assay Kit (Sigma-Aldrich, MAK008-1KT), respectively.

### Histopathology Examination

Mouse liver samples were collected and fixed at 4 °C with 4% of phosphate buffered paraformaldehyde for 24 h. Fixed tissue specimens were embedded in paraffin and cut into 4 mm-thick tissue sections for histopathological analysis. Tissue sections were stained with Hematoxylin-Eosin according to standard protocols. For Sirius Red/Fast Green staining, slides were deparaffinized by heating at 65 °C for 60 min, followed by xylene wash 3 times 5 min each. The slides were then rehydrated in 100%, 90%, 80% and 70% ethanol for 5 min each, followed by ddH₂O wash 2 times 5 min each. Then they were incubated in 0.04% Fast Green (Sigma-Aldrich, F7258) in saturated picric acid for 15 min, washed with distilled water and then incubated in 0.1% Fast Green and 0.08% Sirius Red (Sigma-Aldrich, 365546) in saturated picric acid for 30 min. After that, the slides were washed with 70%, 80%, 90% and 100% ethanol and p-xylene for 5 min each. After air dry, the slides were mounted with DPX Mounting and scoped using Leica DM6B microscope. Collagen fibers appeared red, while the non-collagen proteins were green. The NAFLD activity score (NAS) and the stage of liver fibrosis were assessed by two expert pathologists who were blinded to the treatment group.

### Lewis Adenocarcinoma Cell Line-Induced Lung Cancer Model and Treatment

LLC cells (2.5×10⁵ cells/mouse) were injected into the tail vein of female C57BL/6J mice of 8-weeks old. Mice were randomly divided into vehicle (n=5) and Bobcat339 (n=5) groups. Three days after tumor cell injection, the vehicle group mice received tail vein injection of DMSO and the Bobcat339 group with Bobcat339 at the dose of 2.5 or 3 mg/kg body weight every four days. The animals were sacrificed 20 days after tumor cell injection and lungs were isolated and weighed. The whole lung was fixed in 4% paraformaldehyde overnight at 4 °C and embedded in paraffin for histological analysis and IHC. A second cohort of mice (n=5 in each group) treated as above were used for survival studies using moribund as an experimental endpoint.

### Cell Lines

Human hepatic stellate cell line LX-2 (Sigma-Aldrich, SCC064) and mouse macrophage cell line Raw 264.7 (ATCC, TIB-71), human NSCLC cell line A549 (ATCC, CCL-185), and mouse LLC (ATCC, CRL-1642) were purchased. The human endometriosis stromal cell line was previously described (Chen et al., 2021, Reprod Sci, 28:426-434). LX-2 and Raw 264.7 cells were maintained in DMEM (Gibco, 11965-092) supplemented with 10% FBS and 1% Anti-Anti. Human endometriosis cells were maintained in DMEM-F12 (Gibco, 11330-032) containing 10% FBS and 1% Anti-Anti. A549 cells were grown in F-12K Medium (ATCC, 30-2004) supplemented with 10% FBS and 1% Anti-Anti. LLC cells were grown in DMEM (ATCC, 30-2002) supplemented with 10% FBS and 1% Anti-Anti.

### Conditioned Media Preparation and Treatment

To prepare CM-HSC and CM-Endo, LX-2 and human endometriosis cells were seeded in 24-well plates (2.5×10⁵ cells/well) with their respective growth media and allowed to grow to ~50% confluency. Cells were then washed with PBS and incubated with fresh growth media (500 μl/well) for an additional 24 h. Cell supernatant (CM-HSC/CM-Endo) was collected and immediately used to treat MDMs after 1:1 dilution with their respective growth media. In the control group, only respective growth media were used.

### Cell Transfection

For siRNA transfection in a 24-well plate scale of Raw 264.7 cells, 10 pmol of NT siRNA (AM4636, Ambion) or Tet3 siRNA (4390815/s101483, Ambion) was mixed with 100 μL of OPTI-MEM (Gibco, 31985-070) by gentle pipetting. In parallel, 3 μL of Lipofectamine RNAiMAX (Invitrogen, 13778-150) was mixed with 100 μL of OPTI-MEM by gentle pipetting. Following 5 min of incubation at RT, the two were combined and the resulting 200 μL of transfection solution was added to each well of cells. After 24 h of incubation at 370 C in a 5% humidified CO₂ tissue culture incubator, 300 μL of growth media was added and incubation was continued until cell harvesting.

For siRNA transfection in a 24-well plate scale of MDMs, NT siRNA or TET3 siRNA (4392420/s47239, Ambion) was mixed with 25 μL of OPTI-MEM by gentle pipetting. In parallel, 1.5 μL of Lipofectamine RNAiMAX was mixed with 25 μL of OPTI-MEM by gentle pipetting. Following 5 min of incubation at RT, the resulting 50 μl of transfection solution was added to each well of cells containing 450 μL of growth media.

For let-7a transfection of mouse peritoneal macrophages (PM) in a 24-well plate scale, 10 pmol let-7a (let-7a-5p mimic, Active Motif, MIM0001) or miCon (non-targeting miRNA mimic, Active Motif, MIM9001) was mixed with 100 μL of OPTI-MEM by gentle pipetting. In parallel, 3 μL of Lipofectamine RNAiMAX was mixed with 100 μL of OPTI-MEM by gentle pipetting. Following 5 min of incubation at RT, the resulting 200 μl of transfection solution was added to each well of cells. Following incubation in a CO2 tissue culture incubator for 6 h, 200 μL of growth media were added and incubation was continued to the next day until further treatments with LPS/IFN-γ.

Bobcat339 Treatment of Cultured Cells Bobcat339 powder (Sigma-Aldrich, SML2611) was freshly dissolved in DMSO at a concentration of 5 mM and filtered through a 0.22 micron. Cells grown at a density of 80-90% were incubated in growth media with vehicle (DMSO) or Bobcat339 at a final concentration of 10 μM for 24 h, followed by RNA and protein extraction.

For cultured MDMs, cells were primed with CM-Endo or TGF-β1 (PeproTech, 100-21) for 36-48 h (to increase TET3 expression) before Bobcat339 treatment.

For cultured mouse peritoneal macrophages, cells were primed with 30 ng/ml of TGF-β1 (R&D Systems, 7666-MB0-005) for 48 h (to increase TET3 expression) before Bobcat339 treatment. Bobcat339 (or DMSO) was added at a final concentration of 10 μM, followed by RNA and protein extraction at time points indicated in the figure legends.

For TET protein stability assay of MDMs, cells in 24-well plates were incubated with vehicle or Bobcat339 at a final concentration of 10 μM for 3 h, followed by addition of cycloheximide (CHX, Cell Signaling, 2112) at a final concentration of 50 μg/mL. Proteins were harvested at 0, 2, 4, and 6 hours after addition of CHX.

For TET3 protein stability assay, cells in 24-well plates were incubated with vehicle or Bobcat339 at a final concentration of 10 μM for 3 hours, followed by addition of cycloheximide (CHX, Cell Signaling, 2112) at a final concentration of 50 μg/mL in the presence of 10 μM of Bobcat339. Proteins were harvested at 0, 1, 2, and 3 hours after addition of CHX.

For TET3 expression restoration experiments, cells seeded in 24-well plates were infected with Ad-GFP or Ad-TET3 at 4000 gc/cell. Following 16 h of infection, vehicle or Bobcat339 were added at a final concentration of 10 μM. Protein and RNA were isolated 48 hours later and analyzed.

### Mouse Peritoneal Macrophage Collection

Peritoneal macrophages were collected from the peritoneal cavity of WT or Mye-Tet3 ko mice by injecting 10 mL of PBS followed by gentle abdominal massaging for 5 min. Cells were pelleted by centrifugation at 1500×g at 4 °C for 8 min. To remove red blood cells, cells were resuspended in RBOBCAT339 Lysis Buffer (Biolegend, 420302, diluted to 1×) and allowed to stand for 5 min, followed by addition of PBS to stop the reaction and twice wash with PBS by centrifugation. Macrophages were enriched by plating cells in RPMI 1640 supplemented with 10% FBS and 1% Anti-Anti for 2 h at 37 °C in a 5% humidified CO2 tissue culture incubator. Non-adherent cells were then removed with three PBS washes.

### Proinflammatory Activation of Macrophages and Measurements of IL-1β and IL-6 Proteins

Proinflammatory activation of peritoneal macrophages and MDMs was achieved by treatment with a combination of 10 ng/mL of LPS (Invitrogen, 00-4976-93) and 20 ng/mL of IFN-γ (R&D Systems, 485-MI-100). RNAs were isolated at time points indicated in figure legends. IL-1β and IL-6 protein levels in the supernatant of cultured mouse peritoneal macrophages were measured using ELISA kits (R&D Systems, MLB00C and M6000B-1). For IL-1β, LPS/IFN-γ-primed mouse peritoneal macrophages were incubated with 5 mM ATP (Alfa Aesar, L14522) for 30 min before collection of supernatants for ELISA analysis. Supernatants of cultured mouse peripheral macrophage were collected by centrifugation at 1000×g at 4 °C for 20 min to remove cell debris. To measure IL-1β protein level using ELISA kit (Elabscience, E-EL-H0149c), cell lysates of human MIDMs were used collected. To measure IL-6 protein level using ELISA kit (E-EL-H6165), supernatants of human MDMs were collected. To obtain cell lysates, MDMs of 1×10⁶ in each sample were digested with Trypsin-EDTA (0.25%) followed by centrifugation at 1000×g for 5 min. The supernatants were discarded, and cell pellets were suspended with 150 μL PBS containing protease inhibitors. The resulting cell suspension was incubated in liquid nitrogen for 30 min followed by rapid thawing in a 37 °C water bath. Repeat the above steps three times. The cell lysate was cleared by centrifugation at 1500×g for 10 min at 4 °C to remove insoluble materials. IL-1β and IL-6 protein concentrations were presented after normalization against cell numbers.

### Fluorescent Immunohistochemistry (IHC) of Tissue Sections

FFPE tissue slides were deparaffinized by heating at 65 °C for 60 min, followed by xylene wash 3 times 5 min each. The slides were then rehydrated in 100%, 90%, 80% and 70% ethanol for 5 min each, followed by ddH₂O wash 2 times 5 min each. For antigen retrieval, slides were heated to near boiling in sodium citrate solution (freshly prepared, pH 6.0) for 2 min, followed by incubation in a steamer (BELLA Food Steamer, Model #XJ-10102A) for 15 min. The slides were cooled at RT to below 40 °C, followed by sequential wash in PBS and PBS-T (0.05% Tween 20/PBS) for 5 min each. After permeabilization in 1% Triton X-100/PBS for 15 min, slides were blocked in 5% donkey serum/PBS-T at RT for 1 h and quickly washed once with PBS-T before antibody incubation. For double-staining of CD163/TET3, CD163/TET2, CD163/IL-1β, CD163/IL-6, Mac2/TET3, Mac2/IL-1β, Mac2/IL-6, CD163/PD-L1, and Mac2/PD-L1, slides were incubated with antibodies diluted in 2% donkey serum/PBS-T overnight at 4 °C. Antibodies used are anti-TET3 (GeneTex, GTX121453, diluted at 1:400 for human tissue; Millipore Sigma, ABE290, diluted at 1:500 for mouse tissue), anti-CD163 (Abcam, ab156769, diluted at 1:400 for human tissue; Santa Cruz Biotechnology, sc-58965, diluted at 1:200 for mouse tissue), anti-IL-1β (Proteintech, 26048-1-AP, diluted at 1:200), anti-IL-6 (Abcam, AB6672, diluted at 1:200), anti-Mac2 (Biolegend, 125401, diluted at 1:400), anti-PD-L1 (ThermoFisher Scientific, PA5-88105, diluted at 1:400), and anti-TET2 (21207-1-AP, Proteintech diluted at 1:500). Negative controls were performed by omitting the respective primary antibodies. Note, for CD163/PD-L1 double staining, the permeabilization step (1% Triton X-100/PBS for 15 min) was omitted. The next day, slides were washed 3 times and incubated in 0.4% Triton X-100/PBS with the secondary antibody donkey anti-Rabbit IgG Fluor 594 (Invitrogen, A-21207, diluted at 1:500), goat anti-Mouse lgG Fluor 488 (Invitrogen, A-11029, diluted at 1:500), and goat anti-Rat IgG Flour 488 (Abcam, ab150157, diluted at 1:500) for 1 h at RT. The slides were covered with antifade mounting medium with DAPI (Vector laboratories, H-2000), scoped using a Keyence BZ-X700 fluorescence microscope.

### Fluorescent Immunocytochemistry (ICC) of Human MDMs and Mouse Peritoneal Macrophages

After wash in cold PBS, adherent cells were fixed with 4% paraformaldehyde in PBS for 15 min and permeabilized with 0.4% TritonX-100 for 15 min. Cells were blocked in 5% donkey serum/PBS-T at RT for 30 min and quickly washed once with PBS-T before antibody incubation. Primary antibodies were diluted in 2% donkey serum/PBS-T. For CD163/TET3 double staining, cells were incubated with anti-TET3 (GeneTex, GTX121453, diluted at 1:400 for MDMs; Millipore Sigma, ABE290, diluted at 1:500 for mouse peritoneal macrophages) and anti-CD163 (Abcam, ab156769, diluted at 1:400 for MDMs; Santa Cruz Biotechnology, sc-58965, diluted at 1:200 for peritoneal macrophages) overnight at 4 °C. The next day, cells were washed 3 times with PBS-T and incubated in 0.4% Triton X-100/PBS with donkey anti-Rabbit IgG Fluor 594 (Invitrogen, A-21207, diluted at 1:500) and goat anti-Mouse lgG Fluor 488 (Invitrogen, A-11029, diluted at 1:500) for 1 h at RT. Cells were covered with antifade mounting medium with DAPI (Vector laboratories, H-2000), scoped using a Keyence BZ-X700 fluorescence microscope.

### Immunofluorescent Staining Quantification

Image analysis and fluorescent signal quantification were performed and analyzed using ImageJ. Six tissue sections per mouse were quantified, with 5 mice in each group. Fluorescent signal was quantified as mean fluorescence intensity (MFI) and normalized to CD163⁺ or Mac2⁺ macrophage area. For cultured macrophages, 3 randomly picked fields per group were used for quantification.

qRT-PCR

Total RNAs were extracted from cells using PureLink RNA Mini Kit. 0.5-1 μg total RNA was reverse transcribed to cDNA in a reaction volume of 20 μL using PrimeScript RT Reagent Kit. Real-time quantitative PCR was performed in a 15 μL reaction volume containing 0.5-1 μL of cDNA using SsoAdvanced Universal SYBR Green Supermix in a Bio-Rad iCycler. Gene expression levels were normalized against RPLP0.

For let-7a qPCR, 10 ng of total RNA was reverse transcribed to cDNA in a reaction volume of 10 μl using miRCURY LNA RT Kit (QIAGEN, 339340). Quantitative real-time PCR reactions were carried out using miRCURY LNA SYBR Green PCR Kit (QIAGEN, 339346) and a let-7a-specific primer (Has-Let-7a-5p miRCURY LNA miRNA PCR Assay, QIAGEN, 339306/YP00205727) in a Bio-Rad iCycler. Gene expression levels were normalized against U6 (U6 snRNA v2 miRURY LNA miRNA PCR Assay, QIAGEN, 339306/YPO2119464, QIAGEN).

### Western Blotting

Cells were homogenized in situ using a pipette tip in 2×SDS-sample buffer with 10% β-mercaptoethanol at RT followed by heating at 100 °C for 5 min with occasional vortexing. The samples were loaded onto a 4-15% gradient SDS gel (Bio-rad, 456-8086) (10 μL/well) and transferred to nitrocellulose membranes, followed by Western blot analysis. The antibodies used were anti-TET3 (diluted at 1:1000, GeneTex, GTX121453 for human; diluted at 1:1000, Active Motif, 61395 for mouse), anti-TET2 (diluted at 1:1000, Proteintech, 21207-1-AP for human/mouse), anti-TGF-β1 (diluted at 1:1000, Abcam, ab215715 for human), anti-Lin28A (diluted at 1:1000, Cell Signaling, 3978S), and HRP-conjugated anti-GAPDH (dilution 1:5000; Proteintech, HRP-60004). The secondary antibody was HRP-linked Anti-rabbit IgG (dilution 1:10,000; Rockland, 611-1322).

### Measurements of IL-1β and IL-6 Proteins

IL-1β protein level of cell lysates and IL-6 protein level of cell supernatants were determined using the kits (Elabscience, E-EL-H0149c and E-EL-H6165, respectively) according to the manufacturer's protocols. To obtain cell lysates, MDMs of 1×10⁶ in each sample were digested with Trypsin-EDTA (0.25%) followed by centrifugation at 1000×g for 5 min. The supernatants were discarded, and cell pellets were suspended with 150 μL PBS containing protease inhibitors. Then the cell suspension was incubated in liquid nitrogen for 30 min followed by rapid thawing in a 37 °C water bath. Repeat the above steps three times. The cell lysate solution was harvested after removing debris by centrifugation at 1500×g for 10 min at 4 °C. To harvest supernatants, cell supernatants were centrifuged for 20 min at 1000×g at 4 °C and the supernatants were collected for downstream analyses.

### TUNEL Assay

These were performed using the In Situ Cell Death Detection Kit (Sigma Aldrich, 12156792910). Briefly, cells were fixed with 4% paraformaldehyde in PBS for 1 h at RT and rinsed with PBS, followed by permeabilization with 0.1% Triton X-100 in 0.1% sodium citrate for 2 min on ice. TUNEL reaction mixture was added and incubation was carried out at 37 °C for 60 min. DAPI was added to counter-stain the cells for 1 min. The slides were coverslipped and scoped using a Keyence BZ-X700 fluorescence microscope.

### Apoptosis/Caspase-1 Assay

These were carried out using the Apoptosis/Caspase-1 assay kit (MyBioSource, MBS258046). Briefly, after wash with PBS, cells were incubated with 1×FAM-FLCA working solution in 300 μL growth media at 37 °C for 1 hour, followed by wash with 1× Cellular Wash Buffer three times. Hoechst 33342 was applied to counter-stain the cells for 15 min. Cells were then covered with mounting media and coverslips and scoped using a Keyence BZ-X700 fluorescence microscope.

### RNA Sequencing and Data Analysis

RNA-Seq library preparation and sequencing for RAW 264.7 samples were conducted at Yale Stem Cell Center Genomics Core facility through poly A enrichment (lllumina TruSeq Stranded mRNA Library Prep Kit). Differential expression analysis between two different groups was performed with DESeq2 software. Genes with a false discovery rate (FDR) below 0.05 and absolute fold change over 1.0 were analyzed with Ingenuity Pathway Analysis using IPA software (Qiagen). Horizontal bar chart was made for the canonical pathways.

### Nitric Oxide Assay

Nitric oxide (NO) levels of cells were determined using the kit (Elabscience, E-BOBCAT339-K035-M) according to the manufacturer's protocols. Briefly, MDMs of 5×105 in each sample were pelleted by centrifugation at 400×g for 10 min. Cells were then homogenized in 110 μL of PBS using a disposable pellet pestle followed by centrifugation at 14,000×g at 4 °C for 15 min to remove insoluble materials. 100 μL of supernatant was collected and mixed with 200 μL of Sulphate Solution and 100 μL of Alkali Reagent from the kit. After standing for 15 min at RT, samples were centrifuged at 3100×g for 10 min at RT. 160 μL of supernatant was transferred to a 96-well microplate with addition of 80 μL of chromogenic reagent, followed by oscillation for 2 min and standing at RT for 15 min. The OD value was measured at 550 nm in a microplate reader (FilterMax F5). The concentration of NO was calculated according to the manufacturer's protocol.

### Peritoneal Macrophage Collection

Peritoneal macrophages were collected from the peritoneal cavity of Mye-Tet3 ko mice and WT mice by injecting 10 mL of PBS followed by gentle abdominal massaging for 5 min. Macrophage enrichment was performed by plating cells in RPMI 1640 supplemented with 10% FBS and 1% Anti-Anti for 2 hours at 37 °C in a 5% humidified CO₂ tissue culture incubator. Non-adherent cells were removed with three PBS washes, and cells were harvested in RNA Lysis Buffer of PureLink RNA Mini Kit (Ambion, 12183025) for RNA extraction and qPCR analysis. For cell culture and treatment, cells were cultured in RPMI 1640 supplemented with 10% FBS and 1% Anti-Anti in the presence of 10 ng/mL TGF-β1.

### Blood Chemistry

Blood samples were collected in EDTA tubes (Microtainer with K2EDTA, BD, 365974) by cardiac puncture of terminally anesthetized animals. The tubes were centrifuged at 2,000×g at 4 °C for 20 min, and plasma was collected and stored at −80 °C until use. Blood chemistry were measured according to the manufacturer's instructions. Kits used to measure alanine transaminase (EALT-100) and aspartate transaminase (EASTR-100) were purchased from Bioassay Systems. The bilirubin assay kit (MAK126) was purchased from Sigma Aldrich.

### Flow Cytometry

Total bone marrow cells were harvested from femur bones into the FACS buffer (0.5% FBS in PBS). Cells from spleen were harvested after gently macerating the spleens by a sterile 3 mL syringe plunger in the FACS buffer. Cell suspension was filtered through a 70 μm nylon strainer, centrifuged and resuspended in 5 mL of red blood cell lysis buffer (BioLegend, 420301) to exclude any red blood cells. The cells were resuspended in FACS buffer and stained by antibodies for 15 min in the dark at RT. The antibodies used were CD11b-PE (diluted at 1:100, BioLegend, 101208), NK1.1-APC (diluted at 1:50, BioLegend, 108710), Ly6C-PerCP (diluted at 1:100, BioLegend, 128028) and Ly6G-FITC (diluted at 1:200, BioLegend, 127606). Data were acquired using BD FACSCalibur flow cytometry system and analysis was performed using Flowjo V10.

### Statistical Methods

The number of independent experiments and the statistical analysis for each figure are indicated in the legends. All statistical analyses were performed using GraphPad Prism version 8 for Windows (GraphPad Software, La Jolla California USA, www.graphpad.com) and are presented as mean±SEM. Two-tailed Student's t tests (or as otherwise indicated) were used to compare means between groups. P<0.05 was considered significant.

### Example 6: Breast Cancer Tissues Contained TET3-Overexpressing Cancer-Associated Fibroblasts (CAFs)

Additional studies investigated the levels of TET3 and FAP (fibroblast activation protein), a marker for CAFs, in CAFs. More specifically, IHC protocol was used where FFPE tissue slides were deparaffinized by heating at 65 °C for 60 min, followed by xylene wash 3 times 5 min each. The slides were then rehydrated in 100%, 90%, 80% and 70% ethanol for 5 min each, followed by ddH₂O wash 2 times 5 min each.

For antigen retrieval, slides were heated to near boiling in sodium citrate solution (freshly prepared, pH 6.0) for 2 min, followed by incubation in a steamer (BELLA Food Steamer, Model #XJ-10102A) for 15 min. The slides were cooled at room temperature to below 40 °C, followed by sequential wash in PBS and PBS-T (0.05% Tween 20/PBS) for 5 min each. After permeabilization in 1% Triton X-100/PBS for 15 min, slides were blocked in 5% donkey serum/PBS-T at RT for 1 h and quickly washed once with PBS-T before antibody incubation.

For double-staining of FAP/TET3, slides were incubated with antibodies diluted in 2% donkey serum/PBS-T overnight at 4 °C. Antibodies used are anti-TET3 (GeneTex, GTX121453, diluted at 1:400) and anti-FAP (Invitrogen, BMS168, diluted at 1:400). The next day, slides were washed 3 times and incubated in 0.4% Triton X-100/PBS with the secondary antibody donkey anti-Rabbit IgG Fluor 594 (Invitrogen, A-21207, diluted at 1:500) and goat anti-Mouse lgG Fluor 488 (Invitrogen, A-11029, diluted at 1:500) for 1 h at room temperature. The slides were covered with antifade mounting medium with DAPI (Vector laboratories, H-2000), scoped using a Keyence BZ-X700 fluorescence microscope.

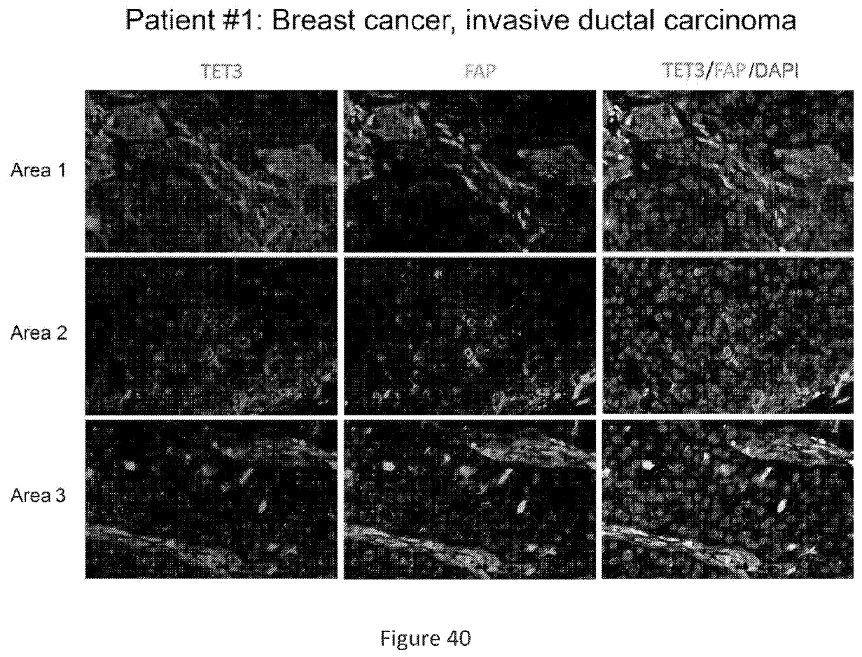

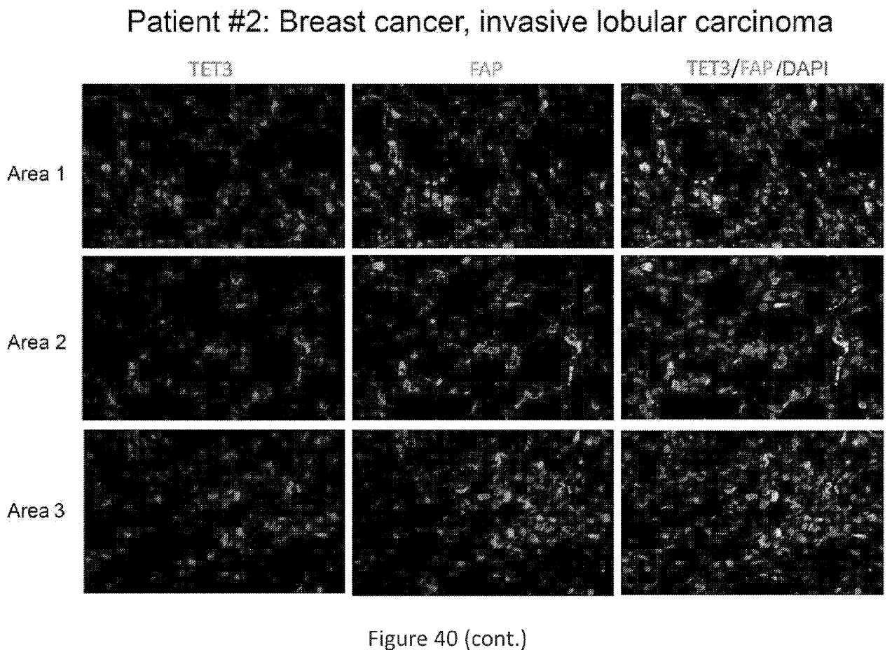

As shown in FIG. 40, TET3/FAP (fibroblast activation protein) double-positive CAF were seen in tissue samples obtained from breast cancer patient.

### Example 7: a Potential New Therapeutic for Ovarian Cancer-Induced Anorexia

Cancer-associated cachexia (CAC) is a clinically challenging syndrome involving anorexia, hypercatabolism, and muscle wasting (Baracos V E et al., 2018, Nat Rev Dis Primers, 4:17105; Petruzzelli M et al., 2016, Genes Dev., 30:489-501). A majority of advanced ovarian cancer patients develop CAC which is a major contributor of morbidity and mortality in these patients (Pin F et al., 2018, J Cachexia Sarcopenia Muscle, 9:685-700; Gadducci A et al., 2001, Anticancer Res., 21:2941-2947). Though CAC and cancer itself are equally deadly, public and professional awareness of CAC remains poor. Various drugs have been evaluated for the treatment of CAC, yet they have demonstrated little or no benefit (Baracos V E et al., 2018, Nat Rev Dis Primers, 4:17105). A growing body of evidence suggests that CAC arises from signals emanating from cancer cells and inflammatory processes that impinge on neural circuits controlling feeding and energy homeostasis. The agouti-related peptide (AGRP)-expressing neurons located in the arcuate nucleus (ARC) of the hypothalamus play a central role in appetite control and energy balance; when activated by energy deficiency (e.g., fasting), these neurons potently drive food intake and decrease resting energy expenditure (Liu T et al., 2012, Front Neurosci., 6:200; Deem J D et al., 2012, FEBS J.). While increased resting energy expenditure is favorable in obesity, it is deleterious in cancer. Notably, a great proportion of patients with ovarian cancer (OC) experience anorexia and elevated resting energy expenditure, suggesting dysfunction of AgRP neurons, a possibility that has not been previously tested and that is tested in the studies described below.

As described above, 1) CRISPR-mediated genetic ablation of TET3, an epigenetic regulator, specifically in AgRP neurons in mice activated these neurons leading to hyperphagia and decreased energy expenditure, 2) Bobcat339, a small molecule compound inhibited TET3 expression in AgRP neurons, 3) Bobcat339 induced hyperphagia and decreases resting energy expenditure in mice when administrated in drinking water or via intraperitoneal injection (i.p.) without showing evidence of liver toxicity, 4) Bobcat339 elicited therapeutic effects in a mouse model of anorexia, and 5) Bobcat339 improved appetite in a mouse model of endometriosis. For this reason, Bobcat339 effectiveness in mitigating OC-induced anorexia and hypermetabolism is tested herein.

CAC can be induced both in immunodeficient and immunocompetent murine models of OC (Pin F et al., 2018, J Cachexia Sarcopenia Muscle, 9:685-700; Carson L F et al., 1998, J Surg Res., 75:97-102; Udumula M P et al., 2021, Mol Metab., 53:101272). OC is immunogenic and the molecular and functional interplay between the patients' immune system and the tumor is important in prognosis (Zhang L et al., 2003, N Engl J Med., 348:203-213; Sato E et al., 2005, Proc Natl Acad Sci USA, 102:18538-18543). A syngeneic (immunocompetent) intraperitoneal ovarian cancer mouse model are also tested (Udumula M P et al., 2021, Mol Metab., 53:101272; Liao J B et al., 2015, J Immunother Cancer, 3:16).

Furthermore, the present studies use the luciferase-expressing mouse ovarian surface epithelial cancer cell line ID8-luc2 (Liao J B et al., 2015, J Immunother Cancer, 3:16) that was originally derived from C57BL/6 mice (Roby K F et al., 2000, Carcinogenesis, 21:585-591). ID8-luc2 allow in vivo imaging of intraperitoneal tumors to monitor tumor growth (Udumula M P et al., 2021, Mol Metab., 53:101272; Liao J B et al., 2015, J Immunother Cancer, 3:16). Agrp-IRES-Cre::LSL-Cas9-GFP mice (C57BL/6 background) that express GFP specifically in AgRP neurons are used, hereafter called AgRP-GFP. 5×10⁶ ID8-luc2 cells in 200 ul of PBS is injected into the peritoneal cavity of AgRP-GFP female mice of 7-8 weeks to establish disseminated abdominal disease similar to that seen in patients with advanced stages of OC. Tumors were readily detected by bioluminescent imaging 10-14 days after inoculation (Udumula M P et al., 2021, Mol Metab., 53:101272; Liao J B et al., 2015, J Immunother Cancer, 3:16). Visible ascites (detected by abdominal swelling) developed at ~3 weeks following implantation (Roby K F et al., 2000, Carcinogenesis, 21:585-591). This is performed once a week. Mice receive an i.p. injection of D-luciferin at 150 mg/kg of body weight 15 min before imaging while under 2% inhaled isoflurane anesthesia. Bioluminescence signals of the tumor are recorded using the Xenogen IVIS system. Food intake is monitored manually on a daily basis and energy expenditure is measured using a computer-controlled indirect calorimetry system.

Initial studies determine whether AgRP neurons are inhibited in OC-bearing mice. Onset of anorexia is defined as two consecutive days of food intake decline with ad libitum food access. OC-bearing (n=10) and sham-treated mice (n=10) are euthanized after a statistical difference is observed between the food intake of OC-bearing mice and sham-treated mice. Mice consume food mainly during the dark-phase and AgRP neurons are activated by food deprivation. AgRP neurons are more active in the afternoon (when caloric deficit is present) than in the morning (Mandelblat-Cerf Y et al., 2015, Elife, 4). Brains from OC-bearing mice and sham-treated mice are isolated in the afternoon, and ARCs are corrected for immunohistochemistry analysis. While FOS is an indirect marker of neuronal activity, AGRP is a specific marker for activated AgRP neurons. Both markers as a readout for AgRP neuronal activity are examined. Fewer FOS-positive AgRP neurons and weaker AgRP signals in the ARC of OC-bearing mice as compared to sham-treated mice are observed indicating that AgRP neurons are inhibited in OC-bearing mice.

Additional studies test whether Bobcat339 alleviates OC-induced anorexia and hypermetabolism. First, studies investigate whether treating mice with Bobcat339 ameliorate established anorexia and hypermetabolism. Thus, OC-bearing mice are randomized into two groups: Control (n=10) and Bobcat339 (n=10). Treatment (i.p. injection of vehicle or Bobcat339) commence one day after the onset of anorexia. The dose of Bobcat339 is 0.05 mg/mouse once a week, because this regimen has been effective in both the anorexia and endometriosis mouse models. Food intake are monitored daily and energy expenditure are determined once a week for three weeks before euthanasia. ARCs are also collected for analysis of FOS and AGRP expression. Second, studies investigate whether Bobcat339 can prevent cancer anorexia and hypermetabolism. To this end, once a week Bobcat339 treatment a day before OC inoculation is started. Food intake are monitored daily and energy expenditure are determined once a week until a statistical difference is seen between the Bobcat339 (n=10) and vehicle (n=10) groups. Mice are sacrificed once statistical differences are observed in both food intake and energy expenditure. Again, ARCs are isolated for FOS and AgRP analysis. Bobcat339 not only prevents cancer anorexia and hypermetabolism from developing but eliminates it after it is established, and that these effects are at least in part via Bc's impact on the AgRP neurons. Finally, with the prevention/elimination of anorexia and hypermetabolism, decreased OC progression by Bobcat339 treatment is also observed.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of treating or preventing a disease or disorder associated with the level of at least one TET protein in a subject in need thereof, wherein the method comprises administering to the subject a therapeutically effective amount of a degrader of TET protein, inhibitor of TET protein, or a combination thereof, or a composition thereof.

2. The method of claim 1, wherein the disease or disorder associated with the level of at least one TET protein is selected from the group consisting of a disease or disorder associated with an increased level of at least one TET, disease or disorder associated with an increased activity of at least one TET, disease or disorder associated with an increased expression of at least one TET, disease or disorder associated with an increased function of at least one TET, and any combination thereof.

3. The method of claim 1, wherein the disease or disorder associated with the level of at least one TET protein is selected from the group consisting of an eating disorder, disease or disorder associated with reduced food intake, anorexia nervosa, cancer-induced anorexia, gynecological disease, endometriosis, anxiety, stress-related disorder, depression, cancer-induced depression, postpartum depression, major depression, depression-related illness, non-alcoholic fatty liver disease (NAFLD), non-alcoholic steatohepatitis (NASH), liver fibrosis, cancer, liver cancer, ovarian cancer, acute myeloid leukemia (AML), pancreatic cancer, glioma, bladder cancer, lung cancer, breast cancer, inflammatory disease or disorder, chronic inflammatory disease or disorder, and any combination thereof.

4. The method of claim 3, wherein the disease or disorder associated with reduced food intake is induced by a treatment of cancer.

5. The method of claim 3, wherein the gynecological disease is an endometriosis.

6. The method of claim 1, wherein the at least one TET protein is selected from the group consisting of TET1 protein, TET2 protein, TET3 protein, and any combination thereof.

7. The method of claim 1, wherein the at least one TET protein is TET3.

8. The method of claim 1, wherein the inhibitor of TET protein is selected from the group consisting of an inhibitor of TET1 protein, inhibitor of TET2 protein, inhibitor of TET3 protein, and any combination thereof.

9. The method of claim 1, wherein the degrader of TET protein is a degrader of TET3 protein.

10. The method of claim 9, wherein the degrader of TET3 protein decreases the level of TET3 protein and simultaneously does not decrease the level of TET2 protein.

11. The method of claim 9, wherein the degrader of TET3 protein is 4-amino-1-[1,1′-biphenyl]-3-yl-5-chloro-2(1H)-pyrimidinone or a derivative, analog, pharmaceutically acceptable salt, hydrate, or prodrug thereof.

12. The method of claim 1, wherein the degrader of TET protein, inhibitor of TET protein, or a combination thereof decreases the activity of at least one TET protein, decreases the level of at least one TET protein, decreases the expression of at least one TET protein, decreases the function of at least one TET protein, decrease the stability of at least one TET protein, increases the degradation of at least one TET protein, or any combination thereof.

13. The method of claim 12, wherein the degrader of TET protein, inhibitor of TET protein, or a combination thereof decreases the activity of at least one TET protein in at least one agouti-related peptide (AgRP) neuron, disease-associated macrophage (DAM), cancer-associated fibroblast (CAF), or any combination thereof, decreases the level of at least one TET protein in at least one AgRP neuron, DAM, CAF, or any combination thereof, decreases the expression of at least one TET protein in at least one AgRP neuron, DAM, CAF, or any combination thereof, decreases the function of at least one TET protein in at least one AgRP neuron, DAM, CAF, or any combination thereof, decrease the stability of at least one TET protein in at least one AgRP neuron, DAM, CAF, or any combination thereof, increases the degradation of at least one TET protein in at least one AgRP neuron, DAM, CAF, or any combination thereof, or any combination thereof.

14. A method of reducing, stopping, or reversing a weight loss in a subject in need thereof, wherein the method comprises administering to the subject a therapeutically effective amount of a degrader of TET protein, inhibitor of TET protein, or a combination thereof or a composition thereof.

15. The method of claim 14, wherein the inhibitor of TET protein is selected from the group consisting of an inhibitor of TET1 protein, inhibitor of TET2 protein, inhibitor of TET3 protein, and any combination thereof.

16. The method of claim 14, wherein the degrader of TET protein is a degrader of TET3 protein.

17. The method of claim 16, wherein the degrader of TET3 protein decreases the level of TET3 protein and simultaneously does not decrease the level of TET2 protein.

18. The method of claim 16, wherein the degrader of TET3 protein is 4-amino-1-[1,1′-biphenyl]-3-yl-5-chloro-2(1H)-pyrimidinone or a derivative, analog, pharmaceutically acceptable salt, hydrate, or prodrug thereof.

19. A method of treating or preventing a disease or disorder associated with the level of at least one disease-associate macrophage (DAM), cancer-associated fibroblast (CAF), or a combination thereof in a subject in need thereof, wherein the method comprises administering to the subject a therapeutically effective amount of a modulator of a DAM, a modulator of a CAF, or a combination thereof.

20. The method of claim 19, wherein the modulator of a DAM induces apoptosis of at least one DAM.

21. The method of claim 19, wherein the modulator of a DAM is 4-amino-1-[1,1′-biphenyl]-3-yl-5-chloro-2(1H)-pyrimidinone or a derivative, analog, pharmaceutically acceptable salt, hydrate, or prodrug thereof.

22. The method of claim 19, wherein the modulator of a DAM further decreases the activity of at least one TET protein, decreases the level of at least one TET protein, decreases the expression of at least one TET protein, decreases the function of at least one TET protein, decrease the stability of at least one TET protein, increases the degradation of at least one TET protein, or any combination thereof.

23. The method of claim 19, wherein the modulator of a CAF induces apoptosis of at least one CAF.

24. The method of claim 19, wherein the modulator of a CAF is 4-amino-1-[1,1′-biphenyl]-3-yl-5-chloro-2(1H)-pyrimidinone or a derivative, analog, pharmaceutically acceptable salt, hydrate, or prodrug thereof.

25. The method of claim 19, wherein the modulator of a CAF further decreases the activity of at least one TET protein, decreases the level of at least one TET protein, decreases the expression of at least one TET protein, decreases the function of at least one TET protein, decrease the stability of at least one TET protein, increases the degradation of at least one TET protein, or any combination thereof.

26. The method of claim 19, wherein the disease or disorder associated with the level of at least one DAM, CAF, or a combination thereof is selected from the group consisting of a disease or disorder associated with increased level of at least one DAM, CAF, or a combination thereof, disease or disorder associated with increased activity of at least one DAM, CAF, or a combination thereof, disease or disorder associated with increased expression of at least one DAM, CAF, or a combination thereof, disease or disorder associated with increased function of at least one DAM, CAF, or a combination thereof, and any combination thereof.

27. The method of claim 19, wherein the disease or disorder associated with the level of at least one DAM, CAF, or a combination thereof is selected from the group consisting of endometriosis, non-alcoholic steatohepatitis (NASH), inflammatory disease or disorder, chronic inflammatory disease or disorder, inflammatory bowel disease (IBD), Alzheimer's disease, Parkinson's disease, cancer, cancer-associated disease or disorder, and any combination thereof.

28. A method of treating or preventing a disease or disorder associated with the level of at least one agouti-related peptide (AgRP) neuron in a subject in need thereof, wherein the method comprises administering to the subject a therapeutically effective amount of a modulator of at least one AgRP neuron.

29. The method of claim 28, wherein the modulator of at least one AgRP neuron increases the level of at least one AGRP peptide, neuropeptide Y (NPY) peptide, vesicular GABA transporter (VGAT) peptide, or a combination thereof, increases the activity of at least one AGRP peptide, NPY peptide, VGAT, or a combination thereof, increases the function of at least one AGRP peptide, NPY peptide, VGAT, or a combination thereof, or increases the expression of at least one AGRP peptide, NPY peptide, VGAT, or a combination thereof.

30. The method of claim 28, wherein the modulator of at least one AgRP neuron is 4-amino-1-[1,1′-biphenyl]-3-yl-5-chloro-2(1H)-pyrimidinone or a derivative, analog, pharmaceutically acceptable salt, hydrate, or prodrug thereof.

31. The method of claim 28, wherein the modulator of at least one AgRP neuron further decreases the activity of at least one TET protein, decreases the level of at least one TET protein, decreases the expression of at least one TET protein, decreases the function of at least one TET protein, decrease the stability of at least one TET protein, increases the degradation of at least one TET protein, or any combination thereof.

32. The method of claim 28, wherein the disease or disorder associated with the level of at least one AgRP neuron is selected from the group consisting of a disease or disorder associated with decreased level of at least one AgRP neuron, disease or disorder associated with decreased activity of at least one AgRP neuron, disease or disorder associated with decreased expression of at least one AgRP neuron, disease or disorder associated with decreased function of at least one AgRP neuron, and any combination thereof.

33. The method of claim 28, wherein the disease or disorder associated with the level of at least one AgRP neuron is selected from the group consisting of eating disorder, mood disorder, cancer-associated disease or disorder, cachexia, cancer-associated cachexia, depression, anxiety, hypophagia, and any combination thereof.

34. A method of modulating at least one pathway involved in transforming growth factor beta (TGF-β) signaling, metabolic reprogramming, apoptosis, or any combination thereof in a subject in need thereof, wherein the method comprises administering to the subject a therapeutically effective amount of a degrader of TET protein, inhibitor of TET protein, or a combination thereof or a composition thereof.

35. The method of claim 34, wherein the method comprises
a) inhibiting at least one pathway involved in TGF-β signaling;
b) inhibiting at least one pathway involved in metabolic reprogramming;
c) activating at least one pathway involved in apoptosis;
d) or any combination thereof.

36. The method of claim 34, wherein the inhibitor of TET protein is selected from the group consisting of an inhibitor of TET1 protein, inhibitor of TET2 protein, inhibitor of TET3 protein, and any combination thereof.

37. The method of claim 34, wherein the degrader of TET protein is a degrader of TET3 protein.

38. The method of claim 37, wherein the degrader of TET3 protein decreases the level of TET3 protein and simultaneously does not decrease the level of TET2 protein.

39. The method of claim 37, wherein the degrader of TET3 protein is 4-amino-1-[1,1′-biphenyl]-3-yl-5-chloro-2(1H)-pyrimidinone or a derivative, analog, pharmaceutically acceptable salt, hydrate, or prodrug thereof.

40. The method of claim 34, wherein the degrader of TET protein, inhibitor of TET protein, or a combination thereof decreases the activity of at least one TET protein, decreases the level of at least one TET protein, decreases the expression of at least one TET protein, decreases the function of at least one TET protein, decrease the stability of at least one TET protein, increases the degradation of at least one TET protein, or any combination thereof.

41. The method of claim 40, wherein the degrader of TET protein, inhibitor of TET protein, or a combination thereof decreases the activity of at least one TET protein in at least one agouti-related peptide (AgRP) neuron, disease-associated macrophage (DAM), cancer-associated fibroblast (CAF), or any combination thereof, decreases the level of at least one TET protein in at least one AgRP neuron, DAM, CAF, or any combination thereof, decreases the expression of at least one TET protein in at least one AgRP neuron, DAM, CAF, or any combination thereof, decreases the function of at least one TET protein in at least one AgRP neuron, DAM, CAF, or any combination thereof, decrease the stability of at least one TET protein in at least one AgRP neuron, DAM, CAF, or any combination thereof, increases the degradation of at least one TET protein in at least one AgRP neuron, DAM, CAF, or any combination thereof, or any combination thereof.

42. A method of reducing or inhibiting the level or activity of at least one TET protein, transforming growth factor beta (TGF-β), interleukin-1 beta (IL-1β), interleukin 6 (IL-6), or any combination thereof in a subject in need thereof, wherein the method comprises administering to the subject a therapeutically effective amount of 4-amino-1-[1,1′-biphenyl]-3-yl-5-chloro-2(1H)-pyrimidinone or a derivative, analog, pharmaceutically acceptable salt, hydrate, or prodrug thereof.

43. The method of claim 42, wherein the method comprises reducing or inhibiting the level or activity of TET3 protein, TGF-β, IL-1β, IL-6, or any combination thereof.

44. The method of claim 42, wherein the method comprises reducing or inhibiting the level or activity of TET3 protein, TGF-β, IL-1β, IL-6, or any combination thereof and simultaneously not effecting the level or activity of TET2 protein.

45. The method of claim 1, wherein the degrader of TET protein, inhibitor of TET protein, or a combination thereof further increases the level of at least one AGRP peptide, NPY peptide, VGAT, or a combination thereof, increases the activity of at least one AGRP peptide, NPY peptide, VGAT, or a combination thereof, increases the function of at least one AGRP peptide, NPY peptide, VGAT, or a combination thereof, or increases the expression of at least one AGRP peptide, NPY peptide, VGAT, or a combination thereof.
